(19) United States
(12) Patent Application Publication
(10) Pub. No.: US 2002/0193377 A1
(43) Pub. Date: Dec. 19, 2002

(54) Quinazolines as MMP-13 inhibitors

(76) Inventors: Charles Andrianjara, Fresnes (FR); Nicole Chantel Barvian, Ann Arbor, MI (US); Bernard Gaudilliere, Nanterre (FR); Henri Jacobelli, Paray Vieille Poste (FR); Daniel Fred Ortwine, Saline, MI (US); William Chester Patt, Chelsea, MI (US); Ly Pham, Grand Rapids, MI (US); Catherine Rose Kostlan, Saline, MI (US); Michael William Wilson, Ann Arbor, MI (US)

Correspondence Address: Claude F. Purchase, Jr., Warner-Lambert Company, 2800 Plymouth Road, Ann Arbor, MI, 48105, US

(21) Appl. No.: 10/075,954

(22) Filed: Feb. 13, 2002

Related U.S. Application Data
(60) Provisional application No. 60/268,661, filed on Feb. 14, 2001.

(51) Int. Cl. C07D 487/02; A61K 31/519; A61K 31/517; A61K 31/525; C07D 239/72

(52) U.S. Cl. 514/248; 514/251; 514/262.1; 514/264.1; 544/236; 544/256; 544/257; 544/279; 544/285; 514/266.3

(57) ABSTRACT

A compound selected from those of formula (I):

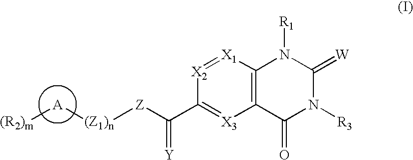

in which:

R₁ represents a group selected from hydrogen, amino, alkyl, alkenyl, aminoalkyl, aryl, arylalkyl, heterocycle, and cycloalkylalkyl, optionally substituted,

W represents oxygen, sulfhur, or ═N—R′, in which R′ is as defined in the description,

X₁, X₂ and X₃ represent nitrogen or —C—R₆ in which R₆ is as defined in the description,

Y represents oxygen, sulfhur, —NH, or —N(C₁-C₆)alkyl,

Z represents oxygen, sulfhur, —NR₇ in which R₇ is as defined in the description, and 59 optionally carbon atom,

n is an integer from 1 to 8 inclusive,

Z₁ represents —CR₈R₉ wherein R₈ and R₉ are as defined in the description,

A represents aromatic or non-aromatic, heterocyclic or non-heterocyclic ring system,

m is an integer from 0 to 7 inclusive, the group(s) R₂ is (are) is as defined in the description,

R₃ represents hydrogen, alkyl, alkenyl, alkynyl, ot a group of formula:

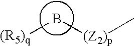

in which Z₂, B, R₅, P and q are as defined in the description,

optionally, the racemic forms thereof, isomers thereof, N-oxydes thereof, and the pharmaceutically acceptable salts thereof, and medicinal products containing the same are useful as specific inhibitors of type-13 matrix metalloprotease.

### FIELD OF THE INVENTION

The present invention relates to novel substituted quinazolines which are useful for preparing medicinal products for treating complaints involving a therapy with a matrix metalloprotease-13 (MMP-13) inhibitor. These medicinal products are useful in particular for treating certain inflammatory conditions such as rheumatoid arthritis or osteoarthritis, as well as certain proliferative conditions such as cancers.

### TECHNOLOGICAL BACKGROUND OF THE INVENTION

Matrix metalloproteases (MMPs) are enzymes which are involved in the renewal of extracellular matrix tissue, such as cartilage, tendons and joints. MMPs bring about the destruction of the extracellular matrix tissue, which is compensated for, in a non-pathological physiological state, by its simultaneous regeneration.

Under normal physiological conditions, the activity of these extremely aggressive peptidases is controlled by specialized proteins which inhibit MMPs, such as the tissue inhibitors of metalloprotease (TIMPs).

Local equilibrium of the activities of MMPs and of TIMPs is critical for the renewal of the extracellular matrix. Modifications of this equilibrium which result in an excess of active MMPs, relative to their inhibitor, induce a pathological destruction of cartilage, which is observed in particular in rheumatoid arthritis and in osteoarthritis.

In pathological situations, an irreversible degradation of articular cartilage takes place, as is the case in rheumatic diseases such as rheumatoid arthritis or osteoarthritis. In these pathologies, the cartilage degradation process predominates, leading to a destruction of the tissue and resulting in a loss of function.

At least twenty different matrix metalloproteases have been identified to date and are subdivided into four groups, the collagenases, the gelatinases, the stromelysins and the membrane-type MMPs (MT-MMPs), respectively.

Matrix metalloprotease-13 (MMP-13) is a collagenase-type MMP which constitutes the predominant collagenase observed during osteoarthritis, in the course of which pathology the chondrocyte directs the destruction of cartilage.

There is a need in the prior art for novel MMP inhibitors, more particularly for MMP-13 inhibitors, in order to prevent and/or correct the imbalance in the renewal of extracellular matrix tissue, such as arthritis, rheumatoid arthritis, osteoarthritis, osteoporosis, periodontal diseases, inflammatory bowel disease, psoriasis, multiple sclerosis, cardiac insufficiency, atherosclerosis, asthma, chronic obstructive pulmonary diseases (COPD), age-related macular degeneration (ARMD) and cancer.

MMP-inhibitor compounds are known. Most of these MMP-inhibitors are not selective for a single MMP, such as those described by Montana and Baxter (2000) or by Clark et al. (2000).

There is also a need in the prior art for novel inhibitors that are active on matrix metalloprotease-13, in order to enrich the therapeutic arsenal that can be used for treating pathologies associated with the destruction of the extracellular matrix and with cancer.

### SUMMARY OF THE INVENTION

The invention relates to a substituted quinazoline of formula (I):

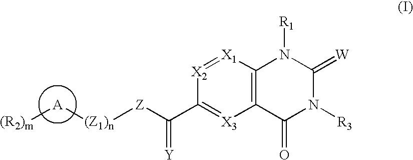

in which:

R₁ represents a group selected from:

hydrogen, amino,

(C₁-C₆)alkyl, (C₃-C₆)alkenyl, (C₃-C₆)alkynyl, mono(C₁-C₆)alkylamino(C₁-C₆)alkyl, di(C₁-C₆)alkylamino(C₁-C₆)alkyl, aryl, aryl(C₁-C₆)alkyl, heterocycle, and 3- to 6-membered cycloalkyl(C₁-C₆)alkyl, these groups being unsubstituted or substituted with one or more groups, which may be identical or different, selected from amino, (C₁-C₆)alkyl, cyano, halo(C₁-C₆)alkyl, C(═O)OR₄, OR₄ and SR₄, in which R₄ represents hydrogen or (C₁-C₆)alkyl,

W represents an oxygen atom, a sulphur atom, or a group ═N—R′, in which R′ represents (C₁-C₆)alkyl, hydroxyl, or cyano,

X₁, X₂ and X₃ represent, independently of each other, a nitrogen atom or a group —C—R₆ in which R₆ represents a group selected from hydrogen, (C₁-C₆)alkyl, amino, mono(C₁-C₆)alkylamino, di(C₁-C₆)alkylamino, hydroxyl, (C₁-C₆)alkoxy, and halogen, with the proviso that not more than two of the groups X₁, X₂ and X₃ simultaneously represent a nitrogen atom,

Y represents a group selected from oxygen atom, sulphur atom, —NH, and —N(C₁-C₆)alkyl,

Z represents:

an oxygen atom, a sulphur atom,

or a group —NR₇ in which R₇ represents a group selected from hydrogen, (C₁-C₆)alkyl, aryl(C₁-C₆)alkyl, cycloalkyl, aryl, and heteroaryl, and

when Y is an oxygen atom, a sulphur atom, or a group —N(C₁-C₆)alkyl, Z optionally represents a carbon atom which is unsubstituted or substituted with a (C₁-C₆)alkyl, an aryl, an aryl(C₁-C₆)alkyl, an aromatic or non-aromatic heterocycle or a cycloalkyl,

n is an integer from 1 to 8 inclusive,

Z₁, represents —CR₈R₉ wherein R₈ and R₉, independently of each other, represent a group selected from hydrogen, (C₁-C₆)alkyl, halo(C₁-C₆)alkyl, halogen, amino, OR₄, SR₄ or C(═O)OR₄ in which R₄ represents a hydrogen or (C₁-C₆)alkyl, and

when n is greater than or equal to 2, the hydrocarbon chain Z₁ optionally contains one or more multiple bonds,

and/or one of the carbon atoms in the hydrocarbon chain Z₁ may be replaced with an oxygen atom, a sulphur atom which is unsubstituted or substituted with one or two oxygen atoms, or a nitrogen atom which is unsubstituted or substituted with a (C₁-C₆)alkyl,

and when one of the carbon atoms in the hydrocarbon chain Z₁ is replaced with a sulphur atom which is unsubstituted or substituted with one or two oxygen atoms, then the group —C(═Y)—Z— optionally may be absent in the general formula (I),

A represents a group selected from:

aromatic or non-aromatic, 5- or 6-membered monocycle comprising from 0 to 4 heteroatoms selected from nitrogen, oxygen and sulphur, and

bicycle, composed of two aromatic or non-aromatic, 5- or 6-membered rings, which may be identical or different, comprising from 0 to 4 heteroatoms selected from nitrogen, oxygen and sulphur,

m is an integer from 0 to 7 inclusive,

the group(s) R₂, which may be identical or different, is (are) selected from (C₁-C₆)alkyl, halogen, —CN, NO₂, SCF₃, —CF₃, —OCF₃, —NR₁₀R₁₁, —OR₁₀, —SR₁₀, —SOR₁₀, —SO₂R₁₀, —(CH₂)ₖSO₂NR₁₀R₁₁, —X₅(CH₂)ₖC(═O)OR₁₀, —(CH₂)ₖC(═O)OR₁₀, —X₅(CH₂)ₖC(═O)NR₁₀R₁₁, —(CH₂)ₖC(═O)NR₁₀R₁₁, and —X₄—R₁₂ in which:

X₅ represents a group selected from oxygen, sulphur optionally substituted by one or two oxygen atoms, and nitrogen substituted by hydrogen or (C₁-C₆)alkyl,

k is an integer from 0 to 3 inclusive,

R₁₀ and R₁₁, which may be identical or different, are selected from hydrogen and (C₁-C₆)alkyl,

X₄ represents a group selected from single bond, —CH₂—, oxygen atom, sulphur atom optionally substituted by one or two oxygen atoms, and nitrogen atom substituted by hydrogen atom or (C₁-C₆)alkyl group,

R₁₂ represents an aromatic or non-aromatic, heterocyclic or non-heterocyclic, 5- or 6-membered ring which is unsubstituted or substituted with one or more groups, which may be identical or different, selected from (C₁-C₆)alkyl, halogen, hydroxyl and amino, and when the ring is heterocyclic, it comprises from 1 to 4 heteroatoms selected from nitrogen, oxygen and sulphur;

R₃ represents a group selected from:

hydrogen,

(C₁-C₆)alkyl, (C₃-C₆)alkenyl, (C₃-C₆)alkynyl, these groups being unsubstituted or substituted with one or more groups, which may be identical or different, selected from amino, cyano, halo(C₁-C₆)alkyl, cycloalkyl, —C(═O)NR₁₀R₁₁, —C(═O)OR₁₀, OR₁₁, and SR₁₀, in which R₁₀ and R₁₁, which may be identical or different, represent hydrogen or (C₁-C₆)alkyl,

and the group of formula:

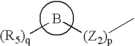

in which p is an integer from 0 to 8 inclusive,

Z₂ represents —CR₁₃R₁₄ wherein R₁₃ and R₁₄, independently of each other, represent a group selected from hydrogen, (C₁-C₆)alkyl, phenyl, halo(C₁-C₆)alkyl, halogen, amino, OR₄, SR₄ and —C(═O)OR₄ in which R₄ represents hydrogen or (C₁-C₆)alkyl, and

when p is greater than or equal to 2, the hydrocarbon chain Z₂ optionally contains one or more multiple bonds,

and/or one of the carbon atoms in the hydrocarbon chain Z₂ may be replaced with an oxygen atom, a sulphur atom which is unsubstituted or substituted with one or two oxygen atoms, a nitrogen atom which is unsubstituted or substituted with a (C₁-C₆)alkyl, or a carbonyl group,

B represents a group selected from:

an aromatic or non-aromatic 5- or 6-membered monocycle comprising from 0 to 4 heteroatoms selected from nitrogen, oxygen and sulphur, and

a bicycle, composed of two aromatic or non-aromatic, 5- or 6-membered rings, which may be identical or different, comprising from 0 to 4 heteroatoms selected from nitrogen, oxygen and sulphur,

q is an integer from 0 to 7 inclusive,

the group(s) R₅, which may be identical or different, is (are) selected from (C₁-C₆)alkyl, halogen, CN, NO₂, CF₃, OCF₃, —(CH₂)ₖNR₁₅R₁₆, —N(R₁₅)C(═O)R₁₆, —N(R₁₅)C(═O)OR₁₆, —N(R₁₅)SO₂R₁₆, —N(SO₂R₁₅)₂, —OR₁₅, —S(O)ₖ₁R₁₅, SO₂—N(R₁₅)—(CH₂)ₖ₂—NR₁₆R₁₇, —(CH₂)ₖSO₂NR₁₅R₁₆, —X₇(CH₂)ₖC(═O)OR₁₅, (CH₂)ₖC(═O)OR₁₅, —C(═O)O—(CH₂)ₖ₂—NR₁₅R₁₆, —C(═O)O—(CH₂)ₖ₂—C(═O)OR₁₈, —X₇(CH₂)ₖC(═O)NR₁₅R₁₆, —(CH₂)ₖC(═O)NR₁₅R₁₆, —R₁₉—C(═O)OR₁₅, —X₆—R₂₀, and —C(═O)—R₂₁—NR₁₅R₁₆ in which:

X₇ represents a group selected from oxygen atom, sulphur atom optionally substituted by one or two oxygen atoms, and nitrogen atom substituted by a hydrogen atom or a (C₁-C₆)alkyl group,

k is an integer from 0 to 3 inclusive,

k1 is an integer from 0 to 2 inclusive,

k2 is an integer from 1 to 4 inclusive,

R₁₅, R₁₆ and R₁₇, which may be identical or different, are selected from hydrogen and (C₁-C₆)alkyl,

R₁8 represents a group selected from (C₁-C₆)alkyl, —R₂₁—NR₁₅R₁₆, R₂₁—NR₁₅—C(═O)—R₂₁—NR₁₆R₁₇, and —C(═O)O—R₂₁—NR₁₅R₁₆ in which R₂₁ represents a linear or branched (C₁-C₆)alkylene group, and R₁₅, R₁₆ and R₁₇ are as defined hereinbefore,

R₁₉ represents a (C₃-C₆)cycloalkyl group,

X₆ represents a group selected from single bond, —CH₂—, oxygen atom, sulphur atom optionally substituted by one or two oxygen atoms, and nitrogen atom substituted by hydrogen atom or (C₁-C₆)alkyl group,

R₂₀ represents an aromatic or non-aromatic, heterocyclic or non-heterocyclic, 5- or 6-membered ring, which is unsubstituted or substituted with one or more groups, which may be identical or different, selected from (C₁-C₆)alkyl, halogen, hydroxyl, oxo, cyano, tetrazole, amino, and —C(═O)OR₄ wherein R₄ represents hydrogen or (C₁-C6)alkyl, and, when the ring is heterocyclic, it comprises from 1 to 4 heteroatoms selected from nitrogen, oxygen and sulphur,

with the proviso that when X₁ represents a nitrogen atom, X₂ cannot represent a carbon atom substituted with a methyl group or with NH—CH₃, optionally, the racemic forms thereof, isomers thereof, N-oxydes thereof, and the pharmaceutically acceptable salts thereof.

The compounds of the present invention are useful as inhibitors, in particular as selective inhibitors, of the enzyme matrix metalloprotease-13 (MMP-13).

The invention also relates to compounds used mainly as intermediates for the synthesis of the compounds of formula (I). These intermediate compounds have the general formula (III) below:

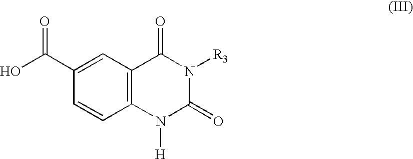

in which R₃ has the same meaning as defined for the compound of formula (I).

The invention also relates to compounds used mainly as intermediates for the synthesis of the compound of formula (I), which have the general formula (IV) below:

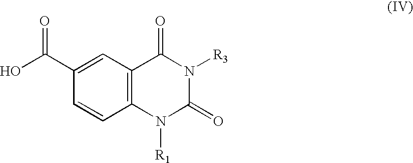

in which R₁ et R₃ have the same meaning as for a compound of formula (I).

The invention also relates to a process for manufacturing the compound of formula (I) in which:

R₂, R₃, Z₁, A, n and m are as defined in the compound of general formula (I),

X₁, X₂, X₃ are each a group —C—R₆ in which R₆ represents a hydrogen atom,

Y is O,

Z is —N—R₇ in which R₇ is as defined in the compound of general formula (I),

and W is O.

This process is characterized in that it comprises the reaction of a compound of formula (II):

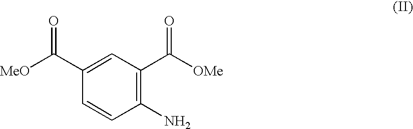

with pyridine and the compound of general formula (V):

O═C═N—R₃ (V)

in which R₃ is as defined above for the compound of formula (I), to give the compound of general formula (VI):

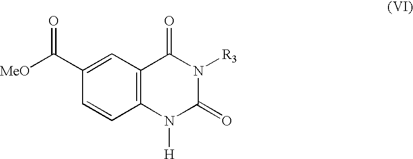

in which R₃ is as defined hereinbefore,

followed by reacting the compound of general formula (VI) in the presence of LiOH to give the compound of general formula (III) in which R₃ is as defined above.

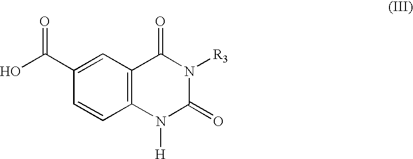

In a subsequent step of the synthetic process, the compound of general formula (III) obtained above is reacted, in the presence of an acid activator such as O-[(ethoxycarbonyl)cyanomethylenamino]-N,N,N′,N′-tetramethyluronium tetrafluoroborate (TOTU) with the compound of general formula (VII):

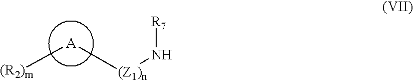

in which R₇ is selected from hydrogen, (C₁-C₆)alkyl, aryl(C₁-C₆)alkyl, cycloalkyl, aryl and heteroaryl, and A, R₂, Z₁, n and m are as defined above for the compound of formula (I), to give the compound of general formula (I) in which R₁, represents hydrogen, X₁, X₂ and X₃ are each —C—R₆ in which R₆ represents hydrogen atom, Y is O, Z is N—R₇, W is O, and A, R₂, Z₁, n and m are as defined hereinbefore.

In particular, when W is O, Y is O and Z is O, the compounds of formula (I) corresponding to this definition may be obtained by reacting a compound of general formula (III):

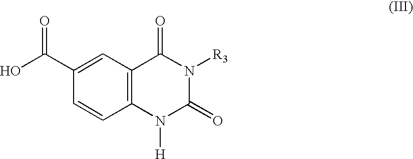

in which R₃ is as defined in the compound of general formula (I),

with a compound of general formula (XVI):

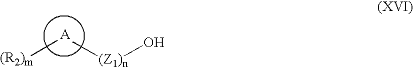

in which Z₁, A, R₂, n and m are as defined in the compound of general formula (I), to give a compound of general formula (XVII):

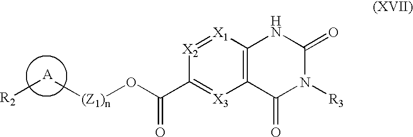

in which A, R₂, R₃, Z₁ m and n are as defined for the compound of general formula (I), and X₁, X₂, and X₃ are each —C—R₆ in which R₆ represents hydrogen atom,

followed by reacting the compound of formula (XVII), in presence of a base, with the compound of general formula (VIII), X—R₁, in which R₁ is as defined for the compound of formula (I) and X is a leaving group such as halogen,

to give the compound of general formula (I) in which X₁, X₂ and X₃ are each —C—R₆ in which R₆ is as defined hereinbefore, W is O Y is O Z is O and R₁, R₂, R₃, Z₁, A, n and m are as defined hereinbefore.

In particular, when X₂ and X₃ are each —C—R₆ in which R₆ represents hydrogen atom, X₁ is N, Z is O and Y is O, the compounds of formula (I) corresponding to this definition may be obtained by reacting a compound of general formula (XIX):

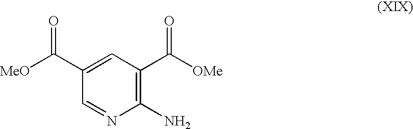

with pyridine and a compound of general formula O═C═N—R₃ (V) in which R₃ is as defined in the compound of formula (I),

to give a compound of general formula (XX):

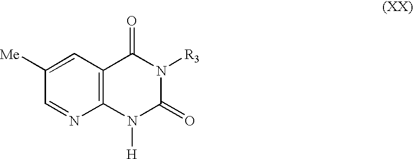

in which R₃ is as defined hereinbefore,

followed by reacting the compound of general formula (XX) in the presence of KMnO₄ to give the compound of general formula (XXI):

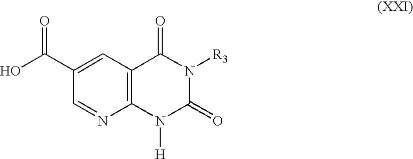

in which R₃ is as defined hereinbefore,

followed by reacting a compound of general formula (XXI) in the presence of SOCl₂ and CHCl₃ to give the compound of general formula (XXII):

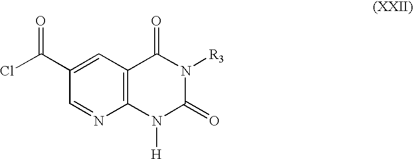

in which R₃ is as defined hereinbefore,

followed by reacting the compound of formula (XXII) with the compound of general formula (XVI):

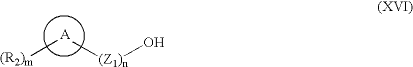

in which A, R₂, Z₁, n and m are as defined in the compound of formula (I),

to give the compound of general formula (I):

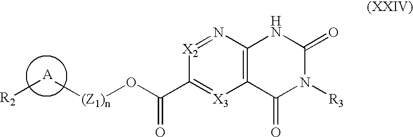

in which A, R₂, R₃, Z₁ m and n are as defined hereinbefore, X₂ and X₃ are each —C—R₆ in which R₆ is as defined hereinbefore, and R₃ are as defined for the compound of general formula (l).

The invention also relates to a pharmaceutical composition comprising a compound of formula (I) and a pharmaceutically acceptable excipient.

The invention also relates to the use of a compound of formula (I) for the preparation of a medicinal product intended for treating a disease or complaint involving therapy by inhibition of matrix metalloprotease, and more particularly of type-13 matrix metalloprotease (MMP-13).

The invention also relates to a method for treating a disease or complaint involving a therapy by inhibition of matrix metalloprotease, and more particularly MMP-13, the said method comprising the administration of an effective amount of a compound of formula (I) to a patient.

### DETAILED DESCRIPTION OF THE INVENTION

The Applicant has identified according to the invention novel compounds that are matrix metalloprotease inhibitors, and more specifically novel compounds that are MMP-13 inhibitors.

One subject of the invention is thus a substituted quinazoline of formula (I):

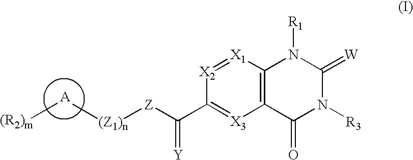

in which R₁, R₂, R₃, X₁, X₂, X₃, W, Y, Z, Z₁, n and m are as defined hereinbefore in the compound of general formula (I),

optionally the racemic forms thereof, isomers forms thereof, N-oxydes thereof, and the pharmaceutically acceptable salts thereof.

The invention relates particularly to the compounds of general formula (I) in which:

R₁ represents hydrogen, (C₁-C₆)alkyl, aryl(C₁-C₆)alkyl or 3- to 6-membered cycloalkyl(C₁-C₆)alkyl,

W represents an oxygen atom or a sulphur atom,

X₁ represents a nitrogen atom or —C—R₆ in which R₆ represents a hydrogen atom,

X₂ and X₃ represent each —C—R₆ in which R₆ represents a hydrogen atom,

Y represents an oxygen atom,

Z represents an oxygen atom or —NR₇ in which R₇ represents a hydrogen atom. The invention also relates to the compounds of general formula (I) in which:

n is an integer from 1 to 6 inclusive,

Z, represents —CR₈R₉ wherein R₈ represents a hydrogen atom and R₉ represents a hydrogen atom or a methyl group, and

when n is greater than or equal to 2, the hydrocarbon chain Z₁ optionally contains a double bond,

or, one of the carbon atoms in the hydrocarbon chain Z₁ may be replaced with an oxygen atom, or a sulphur atom which is unsubstituted or substituted with one or two oxygens,

A represents a group selected from phenyl, pyridyl, thienyl, imidazolyl, furyl, piperidyl, 1,3-benzodioxolyl, benzodioxinyl, benzothienyl, benzofuryl, benzofurazanyl, 2,1,3-benzothiadiazolyl, and indolyl,

m is an integer from 0 to 7 inclusive,

the group(s) R₂, which may be identical or different, is (are) selected from (C₁-C₆)alkyl, halogen, —CN, —CF₃, —OCF₃, —NR₁₀R₁₁, —OR₁₀, —SR₁₀, —SO₂R₁₀, —(CH₂)ₖSO₂NR₁₀R₁₁, —X₅(CH₂)ₖC(═O)OR₁₀, —(CH₂)ₖC(═O)OR₁₀, —X₅(CH₂)ₖC(═O)NR₁₀R₁₁, —(CH₂)ₖC(═O)NR₁₀R₁₁, and —X₄—R₁₂ in which:

X₅ represents O, S or NH,

k is an integer from 0 to 3 inclusive,

R₁₀ and R₁₁, identical or different, are selected from hydrogen and (C₁-C₆)alkyl,

X₄ represents —CH₂—, or an oxygen atom,

R₁₂ represents a phenyl group which is unsubstituted or substituted with one or more groups, which may be identical or different, selected from (C₁-C₆)alkyl, halogen, hydroxyl and amino.

The invention also relates to the compounds of general formula (I) in which R₃ represents hydrogen, (C₁-C₆)alkyl or the group of formula:

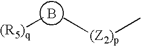

in which p is an integer from 0 to 3 inclusive,

Z₂ represents —CR₁₃R₁₄ wherein R₁₃ and R₁₄, independently of each other, represent a group selected from hydrogen, methyl, or phenyl, and

when p is greater than or equal to 2, the hydrocarbon chain Z₂ optionally contains one double bond,

or one of the carbon atoms in the hydrocarbon chain Z₂ may be replaced with an oxygen atom, a sulphur atom which is unsubstituted or substituted with one or two oxygen atoms, a nitrogen atom which is unsubstituted or substituted with a (C₁-C₆)alkyl, or a carbonyl group,

B represents a group selected from phenyl, pyridyl, thienyl, imidazolyl, furyl, 1,3-benzodioxolyl, benzodioxinyl, benzothienyl, benzofuryl, 2,1,3-benzothiadiazolyl, benzofurazanyl, naphthyl and indolyl,

q is an integer from 0 to 3 inclusive,

the group(s) R₅, which may be identical or different, is (are) selected from (C₁-C₆)alkyl, halogen, CN, NO₂, CF₃, OCF₃, —(CH₂)ₖNR₁₅R₁₆, —N(R₁₅)C(═O)R₁₆, —N(R₁₅)C(═O)OR₁₆, —N(R₁₅)SO₂R₁₆, —N(SO₂R₁₅)₂, —OR₁₅, —S(O)ₖ₁R₁₅, —SO₂—N(R₁₅)—(CH₂)ₖ₂—NR₁₆R₁₇, —(CH₂)ₖSO₂NR₁₅R₁₆, —X₇(CH₂)ₖC(═O)OR₁₅, —(CH₂)ₖC(═O)OR₁₅, —C(═O)O—(CH₂)ₖ₂—NR₁₅R₁₆, —X₇(CH₂)ₖC(═O)NR₁₅R₁₆, and —(CH₂)ₖC(═O)NR₁₅R₁₆ in which:

X₇is S, O or NH,

k is an integer from 0 to 3 inclusive,

k1 is an integer from 0 to 2 inclusive,

k2 is an integer from 1 to 4 inclusive,

R₁₅, R₁₆ and R₁₇, identical or different, are selected from hydrogen and (C₁-C₆)alkyl,

The invention relates more particularly to the compounds of general formula (I) in which:

R₁ represents a group selected from:

hydrogen, amino,

(C₁-C₆)alkyl, (C₃-C₆)alkenyl, (C₃-C₆)alkynyl, mono(C₁-C₆)alkylamino(C₁-C₆)alkyl, di(C₁-C₆)alkylamino(C₁-C₆)alkyl, aryl, aryl(C₁-C₆)alkyl, heterocycle, and 3- to 6-membered cycloalkyl(C₁-C6)alkyl, these groups being unsubstituted or substituted with one or more groups, which may be identical or different, selected from amino, (C₁-C₆)alkyl, cyano, halo(C₁-C₆)alkyl, C(═O)OR₄, OR₄ and SR₄, in which R₄ represents hydrogen or (C₁-C₆)alkyl,

W represents an oxygen atom, a sulphur atom, or a group ═N—R′, in which R′ represents (C₁-C₆)alkyl, hydroxyl, or cyano,

X₁ represents a nitrogen atom or a group —C—R₆ in which R₆ represents hydrogen atom, X₂ and X₃ represent, independently of each other, a group —C—R₆ in which R₆ represents a group selected from hydrogen, (C₁-C₆)alkyl, amino, hydroxyl and halogen,

Y represents an oxygen atom,

Z represents an oxygen atom, or a group —NR₇ in which R₇ represents a group selected from hydrogen, and (C₁-C₆)alkyl,

n is an integer from 1 to 6 inclusive,

Z₁, represents —CR₈R₉ wherein R₈ and R₉, independently of each other, represent a group selected from hydrogen, (C₁-C₆)alkyl and hydroxyl, and

when n is greater than or equal to 2, the hydrocarbon chain Z₁ optionally contains one or more multiple bonds,

or one of the carbon atoms in the hydrocarbon chain Z₁ may be replaced with an oxygen atom, a sulphur atom which is unsubstituted or substituted with one or two oxygen atoms, or a nitrogen atom which is unsubstituted or substituted with a (C₁-C₆)alkyl,

A represents a group selected from phenyl, pyridyl, thienyl, imidazolyl, furyl, 1,3-benzodioxolyl, benzodioxinyl, benzothienyl, benzofuryl, benzofurazanyl, 2,1,3-benzothiadiazolyl, and indolyl,

m is an integer from 0 to 3 inclusive, the group(s) R₂, which may be identical or different, is (are) selected from (C₁-C₆)alkyl, halogen, —CN, —CF₃, —OCF₃, —NR₁₀R₁₁, —OR₁₀, —SR₁₀, —SO₂R₁₀, —(CH₂)ₖSO₂NR₁₀R₁₁, —X5(CH₂)ₖC(═O)OR₁₀, —(CH₂)ₖC(═O)OR₁₀, —X₅(CH₂)ₖC(═O)NR₁₀R₁₁, —(CH₂)ₖC(═O)NR₁₀R₁₁, and —X₄—R₁₂ in which:

X₅ represents O, S or NH,

k is an integer from 0 to 3 inclusive,

R₁₀ and R₁₁, which may be identical or different, are selected from hydrogen and (C₁-C₆)alkyl,

X₄ represents —CH₂—, or an oxygen atom,

R₁₂ represents phenyl which is unsubstituted or substituted with one or more groups,

which may be identical or different, selected from (C₁-C₆)alkyl, halogen, and hydroxyl,

R₃ represents a group selected from hydrogen, (C₁-C₆)alkyl, and the group of formula:

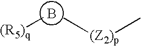

in which p is an integer from 0 to 6 inclusive,

Z₂ represents —CR₁₃R₁₄ wherein R₁₃ and R₁₄, independently of each other, represent a group selected from hydrogen, (C₁-C₆)alkyl, and hydroxy, and

when p is greater than or equal to 2, the hydrocarbon chain Z₂ optionally contains one or more multiple bonds,

or one of the carbon atoms in the hydrocarbon chain Z₂ may be replaced with an oxygen atom, a sulphur atom which is unsubstituted or substituted with one or two oxygen atoms, a nitrogen atom which is unsubstituted or substituted with a (C₁-C₆)alkyl,

B represents a group selected from phenyl, pyridyl, thienyl, imidazolyl, furyl, 1,3-benzodioxolyl, benzodioxinyl, benzothienyl, benzofuryl, 2,1,3-benzothiadiazolyl, benzofurazanyl, naphthyl and indolyl,

q is an integer from 0 to 3 inclusive,

the group(s) R₅, which may be identical or different, is (are) selected from (C₁-C₆)alkyl, halogen, CN, NO₂, CF₃, OCF₃, —(CH₂)ₖNR₁₅R₁₆, —N(R₁₅)C(═O)R₁₆, —N(R₁₅)C(═O)OR₁₆, —N(R₁₅)SO₂R₁₆, —N(SO₂R₁₅)₂, —OR₁₅, —S(O)ₖ₁R₁₅, —SO₂—N(R₁₅)—(CH₂)ₖ₂—NR₁₆R₁₇, —(CH₂)ₖSO₂NR₁₅R₁₆, —X₇(CH₂)ₖC(═O)OR₁₅, —(CH₂)ₖC(═O)OR₁₅, —C(═O)O—(CH₂)ₖ₂—NR₁₅R₁₆, —X₇(CH₂)ₖC(═O)NR₁₅R₁₆, —(CH₂)ₖC(═O)NR₁₅R₁₆, and —X₆—R₂₀ in which:

X₇is S, O or NH,

k is an integer from 0 to 3 inclusive,

k1 is an integer from 0 to 2 inclusive,

k2 is an integer from 1 to 4 inclusive,

R₁₅, R₁₆ and R₁₇, which may be identical or different, are selected from hydrogen and (C₁-C₆)alkyl,

X₆ represents a single bond, —CH₂—, an oxygen atom or a sulphur atom which is unsubstituted or substituted with one or two oxygen atom,

R₂₀ represents an aromatic or non-aromatic, heterocyclic or non-heterocyclic, 5- or 6-membered ring, which is unsubstituted or substituted with one or more groups, which may be identical or different, selected from (C₁-C₆)alkyl, halogen, hydroxyl, and amino, and, when the ring is heterocyclic, it comprises from 1 to 4 heteroatoms selected from nitrogen, oxygen and sulphur.

The invention also relates to the compounds of general formula (I) in which:

R₁ represents a group selected from hydrogen, mono(C₁-C₆)alkylamino(C₁-C₆)alkyl, di(C₁-C₆)alkylamino(C₁-C₆)alkyl, (C₁-C₆)alkyl, (C₃-C₆)alkenyl, (C₃-C₆)alkynyl, aryl, aryl(C₁-C₆)alkyl, and 3- to 6-membered cycloalkyl(C₁-C₆)alkyl,

W represents an oxygen atom, or a sulphur atom,

X₁ represents a nitrogen atom or a —CH group,

X₂ and X₃ represent a—CH group,

Y represents a group selected from oxygen atom, sulphur atom, —NH, and —N(C₁-C₆)alkyl,

Z represents an oxygen atom or a —NH group,

n is an integer from 1 to 3 inclusive,

Z₁, represents —CR₈R₉ wherein R₈ and R₉, independently of each other, represent a group selected from hydrogen, (C₁-C₆)alkyl and hydroxy, and

when n is greater than or equal to 2, the hydrocarbon chain Z₁ optionally contains one double bond,

or one of the carbon atoms in the hydrocarbon chain Z₁ may be replaced with an oxygen atom, a sulphur atom which is unsubstituted or substituted with one or two oxygen atoms, or a —NH group,

A represents a group selected from phenyl, pyridyl, thienyl, imidazolyl, furyl, 1,3-benzodioxolyl, benzodioxinyl, benzothienyl, benzofuryl, 2,1,3-benzothiadiazolyl, benzofurazanyl, naphthyl and indolyl,

m is an integer from 0 to 3 inclusive,

the group(s) R₂, which may be identical or different, is (are) selected from (C₁-C₆)alkyl, halogen, —CN, —CF₃, —OCF₃, —NR₁₀R₁₁, —OR₁₀, —SR₁₀, —SO₂R₁₀, —(CH₂)ₖSO₂NR₁₀R₁₁, —X₅(CH₂)ₖC(═O)OR₁₀, —(CH₂)ₖC(═O)OR₁₀, —X₅(CH₂)ₖC(═O)NR₁₀R₁₁, —(CH₂)ₖC(═O)NR₁₀R₁₁, and —X₄—R₁₂ in which:

X₅ represents O, S or NH,

k is an integer from 0 to 3 inclusive,

R₁₀ and R₁₁, which may be identical or different, are selected from hydrogen and (C₁-C₆)alkyl,

X₄ represents —CH₂—, or an oxygen atom,

R₁₂ represents phenyl which is unsubstituted or substituted with one or more groups, which may be identical or different, selected from (C₁-C₆)alkyl, halogen, and hydroxyl,

R₃ represents a group selected from methyl and the group of formula:

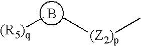

in which p is an integer from 0 to 3 inclusive,

Z₂ represents —CR₁₃R₁₄ wherein R₁₃ and R₁₄, independently of each other, represent a group selected from hydrogen, (C₁-C₆)alkyl, and hydroxy, and

when p is greater than or equal to 2, the hydrocarbon chain Z₂ optionally contains one double bond,

or one of the carbon atoms in the hydrocarbon chain Z₂ may be replaced with an oxygen atom, a sulphur atom which is unsubstituted or substituted with one or two oxygen atoms, a nitrogen atom which is unsubstituted or substituted with a (C₁-C₆)alkyl,

B represents a group selected from phenyl, pyridyl, thienyl, imidazolyl, furyl, 1,3-benzodioxolyl, benzodioxinyl, benzothienyl, benzofuryl, 2,1,3-benzothiadiazolyl, benzofurazanyl, naphthyl and indolyl,

q is an integer from 0 to 3 inclusive,

the group(s) R₅, which may be identical or different, is (are) selected from (C₁-C₆)alkyl, halogen, CN, NO₂, CF₃, OCF₃, —(CH₂)ₖNR₁₅R₁₆, —N(R₁₅)C(═O)R₁₆, —N(R₁₅)C(═O)OR₁₆, —N(R₁₅)SO₂R₁₆, —N(SO₂R₁₅)₂, —OR₁₅, —S(O)ₖ₁R₁₅, —SO₂—N(R₁₅)—(CH₂)ₖ₂—NR₁₆R₁₇, —(CH₂)ₖSO₂NR₁₅R₁₆, —X₇(CH₂)ₖC(═O)OR₁₅, —(CH₂)ₖC(═O)OR₁₅, —C(═O)O—(CH₂)ₖ₂—NR₁₅R₁₆, —X₇(CH₂)ₖC(═O)NR₁₅R₁₆, —(CH₂)ₖC(═O)NR₁₅R₁₆, and —X₆—R₂₀ in which:

X₇ is S, O or NH,

k is an integer from 0 to 3 inclusive,

k1 is an integer from 0 to 2 inclusive,

k2 is an integer from 1 to 4 inclusive,

R₁₅, R₁₆ and R₁₇, which may be identical or different, are selected from hydrogen and (C₁-C₆)alkyl,

X₆ represents a single bond, —CH₂—, an oxygen atom or a sulphur atom which is unsubstituted or substituted with one or two oxygen atom,

R₂₀ represents an aromatic or non-aromatic, heterocyclic or non-heterocyclic, 5- or 6-membered ring, which is unsubstituted or substituted with one or more groups, which may be identical or different, selected from (C₁-C₆)alkyl, halogen, hydroxyl, and amino, and, when the ring is heterocyclic, it comprises from 1 to 4 heteroatoms selected from nitrogen, oxygen and sulphur.

The invention also relates to the compounds of general formula (I) in which:

R₁ represents hydrogen, (C₁-C₆)alkyl, (C₃-C₆)alkenyl, aryl(C₁-C₆)alkyl, 3- to 6-membered cycloalkyl(C₁-C₆)alkyl,

W represents an oxygen atom,

X¹ represents —CH group or nitrogen atom, and X² and X³ represent each —CH group;

Y represents an oxygen atom,

Z represents an oxygen atom or a —NH group,

n is an integer from 1 to 3 inclusive,

Z₁ represents —CR₈R₉ wherein R₈ and R₉, independently of each other, represent a group selected from hydrogen and methyl, and

when n is greater than or equal to 2, the hydrocarbon chain Z₁ optionally contains one double bond,

or one of the carbon atoms in the hydrocarbon chain Z₁ may be replaced with an oxygen atom, a sulphur atom which is unsubstituted or substituted with one or two oxygen atoms, or a —NH group,

A represents a group selected from phenyl, pyridyl, thienyl, imidazolyl, furyl, and 1,3-benzodioxolyl,

m is an integer from 0 to 3 inclusive,

the group(s) R₂, which may be identical or different, is (are) selected from (C₁-C₆)alkyl, 3halogen, —CN, —CF₃, —OCF₃, —NR₁₀R₁₁, —-OR₁₀, —SR₁₀, —SO₂R₁₀, —(CH₂)ₖSO₂NR₁₀R₁₁, —X₅(CH₂)ₖC(═O)OR₁0, —(CH₂)ₖC(═O)OR₁₀, —X₅(CH₂)ₖC(═O)NR₁₀R₁₁, and —(CH₂)ₖC(═O)NR₁₀R₁₁, in which:

X₅ represents O, S or NH,

k is an integer from 0 to 3 inclusive,

R₁₀ and R₁₁, which may be identical or different, are selected from hydrogen and (C₁-C₆)alkyl,

R₃ represents the group of formula:

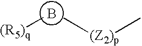

in which p is an integer from 0 to 3 inclusive,

Z₂ represents —CR₁₃R₁₄ wherein R₁₃ and R₁₄, independently of each other, represent a group selected from hydrogen, and methyl, and

when p is greater than or equal to 2, the hydrocarbon chain Z₂ optionally contains one double bond,

or one of the carbon atoms in the hydrocarbon chain Z₂ may be replaced with an oxygen atom, a sulphur atom which is unsubstituted or substituted with one or two oxygen atoms, a nitrogen atom which is unsubstituted or substituted with a (C₁-C₆)alkyl,

B represents a group selected from phenyl, pyridyl, thienyl, imidazolyl, furyl, and 1,3-benzodioxolyl,

q is an integer from 0 to 3 inclusive,

the group(s) R₅, which may be identical or different, is (are) selected from (C₁-C₆)alkyl, halogen, CN, NO₂, CF₃, OCF₃, —(CH₂)ₖNR₁₅R₁₆, —N(R₁₅)C(═O)R₁₆, —N(R₁₅)C(═O)OR₁₆, —N(R₁₅)SO₂R₁₆, —N(SO₂R₁₅)₂, —OR₁₅, —S(O)ₖ₁R₁₅, —SO₂—N(R₁₅)—(CH₂)ₖ₂—NR₁₆R₁₇, —(CH₂)ₖSO₂NR₁₅R₁₆, —X₇(CH₂)ₖC(═O)OR₁₅, —(CH₂)ₖC(═O)OR₁₅, —C(═O)O—(CH₂)ₖ₂—NR₁₅R₁₆, —X₇(CH₂)ₖC(═O)NR₁₅R₁₆, —(CH₂)ₖC(═O)NR₁₅R₁₆, in which:

X₇ is S, O or NH,

k is an integer from 0 to 3 inclusive,

k1 is an integer from 0 to 2 inclusive,

k2 is an integer from 1 to 4 inclusive,

R₁₅, R₁₆ and R₁₇, which may be identical or different, are selected from hydrogen and (C₁-C₆)alkyl.

The invention also relates to the compounds of general formula (I) in which R₁ represents a hydrogen atom or a (C₁-C₆)alkyl group.

The invention also relates to the compounds of general formula (I) in which W represents an oxygen atom, Y represents an oxygen atom, Z represents a NH group, Z₁ represents a methylene group, and n is equal to one.

The invention also relates to the compounds of general formula (I) in which X₁ represents a —CH group or a nitrogen atom, and X₂ and X₃ represent each a —CH group.

The invention also relates to the compounds of general formula (I) in which X₁ and X₃ represent each a —CH group, and X₂ represents a —CH group or a nitrogen atom.

The invention also relates to the compounds of general formula (I) in which X₁ and X₃ represent each a —CH group, and X₂ represents a nitrogen atom.

The invention also relates to the compounds of general formula (I) in which A represents a group selected from phenyl, pyridyl, 1,3-benzodioxolyl and benzofurazanyl, m is equal to 0 or 1, and R₂ represents a group selected from (C₁-C₆)alkoxy, hydroxy, halogen, and (C₁-C₆)thioalkoxy.

The invention also relates to the compounds of general formula (I) in which R₃ represents a group of formula:

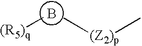

in which:

p is equal to one,

Z₂ represents a methylen group,

B represents a group selected from phenyl, pyridyl, 1,3-benzodioxolyl, and benzofurazanyl,

q is an integer from 0 and 2 inclusive,

and R₅ represents a group selected from halogen, CN, —(CH₂)ₖNR₁₅R₁₆, —S(O)ₖ₁R₁₅, —(CH₂)ₖSO₂NR₁₅R₁₆, —(CH₂)ₖC(═O)OR₁₅, —X₆—R₂₀ and —(CH₂)ₖC(═O)NR₁₅R₁₆, in which:

k is an integer from 0 to 1 inclusive,

k1 is an integer from 0 to 2 inclusive,

R₁₅ and R₁₆, which may be identical or different, are selected from hydrogen and (C₁-C₆)alkyl,

X₆ represents a single bond,

R₂₀ represents a 5-menbered heterocyclic ring comprising from 3 to 4 heteroatoms selected from oxygen and nitrogen and optionally substituted by a methyl group or an oxo group.

Among the groups defined above, the following substituents are particularly preferred:

halogen: F, Cl , Br, I, preferably F, Br and Cl;

(C₁-C₆)alkyl: linear or branched containing from 1 to 6 and preferably from 1 to 3 carbon atoms;

(C₁-C₆)alkoxy: linear or branched containing from 1 to 6 and preferably from 1 to 3 carbon atoms;

(C₃-C₆)alkenyl: containing from 3 to 6 and preferably 3 or 4 carbon atoms, more particularly allyl;

(C₃-C₆)alkynyl: containing from 3 to 6 and preferably 3 or 4 carbon atoms, more particularly propargyl;

aryl: containing from 5 to 10 and preferably 5 or 6 carbon atoms;

heteroaryl: aryl group interrupted with one or several hetero atom selected from nitrogen, oxygen and sulphur. The term “interrupted” means that the hetero atom can replace a carbon atom of the ring. Examples of such groups containing a heteroatom are, inter alia, thienyl, pyridyl, benzofurazanyl;

heterocycle: an aromatic or non-aromatic, 5- or 6-membered monocycle comprising from 1 to 4 heteroatoms selected from nitrogen, oxygen and sulphur.

aryl(C₁-C₆)alkyl in which the alkyl contains from 1 to 6 and preferably from 1 to 4 carbon atoms;

cycloalkyl: containing from 3 to 8 and preferably from 3 to 6 carbon atoms,

cycloalkyl(C₁-C₆)alkyl in which the alkyl contains from 1 to 6 and preferably from 1 to 3 carbon atoms and the cycloalkyl contains from 3 to 6 carbon atoms.

multiple bond represent a double bond or a triple bond.

Among the compounds of the present invention that are preferred are the compounds described below in Examples 1 to Example 227.

More particularly, the preferred compounds of the present invention are compound of formula (I) which are:

4-[6-(4-Methoxy-benzylcarbamoyl)-1-methyl-2,4-dioxo-1,4-dihydro-2H-pyrido[3,4-d]pyrimidin-3-ylmethyl]-benzoic acid

3-Benzyl-1-methyl-2,4-dioxo-1,2,3,4-tetrahydro-pyrido[3,4-d]pyrimidine-6-carboxylic acid (1,3-benzodioxol-5-ylmethyl)-amide

4-[6-(4-Fluoro-benzylcarbamoyl)-1-methyl-2,4-dioxo-1,4-dihydro-2H-quinazolin-3-ylmethyl]-benzoic acid

1-Methyl-2,4-dioxo-3-[4-(5-oxo-4,5-dihydro-1,2,4-oxadiazol-3-yl)-benzyl]-1,2,3,4-tetrahydro-quinazoline-6-carboxylic acid 4-methoxy-benzylamide

4-[6-(4-Methoxy-benzylcarbamoyl)-1-methyl-2,4-dioxo-1,4-dihydro-2H-quinazolin-3-ylmethyl]-benzoic acid hemicalcium salt

Methyl 4-[6-(4-Methoxy-benzylcarbamoyl)-1-methyl-2,4-dioxo-1,4-dihydro-2H-pyrido[3,4-d]pyrimidin-3-ylmethyl]-benzoate

4-[6-(3-Methoxy-benzylcarbamoyl)-1-methyl-2,4-dioxo-1,4-dihydro-2H quinazolin-3-ylmethyl]-benzoic acid

1-Methyl-2,4-dioxo-3-[4-(2H-tetrazol-5-yl)-benzyl]-1,2,3,4-tetrahydro-quinazoline-6-carboxylic acid 4-methoxy-benzylamide

Methyl 2-hydroxy-4-[6-(4-methoxy-benzylcarbamoyl)-1-methyl-2,4-dioxo-1,4-dihydro-2H-quinazolin-3-ylmethyl]-benzoate

3-(4-Chloro-benzyl)-1-methyl-2,4-dioxo-1,2,3,4-tetrahydroquinazoline-6-carboxylic acid 3-methoxy-benzylamide

4-{6-[(1,3-Benzodioxol-5-ylmethyl)-carbamoyl]-1-methyl-2,4-dioxo-1,4-dihydro-2H-quinazolin-3-ylmethyl}-benzoic acid

2-Hydroxy-4-[6-(4-methoxy-benzylcarbamoyl)-1-methyl-2,4-dioxo-1,4-dihydro-2H-quinazolin-3-ylmethyl]-benzoic acid

Methyl 4-[6-(3-methoxy-benzylcarbamoyl)-1-methyl-2,4-dioxo-1,4-dihydro-2H-quinazolin-3-ylmethyl]-benzoate

3-(3-Fluoro-benzyl)-1-methyl-2,4-dioxo-1,2,3,4-tetrahydro-quinazoline-6-carboxylic acid 3-methoxy-benzylamide

4-Pyridylmethyl 3-benzyl-2,4-dioxo-1,2,3,4-tetrahydroquinazoline-6-carboxylate

Methyl 4-{6-[(1,3-benzodioxol-5-ylmethyl)-carbamoyl]-1-methyl-2,4-dioxo-1,4-dihydro-2H-quinazolin-3-ylmethyl}-benzoate

1-Methyl-3-[4-(5-methyl-1,2,4-oxadiazol-3-yl)-benzyl]-2,4-dioxo-1,2,3,4-tetrahydro-quinazoline-6-carboxylic acid 4-methoxy-benzylamide

1-Methyl-3-[4-(3-methyl-1,2,4-oxadiazol-5-yl)-benzyl]-2,4-dioxo-1,2,3,4-tetrahydro-quinazoline-6-carboxylic acid 4-methoxy-benzylamide

3-(3-Fluoro-benzyl)-1-methyl-2,4-dioxo-1,2,3,4-tetrahydro-quinazoline-6-carboxylic acid (2-methoxy-pyridin-4-ylmethyl)-amide

4-[6-(4-Methoxy-benzylcarbamoyl)-1-methyl-2,4-dioxo-1,4-dihydro-2H]-quinazolin-3-ylmethyl]-benzoic acid

1-{4-[6-(4-Methoxy-benzylcarbamoyl)-1-methyl-2,4-dioxo-1,4-dihydro-2H-quinazolin-3-ylmethyl]-phenyl}-cyclopropanecarboxylic acid

4-Pyridylmethyl 3-benzyl-1-methyl-2,4-dioxo-1,2,3,4-tetrahydroquinazoline -6-carboxylate

3-(4-Fluoro-benzyl)-1-methyl-2,4-dioxo-1,2,3,4-tetrahydroquinazoline-6-carboxylic acid 3-methoxy-benzylamide

3-(3,4-Difluoro-benzyl)-1-methyl-2,4-dioxo-1,2,3,4-tetrahydro-quinazoline-6-carboxylic acid 4-methoxy-benzylamide

3-(4-Dimethylcarbamoyl-benzyl)-1-methyl-2,4-dioxo-1,2,3,4-tetrahydroquinazoline-6-carboxylic acid 4-methoxy-benzylamide

1-Methyl-3-[4-(2-methyl-2H-tetrazol-5-yl)-benzyl]-2,4-dioxo-1,2,3,4-tetrahydro-quinazoline-6-carboxylic acid 4-methoxy-benzylamide

3-(4-Bromo-benzyl)-1-methyl-2,4-dioxo-1,2,3,4-tetrahydro-quinazoline-6-carboxylic acid (2-methoxy-pyridin-4-ylmethyl)-amide

3-(3,4-Difluoro-benzyl)-1-methyl-2,4-dioxo-1,2,3,4-tetrahydro-quinazoline-6-carboxylic acid (pyridin-3-ylmethyl)-amide

Benzo[1,3]dioxol-5-ylmethyl-3-benzyl-1-methyl-2,4-dioxo-1,2,3,4-tetrahydroquinazoline-6-carboxylate

3-Benzyl-1-methyl-2,4-dioxo-1,2,3,4-tetrahydroquinazoline-6-carboxylic acid (benzo[1,3]dioxol-5-ylmethyl)amide

1-Methyl-3-(4-methylcarbamoyl-benzyl)-2,4-dioxo-1,2,3,4-tetrahydro-quinazoline-6-carboxylic acid 4-methoxy-benzylamide

3-(3-Fluoro-benzyl)-1-methyl-2,4-dioxo-1,2,3,4-tetrahydro-quinazoline-6-carboxylic acid 4-methoxy-benzylamide

4-[6-(4-Hydroxy-benzylcarbamoyl)-1-methyl-2,4-dioxo-1,4-dihydro-2H-quinazolin-3-ylmethyl]-benzoic acid

Methyl 4-[6-(4-fluoro-benzylcarbamoyl)-1-methyl-2,4-dioxo-1,4-dihydro-2H-quinazolin-3-ylmethyl]-benzoate

3-(4-Chlorobenzyl)-2,4-dioxo-1,2,3,4-tetrahydroquinazoline-6-carboxylic acid (benzo[1,3]dioxol-5-ylmethyl)amide

1-Methyl-3-[4-(1-methyl-1H-tetrazol-5-yl)-benzyl]-2,4-dioxo-1,2,3,4-tetrahydro-quinazoline-6-carboxylic acid 4-methoxy-benzylamide

3-(4-Methoxybenzyl)-1-methyl-2,4-dioxo-1,2,3,4-tetrahydroquinazoline-6-carboxylic acid 4-methoxybenzylamide

4-Pyridylmethyl 3-(benzo[1,3]dioxol-5-ylmethyl)-2,4-dioxo-1,2,3,4-tetrahydroquinazoline-6-carboxylate

Methyl 4-[6-(4-methoxy-benzylcarbamoyl)-1-methyl-2,4-dioxo-1,4-dihydro-2H-quinazolin-3-ylmethyl]-benzoate

1-Methyl-2,4-dioxo-3-pyridin-4-ylmethyl-1,2,3,4-tetrahydro-quinazoline-carboxylic acid 4-methoxy-benzylamide

3-(4-Amino-benzyl)-1-methyl-2,4-dioxo-1,2,3,4-tetrahydro-quinazoline-6-carboxylic acid 4-methoxy-benzylamide

1-Methyl-3-(4-nitro-benzyl)-2,4-dioxo-1,2,3,4-tetrahydro-quinazoline-6-carboxylic acid 4-methoxy-benzylamide

2-Methoxy-4-[6-(4-methoxy-benzylcarbamoyl)-1-methyl-2,4-dioxo-1,4-dihydro-2H-quinazolin-3-ylmethyl]-benzoic acid

1-Methyl-3-(4-methylsulfamoyl-benzyl)-2,4-dioxo-1,2,3,4-tetrahydro-quinazoline-6-carboxylic acid 4-methoxy-benzylamide

1-Methyl-2,4-dioxo-3-(4-sulfamoyl-benzyl)-1,2,3,4-tetrahydro-quinazoline-6-carboxylic acid 4-methoxy-benzylamiide

3-(4-Fluoro-benzyl)-1-methyl-2,4-dioxo-1,2,3,4-tetrahydro-quinazoline-6-carboxylic acid 4-methoxy-benzylamide

3-(4-Fluoro-benzyl)-1-methyl-2,4-dioxo-1,2,3,4-tetrahydro-quinazoline-6-carboxylic acid (pyridin-4-ylmethyl)-amide

3-(4-Methoxy-benzyl)-1-methyl-2,4-dioxo-1,2,3,4-tetrahydro-quinazoline-6-carboxylic acid (pyridin-4-ylmethyl)-amide

2-Methyl-4-[6-(4-methoxy-benzylcarbamoyl)-1-methyl-2,4-dioxo-1,4-dihydro-2H-quinazolin-3-ylmethyl]-benzoic acid

3-(4-Cyano-benzyl)-1-methyl-2,4-dioxo-1,2,3,4-tetrahydro-quinazoline-6-carboxylic acid 4-methoxy-benzylamide

4-{1-Methyl-2,4-dioxo-6-[(pyridin-4-ylmethyl)-carbamoyl]-1,4-dihydro-2H-quinazolin-3-ylmethyl}-benzoic acid

3-(3-fluoro-4-methoxy-benzyl)-1-methyl-2,4-dioxo-1,2,3,4-tetrahydro-quinazoline-6-carboxylic acid 4-methoxy benzylamine

4-[1-Ethyl-6-(4-methoxy-benzylcarbamoyl)-2,4-dioxo-1,4-dihydro-2H-quinazolin-3-ylmethyl]-benzoic acid

3-(Benzo[1,3]dioxol-5-ylmethyl)-2,4-dioxo-1,2,3,4-tetrahydroquinazoline-6-carboxylic acid (benzo[1,3]dioxol-5-ylmethyl)amide

3-(2′-Cyano-biphenyl-4-ylmethyl)-1-methyl-2,4-dioxo-1,2,3,4-tetrahydro-quinazoline-6-carboxylic acid 4-methoxy-benzylamide

4-[1-Methyl-6-(4-methylsulfanyl-benzylcarbamoyl)-2,4-dioxo-1,4-dihydro-2H-quinazolin-3-ylmethyl]-benzoic acid

4-{6-[(Benzofurazan-5-ylmethyl)-carbamoyl]-1-methyl-2,4-dioxo-1,4-dihydro-2H-quinazolin-3-ylmethyl}-benzoic acid

Methyl 2-methyl-4-[6-(4-metboxy-benzylcarbamoyl)-1-methyl-2,4-dioxo-1,4-dihydro-2H-quinazolin-3-ylmethyl]-benzoate

3-(4-Acetylamino-benzyl)-1-methyl-2,4-dioxo-1,2,3,4-tetrahydro-quinazoline-6-carboxylic acid 4-methoxy-benzylamide

3-(Benzo[1,3]dioxol-5-ylmethyl)-1-methyl-2,4-dioxo-1,2,3,4-tetrahydroquinazoline-6-carboxylic acid (benzo[1,3]dioxol-5-ylmethyl)amide

3-(4-Dimethylcarbamoylmethyl-benzyl)-1-methyl-2,4-dioxo-1,2,3,4-tetrahydro-quinazoline-6-carboxylic acid 4-methoxy-benzylamide

Benzo[1,3]dioxol-5-ylmethyl 3-benzyl-2,4-dioxo-1,2,3,4-tetrahydroquinazoline-6-carboxylate

{4-[6-(4-Methoxy-benzylcarbamoyl)-1-methyl-2,4-dioxo-1,4-dihydro-2H-quinazolin-3-ylmethyl]-phenyl}-acetic acid

(4-{1-Methyl-2,4-dioxo-6-[(pyridin-4-ylmethyl)-carbamoyl]-1,4-dihydro-2H-quinazolin-3-ylmethyl}-phenyl)-acetic acid

3-Benzyl-2,4-dioxo-1,2,3,4-tetrahydroquinazoline-6-carboxylic acid 4-methoxybenzylamide

Methyl {4-[6-(4-methoxy-benzylcarbamoyl)-1-methyl-2,4-dioxo-1,4-dihydro-2H-quinazolin-3-ylmethyl]-phenyl}-acetate

3-(3-Fluoro-benzyl)-1-methyl-2,4-dioxo-1,2,3,4-tetrahydro-quinazoline-6-carboxylic acid (pyridin-4-ylmethyl)-amide

2,4-Dioxo-3-(thien-2-ylmethyl)-1,2,3,4-tetrahydroquinazoline-6-carboxylic acid (benzo[1,3]dioxol-5-ylmethyl)amide

1-Methyl-3-(4-methylsulfamoyl-benzyl)-2,4-dioxo-1,2,3,4-tetrahydroquinazoline-6-carboxylic acid 4-methoxy-benzylamide

Methyl 4-{1-methyl-2,4-dioxo-6-[(pyridin-4-ylmethyl)-carbamoyl]-1,4-dihydro-2H-quinazolin-3-ylmethyl}-benzoate

2-Fluoro-4-[6-(4-methoxy-benzylcarbamoyl)-1-methyl-2,4-dioxo-1,4-dihydro-2H-quinazolin-3-ylmethyl]-benzoic acid

3-(4-Cyano-benzyl)-1-methyl-2,4-dioxo-1,2,3,4-tetrahydro-pyrido[3,4-d]pyrimidine-6-carboxylic acid 4-methoxy-benzylamide

4-[6-(3-Methoxy-benzylcarbamoyl)-1-methyl-2,4-dioxo-1,4-dihydro-2H-pyrido[3,4-d]pyrimidin-3-ylmethyl]-benzoic acid

4-[6-(4-Methoxy-benzylcarbamoyl)-1-methyl-2,4-dioxo-1,4-dihydro-2H-quinazolin-3-ylmethyl]-benzoic acid hemimagnesium salt

Example 164: 4-[6-(4-Methoxy-benzylcarbamoyl)-1-methyl-2,4-dioxo-1,4-dihydro-2H-pyrido[2,3-d]pyrimidin-3-ylmethyl]-benzoic acid

3-[4-(N-methylsulfonylamino)-benzyl]-1-methyl-2,4-dioxo-1,2,3,4-tetrahydroquinazoline-6-carboxylic acid 4-methoxy-benzylamide

Ethyl 2-Fluoro-4-[6-(4-methoxy-benzylcarbamoyl)-1-methyl-2,4-dioxo-1,4-dihydro-2H-quinazolin-3-ylmethyl]-benzoate

3-(4-Dimethylsulfamoyl-benzyl)-1-methyl-2,4-dioxo-1,2,3,4-tetrahydroquinazoline-6-carboxylic acid 4-methoxy-benzylamide

and 3-(4-Methoxybenzyl)-1-methyl-2,4-dioxo-1,2,3,4-tetrahydroquinazoline-6-carboxylic acid (benzo[1,3]dioxol-5-ylmethyl)amide.

The invention also relates to the pharmaceutically acceptable salts of the compounds of formula (I). A review of the pharmaceutically acceptable salts will be found in J. Pharm. Sci., 1977, vol. 66:1-19. However, the expression “pharmacologically acceptable salts of a compound of formula (I) with a basic function” means the addition salts of the compounds of formula (I) formed from non-toxic mineral or organic acids such as, for example, hydrobromic acid, hydrochloric acid, sulphuric acid, phosphoric acid, nitric acid, acetic acid, succinic acid, tartaric acid, citric acid, maleic acid, hydroxymaleic acid, benzoic acid, fumaric acid, toluenesulphonic acid, isethionic acid and the like. The various quaternary ammonium salts of the compounds of formula (I) are also included in this category of compounds of the invention. In addition, the expression “pharmacologically acceptable salts of a compound of formula (I) with an acid function” means the usual salts of the compounds of formula (I) formed from non-toxic mineral or organic bases such as, for example, the hydroxides of alkali metals and of alkaline-earth metals (sodium, potassium, magnesium and calcium), amines (dibenzylethylenediamine, trimethylamine, piperidine, pyrrolidine, benzylamine and the like) or quaternary ammonium hydroxides such as tetramethylammonium hydroxide.

As mentioned above, the compounds of formula (I) of the present invention are matrix metalloprotease inhibitors, and more particularly inhibitors of the enzyme MMP-13. In this respect, their use is recommended in the treatment of diseases or complaints involving a therapy by MMP-13 inhibition. By way of example, the use of the compounds of the present invention may be recommended during the treatment of any pathology in which a destruction of extracellular matrix tissue is involved, and most particularly pathologies such as arthritis, rheumatoid arthritis, osteoarthritis, osteoporosis, periodontal diseases, inflammatory bowel disease, psoriasis, multiple sclerosis, cardiac insufficiency, atherosclerosis, asthma, chronic obstructive pulmonary disease (COPD), age-related macular degeneration (ARMD) and certain cancers.

Selectivity of the Compounds of Formula (I) for the Enzyme MMP-13

Most of the matrix metalloprotease inhibitors described in the prior art are non-selective inhibitors, capable of simultaneously inhibiting several matrix metalloproteases. For example, compounds such as CGS-27.023A and AG-3340 (Montana and Baxter (2000)) inhibit both MMP-1, MMP-2, MMP-3, MMP-9 and MMP-13, i.e. these compounds of the prior art inhibit MMPs of both collagenase, gelatinase and stromelysin type.

It has been shown according to the invention that compounds of general formula (I) are selective inhibitors of MMP-13. “Selective inhibitors of MMP-13” refers to a compound of formula (I) which have an IC₅₀ for MMP-13 at least 5 time lower than the IC₅₀ for a MMP distinct from MMP-13, and preferably at least 10 times, 15 times, 20 times, 30 times, 40 times, 50 times, 100 times or 1000 times lower than the IC₅₀ value for a MMP distinct from MMP-13.

A MMP distinct from MMP-13 refers preferably to a matrix metalloprotease selected from MMP-1, MMP-2, MMP-3, MMP-7, MMP-9, MMP-12 and MMP-14.

In particular, it has been shown according to the invention that the compounds of general formula (I), and more particularly the family of compounds given as examples in the present description, have an IC₅₀ value for the enzyme MMP-13 which is often 1 000 times lower than the value of their IC₅₀ for other matrix metalloproteases, in particular MMP-1, MMP-2, MMP-3, MMP-7, MMP-9, MMP-12 and MMP-14.

The result of this is that the compounds of general formula (I) according to the invention are particularly useful in the treatment of complaints mainly associated with a physiological imbalance between the MMP-13 enzymes and their natural tissue inhibitors.

Pharmaceutical Formulation of the Compounds of the Invention

A subject of the present invention is also a pharmaceutical composition comprising a compound of general formula (I) as defined above and a pharmaceutically acceptable excipient.

The invention also relates to the use of a compound of general formula (I) as defined above for the preparation of a medicinal product intended for treating a disease or complaint involving therapy by inhibition of matrix metalloprotease, and more particularly a disease or complaint involving therapy by inhibition of type-13 matrix metalloprotease (MMP-13) such as arthritis, rheumatoid arthritis, osteoarthritis, osteoporosis, periodontal diseases, inflammatory bowel disease, psoriasis, multiple sclerosis, cardiac insufficiency, atherosclerosis, asthma, chronic obstructive pulmonary disease (COPD), age-related macular degeneration (ARMD) and cancers.

The invention also relates to a method for treating a pathology associated with an imbalance in the activity of MMPs, and more specifically of MMP-13, the said method comprising a step during which a pharmaceutically effective amount of an MMP-inhibitor compound according to the invention, or a pharmaceutical composition containing this compound, is administered to a patient requiring such a treatment.

Among the various pathologies associated with an imbalance in MMP activity, an MMP-13-inhibitor compound of general formula (I) according to the invention is particularly useful for treating all pathologies brought about by a degradation of extracellular matrix tissue, and more particularly for treating rheumatoid arthritis, osteoarthritis, osteoporosis, periodontal diseases, inflammatory bowel disease, psoriasis, multiple sclerosis, cardiac insufficiency, atherosclerosis, asthma, chronic obstructive pulmonary disease (COPD), age-related macular degeneration (ARMD) and cancer. In an entirely preferred manner, a compound of general formula (I) as defined according to the invention will be used, preferably to treat arthritis, osteoarthritis and rheumatoid arthritis.

The compounds of the invention are administered in the form of compositions that are suitable for the nature and gravity of the complaint to be treated. The daily dosage in man is usually between 2 mg and 1 g of product which may be absorbed in one or more dosage intakes. The compositions are prepared by methods that are common to those skilled in the art and generally comprise 0.5% to 60% by weight of active principle (compound of formula I) and 40% to 99.5% by weight of pharmaceutically acceptable vehicle.

The compositions of the present invention are thus prepared in forms that are compatible with the desired route of administration. By way of example, the following pharmaceutical forms may be envisaged, although the list given below is not limiting:

1) Forms for Oral Administration

Drinkable solutions, suspensions, sachets of powder for drinkable solution, sachets of powder for drinkable suspension, gastro-resistant gel capsules, sustained-release forms, emulsions, HPMR capsules or gel capsules, lyophilizates to be melted under the tongue.

2) Forms for Parenteral Administration

Intravenous Route

Aqueous solutions, water/cosolvent solutions, solutions using one or more solubilizing agents, colloidal suspensions, emulsions, nanoparticulate suspensions which can be used for the injection of sustained-release forms, dispersed forms and liposomes.

Subcutaneous/Intramuscular Route

In addition to the forms which can be used intravenously and which can also be used for the subcutaneous and intramuscular routes, other types of forms such as suspensions, dispersed forms, sustained-release gels and sustained-release implants may also be used.

3) Forms for Topical Administration

Among the most common topical forms that are distinguished are creams, gels (aqueous phases gelled with polymers), patches, which are dressings to be stuck directly onto the skin and which can be used to treat dermatosis without percutaneous penetration of the active substance, sprays, emulsions and solutions.

4) Forms for Pulmonary Administration

Forms such as solutions for aerosols, powders for inhalers and other suitable forms are distinguished in this category.

5) Forms for Nasal Administration

This especially relates herein to solutions for drops.

6) Forms for Rectal Administration

Suppositories and gels will be selected, inter alia.

It is also possible to envisage using forms allowing the administration of ophthalmic solutions or allowing the vaginal administration of the active principle.

Another important category of pharmaceutical form which may be used in the context of the present invention relates to forms for improving the solubility of the active principle. By way of example, it may be envisaged to use aqueous solutions of cyclodextrin, and more particularly forms comprising hydroxypropyl-β-cyclodextrin. A detailed review of this type of pharmaceutical form is presented in the article published under the reference Journal of Pharmaceutical Sciences, 85 (11), 1142-1169 (1996), and incorporated into the present patent application by reference.

The various pharmaceutical forms recommended above are described in detail in the book “Pharmacie galenique” by A. Lehir (published by Masson, 1992 (6th edition)), which is incorporated into the present patent application by reference.

Intermediate Compounds

The present invention also relates to an intermediate compound of general formula (III)

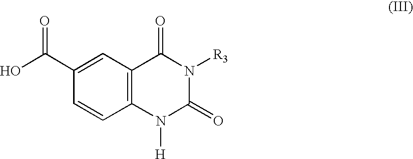

in which R³ has the same meaning as for the compound of general formula (1).

According to another aspect, the present invention also relates to an intermediate compound of general formula (IV).

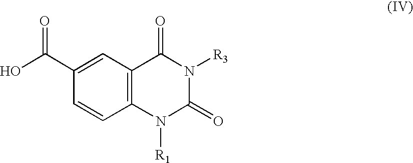

in which R₁ and R₃ have the same meaning as that defined above for the compound of general formula (I).

Processes for Synthesizing the Compounds of General Formula (I)

Throughout this application the following abbreviations have the meanings listed below:

DEAD: Diethyl azodicarboxylate

DIPEA: N,N-diisopropylethylamine

DMF: N,N-dimethylformamide

NMP: 1-methyl-2-pyrrolidinone

THF: tetrahydrofuran

TOTU: O-[(ethoxycarbonyl)cyanomethylenamino]-N,N,N′,N′-tetramethyluronium tetrafluoroborate

EDCI: 1-(3-dimethylaminopropyl)-3-ethylcarbodiimide hydrochloride

HOBT: 1-hydroxybenzotriazole hydrate

The compounds according to the present invention can be obtained by carrying out several synthetic processes. Some of these synthetic processes are described below:

A) General Process

A general process for the synthesis of the compounds of general formula (I) is described in the following scheme:

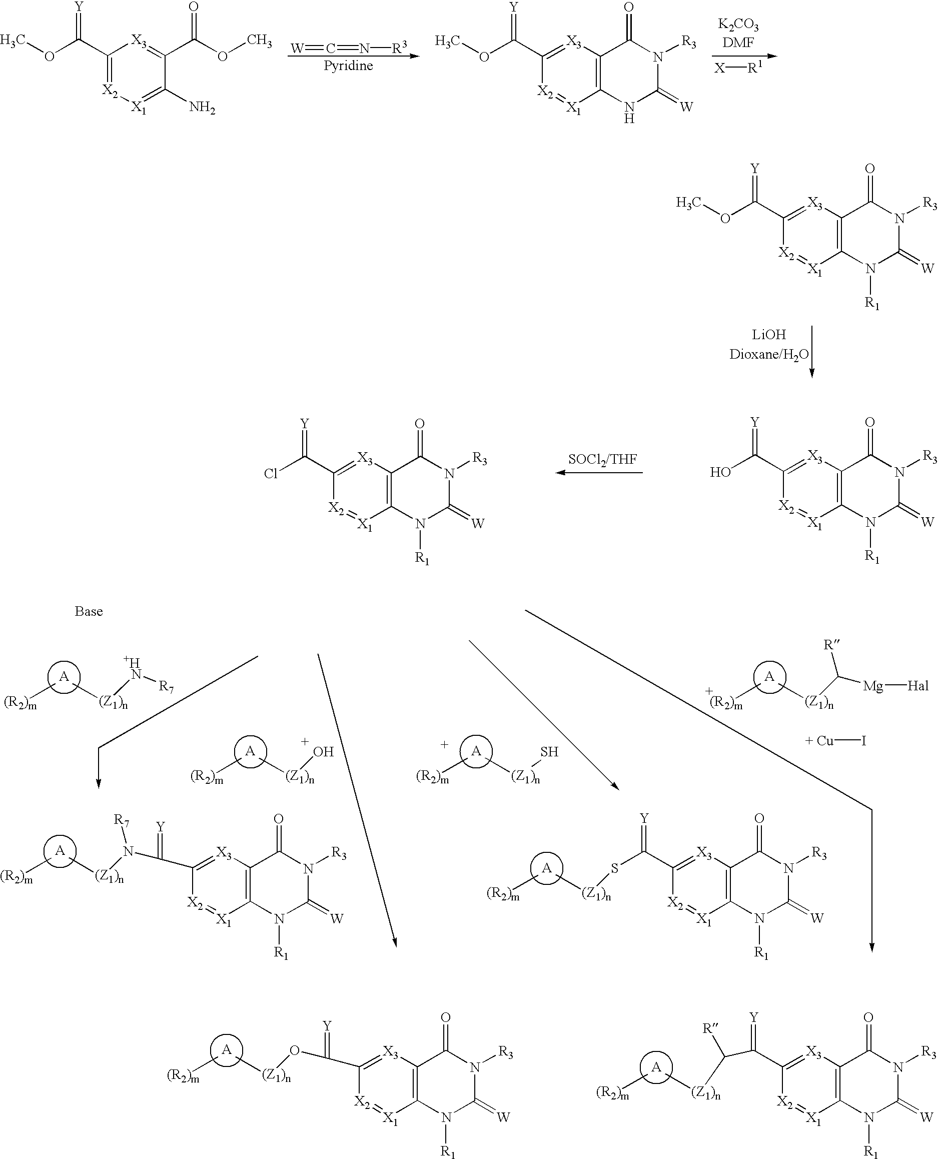

in which R₇ is hydrogen, (C₁-C₆) alkyl, cycloalkyl, aryl or heteroaryl, R″ is (C₁-C₆)alkyl, aryl, aryl(C₁-C₆)alkyl, aromatic or non-aromatic heterocycle or cycloalkyl and R₁, R₂, R₃, X₁, X₂, X₃, A, W, Y , Z₁, n and m have the same meaning as that defined above for the compound formula (I).

B) Synthetic Process No. 1

The compounds of the present invention may be obtained firstly by the method represented in Scheme 1 below.

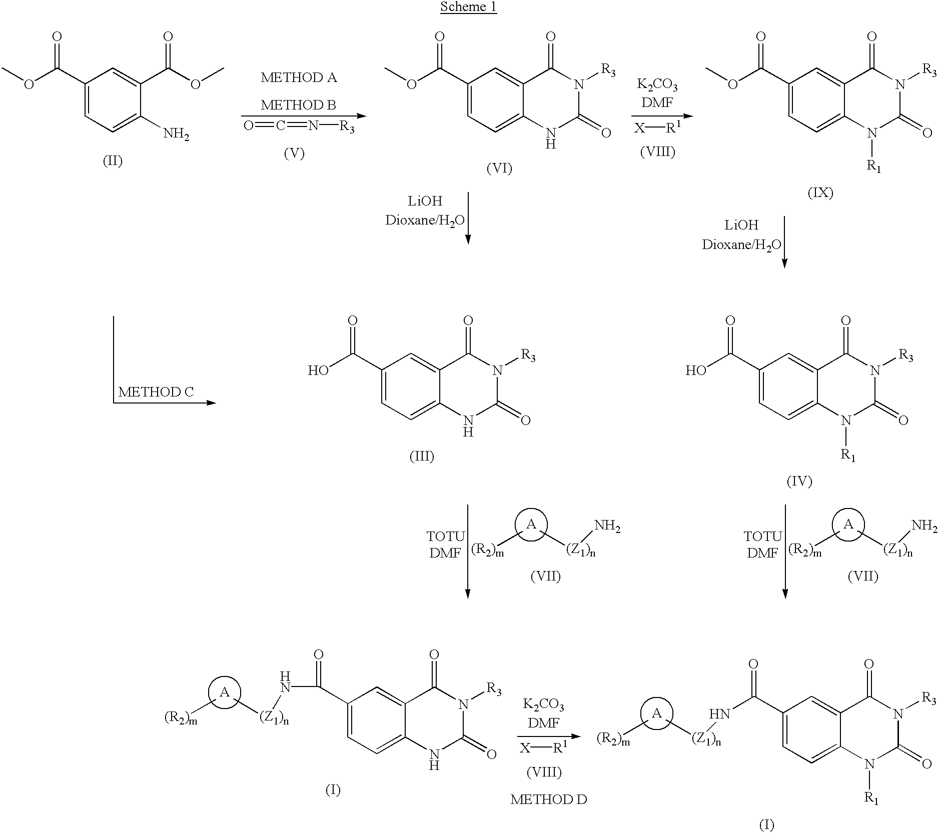

in which each of the generic substituents is as defined for the compound of general formula (I).

The intermediate compound of formula (II) which constitutes the starting material for the synthetic process illustrated by Scheme 1 above may be prepared in accordance with Scheme 2 below:

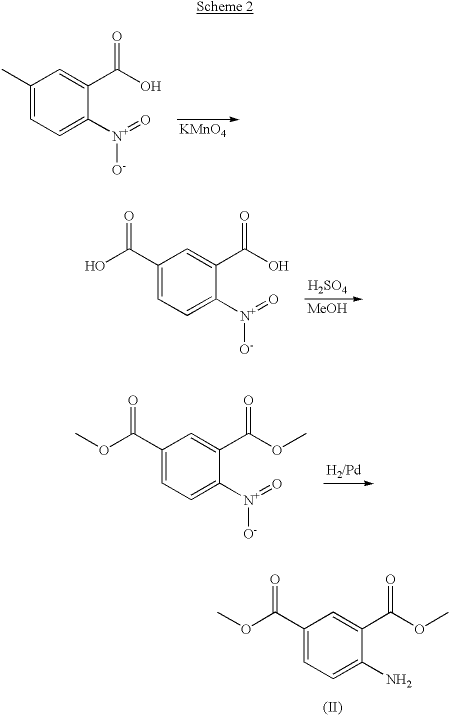

The intermediate compound of formula (II) which constitutes the starting material in the process to synthesize the compounds of general formula (I) according to the invention as illustrated in Scheme 1 above may also be prepared according to the process illustrated in Scheme 3 below.

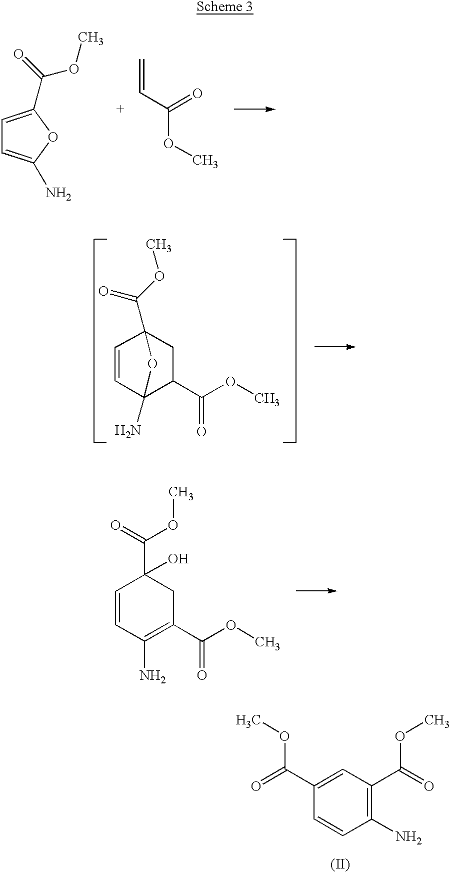

The compound of general formula (III) may be prepared, in accordance with the process described in Scheme 1 above, from the compound of formula (II), according to the synthetic Scheme 4 (Method A) below:

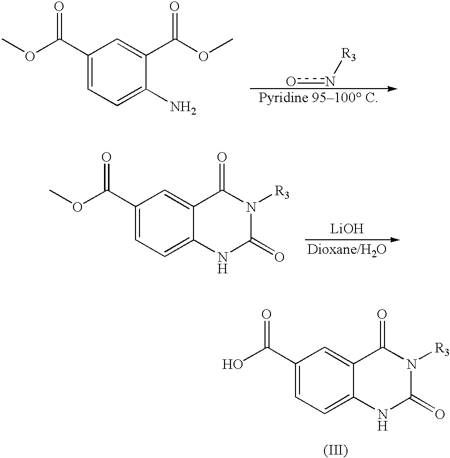

in which R₃ is as defined above for the compound of general formula (I).

According to another aspect, the intermediate compound of formula (III) may be prepared, in accordance with the synthetic process illustrated in Scheme 1 above, according to Method B, as illustrated in Synthetic Scheme 5 below:

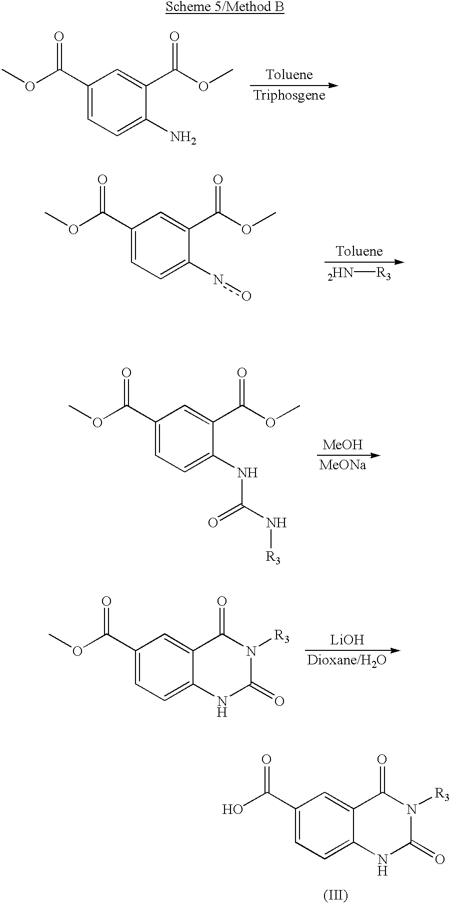

in which R₃ is as defined for the compound of general formula (I).

According to yet another aspect, an intermediate compound of general formula (III), in which R₃ is a benzyl radical, may be obtained, in accordance with the synthetic process illustrated in Scheme 1 above, according to Method C illustrated in Synthetic Scheme 6

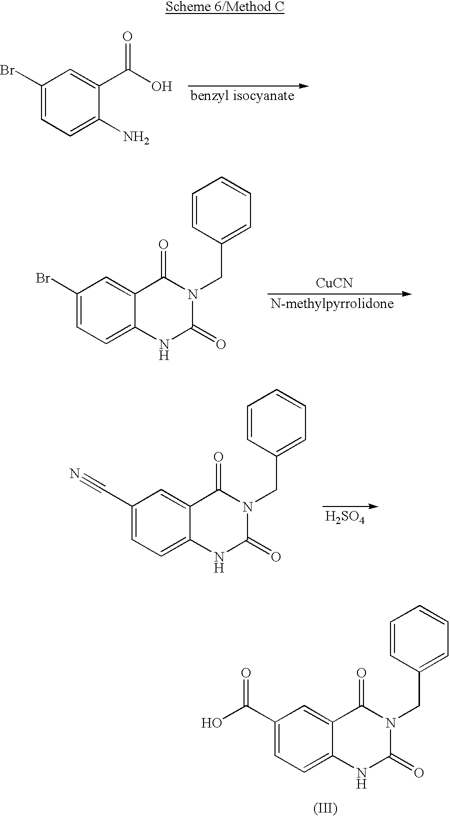

Consequently, a subject of the invention is also a process for manufacturing a compound of general formula (I):

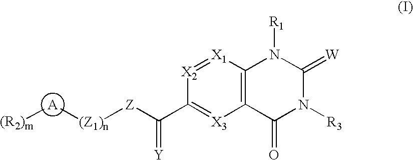

in which R₁, R₂, R₃, Z₁, A, n and m are as defined in the summary of the invention, X₁, X₂ and X₃ are CH, Y is O, Z is N—R₇ and W is O,

the said process being characterized in that it comprises the reaction of a compound of formula (I):

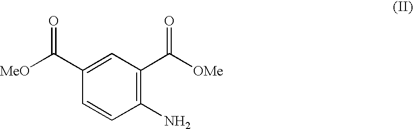

with pyridine and the compound of general formula (V):

O═C═N—R₃, (V)

in which R₃ is as defined in the summary of the invention, to give the compound of general formula (VI):

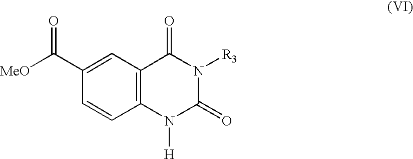

in which R₃ is as defined hereinbefore,

followed by reacting the compound of general formula (VI) in the presence of LiOH to give the compound of general formula (III) in which R₃ is as defined in the summary of the invention.

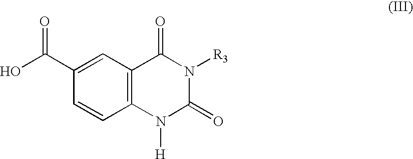

The above process is also characterized in that the compound of general formula (III) in which R₃ is as defined for the compound of general formula (I), is reacted, in the presence of an acid activator such as TOTU, with the compound of general formula (VII):

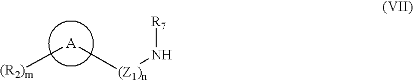

in which R₇ is selected from hydrogen, (C₁-C₆)alkyl, aryl(C₁-C₆)alkyl, cycloalkyl, aryl and heteroaryl, and A, R₂, Z₁, m and n are as defined for the compound of general formula (I),

to give the compound of general formula (I) in which R₁ represents H, X₁, X₂ and X₃ are CH, Y is O, Z is N—R₇, W is O, and A, R₂, R₃, Z₁, m and n are as defined hereinbefore.

The present invention also relates to a process for manufacturing a compound of general formula (I) in which R₁, R₂, R₃, A, Z₁, m and n are as defined for the compound of general formula (I), X₁, X₂ and X₃ are CH, W is O, Y is O and Z is N—R₇, the said process being characterized in that a compound of general formula (VI):

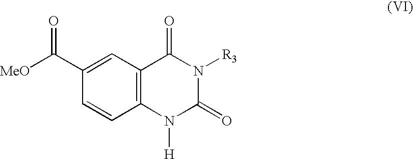

in which R₃ is as defined in the summary of the invention, is reacted, in the presence of a base, with compound (VIII) of general formula X—R₁, in which R₁ is as defined in the summary of the invention and X is a leaving group such as halogen, to give the compound of general formula (IX):

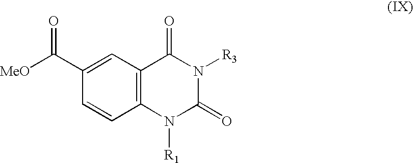

in which R₁ and R₃ are as defined hereinbefore.

The above process is also characterized in that the compound of general formula (IX):

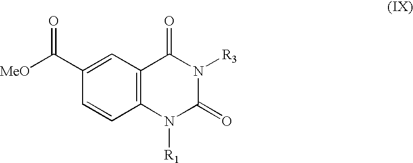

is reacted in the presence of LiOH to give the compound of general formula (IV):

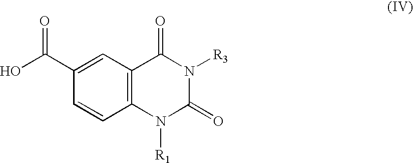

in which R₁ and R₃ are as defined hereinbefore.

The above process is also characterized in that the compound of general formula (IV):

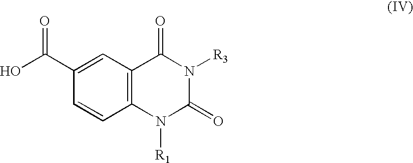

in which R₃ is as defined in the compound of general formula (I), is reacted, in the presence of an acid activator such as TOTU, with the compound of general formula (VII)

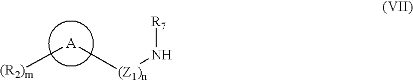

in which R₇ is selected from hydrogen, (C₁-C₆)alkyl, aryl(C₁-C₆)alkyl, cycloalkyl, aryl and heteroaryl, and A, R₂, Z₁, m and n are as defined in the summary of the invention, to give the compound of general formula (I):

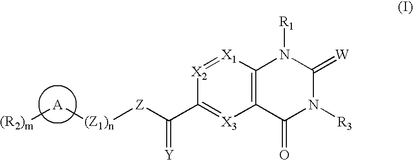

in which R₁, R₂, R₃, A, Z₁, m and n are as defined in the summary of the invention, X₁, X₂ and X₃ are CH, W is O, Y is O and Z is N—R₇.

Another subject of the present invention is a process for manufacturing the compound of general formula (I) in which R₁, R₂, R₃, W, X₁, X₂, X₃, A, Z₁, m and n are as defined for the compound of general formula (I), Y is O and Z is N—R₇, characterized in that a compound of general formula (I) in which R₁ is H,

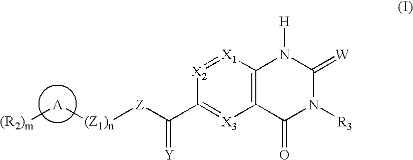

is reacted, in the presence of a base, with a compound (VIII) of general formula X—R₁, in which R₁ is as defined in the summary of the invention and X is a leaving group such as halogen, to give the compound of general formula (I) in which R₁ is as defined in the summary of the invention.

C. Synthetic Process No. 2

The compounds of the present invention can also be obtained by the method represented in Scheme 7 below:

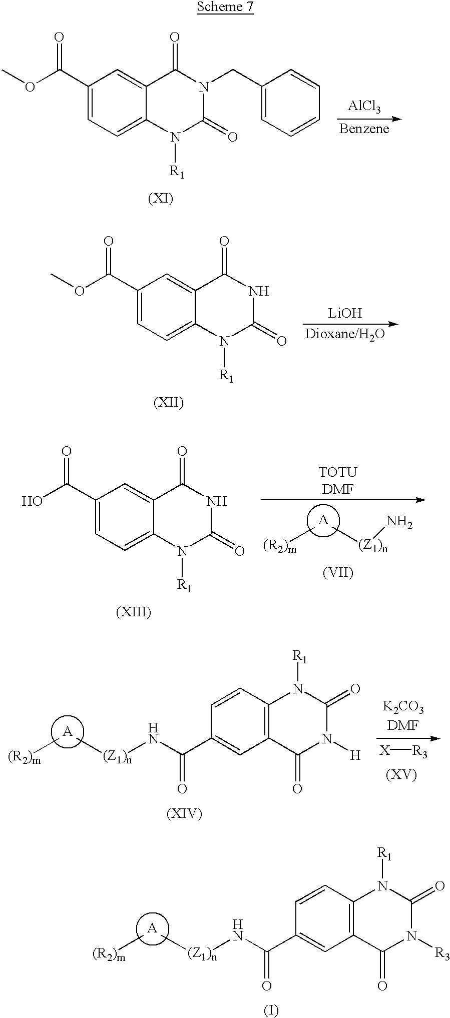

in which each of the generic substituents is as defined for the compound of general formula (I).

The present invention also relates to a process for manufacturing a compound of general formula (I) in which X₁, X₂ and X₃ are CH, W is O, Y is O, Z is N—R₇, R₁, R₃, A, R₂, Z₁, m and n are as defined for the compound of general formula (I) characterized in that a compound of general formula (XI):

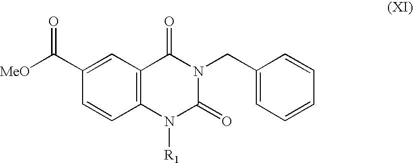

in which R₁ is as defined hereinbefore, is reacted with AlCl₃ in a solvent such as benzene, to give the compound of general formula (XII):

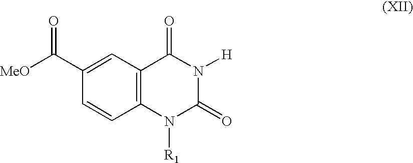

in which R₁ is as defined hereinbefore.

The process for manufacturing a compound of general formula (I) above is also characterized in that it comprises a step in which the compound of general formula (XII) is reacted in the presence of LiOH and a mixture of dioxane/H₂O to give the compound of general formula (XIII):

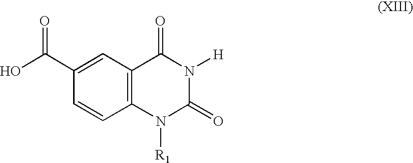

in which R₁ is as defined hereinbefore.

The process for manufacturing a compound of general formula (I) above is also characterized in that it comprises a step in which the compound of general formula (XIII) is reacted, in the presence of an acid activator such as TOTU with the compound of general formula (VII):

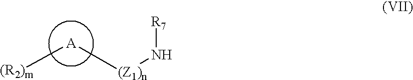

in which R₇ is selected from hydrogen, (C₁-C₆)alkyl, aryl(C₁-C₆)alkyl, cycloalkyl, aryl and heteroaryl, and A, R₂, Z₁, m and n are as defined in the summary of the invention, to give the compound of general formula (XIV) in which X₁, X₂ and X₃ are CH, W is O, Y is O, and R₇, R₁, A, R₂, Z₁, m and n are as defined hereinbefore:

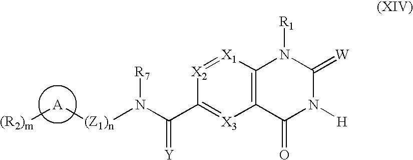

The process for manufacturing a compound of general formula (I) above is also characterized in that it comprises a step in which the compound of general formula (XIV) is reacted with compound (XV) of general formula X—R₃, in which R₃ is as defined in the summary of the invention and X is a leaving group such as halogen, to give the compound of general formula (I) in which X₁, X₂ and X₃ are CH, W is O, Y is O, Z is N—R₇, and R₇, R₁, A, R₂, Z₁, m and n are as in the compound of general formula (I).

D. Preparation process No. 3

The compounds of general formula (I) of the present invention may also be obtained by the method represented in Scheme 8 below:

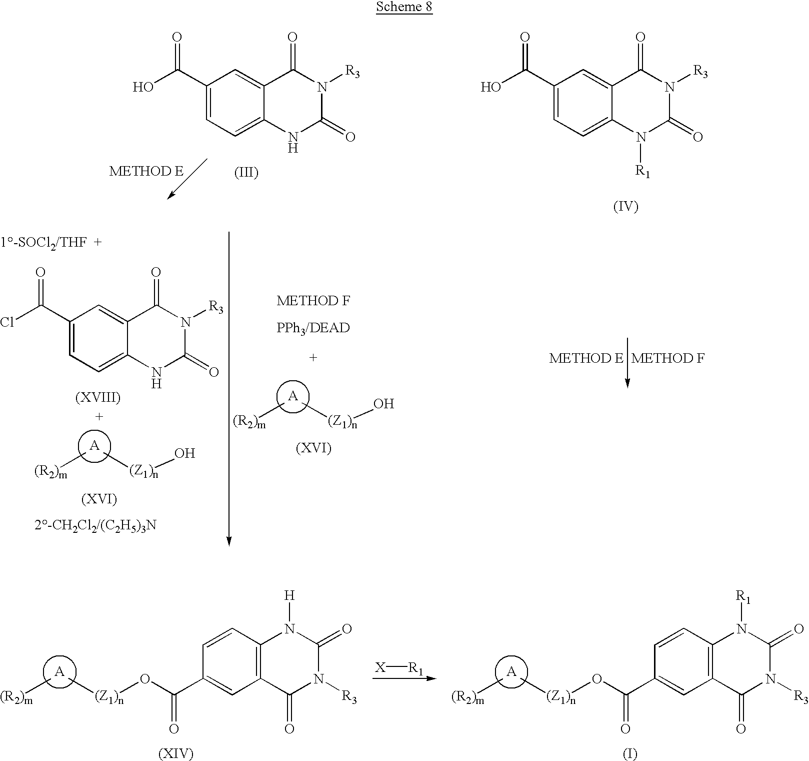

In this scheme, each generic substituent is as defined for the compound of general formula (I) above.

Thus, the present invention also relates to a process for manufacturing a compound of general formula (I) as defined above in which X₁, X₂ and X₃ are CH, W is O, Y is O and Z is O, characterized in that a compound of general formula (III):

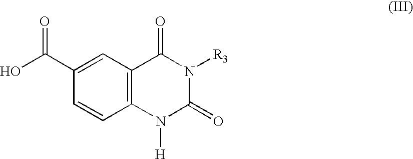

in which R₃ is as defined in the compound of general formula (I), is reacted with a compound of general formula (XVI):

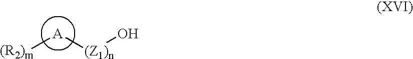

in which and A, R₂, Z₁, m and n are as defined in the compound of general formula (I), to give a compound of general formula (XVII):

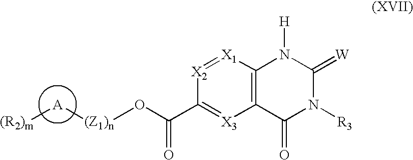

in which A, R₂, R₃, Z₁, m and n are as defined in the summary of the invention, X₁, X₂ and X₃ are CH, and W is O.

According to the process for manufacturing a compound of general formula (I) above, the said process also comprises a step in which the compound of formula (XVII) is reacted, in the presence of a base, with compound (VIII) of general formula X—R₁, in which R₁ is as defined in the summary of the invention and X is a leaving group such as halogen, to give the compound of general formula (I) in which X₁, X₂ and X₃ are CH, W is O, Y is O, Z is O, and A, R₂, R₃, R₁, Z₁, m and n are as defined in the summary of the invention

The present invention also relates to a process for manufacturing a compound of general formula (I) as defined above, characterized in that it comprises a step in which a compound of general formula (IV) is reacted with a compound of general formula (XVI) to give a compound of general formula (I) in which X₁, X₂ and X₃ are CH, W is O, Y is O and Z is O.

E. Preparation process No. 4

The compounds of the present invention, and most particularly the compounds of the invention which constitute pyridine esters, may be obtained by the method represented in Scheme 9 below:

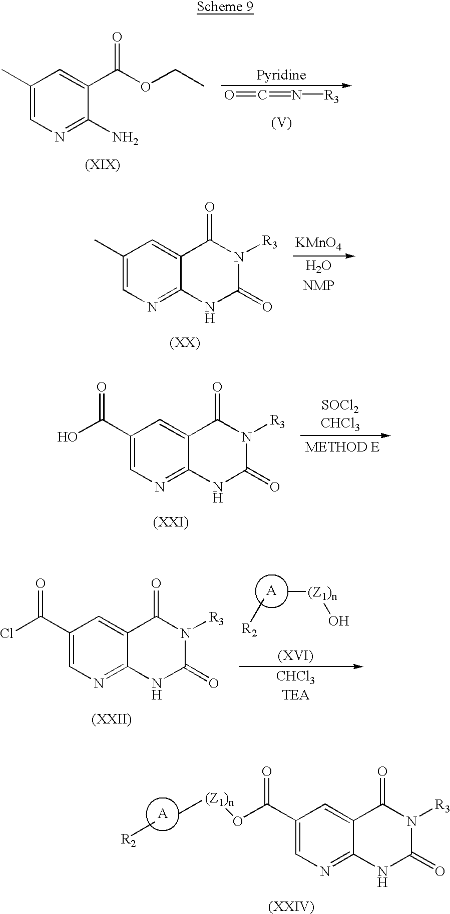

in which each of the generic substituents on the intermediate compounds has the same meaning as for the compound of general formula (I) as defined in the summary of the invention.

Consequently, a subject of the present invention is also a process for manufacturing a compound of general formula (I) in which X₂ and X₃ are CH, X₁ is N, Z is O and Y is O, characterized in that the said process comprises a step in which a compound of general formula (XIX):

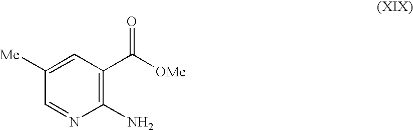

is reacted with pyridine and a compound (V) of general formula O═C═N—R₃ in which R₃ is as defined in the compound of general formula (I), to give a compound of general formula (XX):

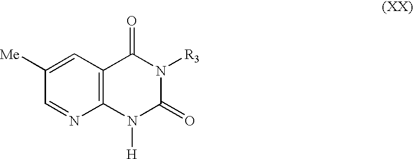

in which R₃ is as defined hereinbefore.

The process for manufacturing a compound of general formula (I) above is also characterized in that it comprises a step in which the compound of general formula (XX) is reacted in the presence of KMnO₄ to give the compound of general formula (XXI):

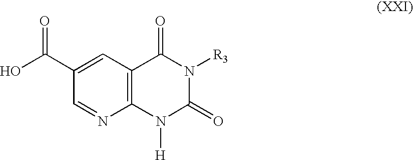

in which R₃ is as defined hereinbefore.

The above process is also characterized in that it comprises a step in which a compound of general formula (XXI) is reacted in the presence of SOCl₂ and CHCl₃ to give the compound of general formula (XXI):

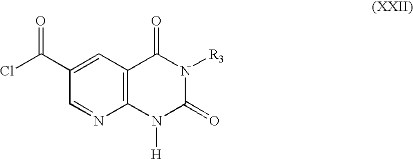

in which R₃ is as defined hereinbefore.

The process for manufacturing a compound of general formula (I) according to the invention is also characterized in that it comprises a step in which the compound of formula (XXII) is reacted with the compound of general formula (XVI):

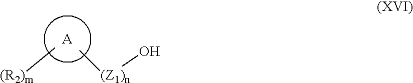

in which A, R₂, Z₁, m and n are as defined in the compound of general formula (I), to give the compound of general formula (XXIV) in which X₂ and X₃ are CH and A, n, m, Z₁, R₂ and R₃ are as defined in the summary of the invention/

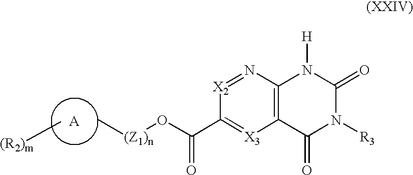

The compounds of the present invention which constitute pyridine amide can also be obtained by the method represented in scheme 10 below:

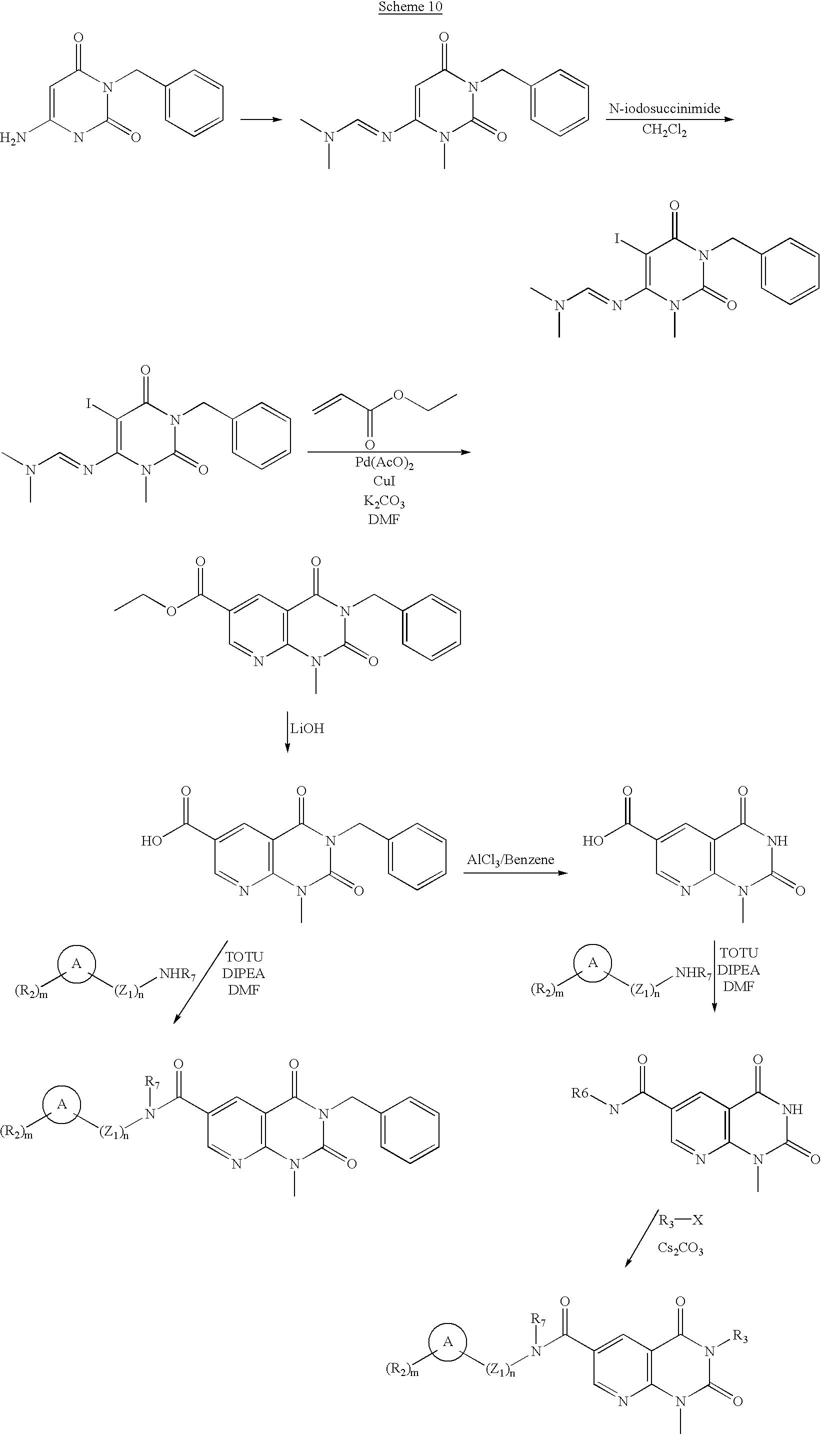

Consequently, a subject of the present invention is also a process for manufacturing a *compound of general formula (I) in which X₂ and X₃ are CH, X₁ is N, Z is —NR₇ in which R₇ is as defined in the compound of formula (I), W is O, and Y is O, characterized in that the said process comprises a step in which a compound of general (XXV):

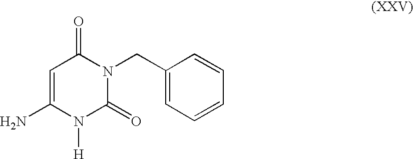

is reacted in a first step with N,N′-dimethylformamide dimethyl acetal under reflux of DMF, and in a second step with N-iodosuccinimide, to give a compound of formula (XXVI):

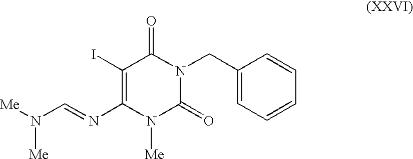

followed by reacting th compound of formula (XXVI) whith ethyl acrylate in the presence of palladium diacetate, CuI and a base, to give the compound of general formula (XXVII):

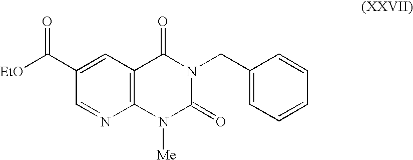

followed by reacting the compound of formula (XXVII) in the presence of LiOH to give the compound of general formula (XXVIII):

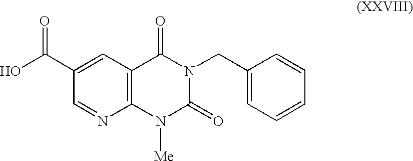

the said compound of formula (XXVIII):

either is reacted, in the presence of an acid activator such as TOTU, with the compound of formula (VII):

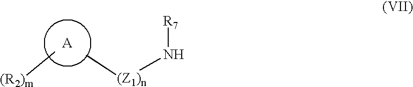

in which R₇ is selected from hydrogen, (C₁-C₆)alkyl, aryl(C₁-C₆)alkyl, cycloalkyl, aryl and heteroaryl, and A, R₂, Z₁, m and n are as defined in the summary of the invention, to give the compound of general formula (XXIX):

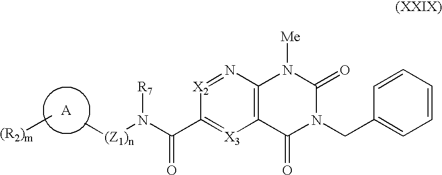

in which A, R₂, R₇, Z₁, m and n are as defined hereinbefore, and X₂ and X₃ represents each —CH group,

or is reacted in a first step with AlCl₃ in the presence of benzene, and in a second step in the presence of an acid activator such as TOTU, with the compound of formula (VII):

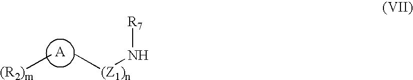

in which R₇ is selected from hydrogen, (C₁-C₆)alkyl, aryl(C₁-C₆)alkyl, cycloalkyl, aryl and heteroaryl, and A, R₂, Z₁, m and n are as defined in the summary of the invention, to give the compound of general formula (XXX):

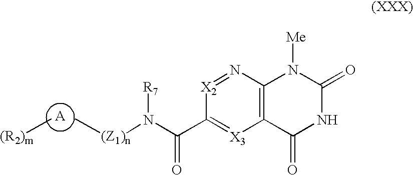

in which A, R₂, R₇, Z₁, m and n are as defined hereinbefore, and X₂ and X₃ represents each —CH group,

followed by reacting the compound of formula (XXX) with a compound of formula R₃—X in which R₃ is as defined in the compound of general formula (I), in the presence of a base, to give the compound of formula (XXXI):

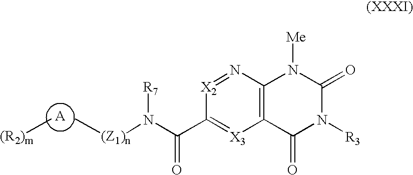

The compounds of the present invention which constitute pyridine amide, and particularly pyrido[3,4-d]pyrimidine derivatives, can also be obtained by the method represented in scheme 11 below:

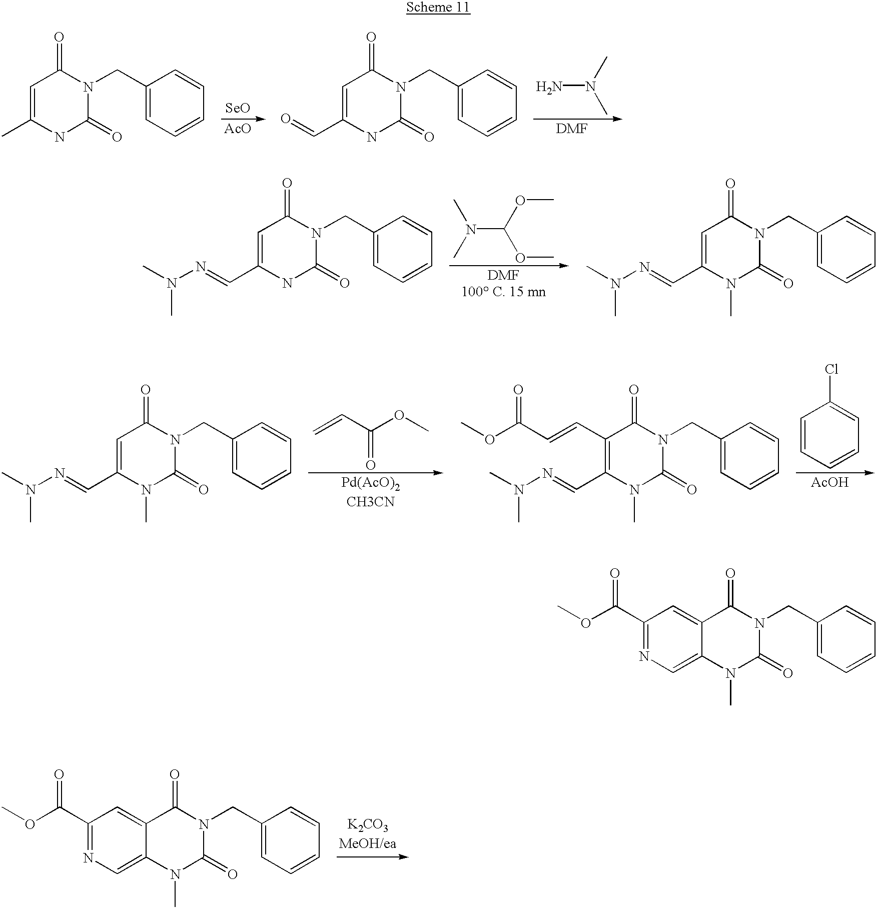

Consequently, a subject of the present invention is also a process for manufacturing a compound of general formula (I) in which X₁ and X₃ are CH, X₂ is N, Z is —NR₇ in which R₇ is as defined in the compound of formula (I), W is O, and Y is O, characterized in that the said process comprises a step in which a compound of general (XXXII):

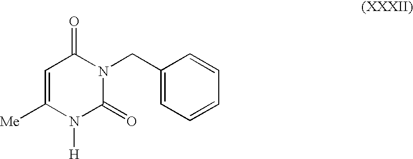

is reacted in a first step with selenium dioxide in the presence of acetic acid, in a second step with dimethylhydrazine, and in a third step with N,N′-dimethylformamide dimethylacetal under reflux of DMF, to give a compound of formula (XXXIII):

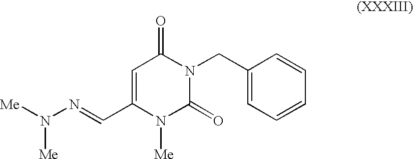

followed by reacting th compound of formula (XXXIII) whith methyl acrylate in the presence of palladium diacetate, to give the compound of general formula (XXXIV):

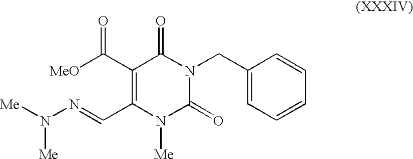

followed by reacting the compound of formula (XXXIV) whith chlorobenzene and acetic acid to give the compound of formula (XXXV):

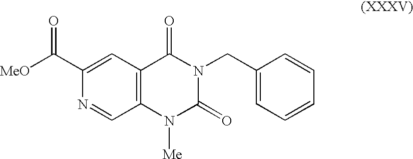

followed by reacting the compound of formula (XXXV) in the presence of a base to give the compound of general formula (XXXVI):

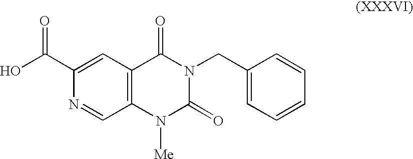

the said compound of formula (XXXVI):

either is reacted, in the presence of an acid activator such as TOTU, with the compound of formula (VII):

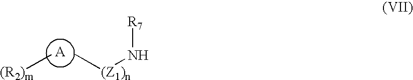

in which R₇ is selected from hydrogen, (C₁-C₆)alkyl, aryl(C₁-C₆)alkyl, cycloalkyl, aryl and heteroaryl, and A, R₂, Z₁, m and n are as defined in the summary of the invention, to give the compound of general formula (XXXVII):

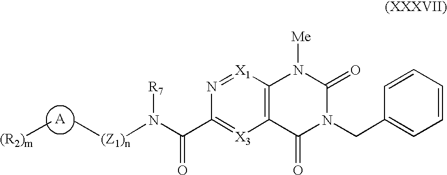

in which A, R₂, R₇, Z₁, m and n are as defined hereinbefore, and X₁ and X₃ represents each —CH group,

or is reacted in a first step with AlCl₃ in the presence of benzene, and in a second step in the presence of an acid activator such as TOTU, with the compound of formula (VII):

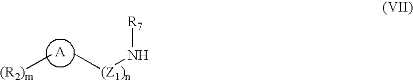

in which R₇ is selected from hydrogen, (C₁-C₆)alkyl, aryl(C₁-C₆)alkyl, cycloalkyl, aryl and heteroaryl, and A, R₂, Z₁, m and n are as defined in the summary of the invention, to give the compound of general formula (XXXVIII):

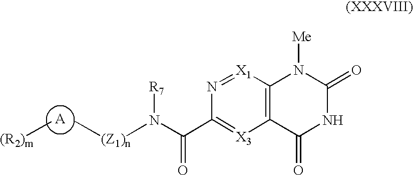

in which A, R₂, R₇, Z₁, m and n are as defined hereinbefore, and X₁ and X₃ represents each —CH group,

followed by reacting the compound of formula (XXXVIII) with a compound of formula R₃—X in which R₃ is as defined in the compound of general formula (I), in the presence of a base, to give the compound of formula (XXXIX):

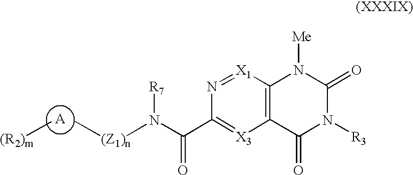

The present invention is also illustrated, without being limited thereby, in the examples which follow.

### EXAMPLES

### Preparation A: Dimethyl 4-aminoisophthalate

Preparation According to Scheme 2

Step 1-2: 4-Nitroisophthalic Acid

25 g (138 mmol) of 5-methyl-2-nitrobenzoic acid are suspended in 300 ml of water. 5 g (89.1 mmol) of KOH are added for dissolution. The medium is heated to 90° C. and 158 g of KMnO₄ (414 mmol) are added portionwise, rinsing with H₂O. After 3 hours, the reaction medium is filtered through Celite and the filtrate is acidified to pH 1 with concentrated HCl. The precipitate obtained is filtered off and dried under vacuum.

Weight=15.3 g; Yield=53%

NMR: DMSO ¹H δ(ppm) 5.62-5.70 (d,1H); 7.88 (d,1H); 8.16 (s,1H)

Step 2-2: Dimethyl 4-nitroisophthalate

12.75 g (60.4 mmol) of 4-nitroisophthalic acid from the above stage and 13 ml of H₂SO₄ and 100 ml of methanol are maintained at reflux overnight. After cooling, the methanol is removed under vacuum. The residue is dissolved in 400 ml of EtOAc. The organic phase is washed with 50 ml of H₂O and then with 50 ml of 5% NaHCO₃ solution. Drying over MgSO₄ and concentration under vacuum gives a crystalline residue.

Weight=12.17 g Yield=84%

NMR: DMSO ¹H δ(ppm) 3.86 (s,3H); 3.91 (s,3H); 8.16 (d,1H); 8.29-8.34 (m,2H)

Step 3-2: Dimethyl 4-aminoisophthalate

The compound from the above stage is reduced with hydrogen in the presence of palladium as catalyst.

Filtration through Celite and concentration gives:

Weight=5.12 g Yield=70%

m.p.=127-128° C.

NMR: CDCl₃ ¹H δ(ppm) 3.87 (s,3H); 3.88 (s,3H); 6.30 (brs,2H); 6.65 (d,1H); 7.89 (dd,1H); 8.57 (d,1H)

Preparation According to Scheme 3—J. Org. Chem., 1997, 62 (12), 4088-4096

Step 1-3: Dimethyl 4-amino-1-hydroxycyclohexa-3,5-diene-1,3-dicarboxylate

526 ml of benzene and 250 ml of methyl acrylate are introduced into a 1-liter three-necked flask fitted with a reflux condenser, placed under inert atmosphere and protected from moisture, followed by 10 g (70.8 mmol) of methyl 5-amino-2-furan carboxylate. The mixture is brought to reflux and maintained for 24 hours. The reaction medium is concentrated to dryness on a rotavapor at 50° C. under a vacuum of 20 mm Hg. The residue obtained is purified by flash chromatography using dichloromethane progressively enriched with ethyl acetate as solvent. The product is obtained as follows:

Weight=15 g of a yellow precipitate Yield=93%

TLC: CH₂Cl₂/EtOAc 70/30 v/v Rf=0.35

m.p.=101.3° C.

NMR: CDCl₃ ¹H δ(ppm) 2.87 (d,1H); 2.93 (d,1H); 3.20 (s,1H); 3.71 (s,3H); 3.82 (s,3H) 6.02 (d,1H); 5.60-6.40 (brs,2H); 6.17 (d,1H)

Step 2-3: Dimethyl 4-aminoisophthalate

15 g (66 mmol) of compound obtained in Step 1-3 and 600 ml of benzene are introduced into a 1-liter three-necked flask fitted with a reflux condenser, placed under an inert atmosphere and protected from moisture. 13.8 g (12 ml, 98 mmol) of BF₃ etherate are added with stirring. The mixture is refluxed for 2 minutes and then cooled to room temperature and, after addition of saturated NaHCO₃ solution (pH 9), the phases are separated by settling. The aqueous phase is re-extracted twice with dichloromethane. The organic phases are combined and dried over Na₂SO₄. After removal of the solvents under vacuum, the 13.8 g of residue are purified by chromatography using dichloromethane as elution solvent. The product is obtained as follows:

Weight=8.5 g of a crystallyne residue Yield=62%

TLC: CH₂Cl₂. Rf=0.30

m.p.=130.1° C.

NMR: CDCl₃¹H δ(ppm) 3.87 (s,3H); 3.88 (s,3H); 6.30 (brs,2H); 6.65 (d,1H); 7.89 (dd,1H); 8.57 (d,1H)

### Preparation B: 3-Benzyl-2,4dioxom-1,2,3,4-tehydroquinazoline-6-carboxylic Acid

Preparation According to Scheme 4

Step 1-4: Methyl 3-benzyl-2,4-dioxo-1,2,3,4-tetrahydroquinazoline -6-carboxylate

4 g (19.1 mmol) of compound of preparation A and 40 ml of pyridine are successively introduced into a 50 ml three-necked flask fitted with a reflux condenser and protected from moisture, followed by addition of 3.2 g (24 mmol) of benzyl isocyanate. The colourless solution is stirred and heated at 95-100° C. After 6 hours at this temperature, 1 ml of benzyl isocyanate is added and stirring is then continued at 100° C. overnight. The next day, the reaction medium is cooled and poured into 400 ml of a water+ice mixture, it is left stirring for about 30 minutes and the precipitate obtained is then filtered off. The product is re-slurried at reflux in 150 ml of ethanol. After cooling, the product is filtered off. The product is obtained as follows:

Weight=3.7 g Yield=62%

NMR: DMSO ¹H δ(ppm): 3.75 (s,3H); 4.95 (s,2H); 7.1-7.2 (m,6H); 8.05 (d,1H); 8.35 (s,1H); 11.8 (bs,1H)

Step 2-4: 3-Benzyl-2,4-dioxo-1,2,3,4-tetrahydroquinazoline-6-carboxylic acid

1.5 g (4.84 mmol) of methyl 3-benzyl-2,4-dioxo-1,2,3,4-tetrahydroquinazoline-6-carboxylate, 14 ml of dioxane and 48 ml of H₂O are introduced into a 100 ml round-bottomed flask fitted with a reflux condenser. 0.41 g (9.68 mmol) of hydrated lithium hydroxide is added to the suspension with stirring. The mixture is brought to reflux and maintained for about 1 hour (solution). After cooling in an ice bath, the medium is acidified to pH 1 with concentrated hydrochloric acid. The very fine precipitate obtained is filtered off, to give:

Weight: 1.3 g Yield=96%

NMR: DMSO ¹H δ(ppm): 5.1 (s,2H); 7.2-7.35 (m,6H); 8.15 (d,1H); 8.48 (s,1H); 11.85 (s,1H); 13.1 (bs,1H)

Preparation According to Scheme 5

Step 1-5: Dimethyl 4-(3-benzylureido)isophthalate

10 g (48 mmol) of compound of Preparation A, 200 ml of anhydrous toluene, about 100 mg of animal charcoal and then 12 g (40 mmol) of triphosgene are introduced into a 1-liter one-necked flask fitted with a reflux condenser and protected from moisture. The suspension is stirred and maintained at the reflux point of the toluene for 2 hours. The reaction medium is filtered through infusorial earth and then concentrated to dryness at 50° C. under a vacuum of about 20 mm Hg. The residue obtained is dissolved in 200 ml of anhydrous toluene and stirred.

4.7 ml (43 mmol) of benzylamine are added to this solution over a few minutes. A precipitate is immediately formed. 200 ml of toluene are added to facilitate stirring, and the mixture is maintained at room temperature overnight. The next day, the precipitate is filtered off and washed successively with toluene and ether. After drying under vacuum, the product is obtained as follows:

Weight 13.9 g Yield =84.6%

TLC: CH₂Cl₂/acetone 98/2 Rf=0.35

m.p.=181.9° C.

NMR: DMSO ¹H δ(ppm) 3.8 (s,3H); 3.9 (s,3H); 4.3 (s,2H); 7.2-7.4 (m,5H); 8.0 (d,1H); 8.3 (s,1H); 8.5 (s,1H); 8.55 (d,1H); 10.2 (s,1H)

Step 2-5: Methyl 3-benzyl-2,4-dioxo-1,2,3,4-tetrahydroquinazoline -6-carboxylate

13.7 g (40 mmol) of compound obtained in Step 1-5, 300 ml of methanol and then 1.3 g (24 mmol) of sodium methoxide are introduced into a 1-litre one-necked flask fitted with a reflux condenser and protected from moisture. The white suspension is maintained at reflux for 3 hours (the suspension changes form). Half of the methanol is removed on a rotavapor at 50° C. under vacuum. The mixture is cooled and acidified to pH 4 with 2 ml of concentrated hydrochloric acid. It is left stirring for 15 minutes while cold and the crystalline residue obtained is then filtered off.

Weight=12 g Yield=96.7%

TLC: CH₂Cl₂/acetone 98/2

Rf=0.05-0.2

m.p.=248.1° C.

NMR: DMSO ¹H δ(ppm) 3.9 (s,3H); 5.1 (s,2H); 7.2-7.4 (m,6H); 8.15 (d,1H); 8.45 (s,1H); 11.9 (bs,1H)

Step 3-5: 3-benzyl-2,4-dioxo-1,2,3,4-tetrahydroquinazoline-6-carboxylic acid

The product is obtained according to the procedure of Step 2-4 of Preparation B using the compound obtained in preceding Step 2-5.

Preparation According to Scheme 6

Step 1-6: 3-Benzyl-6-bromo-1H-quinazoline-2,4-dione

10 g (46.3 mmol) of 2-amino-5-bromobenzoic acid, 100 ml of anhydrous pyridine and 6.16 g (46.3 mmol) of benzyl isocyanate are introduced into a 250 ml one-necked flask fitted with a reflux condenser and protected from moisture. The solution is maintained at reflux with stirring for 36 hours. The reaction mixture is cooled and H₂O is added until the start of precipitation. The mixture is left to crystallize for about 1 hour and the precipitate obtained is then filtered off and washed. The 8 g of crude product are purified by reslurrying in refluxing ethanol.

Weight: 3.4 g

NMR:=DMSO ¹H δ(ppm): 4.9 (s,2H); 7.0 (d,1H); 7.03-7.2 (m,5H); 7.65 (d,1H); 7.85 (s,1H); 11.5 (s,1H)

Step 2-6: 3-Benzyl-2,4-dioxo-1,2,3,4-tetrahydroquinazoline-6-carbonitrile

2.5 g (7.5 mmol) of compound of Step 1-6, 1.215 g (13.6 mmol) of copper cyanide and 22.5 ml of 1-methyl-2-pyrrolidinone are introduced into a 50 ml three-necked flask fitted with a reflux condenser and protected from moisture. The beige-coloured solution obtained is refluxed at an internal temperature of 200° C. for 1 h 30 min. The reaction medium is concentrated to dryness at 80° C. under a vacuum <1 mm Hg. The residue is taken up in 300 ml of 2N NH₄OH and extracted 3 times with dichloromethane. The presence of an insoluble material is noted, this material being taken up twice in 20 ml of a 50/50 v/v MeOH/CH₂Cl₂ mixture. The organic phases are combined and washed with H₂O. After drying over Na₂SO₄ and concentration under vacuum, the black residue obtained is crystallized from 10 ml of CH₂Cl₂. The product is obtained as follows:

Weight: 1.2 g Yield=60%

TLC: CH₂Cl₂/MeOH 90/10 Rf=0.50

NMR: DMSO ¹H δ(ppm): 4.82 (s,2H); 6.97-7.12 (m,6H); 7.80 (d,1h); 8.1 (s,1H); 11.75 (bs,1H)

Step 3-6: 3-Benzyl-2,4-dioxo-1,2,3,4-tetrahydroquinazoline-6-carboxylic acid

1.4 g (5.05 mmol) of compound of Step 2-6 and 35 ml of H₂O are introduced into a 100 ml one-necked flask fitted with a reflux condenser, followed by cautious addition of 35 ml of H₂SO₄. The suspension is maintained at reflux with stirring for 3 hours. After cooling, the beige-coloured precipitate is filtered off and washed to neutrality with H₂O and then with methanol.

Weight: 1.5 g Yield=100%

TLC: CH₂Cl₂/MeOH 90/10 Rf=0.10

m.p.=360° C.

### Preparation C: 3-Benzyl-1-methyl-2,4-dioxo-1,2,3,4-tetrahydroquinazoline-6-carboxylic acid

Step 1: Methyl 3-benzyl-1-methyl-2,4-dioxo-1,2,3,4-tetrahydroquinazoline-6-carboxylate

11.8 g (38.0 mmol) of Preparation B, 120 ml of dimethylformamide and 7.9 g (57 mmol) of K₂CO₃ are introduced into a 250 ml three-necked flask. The suspension is stirred for 15 minutes at room temperature. 27 g (12 ml, 190 mmol) of iodomethane are added over 2 minutes. The suspension is stirred at room temperature for 30 to 45 minutes. The solvent is removed under vacuum and the residue is taken up in 500 ml of dichloromethane and washed with 3 times 300 ml of water. The organic phase is dried and the solvent is removed. The product is obtained as follows:

Weight: 12 g Yield=97.4%

TLC: CH₂Cl₂/acetone 98/2 Rf=0.60

m.p.=179.3° C.

NMR: DMSO ¹H δ(ppm) 3.6 (s,3H); 3.90 (s,3H); 5.1 (s,2H); 7.2-7.4 (m,5H); 7.55 (d,1H); 8.25 (d,1H); 8.6 (s,1H)

Step 2: 3-Benzyl-1-methyl-2,4-dioxo-1,2,3,4-tetrahydroquinazoline-6-carboxylic acid

The product is obtained with a yield of 100% (10 g) according to the procedure of Step 2-4 of Preparation B using 9.5 g (29.3 mmol) of compound obtained in Step 1.

TLC: CH₂Cl₂/MeOH 90/10 Rf=0.50

m.p.=227.2° C.

NMR: DMSO ¹H δ(ppm) 3.55 (s,3H); 5.15 (s,2H); 7.2-7.4 (m,5H); 7.55 (d,1H); 8.25 (d,1H); 8.6 (s,1H); 13.2 (bs,1H)

### Preparation D: 1-Methyl-3-fluorobenzyl)-2,4-dioxo-1,2,3,4-teatrahydroquinazoline-6-carboxylic Acid

Step 1: Methyl 3-(3-fluorobenzyl)-2,4-dioxo-1,2,3,4-tetrahydroquinazoline-6-carboxylate

5.5 g (26.3 mmol) of compound of the Preparation A and 50 ml of pyridine are introduced into a round-bottomed flask. 5.0 g (33.1 mmol) of 3-fluorobenzyl isocyanate are added. The mixture is maintained at reflux for 6 hours and 0.8 g (5.3 mmol) of 3-fluorobenzyl isocyanate is added in one portion. The mixture is heated overnight at reflux. The mixture is cooled and the product is precipitated with the addition of water and filtered. The product is reslurryed in hot ethanol and filtered to provide 6.7 g (yield:78%) of the desired compound.

MS: m/z (APCI, AP+) 329.1 [M]⁺

CHN Analysis: Calcd (%): C, 62.20; H, 3.99; N, 8.53.

Found (%): C, 62.09; H, 3.85; N, 8.42.

Step 2: Methyl 1-methyl-3-(3-fluorobenzyl)-2,4-dioxo-1,2,3,4-tetrahydroquinazoline-6-carboxylate

1.8 g (5.5 mmol) of the product from the preceding Step 1 is dissolved in 30 ml of dimethylformamide and 1.8 g (8.1 mmol) of cesium carbonate is added. The mixture is stirred 10 minutes before adding iodomethane 1.1 g (8.1 mmol). Stirring is continued overnight at room temperature. Water (60 ml) is added and the product is extracted with ethyl acetate (2×30 ml). The organic extracts are combined and washed with saturated aqueous NaCl solution (4×20 ml), and dried MgSO₄. Slurried solid product in hot ethyl acetate and filtered to obtain 1.7 g (yield: 90%) of the desired compound.

MS: m/z (APCI, AP+) 343.1 [M]⁺

CHN Analysis: Calcd (%): C, 63.16; H, 4.42; N, 8.18.

Found (%): C, 63.02; H, 4.26; N, 8.06.

Step 3: 1-Methyl-3-(3-fluorobenzyl)-2,4-dioxo-1,2,3,4-tetrahydroquinazoline-6-carboxylic Acid

0.71 g of the compound (yield:76%) is obtained according to the procedure of the Step 2-4 of Preparation B using the compound obtained in the preceding Step 2.

MS: m/z (APCI, AP+) 329.0 [M]⁺

CHN Analysis: Calcd (%): C, 62.20; H, 3.99; N, 8.53. Found (%): C, 61.94; H, 3.78; N, 8.57.

### Preparation E: 1-Ethyl-1-3-fluorobenzyl)-2,4-dioxo-1,2,3,4-tetrahydroquinazoline-6-carbolic acid

Step 1: Methyl 1-ethyl-3-(3-fluorobenzyl)-2,4-dioxo-1,2,3,4-tetrahydroquinazoline-6-carboxylate

2.0 g (6.1 mmol) of the compound of Step 1 of Preparation D are dissolved in 30 ml of dimethylformamide and 1.96 g (9.2 mmol) of cesium carbonate is added. The mixture is stirred 10 minutes before adding 1.4 g (9.2 mmol) of iodoethane. Stirring is continued overnight at room temperature. Water (60 ml) is added and the product is extracted with ethyl acetate (2×30 ml). The organic extracts are combined and washed with saturated aqueous NaCl solution (4×20 ml), and dried MgSO₄. Slurried solid product in hot ethyl acetate and filtered to obtain 1.4 g (yield: 67%) of the desired compound.

MS: m/z (APCI, AP+) 357.1 [M⁻]⁺

CHN Analysis: Calcd (%): C, 64.04; H, 4.81; N, 7.86. Found (%): C, 63.72; H, 4.68; N, 7.75.

Step 2: 1-Ethyl-3-(3-fluorobenzyl)-2,4-dioxo-1,2,3,4-tetrahydroquinazoline-6-carboxylic acid

1.1 g of the compound (yield: 71%) is obtained according to the procedure of the Step 2-4 of Preparation B using the compound obtained in the preceding Step 1.

MS: m/z (APCI, AP+) 343.0 [M⁻]⁺

CHN Analysis: Calcd (%): C, 63.16; H, 4.42; N, 8.18. Found (%): C, 63.06; H, 4.41; N, 8.03.

Examples 1 to 461 illustrate, without limiting it, the synthesis of particularly active compounds of formula (I) according to the invention.

### Example 1

### 3-Benzyl-2,4-dioxo-1,2,3,4-tetrahydroquinazoline-6-carboxylic acid benzylamide

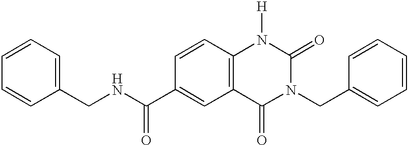

0.150 g (0.51 mmol) of compound of Preparation B and 8.0 ml of anhydrous dimethylformamide are introduced into a stirred 25 ml one-necked flask protected from moisture. 0.054 g (56 μl, 0.51 mmol) of benzylamine and 0.17 g (0.51 mmol) of TOTU are added to this solution. The solution is cooled in a bath to 0° C. 0.132 g (0.18 ml, 1.02 mmol) of N,N-diisopropylethylamine is then added. The mixture is warmed to room temperature and stirred overnight. After monitoring by TLC (90/10 CH₂Cl₂/MeOH), the DMF is removed under vacuum. The crystalline residue obtained is taken up in dichloromethane with the amount of methanol required for total dissolution. The organic phase is washed successively with 40 ml of 1N HCl, 40 ml of H₂O, 40 ml of saturated NaHCO₃ solution and finally 40 ml of H₂O. The organic phase is dried over Na₂SO₄ and the solvents are removed under vacuum. 0.140 g of product is obtained, which is recrystallized from 30 ml of acetonitrile:

Weight: 0.110 g Yield—56%

TLC: CH₂Cl₂/MeOH 90/10 Rf=0.65

NMR: DMSO ¹H δ(ppm): 4.45 (d,2H); 5.1 (s,2H); 7.1-7.4 (m,11H); 8.1 (d,1H); 8.5 (s,1H); 9.15 (m,1H); 11.75 (bs,1H)

IR: 3425,2364,1722,1640,1509,1442,1304,1261,1078,927,845 cm⁻¹

m.p.=241.2° C.

HPLC: 98.3%

### Example 2

### 3-Benzyl-2,4-dioxo-1,3,4-tetrahydroquinazoline-6-carboxylic acid (4-pyridylmethyl)amide

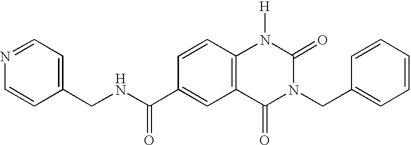

The product is obtained with a yield of 46% (0.090 g) according to the procedure of Example 1 using 4-picolylamine, and after recrystallization from a 50/50 EtOAc/EtOH mixture.

TLC: CH₂Cl₂/MeOH 90/10 Rf=0.60

NMR: DMSO ¹H δ(ppm): 4.5 (d,2H); 5.1 (s,2H); 7.2-7.4 (m,8H); 8.15 (d,1H); 8.5 (d,2H); 8.55 (s,1H); 9.25 (t,1H); 11.75 (s,1H)

IR: 3250,1725,1669,1642,1623,1450,1345,1301,1075,1006, 830 cm⁻¹

m.p.=305.2° C.

HPLC: 95.1%

### Example 3

### 3Benzyl-2,4-dioxo-1,2,4-tetrahydroquinazoline-6-carboxylic acid (benzo[1,3]dioxol-5-ylmethyl)amide

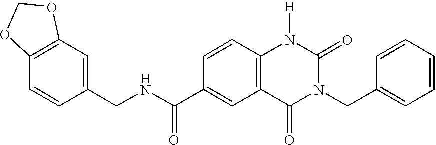

The product is obtained with a yield of 64% (0.140 g) according to the procedure of Example 1 using piperonylamine, and after crystallization from acetonitrile.

TLC: CH₂Cl₂/MeOH 90/10 Rf=0.65

NMR: DMSO ¹H δ(ppm): 4.35 (d,2H); 5.1 (s,2H); 5.95 (s,2H);6.7-6.95 (m,3H); 7.15-7.4 (m,6H); 8.15 (d,1H); 8.5 (s,1H); 9.1 (t,1H); 11.7 (bs,1H)

IR: 3200,1727,1636,1493,1444,1299,1261,1041,938,841,763,726 cm⁻¹

m.p.=256° C.

HPLC: 99%

### Example 4

### 3-Benzyl-2,4-dioxo-1,2,3,4-tetrahydroquinazoline-6-carboxylic acid (2-thienylmethyl)amide

The product is obtained with a yield of 40% (0.080 g) according to the procedure of Example 1, but using 2-thienylmethylamine, and after a crystallization from acetonitrile.

TLC: CH₂Cl₂/MeOH 90/10 Rf=0.65

NMR: DMSO ¹H δ(ppm): 4.35 (d,2H); 4.85 (s,2H); 6.7-6.85 (m,2H); 6.95-7.2 (m,7H); 7.9 (d,1H); 8.3 (s,1H); 9.05 (t,1H); 11.55 (bs,1H)

IR: 1729,1637,1511,1444,1346,1298,1261,1072,845,763 cm⁻¹

m.p.=236.3° C.

HPLC: 98.7%

### Example 5

### 3-Benzyl-2,4-dioxo-1,2,3,-tetrahydroquinazoline-6-carboxylic acid (3-pyridylmethyl)amide

The product is obtained with a yield of 66% (0.130 g) according to the procedure of Example 1, but using 3-(aminomethyl)-pyridine, and after a crystallization from acetonitrile.

TLC: CH₂Cl₂/MeOH 95/5 Rf=0.40

NMR: DMSO ¹H δ(ppm): 4.5 (d,2H); 5.15 (s,2H); 7.15-7.4 (m,7H); 7.7 (d,1H); 8.15 (d,1H); 8.45 (d,1H); 8.55 (d,2H); 9.25 (t,1H); 11.8 (s,1H)

IR: 3345,1716,1670,1638,1621,1450,1433,1348,1298,1068,829,774 cm⁻¹

m.p.=252.3° C.

HPLC: 97.4%

### Example 6

### 3-Benzyl-2,4-dioxo-1,2,3,4-tetrahydroquinazoline-6-carboxylic acid 4-methoxybenzylamide

The product is obtained with a yield of 47.2% (0.100 g) according to the procedure of Example 1, but using 4-methoxybenzylamine, and after a crystallization from acetonitrile.

TLC: CH₂Cl₂/MeOH 95/5 Rf=0.45

NMR: DMSO ¹H δ(ppm): 3.7 (s,3H); 4.4 (d,2H); 5.1 (s,2H); 6.9 (d,2H); 7.2-7.4 (m,8H); 8.15 (d,1H); 8.5 (s,1H); 9.15 (t,1H); 11.8 (bs,1H)

IR: 3400,3210,1727,1638,1513,1441,1300,1253,1173,1040,843, 760 cm⁻¹

m.p.=269° C.

HPLC: 100%

### Example 7

### 3-Benzyl-2,4-dioxo-1,2,3,4-tetrahydroquinzaoline-6carboxylic acid 4-chlorobenzylamide

The product is obtained with a yield of 19% (0.040 g) according to the procedure of Example 1, but using 4-chlorobenzylamine, and after a crystallization from acetonitrile.

TLC: CH₂Cl₂/NMeOH 95/5 Rf=0.45

NMR: DMSO ¹H δ(ppm): 4.5 (d,2H); 5.1 (s,2H); 7.2-7.45 (m,10H); 8.15 (d,1H); 8.5 (s,1H); 9.25 (t,1H); 11.8 (bs,1H)

IR: 3365,3200,1726,1638,1551,1512,1444,1305,1263,1012,844, 763 cm⁻¹

m.p.=280.6° C.

HPLC: 98.1%

### Example 8

### 3-Benzyl-2,4-dioxo-1,2,3,4-tetrahydroquinazoline-6-carboxylic acid 4-methylbenzylamide

The product is obtained with a yield of 19% (0.040 g) according to the procedure of Example 1, but using 4-methylbenzyl amine, and after a crystallization from acetonitrile.

TLC: CH₂Cl₂/MeOH 95/5 Rf=0.40

NMR: DMSO ¹H δ(ppm): 2.3 (s,3H); 4.4 (d,2H); 5.1 (s,2H); 7.0-7.4 (m,10H); 8.15 (d,1H); 8.55 (s,1H); 9.1 (t,1H); 11.8 (bs,1H)

IR: 3280,1720,1671,1640,1623,1550,1278,848,774,744 cm⁻¹

m.p.=267.8° C.

HPLC: 98.7

### Example 9

### 3-Benzyl-1-methyl-2,4-dioxo-1,2,3,4-tetrahydroquinazoline-6-carboxylic acid (benzo[1,3]dioxol-5-ylmethyl)amide

0.500 g (1.61 mmol) of compound of Preparation C in 25 ml of anhydrous dimethylformamide are introduced into a stirred 50 ml one-necked flask protected from moisture. 0.244 g (0.201 ml, 1.61 mmol) of piperonylamine and 0.531 g (1.61 mmol) of TOTU are added to this solution. The solution is cooled in a cold bath to 0° C. 0.415 g (0.564 ml, 3.22 mmol) of N,N-diisopropylethylamine is then added. The mixture is warmed to room temperature and stirred overnight. After monitoring by TLC (90/10 CH₂Cl₂/MeOH), DMF is removed under vacuum. The crystalline residue obtained is taken up in dichloromethane. The organic phase is washed successively with 1N HCl, H₂O, saturated NaHCO₃ and finally H₂O. The organic phase is dried over Na₂SO₄ and the solvent is removed under vacuum. 0.540 g of product, recrystallized from 30 ml of acetonitrile, is obtained as follows:

Weight: 0.390 g Yield=54.6%

TLC: CH₂Cl₂/acetone 90/10 Rf=0.40

NMR: DMSO ¹H δ(ppm): 3.55 (s,3H); 4.35 (d,2H); 5.15 (s,2H); 6.0 (s,2H); 6.75-6.95 (m,3H); 7.2-7.4 (m,5H); 7.55 (d,1H); 8.25 (d,1H); 8.65 (s,1H); 9.2 (t,1H)

IR: 3303,1703,1656,1637,1498,1444,1322,1254,1040,932,845 cm⁻¹

m.p.=215.1° C.

HPLC: 99.5%

### Example 10

### 3-Benzyl-1-methyl-2,4-dioxo-1,2,3,4-tetrahydroquinazoline-6carboxylic acid benzylamide

The product is obtained with a yield of 56.8% (0.110 g) according to the procedure of Example 9, but using benzylamine, and after a crystallization from acetonitrile.

TLC: CH₂Cl₂/acetone 90/10 Rf=0.55

NMR: CDCl₃¹H δ(ppm) 3.65 (s,3H); 4.65 (d,2H); 5.3 (s,2H); 6.55 (m,1H); 7.2-7.6 (m,11H); 8.3 (d,1H); 8.5 (s,1H);

IR: 1708,1655,1641,1616,1507,1478,1326,1246,930,750 cm⁻¹

m.p.=198.9° C.

HPLC: 100%

### Example 11

### Methyl 4-({[1-(3-benzyl-1-methyl-2,4-dioxo-1,2,3,4-tetrahydroquinazolin-6-yl)methanoyl]amino}methyl)benzoate

The product is obtained with a yield of 61.5% (0.135 g) according to the procedure of Example 9, but using methyl 4-(aminomethyl)benzoate hydrochloride and 3.5 equivalents of N,N-diisopropylethylamine. The crude product is purified by chromatography on silica, using a 95/5 CH₂Cl₂/MeOH gradient, followed by a solidification in ether.

TLC: CH₂Cl₂/MeOH 95/5 Rf=0.36

NMR: DMSO ¹H δ(ppm): 3.55 (s,3H); 3.85 (s,3H); 4.55 (d,2H); 5.15 (s,2H); 7.2-7.35 (m,5H); 7.45 (d,2H); 7.6 (d,1H); 7.95 (d,2H); 8.3 (d,1H); 8.65 (s,1H); 9.35 (t,1H)

IR: 1723,1706,1657,1642,1617,1506,1477,1284,1109,749 cm⁻¹

m.p.=196° C.

HPLC: 100%

### Example 12

### 3-Benzy-1-methyl-2,4-dioxo-1,3,3,4-tetrahydroquinazoline-6-carboxylic acid 4-hydroxy-3-methoxybenzylamide

The product is obtained with a yield of 42% (0.090 g) according to the procedure of Example 9, but using 4-hydroxy-3-methoxybenzylamine hydrochloride and 3.5 equivalents of N,N-diisopropylethylamine. The crude product is purified by chromatography on silica, using a 95/5 CH₂Cl₂/MeOH gradient, followed by a solidification in ether.

TLC: CH₂Cl₂/MeOH 95/5 Rf=0.59

NMR: DMSO ¹H δ(ppm): 3.55 (s,3H); 3.75 (s,3H); 4.4 (d,2H); 5.15 (s,2H); 6.75 (s,2H); 6.95 (s,1H); 7.2-7.40 (m,6H); 7.55 (d,1H); 8.3 (d,1H); 8.65 (s,1H); 8.8 (s,1H); 9.15 (t,1H)

IR: 1707,1655,1618,1502,1477,1277,704 cm⁻¹

m.p.=183° C.

HPLC: 87.1%

### Example 13

### 3-Benzyl-1-methyl-2,4-dioxo-1,2,3,4-tetrahydroquinazoline-6-carboxylic acid 4-methoxybenzylamide

The product is obtained with a yield of 77.7% (0.320 g) according to the procedure of Example 9, but using 4-methoxybenzylamine. The crude product is purified by chromatography on silica, using 97/3 CH₂Cl₂/MeOH as eluent. The desired fractions are combined and concentrated. The product is solidified in ether and then filtered off

TLC: CH₂Cl₂/MeOH 90/10 Rf=0.8

NMR: DMSO ¹H δ(ppm): 3.55 (s,3H); 3.75 (s,3H); 4.45 (d,2H); 5.2 (s,2H); 6.9 (d,2H); 7.2-7.4 (m,7H); 7.6 (d,1H); 8.3 (d,1H); 8.65 (s,1H); 9.25 (t,1H)

IR: 1705,1660,1636,1505,1251,750 cm⁻¹

m.p.=191° C.

HPLC: 97.3%

### Example 14

### 3-Benzyl-1-methyl-2,4-dioxo-1,2,3,4-tetrahydroquinazoline-6-carboxylic acid (4-pyridylmethyl)amide

The product is obtained with a yield of 67.7% (0.130 g) according to the procedure of Example 9, but using 4-picolylamine. The crude product is purified by chromatography on silica, using 95/5 CH₂Cl₂/MeOH as eluent. The desired fractions are combined and concentrated. The product is solidified in ether and then filtered off.

TLC: CH₂Cl₂/MeOH 90/10 Rf=0.18

NMR: DMSO ¹H δ(ppm): 3.60 (s,3H); 4.55 (d,2H); 5.15 (s,2H); 7.2-7.4 (m,7H); 7.6 (d,1H); 8.3 (d,1H); 8.5 (d,2H); 8.65 (s,1H); 9.35 (t,1H)

IR: 1705,1658,1634,1508,1332,831,749,705 cm⁻¹

m.p.=172° C.

HPLC: 98.8%

### Example 15

### 1-Methyl-2,4-dioxo-3-phenethyl-1,2,3,4-tetrahydroquinazoline-6-carboxylic acid (benzo[1,3]dioxol-5-ylmethyl)amide

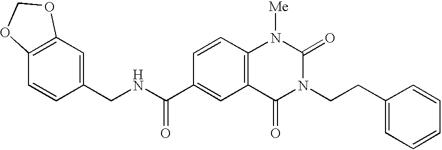

Step 1: Methyl-2,4-dioxo-3-phenethyl-1,2,3,4-tetrahydroquinazoline-6-carboxylate

0.750 g (3.6 mmol) of compound of Preparation A and 7.5 ml of pyridine are introduced into a round-bottomed flask. 0.530 g (0.5 ml; 3.6 mmol) of phenethyl isocyanate is added.

The mixture is maintained at 100° C. overnight. Since the reaction is incomplete, a second addition of phenethyl isocyanate, i.e. 2 equivalents, is carried out. After precipitation with H₂O, filtration and purification by reslurrying in hot ethanol, the product is obtained as follows:

Weight:0.640 g Yield=54.9%

NMR: DMSO ¹H δ(ppm): 2.85-2.95 (m,2H); 4.90 (s,3H); 4.05-4.15 (m,2H); 7.15-7.3 (m,6H); 8.15 (d,1H); 8.45 (s,1H); 11.8 (bs,1H)

Step 2: 2,4-Dioxo-3-phenethyl-1,2,3,4-tetrahydroquinazoline-6-carboxylic acid

The product from the preceding step is hydrolysed to the acid according to the procedure of Step 2-4 of Preparation B to provide 0.500 g of the desired compound (yield: 80%).

NMR: DMSO ¹H δ(ppm) 2.85-2.95 (m,2H); 4.05-4.15 (m,2H); 7.15-7.3 (m,6H); 8.15 (d,1H); 8.45 (s,1H); 11.75 (s,1H); 13.05 (bs,1H)

Step 3: 2,4-Dioxo-3-phenethyl-1,2,3,4-tetrahydroquinazoline-6-carboxylic acid (benzo[1,3]dioxol-5-ylmethyl)amide

The product is obtained with a yield of 57.8% (0.205 g) according to the procedure of Example 1, using 250 mg (0.8 mmol) of the compound obtained in the preceding Step 2 and piperonylamine.

NMR: DMSO ¹H δ(ppm): 2.9 (t,2H); 4.1 (t,2H); 4.4 (d,2H); 5.95 (s,2H); 6.75-6.95 (m,3H); 7.15-7.35 (m,6H); 8.1 (d,1H); 8.5 (s,1H); 9.1 (t,1H); 11.65 (bs,1H)

IR: 3249,1704,1658,1636,1488,1251,810,753 cm⁻¹

m.p.=296° C.

HPLC: 99.5%

Step 4: 1-Methyl-2,4-dioxo-3-phenethyl-1,2,3,4-tetrahydroquinazoline-6-carboxylic acid (benzo[1,3]dioxol-5-ylmethyl)amide

0.190 g (0.46 mmol) of the product from the preceding Step 3, 2 ml of dimethylformamide and 0.095 g (0.68 mmol) of K₂CO₃ are introduced into a 25 ml round-bottomed flask. The mixture is stirred for 15 min at room temperature and 0.325 g (0.15 ml, 2.29 mmol) of iodomethane is then added. Stirring is continued for 30 to 45 minutes. The solvent is removed under vacuum. The residue is taken up in dichloromethane and washed with H₂O. The organic phase is separated out after settling and dried over Na₂SO₄. After concentration under vacuum, the product is purified by chromatography on silica, using a 98/2 CH₂Cl₂/MeOH gradient, and then solidified in ether to provide 0.080 g of the desired compound (yield: 76%).

NMR: DMSO ¹H δ(ppm): 2.9 (t,2H); 3.55 (s,3H); 4.15 (t,2H); 4.4 (d,2H); 5.95 (s,2H); 6.8-6.95 (m,3H); 7.15-7.35 (m,5H); 7.55 (d,1H); 8.25 (d,1H); 8.6 (s,1H); 9.15 (t,1H)

IR: 3272,1705,1664,1635,1501,1254,1041,751,698 cm⁻¹

m.p.=183° C.

HPLC: 99.7%

### Example 16

### 3-(4-Methoxybenzyl)-2,4-dioxo-1,2,3,4-tetrahydroquinazoline-6-carboxylic acid (benzo[1,3]dioxol-5-ylmethyl)amide

Step 1: Methyl 3-(4-methoxybenzyl)-2,4-dioxo-1,2,3,4-tetrahydroquinazoline-6-carboxylate

The product is obtained with a yield of 61.3% (0.750 g) according to the procedure of Step 1 of Example 15, but using 4-methoxybenzyl isocyanate:

NMR: DMSO ¹H δ(ppm): 3.7 (s,3H); 3.8 (s,3H); 5.0 (s,2H); 6.8-6.85 (m,2H); 7.2-7.3 (m,3H); 8.1-8.2 (m,1H); 8.5 (s,1H); 11.9 (bs,1H)

Step 2: 3-(4-Methoxybenzyl)-2,4-dioxo-1,2,3,4-tetrahydroquinazoline-6-carboxylic acid

The product from the preceding Step 1 is hydrolysed to the acid according to the procedure of Step 2-4 of Preparation B to provide 0.680 g of the desired compound (yield: 94.8%).

NMR: DMSO ¹H δ(ppm): 3.7 (s,3H); 5.0 (s,2H); 6.8-7.9 (m,2H); 7.2-7.3 (m,3H); 8.1-8.2 (m,1H); 8.5 (s,1H); 11.8 (s,1H); 13.1 (bs,1H)

Step 3: 3-(4-Methoxybenzyl)-2,4-dioxo-1,2,3,4-tetrahydroquinazoline-6-carboxylic acid (benzo[1,3]dioxol-5-ylmethyl)amide

The product is obtained with a yield of 79.9% (0.220 g) according to the procedure of Example 9, using 200 mg (0.6 mmol) of the compound obtained in the preceding Step 2 and piperonylamine. The crude product is solidified in dichloromethane.

NMR: DMSO ¹H δ(ppm): 3.7 (s,3H); 4.35 (d,2H); 5.0 (s,2H); 5.95 (s,2H); 6.75-6.9 (m,5H); 7.2-7.3 (m,3H); 8.1 (d,1H); 8.5 (s,1H); 9.1 (t,1H); 11.75 (s,1H)

IR: 1720,1648,1634,1504,1442,1300,1250,1036,766 cm⁻¹

m.p.=252° C.

HPLC: 96.2%

### Example 17

### 3-(4-Methoxybenzyl)-1-methyl-2,4-dixo-1 3,4-tetrahydroquinazoline-6-carboxylic acid (benzo[1,3]dioxo-5-ylmethyl)amide

The alkylation with methyl iodide of the product obtained in Example 16 is carried out using the procedure described in Example 15, Step 4. After crystallization from ether, 0.080 g of the product is obtained (yield: 70.4%).

NMR: DMSO ¹H δ(ppm): 3.55 (s,3H); 3.7 (s,3H); 4.4 (d,2H); 5.05 (s,2H); 5.95 (s,2H); 6.8-6.95 (m,5H); 7.3 (d,2H); 7.55 (d,1H); 8.25 (d,1H); 8.6 (s,1H); 9.2 (t,1H)

IR: 3265,1704,1662,1634,1504,1443,1320,1248,1040,771 cm⁻¹

m.p.=178° C.

HPLC: 99.2%

### Example 18

### 3-(4-Methoxybenzyl)-1-methyl-2,4-dioxo-1,2,3,4-tetrahydroquinazoline-6-carboxylic acid 4-methoxybenzylamide

Step 1: 3-(4-Methoxybenzyl)-2,4-dioxo-1,2,3,4-tetrahydroquinazoline-6-carboxylic acid (4-methoxybenzyl)amide

The product is obtained with a yield of 82% (0.270 g) according to the procedure of Example 9, using 240 mg (0.74 mmol) of the compound obtained in Step 2 of Example 16 and 4-methoxybenzylamine

NMR: DMSO ¹H δ(ppm): 3.7 (2s,6H); 4.4 (d,2H); 5.0 (s,2H); 6.8-6.95 (m,4H); 7.2-7.35 (m,5H); 8.15 (d,2H); 8.5 (s,1H); 9.15 (t,1H); 11.75 (bs,1H)

Step 2: 3-(4-Methoxybenzyl)-1-methyl-2,4-dioxo-1,2,3,4-tetrahydroquinazoline-6-carboxylic acid 4-methoxybenzylamide

The product is obtained with a yield of 94.4% (0.260 g) according to the procedure of Example 15 Step 4, using the compound obtained in the preceding in Step 1.

NMR: DMSO ¹H δ(ppm): 3.6 (s,3H); 3.7 (dd,6H); 4.45 (d,2H); 5.1 (s,2H); 6.8-6.95 (m,4H); 7.25-7.40 (m,4H); 7.55 (d,1H); 8.25 (d,1H); 8.65 (s,1H); 9.2 (t,1H)

IR: 1705,1655,1641,1614,1510,1247,1175,1033 cm⁻¹

m.p.=195° C.

HPLC: 99.5%

### Example 19

### 3-(Naphth-1-ylethyl)-2,4-dioxo-1,2,3,4-tetrahydroquinazoline-6-carboxylic acid (benzo[1,3]dioxol-5-ylmethyl)amide

The product is obtained according to the procedure of Example 16, Step 1 to 3, using 1-(1-naphthyl)ethyl isocyanate in the Step 1.

NMR: DMSO ¹H δ(ppm): 1.95 (d,3H); 4.35 (d,2H); 6.0 (s,2H); 6.7-6.8 (m,2H); 6.8-6.9 (m,2H); 7.2 (d,1H); 7.4-7.5 (m,2H); 7.6 (t,1H); 7.85-8.0 (m,5H); 8.10 (d,1H); 8.10 (d,1H); 8.45 (s,1H); 9.10 (t,1H); 11.6 (bs,1H)

### Example 20

### 2,4-Dioxo-3-(pyrid-4-ylmethyl)-1,2,3,4-tetrahydroquinazoline-6-carboxylic acid (benzo[1,3]dioxol-5ylmethyl)amide

Step 1: Dimethyl 4-(3-pyrid-4-ylmethylureido)isophthalate

The product is obtained with a yield of 94.2% according to the procedure of Step 1-5 of Preparation B, using the compound obtained in the Preparation A and 4-pyridine methylamine.

NMR: DMSO ¹H δ(ppm): 3.8 (s,3H); 3.9 (s,3H); 4.3 (d,2H); 7.30-7.35 (m,2H); 80-8.1 (m,1H); 8.4 (t,1H); 8.5-8.6 (m,4H); 10.3 (s,1H)

Step 2: Methyl 2,4-dioxo-3-(pyrid-4-ylmethyl)-1,2,3,4-tetrahydroquinazoline-6-carboxylate

The product is obtained according to the procedure of Step 2-5 of Preparation B, using the compound obtained in the preceding Step 1.

NMR: DMSO ¹H δ(ppm): 3.85 (s,3H); 5.1 (s,2H); 7.20-7.30 (m,3H); 8.2 (d,1H); 8.4-8.5 (m,3H); 11.95 (bs,1H)

Step 3: 2,4-Dioxo-3-(pyrid-4-ylmethyl)-1,2,3,4-tetrahydroquinazoline-6-carboxylic acid

The product is obtained according to the procedure of Step 2-4 of Preparation B, using the compound obtained in the preceding Step 2.

NMR: DMSO ¹H δ(ppm): 5.1 (s,2H); 7.20-7.30 (m,3H); 8.2 (d,1H); 8.4-8.5 (m,3H); 11.9 (s,1H); 13.1 (bs,1H)

Step 4: 2,4-Dioxo-3-(pyrid-4-ylmethyl)-1,2,3,4-tetrahydroquinazoline-6-carboxylic acid (benzo[1,3]dioxol-5-ylmethyl)amide

The product is obtained with a yield of 26.7% (0.850 g) according to the procedure of Example 1, using the compound obtained in the preceding Step 3 and piperonylamine. After filtering off an insoluble material, the dimethylformamide is removed under vacuum. The residue is solidified in dichloromethane.

TLC: CH₂Cl₂/MeOH 95/5 Rf=0.40

NMR: DMSO ¹H δ(ppm): 4.40 (d,2H); 5.0 (s,2H); 5.95 (s,2H); 6.80-6.9 (m,3H); 7.2-7.30 (m,3H); 8.1-8.2 (m,1H); 8.4-8.5 (m,3H); 9.1 (t,1H); 11.8 (s,1H)

IR: 3267,1713,1645,1626,1444,1313,1040,920,769 cm⁻¹

m.p.=291.2° C.

HPLC: 87.7%

### Example 21

### 2,1: 2,4-Dioxo3-(thien-2-ylmethyl)-1,2,3,4-tetrahydroquinazoline-6-carboxylic acid benzylamide

Step 1: Methyl N-benzyl-6-(3-thien-2-ylmethylureido)isophthalate

The product is obtained according to the procedure of Step 1-5 of Preparation B, using the compound obtained in the Preparation A and 2-thiophene methylamine.

NMR: DMSO ¹H δ(ppm): 3.8 (s,3H); 3.9 (s,3H); 4.5 (d,2H); 6.9-7.0 (m,2H); 7.4 (m,1H); 8.0-8.05 (m,1H); 8.4 (t,1H); 8.5 (s,1H); 8.6-8.65 (m,1H); 10.15 (s,1H)

Step 2: Methyl 2,4-dioxo-3-(thien-2-ylmethyl)-1,2,3,4-tetrahydroquinazoline-6-carboxylate

The product is obtained according to the procedure of Step 2-5 of Preparation B, using the compound obtained in the preceding Step 1.

NMR: DMSO ¹H δ(ppm): 3.8 (s,3H); 5.25 (s,2H); 6.9 (d,1H); 7.1 (s,1H); 7.25 (d,1H); 7.4 (d,1H); 8.1-8.15 (m,1H); 8.5 (s,1H); 11.9 (bs,1H)

Step 3: 2,4-Dioxo-3-(thien-2-ylmethyl)-1,2,3,4-tetrahydroquinazoline-6-carboxylic acid

The product is obtained according to the procedure of Step 2-4 of Preparation B, using the compound obtained in the preceding Step 2.

NMR: DMSO ¹H δ(ppm): 5.25 (s,2H); 6.95 (d,1H); 7.15 (d,1H); 7.2-7.3 (m,1H); 7.4 (d,1H); 8.1-8.2 (m,1H); 8.5 (s,1H); 11.9 (s,1H); 13.1 (bs,1H)

Step 4: 2,4-Dioxo-3-(thien-2-ylmethyl)-1,2,3,4-tetrahydroquinazoline-6-carboxylic acid benzylamide

The product is obtained with a yield of 61.9% (0.160 g) according to the procedure of Example 1, using the compound obtained in the preceding Step 3 and benzylamine.

TLC: CH₂Cl₂/MeOH 95/5 Rf=0.8

NMR: DMSO ¹H δ(ppm): 4.50 (d,2H); 5.2 (s,2H); 6.90-7.4 (m,9H); 8.15 (d,1H); 8.6 (s,1H); 9.2 (t,1H); 11.8 (s,1H)

IR: 3185,1730,1646,1633,1512,1446,1292,1260,845,763 cm⁻¹

m.p.=264.8° C.

HPLC: 99.5%

### Example 22

### 1-Methyl-2,4,-dioxo-3-(thien-2-ylmethyl)-1,2,3,4-tetrahydroquinazoline-6-carboxylic acid benzylamiide

The product is obtained with a yield of 87% (0.090 g) according to the procedure of Step 4 of Example 15, using the compound obtained in the Example 21.

TLC: CH₂Cl₂/MeOH 95/5 Rf=0.8

NMR: DMSO ¹H δ(ppm): 3.6 (s,3H); 4.50 (d,2H); 5.3 (s,2H); 6.90-7.0 (m,1H); 7.2-7.25 (m,7H); 7.55 (d,1H); 8.3 (d,1H); 8.7 (s,1H); 9.25 (t,1H)

IR: 3257,1704,1657,1637,1513,1480,1325,1251,829,787 cm⁻¹

m.p.=223.7° C.

HPLC: 99.9%

### Example 23

### 2,4-Dioxo-3-(thien-2-ylmethyl)-1,2,3,tetradroquinazoline-6-carboxylic acid (benzo[1,3]dioxol-5-ylmethyl)amide

The product is obtained with a yield of 59% (0.170 g) according to the procedure of Example 1, using the compound obtained in Step 3 of Example 21 and piperonylamine. The crude product is solidified in dichloromethane:

TLC: CH₂Cl₂/MeOH 95/5 Rf=0.4

NMR: DMSO ¹H δ(ppm): 4.40 (d,2H); 5.25 (s,2H); 6.0 (s,2H); 6.75-7.0 (m,4H); 7.1 (s,1H); 7.25 (d,1H); 7.40 (d,1H); 8.2 (d,1H); 8.55 (s,1H); 9.20 (t,1H); 11.8 (s,1H)

IR: 3185,1727,1632,1502,1445,1300,1259,1040,936,846,765 cm⁻¹

m.p.=270.1° C.

HPLC: 95.2%

### Example 24

### 1-Methyl-2,4-dioxo-3-(thien-2-ylmethyl)-1,2,4,4-tetrahydroquinazoline-6-carboxylic acid (benzo[1,3]dioxol-5-ylmethyl)amide

The product is obtained with a yield of 79.7% (0.085 g) according to the procedure of Step 4 of Example 15, using the compound obtained in the Example 23.

TLC: CH₂Cl₂/MeOH 95/5 Rf=0.8

NMR: DMSO ¹H δ(ppm): 3.6 (s,3H); 4.40 (d,2H); 5.30 (s,2H); 6.0 (s,2H); 6.8-7.0 (m,4H); 7.2 (d,1H); 7.40 (d,1H); 7.5-7.6 (m,1H); 8.2-8.30 (m,1H); 8.6 (s,1H); 9.20 (t,1H)

IR: 3251,1705,1659,1635,1501,1446,1328,1253,1041,926,784 cm⁻¹

m.p.=224.2° C.

HPLC: 99.8%

### Example 25

### 3-(4-Chlorobenzyl)-2,4-dioxo-1,2,3,4-tetrahydroquinazoline-6-carboxylic acid (benzo[1,3]dioxol-5-ylmethyl)amide

The product is obtained with a yield of 67.8% (0.170 g) according to the procedure of Example 15 Steps 1 to 3, using in the first step the compound obtained in the Preparation A and 4-chlorobenzyl isocyanate. The product is obtained after solidification in dichloromethane.

NMR: DMSO ¹H δ(ppm): 4.35 (t,2H); 5.1 (s,2H); 5.95 (s,2H); 6.75-6.9 (m,3H); 7.25 (d,1H); 7.35 (s,4H); 8.15 (d,1H); 8.5 (s,1H); 9.15 (t,1H); 11.8 (bs,1H)

IR: 3265,1734,1653,1633,1504,1440,1254,1041,811,761 cm⁻¹

m.p.=290° C.

HPLC: 99.2%

### Example 26

### 3-(4Chlorobenzyl)-1-methyl-2,4-dioxo-1,2,3,4-tetrahydroquinazoline-6-carboxylic acid (benzo[1,3]dioxol-5-ylmethyl)amide

The product is obtained with a yield of 88.9% (0.085 g) according to the procedure of Example 15 Step 4, using the compound obtained in Example 25. The product is isolated after crystallization in ether.

NMR: DMSO ¹H δ(ppm): 3.55 (s,3H); 4.40 (t,2H); 5.15 (s,2H); 5.95 (s,2H); 6.75-6.9 (m,3H); 7.35 (s,4H); 7.55 (d,1H); 8.25 (d,1H); 8.65 (s,1H); 9.20 (t,1H)

IR: 3249,1704,1658,1636,1488,1251,810,753 cm⁻¹

m.p.=231° C.

HPLC: 99.6%

### Example 27

### 1,3-Dimethyl-2,4-dioxo-1,2,3,4-tetrahydroquinazoline-6-carboxylic acid (benzo[1,3]dioxol-5-ylmethyl)amide

The product is obtained (0.035 g) according to the procedure of Example 20 Steps 1 to 4, using in the first step the compound obtained in the Preparation A and monomethylamine, and in Step 4, piperonylamine for the amidation.

TLC: CH₂Cl₂/MeOH 90/10 Rf=0.50

NMR: DMSO ¹H δ(ppm): 3.35 (s,3H); 3.55 (s,3H); 4.40 (d,2H); 6.0 (s,2H); 6.75-6.95 (m,3H); 7.55 (d,1H); 8.25 (d,1H); 8.6 (s,1H); 9.25 (t,1H)

IR: 1703,1649,1501,1486,1256,1037,923 cm⁻¹

m.p.=279° C.

HPLC: 97.3%

### Example 28

### 3-(Benzo[1,3]dioxol-5-ylmethyl)-2,4-dioxo-1,2,3,4-tetrahydroquinazoline-6-carboxylic acid (benzo[1,3]dioxol-5-ylmethyl)amide

The product is obtained with a yield of 36% (0.040 g) according to the procedure of Example 20 Steps 1 to 4, using in the first step the compound obtained in the Preparation A and piperonylamine, and in Step 4, piperonylamine for the amidation.

Step 1: Dimethyl 4-(3-benzo[1,3]dioxol-5-ylmethylureido)isophthalate

NMR: CDCl3 ¹H δ(ppm): 3.9 (s,6H); 4.4 (s,2H); 5.1 (t,1H); 6.70-6.85 (m,3H); 6.95 (s,2H); 8.1-8.2 (m,1H); 8.6-8.7 (m,2H); 10.6 (bs,1H)

Step 2: Methyl 3-(benzo[1,3]dioxol-5-ylmethyl)-2,4-dioxo-1,2,3,4-tetrahydroquinazoline-6-carboxylate

NMR: DMSO ¹H δ(ppm): 3.8 (s,3H); 5.0 (s,2H); 5.9 (s,2H); 6.8 (s,2H); 6.9 (s,1H); 7.25 (d,1H); 8.15 (d,1H); 8.5 (s,1H); 11.8 (bs,1H)

Step 3: 3-(Benzo[1,3]dioxol-5-ylmethyl)-2,4-dioxo-1,2,3,4-tetrahydroquinazoline-6-carboxylic acid

NMR: DMSO ¹H δ(ppm): 5.0 (s,2H); 6.0 (s,2H); 6.8 (s,2H); 6.9 (s,1H); 7.3 (d,1H); 8.2 (d,1H); 8.5 (s,1H); 11.85 (s,1H); 13.05 (bs,1H)

Step 4: 3-(Benzo[1,3]dioxol-5-ylmethyl)-2,4-dioxo-1,2,3,4-tetrahydroquinazoline-6-carboxylic acid (benzo[1,3]dioxol-5-ylmethyl)amide

TLC: CH₂Cl₂/MeOH 95/5

Rf=0.70

NMR: DMSO ¹H δ(ppm): 4.40 (s,2H); 5.0 (s,2H); 5.9 (s,4H); 6.75-6.95 (m,6H); 7.2-7.30 (m,1H); 8.05-8.15 (m,1H); 8.45-8.55 (m,1H); 9.1 (m,1H); 10.3 (m,1H)

IR: 3271,1739,1649,1630,1503,1440,1250,1041,926,759 cm⁻¹

m.p.=245.2° C.

HPLC: 81.5%

### Example 29

### 3-(Benzo[1,3]dioxol-5-ylmethyl)-1-methyl-2,4-dioxo-1,2,3,4-tetrahydroquinazoline-6carboxylic acid (benzo[1,3]dioxol-5-ylmethyl)amide

The product is obtained with a yield of 40.5% (0.050 g) according to the procedure of Example 15 Step 4, using the compound obtained in the Example 28.

TLC: CH₂Cl₂/MeOH 90/10 Rf=0.80

NMR: DMSO ¹H δ(ppm): 3.55 (s,3H); 4.35 (s,2H); 5.0 (s,2H); 6.0 (s,4H); 6.80-7.0 (m,6H); 7.5 (d,1H); 8.25 (d,1H); 8.6 (s,1H); 9.15-9.2 (m,1H)

IR: 3302,1703,1663,1630,1490,1247,1041,929,807,785 cm⁻¹

m.p.=197.5° C.

HPLC: 100%

### Example 30

### 3-Benzyl-1-ethyl-2,4-dioxo-1,2,3,4-tetraquinazoline-6-carboxylic acid (benzo[1,3]dioxol-5-ylmethyl)amide

0.150 g (0.35 mmol) of compound of Example 2, and then 3 ml of anhydrous DMF are introduced into a stirred round-bottomed flask protected from moisture. 0.075 g (0.525 mmol) of K₂CO₃ is added to the stirred solution. The mixture is stirred for 15 minutes and 0.273 g (0.14 ml, 1.75 mmol) of iodoethane is then added. Stirring is continued for about 1 hour. After removing the solvent under vacuum, the residue is dissolved in 50 ml of dichloromethane and washed with 2×50 ml of H₂O. After drying over Na₂SO₄ and concentration under vacuum, the product is crystallized from 8 ml of acetonitrile. The product is obtained as follows:

Weight: 0.070 g Yield=43.7%

TLC: CH₂Cl₂/MeOH 95/5 Rf=0.70

NMR: DMSO ¹H δ(ppm): 1.25 (t,3H); 4.2 (q,2H); 4.4 (d,2H); 5.15 (s,2H); 5.95 (s,2H); (s,2H); 6.75-6.95 (m,3H); 7.2-7.4 (m,5H); 7.65 (d,1H); 8.25 (d,1H); 8.65 (s,1H); 9.15 (t,1H);

IR: 1701,1658,1633,1506,1488,1458,1246,1217,1038,926,803 cm⁻¹

m.p.=176.5° C.

HPLC: 99%

### Example 31

### 3-Benzyl-1-cyclopropylmethyl-2,4-dioxo-1,2,3,4-tetrahydroquinazoline-6-carboxylic acid (benzo[1,3]dioxol-5-ylmethy)amide

The product is obtained with a yield of 76.8% (0.130 g) according to the procedure of Example 30, using cyclopropylmethyl bromide. The product is obtained after solidification in diisopropyl ether.

TLC: CH₂Cl₂/MeOH 95/5 Rf=0.70

NMR: DMSO ¹H δ(ppm): 0.4-0.55 (m,4H); 1.25 (m,1H); 4.1 (d,2H); 4.35 (d,2H); 5.15 (s,2H); 5.95 (s,2H); 6.85 (m,3H); 7.3 (m,5H); 7.7 (d,1H); 8.25 (d,1H); 8.65 (s,1H); 9.2 (t,1H)

IR: 1703,1656,1 641,1504,1467,1307,1261,1241,1043,936,845,748 cm⁻¹

m.p.=184.4° C.

HPLC: 97.2%

### Example 32

### 3-Benzyl-1-isobutyl-2,4-dioxo-1,2,3,4-tetrahydroquinazoline-6-carboxylic acid (benzo[1,3]dioxol-5-ylmethyl)amide

The product is obtained with a yield of 35.3% (0.060 g) according to the procedure of Example 30, using isobutyl bromide.

TLC: CH₂Cl₂/MeOH 95/5 Rf=0.65

NMR: CDC3 ¹H δ(ppm): 1.0 (d,6H); 2.15 (m,1H); 4.0 (d,2H); 4.5 (d,2H); 4.25 (s,2H); 5.95 (s,2H); 6.55 (m,1H); 6.8 (m,3H); 7.25 (m,4H); 7.45 (d,2H); 8.25 (t,1H); 8.45 (s,1H)

IR: 1705,1660,1643,1548,1502,1456,1303,1260,1245,1043,923 cm⁻¹

m.p.=146.0° C.

HPLC: 96.8%

### Example 33

### 1-Methyl-2,4-dioxo-1,2,3,4-tetrahydrquinazoline-6-carboxylic acid (benzo[1,3]-dioxol-5-ylmethyl)amide

Step 1: Methyl 1-methyl-2,4-dioxo-1,2,3,4-tetrahydroquinazoline-6-carboxylate

0.870 g (2.7 mmol) of compound obtained in Step 1 of Preparation C, 20 ml of benzene and 2.1 g (16.1 mmol) of AlCl₃ are maintained at 50° C. for 7 hours. After cooling, the medium is precipitated on a water/ice mixture. The insoluble material is dissolved in dichloromethane and purified by flash chromatography, eluting with a gradient of CH₂Cl₂/acetone. 0.510 g of the desired compound is obtained

Step 2: 1-Methyl-2,4-dioxo-1,2,3,4-tetrahydroquinazoline-6-carboxylic acid (benzo[1,3]dioxol-5-ylmethyl)amide

The saponification of the compound obtained in the preceding Step 1 is carried out with LiOH in a dioxane/H₂O mixture as for the preceding examples. Amidation with piperonylamine gives 0.160 g of the desired product.

TLC: CH₂Cl₂/MeOH 90/10 Rf=0.45

NMR: DMSO ¹H δ(ppm) 3.45 (s,3H); 4.4 (d,2H); 6.0 (s,2H); 6.75-6.95 (m,3H); 7.5 (d,1H); 8.25 (d,1H); 8.55 (s,1H); 9.2 (t,1H); 11.7 (s,1H)

IR: 3290,1697,1635,1503,1484,1324,1258,1040,844 cm⁻¹

m.p.=279° C.

HPLC: 98.7%

### Example 34

### Methyl 4-[6-(4-methoxy-benzylcarbamoyl)-1-methyl-2,4-dioxo-1,4-dihydro-2H-quinazolin-3-ylmethyl]-benzoate

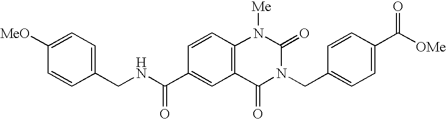

Step 1: 1-Methyl-2,4-dioxo-1,2,3,4-tetrahydro-quinazoline-6-carboxylic acid 4-methoxy-benzylamide:

Preparation identical to that of Example 33, using 1-Methyl-2,4-dioxo-1,2,3,4-tetrahydro-quinazoline-6-carboxylic acid (NMR: DMSO ¹H δ(ppm) 3.50 (s,3H); 7.5 (d,1H); 8.20 (d,1H); 8.50 (s,1H); 11.75 (bs,1H); 13.1 (bs,1H)) and 4 methoxy-benzylamine in DMF with TOTU and DIPEA. The product is obtained as follows:

NMR: DMSO ¹H δ(ppm) 3.50 (s,3H); 3.70 (s,3H); 4.40 (d,2H); 6.90 (d,2H); 7.25 (d,2H); 7.50 (d,1H); 8.20 (d,1H); 8.55 (s,1H); 9.20 (t,1H); 11.65 (bs,1H);

Step 2: Methyl 4-[6-(4-methoxy-benzylcarbamoyl)-1-methyl-2,4-dioxo-1,4-dihydro-2H-quinazolin-3-ylmethyl]-benzoate

0.8 g (2.36 mmoles) of the product obtained in the preceding Step 1 and 8 ml anhydrous DMF are stirred with 1.15 g (3.54 mmol) of cesium carbonate. Stirring is continued for 15 minutes and then 0.81 g (3.54 mmol) of methyl-4-(bromomethyl)benzoate is added. The mixture is maintained at 90° C. for 1 hr 15 min and then stirred overnight. 15 ml of water are added and then extracted with dichloromethane. The organic phase is washed with water and concentrated to dryness on a rotavapor. The product obtained is purified with flash chromatography eluting with a gradient of CH₂Cl₂/MeOH to provide 0.220 g of the desired product.

TLC: CH₂Cl₂/MeOH 90/10 Rf=0.85

NMR: DMSO ¹H δ(ppm): 3.55 (s,3H); 3.7 (s,3H); 3.85 (s,3H); 4.4 (d,2H); 5.25 (s,2H); 6.9 (d,2H); 7.25 (d,2H); 7.45 (d,2H); 7.55 (d,1H); 7.9 (d,2H); 8.25 (dd,1H); 8.6 (s,1H); 9.2 (t,1H)

IR: 3387,1709,1658,1642,1508,1286,1248,1110,1032,835,750 cm⁻¹

m.p=189.2 ° C.

HPLC: 96.5%

### Example 35

### 4-[6-(4-Methoxy-benzylcarbamoyl)-1-methyl-2,4-dioxo-1,4-dihydro-2H-quinazolin-3-ylmethyl]-benzoic acid

0.16 g (3.3 mmoles) of the product obtained in Example 34 is hydrolysed in a mixture of 1.2 ml of dioxane and 4.2 ml of water with 28mg of LiOH monohydrate. The mixture is maintained at reflux for 10 minutes to complete the reaction. After acidification at pH 1 with concentrated HCl, the precipitate is filtered off to provide 0.120 g of the desired compound.

TLC: CH₂Cl₂/MeOH 90/10 Rf=0.50

NMR: DMSO ¹H δ(ppm): 3.55 (s,3H); 3.75 (s,3H); 4.4 (d,2H); 5.20 (s,2 H); 6.9 (d,2H); 7.25 (d,2H); 7.40 (d,2H); 7.60 (d,1H); 7.85 (d,2H); 8.25 (dd,1H); 8.65 (s,1H); 9.2 (t,1H) 12.9 (bs,1H)

IR: 3378,1702,1658,1645,1616,1506,1297,1248,1125,839,788,751 cm⁻¹

m.p=262.5° C.

HPLC: 100%

### Example 36

### 1-Methyl-2,4-dioxo-3-((E)-3phenylally)-1,2,3,4-tetrahydroquinazoline-6-carboxylic acid (benzo[1,3]dioxol-5-ylmethyl)amide

0.100 g (0.28 mmol) of compound of Example 33 and 1 ml of anhydrous DMF are stirred with 0.060 g (0.42 mmol) of K₂CO₃. The mixture is maintained for 15 min, followed by addition of 0.085 g (0.42 mmol) of cinnamyl bromide. The mixture is maintained at 70° C. for 2 hours. After concentration under vacuum, the residue is taken up in dichloromethane, washed with H₂O and then dried over Na₂SO₄. The solvent is removed and the product is purified by flash chromatography, eluting with a 95/5 gradient of CH₂Cl₂/MeOH. A solidification in ether provides 0.070 g (yield=51%) of the desired compound.

TLC: CH₂Cl₂/MeOH 95/5 Rf=0.46

NMR: DMSO ¹H δ(ppm): 3.55 (s,3H); 4.4 (d,2H); 4.75 (d,2H); 6.0 (s,2H); 6.3-6.4 (m,1H); 6.6 (d,1H); 6.80-6.95 (m,3H); 7.2-7.35 (m,3H); 7.4 (d,2H); 7.55 (d,1H); 8.25 (d,1H); 8.65 (s,1H); 9.25 (t,1H)

IR: 1659,1643,1503,1477,1246,754 cm⁻¹

m.p.=174° C.

HPLC: 98.4%

### Example 37

### Benzyl 3-benzyl-2,4-dioxo-1,2,3,4-tetrahydroquinazolne-6-carboxylate

A mixture of 0.5 g (1.7 mmol) of the compound of Preparation B, 0.44 g (1.7 mmol) of triphenylphosphine and 0.44 ml (4.3 mmol) of benzyl alcohol is stirred in 20 ml of THF. A solution of 0.27 ml (1.7 mmol) of DEAD in 10 ml of THF is added dropwise with stirring. Stirring is continued overnight at room temperature. The precipitate formed is filtered through Celite and the filtrate is concentrated under vacuum. The residue is dissolved in 50 ml of ethyl acetate and washed successively with H₂O and then with saturated NaCl solution. After drying over MgSO₄ and concentration under vacuum, the crude product obtained is purified by flash chromatography on silica, eluting with a 50/50 mixture of hexane/EtOAc. The desired fractions are combined and the solvent is removed under vacuum to provide 0.190 g (yield=29%) of the desired crystalline compound.

MS: m/z 387.2 (M+H)+

NMR: DMSO ¹H δ(ppm): 5.06 (s,2H); 5.34 (s,2H); 7.22-7.46 (m,10H); 8.20 (d,1H); 8.48 (s,1H); 11.89 (s,1H)

CHN (C₂₃H₁₈N₂O₄) calc (%): C=71.49, H=4.70, N=7.25 Found (%):C=71.28,H=4.94,N=7.11

### Example 38

### Benzyl 3-beanzyl-1methyl-2,4-dioxo-1,2,3,4-tetrahydrquinazoline-6-carboxylate

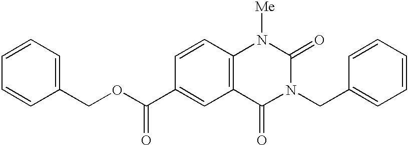

0.084 g (0.217 mmol) of the product of Example 37 is stirred with anhydrous THF in apparatus protected from moisture and under an inert atmosphere. 0.14 ml of 1.6M BuLi in hexane (0.224 mmol) is introduced. The mixture is stirred for 10 minutes, followed by addition of 0.04 ml (0.642 mmol) of methyl iodide. The THF is removed under vacuum. The residue is dissolved in EtOAc and washed successively with H₂O and then with saturated NaCl solution. After drying over MgSO₄ and concentration under vacuum, the crude product obtained is purified by flash chromatography on silica, eluting with a 50/50 mixture of hexane/EtOAc. The desired fractions are combined and the solvent is removed under vacuum. The pale yellow product is solidified in ether:

Weight: 0.049 g Yield=56% MS: m/z 401.2 (M+H)+

NMR: DMSO ¹H δ(ppm): 3.31 (s,3H); 5.12 (s,2H); 5.37 (s,2H); 7.21-7.60 (m,11H); 8.28 (d,1H); 8.58 (s,1H)

CHN (C₂₄H₂₀N₂O₄) calc (%): C=71.99, H=5.03, N=7.00 Found (%): C=71.71, H=5.25, N=6.87

### Example 39

### 4-Pyridylmethyl 3-benzyl-2,4-dioxo-1,2,3,4-tetrahydroqinazoline-6-carboxylate

The compound is obtained according to the procedure of Example 37, but using dichloromethane as solvent, the product is obtained as follows:

MS: m/z 388.2 (M+H)+

NMR: DMSO ¹H δ(ppm): 5.07 (s,2H); 5.41 (s,2H); 7.20-7.32 (m,6H); 7.43 (d,2H); 8.26 (d,1H); 8.53-8.58 (m,3H); 11.93 (s,1H)

CHN (C₂₂H₁₇N₃O₄.0.3H₂O) calc (%): C=67.27, H=4.52, N=10.70 found (%): C=67.32, H=4.40, N=10.47

### Example 40

### 4-Pyridylmethyl 3-benzyl-1-methyl-2,4-dioxo-1,3,3,4 -tetrahydroquinazoline-6-carboxylate

The compound is obtained according to the procedure of Example 37, but using the compound of Preparation C and 4-pyridylcarbinol.

MS: m/z 402.3 (M+H)+

NMR: DMSO ¹H δ(ppm): 3.55 (s,3H); 5.14 (s,2H); 5.42 (s,2H); 5.42 (s,2H); 7.23-7.33 (m,5H); 7.43-7.45 (m,2H); 7.60 (d,1H); 8.32-8.36 (m,1H), 8.57-8.64 (m,3H)

CHN (C₂₃H₁₉N₃O₄.0.14 H₂O): calc (%): C=68.39, H=4.81, N=10.40 found (%): C=68.40, H=4.71, N=10.38

### Example 41

### Benzo[1,3]dioxol-5-ylmethyl3-benzyl-2,4-dioxo-1,2,3,4-tetrahydroquinazoline-6-carboxylate

0.100 g (0.337 mmol) of compound of Preparation B and 1 ml of anhydrous THF are placed in a round-bottomed flask protected from moisture. The suspension is stirred and 0.24 g (0.150 ml, 2.025 mmol) of thionyl chloride is added. The mixture is refluxed for 1 h 30 min. After cooling, the solution is concentrated to dryness on a rotavapor. The 0.110 g of acid chloride obtained is used in the next stage without further purification. 0.080 g (0.51 mmol) of piperonyl alcohol, 1 ml of dichloromethane and 0.051 g (0.070 ml, 0.51 mmol) of triethylamine are introduced into a round-bottomed flask protected from moisture. The solution is cooled to 0° C. The above acid chloride suspended in 2.5 ml of dichloromethane is added to the solution. The mixture is stirred at room temperature for 48 hours. The precipitate obtained is filtered off. The 0.050 g is purified by recrystallization from acetonitrile.

Weight: 0.025 g Yield=17%

TLC: CH₂Cl₂/MeOH 95/5 Rf=0.85

NMR: DMSO ¹H δ(ppm): 5.1 (s,2H); 5.25 (s,2H); 6.05 (s,2H); 6.9-7.4 (m,9H); 8.2 (d,1H); 8.5 (s,1H); 11.9 (bs,1H)

IR: 1715,1650,1624,1446,1285,1262,1080,928,865,764 cm⁻¹

m.p.=238.5° C.

HPLC: 99.7%

### Example 42

### Benzo[1,3]dioxol-5-ylmethyl3-benzyl-1-methyl-2,4-dioxo-1,2,3,4-tetrahydroquinazoline-6-carboxylate

The compound is obtained (0.140 g) according to the procedure of Example 41, but using the compound of Preparation C and piperonyl alcohol.

TLC: CH₂Cl₂/MeOH 95/5 Rf=0.85

NMR: DMSO ¹H δ(ppm): 3.55 (s,3H); 5.15 (s,2H); 5.30 (s,2H); 6.05 (s,2H); 6.9-7.4 (m,8H); 7.6 (d,1H); 8.25 (d,1H); 8.6 (s,1H)

IR: 1716,1703,1659,1618,1447,1294,1227,1103,935,813,763 cm⁻¹

m.p.=199.5° C.

HPLC: 98.8%

### Example 43

### Benzyl 1-benzyl-2,4-dioxo-3-pyrid-4-ylmethyl-1,2,3,4-tetrahydroquinazoline-6carboxylate

0.5 g (1.7 mmol) of compound obtained in the Step 3 of Example 20 in 15 ml of anhydrous THF is stirred and 0.2 ml (1.7 mmol) of benzyl chloride and 1.2 g (8.7 mmol) of K₂CO₃ are added. The mixture is stirred overnight at room temperature and treated as usual to provide the desired compound.

MS: m/z 478.2 (M+H)+

NMR: DMSO ¹H δ(ppm): 5.19 (s,2H); 5.35 (s,2H); 5.39 (s,2H); 7.25-7.45 (m,13H); 8.19 (d,1H); 8.47-8.49 (m,2H); 8.62 (s,1H)

CHN (C₂₉H₂₃N₃O₄) calc (%): C=72.94, H=4.85, N=8.80 Found (%): C=72.58, H=4.79, N=8.57

### Example 44

### 4-Pyridylmethyl 2,4-dioxo-3-(thien-2-ylmethyl)-1,2,3,4 -tetrahydroquinazoline-6-carboxylate

0.69 g (2.3 mmol) of compound obtained in Step 3 of Example 21 is treated according to the procedure of Example 37, using 4-pyridylcarbinol. The product is obtained as follows:

MS: m/z 394.2 (M+H)+

NMR: DMSO ¹H δ(ppm): 5.21 (s,2H); 5.40 (s,2H); 6.93 (d,1H); 7.11 (m,1H); 7.28 (d,1H); 7.40 (d,1H); 7.40 (m,2H); 8.24 (d,1H); 8.49-8.59 (m,3H)

CHN (C₂₀H₁₅N₃O₄S.0.13 CH₂Cl₂.0.03 (ether)) Calc (%): C=59.81 H=3.86, N=10.33; Found (%): C=59.79, H=3.82, N=10.32

### Example 45

### 4-Pyridylmethyl3-(benzo[1,3]dioxol-5-ylmethyl)-2,4-dioxo-1,2,3,4-tetrahydroquinazoline6-carboxylate

The compound is obtained (0.040 g) according to the procedure of example 37, but using the compound obtained in the Step 3 of Example 28 and 4-pyridylcarbinol. The product is crystallized from methanol:

TLC: CH₂Cl₂/MeOH 90/10 Rf=0.70

NMR: DMSO ¹H δ(ppm): 5.0 (s,2H); 5.70 (s,2H); 6.0 (s,2H); 6.85 (s,2H); 7.0 (s,1H); 7.4 (d,1H); 7.95-8.05 (m,2H); 8.3-8.35 (m,1H); 8.60 (s,1H); 8.8-8.95 (m,2H); 12.0 (m,1H)

IR: 1710,1670,1622,1501,1440,1279,1236,1041,923;764 cm⁻¹

m.p.=204.4° C.

HPLC: 92.4%

### Example 46

### Benzyl 3-benzyl-2,4-dioxo-1,2,3,4-tetrahydroprido[2,3-d]pyrimidine-6-carboxylate

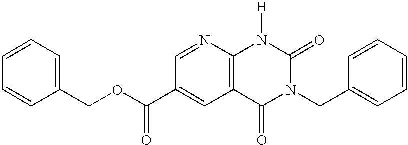

Step 1: 3-Benzyl-6-methyl-1H-pyrido[2,3-d]pyrimidine-2,4-dione

20 g (111 mmol) of ethyl 2-amino-5-methylnicotinate and 200 ml of pyridine are brought to reflux. 13.7 ml (111 mmol) of benzyl isocyanate are added. Refluxing is continued overnight. After cooling, the precipitate is filtered off and washed with 2×100 ml of ethanol and 2×100 ml of ether.

Weight: 10 g in two crops Yield=34%

TLC: CH₂Cl₂/MeOH 90/10 Rf=0.5

NMR: DMSO ¹H δ(ppm): 2.2 (s,3H); 5.0 (s,2H); 7.15-7.35 (m,5H); 8.1 (s,1H); 8.5 (s,1H)

m.p.=279° C.

HPLC: 97%

Step 2: 3-Benzyl-2,4-dioxo-1,2,3,4-tetrahydropyrido[2,3-d]pyrimidine-6-carboxylic acid

3.0 g (11.2 mmol) of the product of the preceding Step 1, 100 ml of H₂O, 7.1 g (44.9 mmol) KMnO₄ and 10 ml of NMP are introduced into a round-bottomed flask. The reaction medium is refluxed overnight. The medium is filtered while hot. The filtrate crystallizes after cooling. After filtering off the new precipitate, the filtrate is treated with 40 ml of Amberlite IR 120 (+) resin. The resin and acid mixture is filtered and the acid is extracted by washing with a 70/30 mixture of CH₂Cl₂/MeOH. The solvent is removed under vacuum to provide 0.32 g of a white solid (yield=10%).

NMR: DMSO ¹H δ(ppm): 5.0 (s,2H); 7.15-7.25 (m,5H); 8.65 (s,1H); 9.1 (s,1H); 12.4 (s,1H)

Step 3: Benzyl 3-benzyl-2,4-dioxo-1,2,3,4-tetrahydropyrido[2,3d]pyrimidine-6-carboxylate

The esterification of the compound of the preceding Step 2 is carried out by the procedure described in Example 37, using benzyl alcohol. After solidification in methanol, 0.040 g of the desired product is obtained (yield=31%):

TLC: CH₂Cl₂/MeOH 95/5 Rf=0.8

NMR: CDCl₃ ¹H δ(ppm): 5.2 (s,2H); 5.4 (s,2H); 7.2-7.6 (m,10H); 9.05 (s,1H); 9.3 (s,1H); 10.9 (s,1H)

m.p.=223° C.

HPLC: 93.1%

### Example 47

### 4-Pyridylmethyl 3-benzyl-2,4-dioxo-1,2,3,4-tetrahydropyrido[2,3-α]pyrimidine-6-carboxylate.

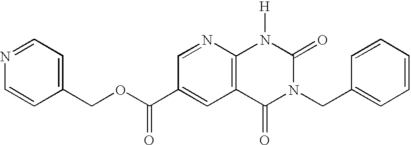

The compound is obtained with a yield of 20% (0.050 g) according to the procedure described in Example 37, but using the compound obtained in the Step 2 of example 46 and 4-pyridylcarbinol.

TLC: EtOAc/NH₄OH 99/1 Rf=0.6

NMR: DMSO ¹H δ(ppm): 5.05 (s,2H); 5.4 (s,2H); 7.15-7.41 (m,5H); 7.45 (d,2H); 8.55 (d,2H); 8.7 (s,1H); 9.15 (s,1H); 12.55 (s,1H)

m.p.=280° C.

HPLC: 97%

### Example 48

### 3-Benzyl-4-oxo-2-thioxo-1,2,3,4-tetrahydroquinazoline-6-carboxylic acid (benzo[1,3]dioxol-5-ylmethyl)amide

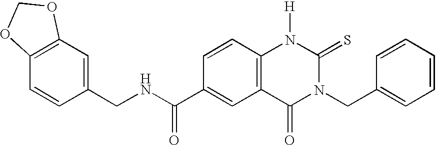

The synthesis is carried out according to Synthetic Scheme 1, using benzyl isothiocyanate during the cyclization to the 4-oxo-2-thioxoquinazoline. After saponification and amidation with piperonylamine, the expected compound is obtained.

Weight: 0.100 g TLC: CH₂Cl₂/MeOH 95/5 Rf=0.64

NMR: DMSO ¹H δ(ppm): 4.4 (d,2H); 5.65 (s,2H); 5.95 (s,2H); 6.75-6.95 (m,3H); 7.2-7.4 (m,5H); 7.45 (d,1H); 8.2 (d,1H); 8.55 (s,1H); 9.2 (t,1H); 13.2 (bs,1H)

IR: 1698,1636,1619,1528,1446,1194,1037,768

m.p.=249° C.

HPLC: 97.2%

### Example 49

### 4-[6-(4-Hydroxy-benzylcarbamoyl)-1-methyl-2,4-dioxo-1,4-dihydro-2H-quinazolin-3-ylmethyl]-benzoic acid

Into a stirred round-bottomed flask protected from moisture, 0.7 g (1.44 mmol) of compound of Example 34 and 70 ml of anhydrous dichloromethane are introduced. The mixture is stirred and 1.4 ml (14.4 mmol) of BBr₃ in 7 ml of dichloromethane are added dropwise. After 2 hours of stirring at room temperature the reaction is complete. After an usual treatment, 0.280 g of the desired product is obtained (yield=42%).

TLC: CH₂Cl₂/MeOH 90/10 Rf=0.15

NMR: DMSO ¹H δ(ppm): 3.55 (s,3H); 4.35 (d,2H); 5.2 (s,2H); 6.65 (d,2H); 7.10 (d,2H); 7.40 (d,2H); 7.55 (d,1H); 7.85 (d,2H); 8.25 (d,1H); 8.60 (s,1H); 9.15 (t,1H); 9.2 (s,1H); 12.8 (bs,1H)

IR: 3403, 2553, 1697, 1658, 1615, 1507, 1482, 1423, 1247, 1109, 829, 752 cm⁻¹

M.P.=174.0° C.

HPLC: 97.06%

### Example 50

### 3-(4-Dimethylcarbamoyl-benzyl)-1-methyl-2,4-dioxo-1,2,3,4-tetrahydro quinazoline-6-carboxylic acid 4-methoxy-benzylamide

0.3 g (0.64 mmol) of the compound of Example 35 is treated with a 2M solution of dimethylamine in THF according to the procedure described in Example 1. The crude product is purified by chromatography on silica gel and concretized in ether to provide 0.160 g of the desired compound (yield: 49.9%).

TLC: CH₂Cl₂/MeOH 90/10 Rf=0.70

NMR:.CDCl3 ¹H δ(ppm): 2.90 (s,3H); 3.05 (s,3H); 3.60 (s,3H); 3.80 (s,3H); 4.60 (d,2H); 5.25 (s,2H); 6.60 (t,1H); 6.85 (d,2H); 7.3 (m,5H); 7.45 (d,2H); 8.25 (d,1H); 8.50 (s,1H).

IR: 3378, 1710, 1654, 1641, 1618, 1508, 1476, 1246, 752 cm⁻¹

M.P.=189 ° C.

HPLC: 97%

### Example 51

### 1-Methyl-3-(4-methylcarbamoyl-benzyl)-2,4-dioxo-1,2,3,4-tetrahydro -quinazoline-6-carboxylic acid 4-methoxy-benzylamide

The compound is obtained according to the procedure of Example 50 but using methylamine.

TLC: CH₂Cl₂/MeOH 90/10 Rf=0.55

NMR: DMSO ¹H δ(ppm): 2.75 (d,3H); 3.55 (s,3H); 3.70 (s,3H); 4.40 (d,2H); 5.20 (s,2H); 6.85 ( d,2H); 7.25 (d,2H); 7.35 (d,2H); 7.55 (d,1H); 7.75 (d,2H); 8.25 (q,1H); 8.35 (d,1H); 8.60 (s,1H); 9.2 (t,1H).

IR: 3338, 1708, 1654, 1616, 1548, 1507, 1329, 1245, 1036, 825, 751 cm⁻¹

M.P.=255.1° C.

HPLC: 97.0%

### Example 52

### 3-Allyl-1-methyl-2,4-dioxo-1,2,3,4-tethydro-quinazoline-6-carboxylic acid 4-methoxy-benzylamiide

The compound is obtained according to the procedure of the Step 2 of the Example 34 but using as substrates the compound obtained in the Step 1 of the Example 34 and 3-allyl bromide.

NMR: DMSO ¹H δ(ppm): 3.55 (s,3H); 3.8 (s,3H); 4.4 (d,2H); 4.55 (d,2H); 5.10-5.20 (m,2H); 5.80-5.95 (m,1H); 6.9 (d,2H); 7.25 (d,2H); 7.55 (d,1H); 8.25 (d,1H); 8.6 (s,1H); 9.25 (t,1H)

IR: 1703, 1642, 1615, 1508, 1477, 1246, 765 cm⁻¹

M.P.=207° C.

HPLC:98.9%

### Example 53

### 1-Methyl-2,4-dioxo-3-(2-pyrrol-1-yl-ethyl)-1,2,3,4-tetrahydro-quinazoline-6-carboxylic acid 4-methoxy-benzylamide

The compound is obtained according to the procedure of the Step 2 of the Example 34 but using as substrates the compound obtained in the Step 1 of the Example 34 and 1(2-bromoethyl)pyrrole.

NMR: DMSO ¹H δ(ppm): 3.55 (s,3H); 3.7 (s,3H); 4.15 (m,2H); 4.25 (m,2H); 4.40 (d,2H); 5.90 (s,2H); 6.7 (s,2H); 6.90 (d,2H); 7.25 (d,2H); 7.55 (d,1H); 8.25 (d,1H); 8.55 (s,1H); 9.2 (t,1H)

IR: 3338, 1708, 1655, 1640, 1508, 1478, 1251, 117, 1032, 835, 734 cm⁻¹

M.P.=147° C.

HPLC:96.6%

### Example 54

### 1-Methyl-2,4-dioxo-3-(prop-2-ynyl)-1,2,3,4-tetrahydro-quinazolne-6-carboxylic acid 4-methoxy-benzylamide

The compound is obtained according to the procedure of the Step 2 of the Example 34 but using as substrates the compound obtained in the Step 1 of the Example 34 and prp-2-ynyl bromide.

NMR: DMSO ¹H δ(ppm): 3.15 (s,1H); 3.55 (s,3H); 3.7 (s,3H); 4.40 (d,2H); 4.70 (s,2H); 6.90 (d,2H); 7.25 (d,2H); 7.55 (d,1H); 8.25 (d,1H); 8.60 (s,1H); 9.25 (t,1H).

IR: 3265, 1710, 1667, 1635, 1501, 1326, 1249, 1036, 825, 783, 752 cm⁻¹

M.P.=206° C.

HPLC: 97.7%

### Example 55

### 1Methyl-3-(3-methyl-but-2-enyl)-2,4-dioxo-1,2,3,4-tetrahydro-quinazoline-6-carboxylic acid 4-methoxy-benzylamide

The compound is obtained according to the procedure of the Step 2 of the Example 34 but using as substrates the compound obtained in the Step 1 of the Example 34 and 1-bromo-3-methyl-but-2-ene.

NMR: DMSO ¹H δ(ppm): 1.65 (s,3H); 1.75 (s,3H); 3.50 (s,3H); 3.7 (s,3H); 4.40 (d,2H); 4.55 (d,2H); 5.20 (t,1H); 6.90 (d,2H); 7.25 (d,2H); 7.55 (d,1H); 8.25 (d,1H); 8.60 (s,1H); 9.25 (t,1H)

IR : 3282, 1705, 1659, 1634, 1500, 1314, 1246, 826 cm⁻¹

M.P.=187° C.

HPLC: 96.9%

### Example 56

### 1-Methyl-2,4-dioxo-3-(pyridin-2-ylmethyl)-1,2,3,4-tetrahydro-quinazoline-6-carboxylic acid 4-methoxy-benzylamide

The compound is obtained according to the procedure of the Step 2 of the Example 34 but using as substrates the compound obtained in the Step 1 of the Example 34 and 2-(bromomethyl)pyridine.

NMR: DMSO ¹H δ(ppm): 3.55 (s,3H); 3.7 (s,3H); 4.40 (d,2H); 5.25 (s,2H); 6.90 (d,2H); 7.25 (m,3H); 7.35 (d,1H); 7.60 (d,1H); 7.70 (m,1H); 8.25 (d,1H); 8.40 (d,1H); 8.60 (s,1H); 9.2 (t,1H)

IR: 1702, 1658, 1643, 1618, 1508, 1476, 1331, 1248, 751 cm⁻¹

M.P.=156° C.

HPLC: 99.5%

### Example 57

### 3-Carbamoylmethyl-1-methyl-2,4-dioxo-1,2,3,4-tetrahydro-quinazoline-6-carboxylic acid 4-methoxy-benzylamide

The compound is obtained according to the procedure of the Step 2 of the Example 34 but using as substrates the compound obtained in the Step 1 of the Example 34 and 2-chloro-acetamide.

NMR: DMSO ¹H δ(ppm): 3.55 (s,3H); 3.7 (s,3H); 4.40 (d,2H); 4.50 (s,2H); 6.90 (d,2H); 7.20 (s,1H); 7.25 (d,2H); 7.55 (d,1H); 7.65 (s,1H); 8.25 (d,1H); 8.60 (s,1H); 9.25 (t,1H)

IR: 1655,1531,1508,1477,1303,1249,752 cm⁻¹

M.P.=269° C.

HPLC: 99.2%

### Example 58

### 1-Methyl-2,4-dioxo-3-(pyridin-3-ylmethyl)-1,2,3,4-tetrahydro-quinazoline-6-carboxylic acid 4-methoxy-benzylamide

The compound is obtained according to the procedure of the Step 2 of the Example 34 but using as substrates the compound obtained in the Step 1 of the Example 34 and 3-(bromomethyl)pyridine.

NMR: DMSO ¹H δ(ppm): 3.55 (s,3H); 3.7 (s,3H); 4.40 (d,2H); 5.20 (s,2H); 6.85 (d,2H); 7.20-7.40 (m,3H); 7.55 (d,1H); 7.75 (d,1H); 8.25 (m,1H); 8.45 (d,1H); 8.60 (m,2H); 9.20 (t,1H)

IR: 1699, 1660, 1615, 1500, 1479, 1249, 1032, 752, 712 cm⁻¹

M.P.=140° C.

HPLC: 89.6%

### Example 59

### 1-Methyl-3-(1-methyl-piperidin-3-ylmethyl)-2,4-dioxo-1,2,3,4-tetrahydro-quinazoline-6-carboxylic acid 4-methoxy-benzylamide

The compound is obtained according to the procedure of the Step 2 of the Example 34 but using as substrates the compound obtained in the Step 1 of the Example 34 and 3-bromomethyl-1-methyl-piperidine

NMR: DMSO ¹H δ(ppm): 0.85-1.00 (m,1H); 1.30-1.45 (m,1H); 1.55-2.05 (m,5H); 2.10 (s,3H); 2.60 (m,2H); 3.55 (s,3H); 3.75 (s,3H); 3.85 (d,2H); 4.40 (d,2H); 6.90 (d,2H); 7.25 (d,2H); 7.50 (d,1H); 8.25 (d,1H); 8.60 (s,1H); 9.25 (t,1H)

IR: 2926, 1655, 1641, 1508, 1247, 788 cm⁻¹

M.P.=174° C.

HPLC: 99.3%

### Example 60

### 3-(4-Cyano-benzyl)-1-methyl-2,4-dioxo-1,2,3,4-tetrahydro-quinazoline-6-carboxylic acid 4-methoxy-benzylamide

The compound is obtained according to the procedure of the Step 2 of the Example 34 but using as substrates the compound obtained in the Step 1 of the Example 34 and 4-(bromomethyl)benzonitrile

NMR: DMSO ¹H δ(ppm): 3.55 (s,3H); 3.75 (s,3H); 4.40 (d,2H); 5.20 (s,2H); 6.90 (d,2H); 7.25 (d,2H); 7.45-7.60 (m,3H); 7.75 (d,2H); 8.25 (d,1H); 8.60 (s,1H); 9.20 (t,1H)

IR: 3411, 2216, 1708, 1649, 1616, 1251, 839, 765 cm⁻¹

M.P.=222° C.

HPLC 97.2%

### Example 61

### 3-(3-Cyano-benzyl)-1-methyl-2-dioxo-1,2,3,4-tetrahydro-quinazoline-6-carboxylic acid 4-methoxy-benzylamide

The compound is obtained according to the procedure of the Step 2 of the Example 34 but using as substrates the compound obtained in the Step 1 of the Example 34 and 3-(bromomethyl)-benzonitrile.

TLC: CH₂Cl₂/MeOH 90/10 Rf=0.80

NMR: DMSO ¹H δ(ppm) : 3.45 (s,3H); 3.70 (s,3H); 4.45 ( d,2H); 5.15 (s,2H); 6.90 (d,2H); 7.25 (d,2H); 7.55 (m,2H); 7.70 (m,2H); 7.80 (s,1H); 8.25 (d,1H); 8.65 (s,1H); 9.20 (t,1H).

IR: 1708, 1660, 1618, 1503, 1477, 1335, 1247, 1160, 952, 760, 718 cm⁻¹

M.P.=201° C.

HPLC 97.1%

### Example 62

### 3-(2-Methoxy-ethyl)-1-methyl-2,4-dioxo-1,2,3,4-tetrahydro-quinazoline-6-carboxylic acid 4-methoxy-benzylamide

The compound is obtained according to the procedure of the Step 2 of the Example 34 but using as substrates the compound obtained in the Step 1 of the Example 34 and 1-bromo-2-methoxy-ethane.

NMR: DMSO ¹H δ(ppm): 3.25 (s,3H); 3.55 (m,5H); 3.70 (s,3H); 4.15 (t,2H); 4.40 (d,2H); 6.90 (d,2H); 7.25 (d,2H); 7.55 (d,1H); 8.25 (d,1H); 8.60 (s,1H); 9.20 (t,1H)

IR: 3274, 1709, 1660, 1633, 1514, 1249, 1030, 823 cm⁻¹

M.P.=200° C.

HPLC: 99.2%

### Example 63

### 3-Methoxy-benzyl)-1-methyl-2,4-dioxo-1,2,3,4-tetrahydro-quinazoline-6-carboxylic acid 4-methoxy-benzylamide

The compound is obtained according to the procedure of the Step 2 of the Example 34 but using as substrates the compound obtained in the Step 1 of the Example 34 and 3-(bromomethyl)-1-methoxyphenyl.

NMR: DMSO ¹H δ(ppm): 3.55 (s,3H); 3.70 (s,6H); 4.40 (d,2H); 5.10 (s,2H); 6.75-6.90 (m,5H); 7.15-7.30 (m,3H); 7.55 (d,1H); 8.25 (d,1H); 8.60 (s,1H); 9.20 (t,1H)

IR: 3387, 1704, 1657, 1640, 1616, 1509, 1250, 766 cm⁻¹

M.P.=154° C.

HPLC: 99.4%

### Example 64

### 3-Cyclopropylmethyl-1-methyl-2,4-dioxo-1,2,3,4-tetrahydro-quinazoline-6-carboxylic acid 4-methoxy-benzylamide

The compound is obtained according to the procedure of the Step 2 of the Example 34 but using as substrates the compound obtained in the Step 1 of the Example 34 and bromomethylcyclopropyl.

NMR: DMSO ¹H δ(ppm): 0.40 (m,4H); 1.2 (m,1H); 3.55 (s,3H); 3.70 (s,3H); 3.85 (d,2H); 4.40 (d,2H); 6.90 (d,2H); 7.25 (d,2H); 7.55 (d,1H); 8.25 (m,1H); 8.60 (d,1H); 9.20 (t,1H).

IR : 3282, 1703, 1657, 1634, 1502, 1258, 1028, 829, 752 cm⁻¹

M.P.=209° C.

HPLC: 98.2%

### Example 65

### 1-Methyl-3-(2-morpholin-4-yl-ethyl)-2,4-dioxo-1,2,3,4-tetrahydro-quinazoline-6-carboxylic acid 4-methoxy-benzylamide

The compound is obtained according to the procedure of the Step 2 of the Example 34 but using as substrates the compound obtained in the Step 1 of the Example 34 and 4-(2-bromoethyl)morpholine.

NMR: DMSO ¹H δ(ppm): 2.40 (m,4H); 2.55 (m,2H); 3.50 (m,7H); 3.75 (s,3H); 4.10 (t,2H); 4.40 (d,2H); 6.90 (d,2H); 7.25 (d,2H); 7.55 (d,1H); 8.25 (d,1H); 8.60 (s,1H) 9.20 (t,1H)

IR: 3419, 1707, 1656, 1612, 1506, 1475, 1246, 1111, 752 cm⁻¹

M.P.=135° C.

HPLC: 98.5%

### Example 66

### 3-Cyclohexylmethyl-1-methyl-2,4-dioxo-1,2,3,4-tetrahydro-quinazoline-6-carboxylic acid 4-methoxy-benzylamide

The compound is obtained according to the procedure of the Step 2 of the Example 34 but using as substrates the compound obtained in the Step 1 of the Example 34 and (bromomethyl)cyclohexane.

NMR: DMSO ¹H δ(ppm): 0.9-1.20 (m,5H); 1.5-1.85 (m,6H); 3.55 (s,3H); 3.70 (s,3H); 3.80 (d,2H); 4.40 (d,2H); 6.90 (d,2H); 7.25 (d,2H); 7.50 (d,1H); 8.25 (m,1H); 8.60 (s,1H); 9.20 (t,1H)

IR: 3378, 2918, 1703, 1654, 1640, 1508, 1478, 1329, 1244, 789, 767 cm⁻¹

M.P.=183° C.

HPLC:99.0%

### Example 67

### 1-Methyl-2,4-dioxo-3-(3-phenyl-propyl)-1,2,3,4-tetrahydro-quinazoline-6-carboxylic acid 4-methoxy-benzylamide

The compound is obtained according to the procedure of the Step 2 of the Example 34 but using as substrates the compound obtained in the Step 1 of the Example 34 and 3-phenylpropyl bromide.

NMR: DMSO ¹H δ(ppm): 1.90 (m,2H); 2.65 (t,2H); 3.50 (s,3H); 3.70 (s,3H); 4.0 (t,2H); 4.40 (d,2H); 6.85 (d,2H); 7.10-7.30 (m,7H); 7.50 (d,1H); 8.20 (m,1H); 8.60 (s,1H); 9.20 (t1H).

IR: 3395, 1704, 1641, 1615, 1509, 1477, 1327, 1245, 1032, 749 cm⁻¹

M.P.=167° C.

HPLC: 98.8%

### Example 68

### 3-(4-Fluoro-benzyl)-1-methyl-2,4-dioxo-1,2,3,4-tetrahydro-quinazoline-6-carboxylic acid 4methoxy-benzylamide

The compound is obtained according to the procedure of the Step 2 of the Example 34 but using as substrates the compound obtained in the Step 1 of the Example 34 and 4-(bromomethyl)-fluorobenzene.

NMR: DMSO ¹H δ(ppm): 3.55 (s,3H); 3.70 (s,3H); 4.40 (d,2H); 5.1 (s,2H); 6.90 (d,2H); 7.10 (t,2H); 7.25 (d,2H); 7.40 (m,2H); 7.50 (d,1H); 8.25 (m,1H); 8.60 (s,1H); 9.20 (t,1H)

IR: 3395, 1704, 1641, 1615, 1509, 1477, 1327, 1245, 1032, 749 cm^{31 1}

M.P.=180° C.

HPLC: 99.4%

### Example 69

### 3-[2-(4-Diethylamino-phenyl)-2-oxo-ethyl]-1-methyl-2,4-dioxo-1,2,3,4-tetrahydro-quinazoline-6-carboxylic acid 4-methoxy-benzylamide

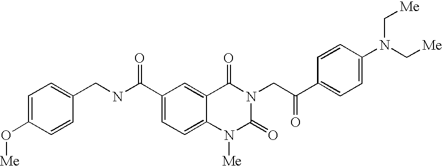

The compound is obtained according to the procedure of the Step 2 of the Example 34 but using as substrates the compound obtained in the Step 1 of the Example 34 and 2-Chloro-1-(4-diethylamino-phenyl)-ethan-1-one.

NMR: DMSO ¹H δ(ppm): 1.15( t,6H); 3.30-3.50 (m,4H); 3.60(s,3H); 3.75 (s,3H); 4.45 (d,2H); 5.35 (s,2H); 6.75 (d,2H); 6.90 (d,2H); 7.30 (d,2H); 7.65 (d,1H); 7.90 (d,2H); 8.30 (m,1H); 8.60 (s,1H); 9.25 (t,1H)

IR: 3370, 1670, 1655, 1596, 1504, 1258, 1242, 1190, 808 cm⁻¹

M.P.=237° C.

HPLC: 97.0%

### Example 70

### Ethyl [6-(4-methoxy-benzylcarbamoyl)-1-methyl-2,4-dioxo-1,4-dihydro-2H-quinazolin-3-yl]-acetate

The compound is obtained according to the procedure of the Step 2 of the Example 34 but using as substrates the compound obtained in the Step 1 of the Example 34 and ethyl 2-chloro-acetate.

NMR: DMSO ¹H δ(ppm): 1.20 (t,3H); 3.60 (s,3H); 3.70 (s,3H); 4.15 (q,2H); 4.40 (d,2H); 4.70 (s,2H); 6.90 (d,2H); 7.25 (d,2H); 7.60 (d,1H); 8.30 (m,1H); 8.60 (s,1H); 9.20 (t,1H)

IR: 1711, 1668, 1637, 1508, 1247, 1212, 1032, 835, 752 cm⁻¹

M.P.=170° C.

HPLC: 97.7%

### Example 71

### 3-(2-Hydroxy-ethyl)-1-methyl-2,4-dioxo-1,2,3,4-tetrahydro-quinazoline-6-carboxylic acid 4-methoxy-benzylamide

The compound is obtained according to the procedure of the Step 2 of the Example 34 but using as substrates the compound obtained in the Step 1 of the Example 34 and 2-bromoethan-1-ol.

NMR: DMSO ¹H δ(ppm): 3.50-3.65 (s,5H); 3.70 (s,3H); 4.05 (t,2H); 4.40 (d,2H); 4.80 (t,1H); 6.90 (d,2H); 7.25 (d,2H); 7.50 (s,1H); 8.25 (m,1H); 8.60 (s,1H); 9.25 (t,1H)

IR: 3290, 1702, 1654, 1639, 1619, 1509, 1327, 1240, 1071, 835, 753 cm⁻¹

M.P.=168° C.

HPLC: 96.7%

### Example 72

### Methyl 3-[6-(4-methoxy-benzylcarbamoyl)-1-methyl-2,4-dioxo-1,4-dihydro-2H-quinazolin-3-yl]-propionate

The compound is obtained according to the procedure of the Step 2 of the Example 34 but using as substrates the compound obtained in the Step 1 of the Example 34 and methyl 3-bromo-propanoate.

NMR: DMSO ¹H δ(ppm) : 2.60 (t,2H); 3.50 (s,3H); 3.60 (s,3H); 3.70 (s,3H); 4.20 (t,2H); 4.40 (d,2H); 6.90 (d,2H); 7.25 (d,2H); 7.50 (d,1H); 8.25 (dd,1H); 8.60 (s,1H); 9.25 (t,1H) IR: 3411, 2361, 1704, 1656, 1644, 1618, 1508, 1478, 1328, 1244, 853, 766 cm⁻¹

M.P.=154.8° C.

HPLC: 95.1%

### Example 73

### 3-[6-(4-Methoxy-benzylcarbamoyl)-1-methyl-2,4-dioxo-1,4-dihydro-2H-quinazolin-3-yl]-propionic acid

The compound is obtained according to the procedure of the Step 2-4 of the Preparation B, but using as substrates the compound obtained in the Example 72.

TLC: CH₂Cl₂/MeOH 90/10 Rf=0.25

NMR: DMSO ¹H δ(ppm) : 2.50 (t,2H); 3.55 (s,3H); 3.70 (s,3H); 4.15 (t,2H); 4.40 (d,2H); 6.85 (d,2H); 7.25 (d,2H); 7.50 (d,1H); 8.25 (dd,1H); 8.55 (s,1H); 9.15 (t,1H); 12.3 (bs,1H)

IR: 3395, 2353, 1701, 1656, 1639, 1508, 1478, 1244, 1040, 839, 799, 754 cm⁻¹

M.P.=201.5° C.

HPLC: 96.4%

### Example 74

### Ethyl 4-[6-(4methoxy-benzylcarbamoyl)-1-methyl-2,4-dioxo-1,4-dihydro-2H-quinazolin-3-yl]-butyrate

The compound is obtained according to the procedure of the Step 2 of the Example 34 but using as substrates the compound obtained in the Step 1 of the Example 34 and ethyl 4-bromobutyrate.

NMR: DMSO ¹H δ(ppm) : 1.10 (t,3H); 1.90 (q,2H); 2.30 (t,2H); 3.55 (s,3H); 3.70 (s,3H); 4.00 (bs,4H); 4.45 (d,2H); 6.90 (d,2H); 7.25 (d,2H); 7.50 (d,1H); 8.20 (dd,1H); 8.60 (s,1H); 9.15 (t,1H)

IR: 3378, 2943, 1704, 1657, 1647, 1617, 1509, 1477, 1246, 1178, 1030, 751 cm⁻¹

M.P.=138.9° C.

HPLC: 99.1%

### Example 75

### 4-[6-(4-Methoxy-benzylcarbamoyl)-1-methyl-2,4-dioxo-1,4-dihydro-2-quinazolin-3-yl]-butyric acid

The compound is obtained according to the procedure of the Step 2-4 of the Preparation B, but using as substrates the compound obtained in the Example 74.

TLC: CH₂Cl₂/MeOH 90/10 Rf=0.50

NMR: DMSO ¹H δ(ppm): 1.80 ( q,2H); 2.25 (t,2H); 3.50 (s,3H); 3.70 (s,3H); 4.0 (t,2H); 4.40 (d,2H); 6.90 (d,2H); 7.25 (d,2H); 7.50 (d,1H); 8.25 (dd,1H); 8.60 (s,1H); 9.20 (t,1H); 12.0 (bs,1H)

IR: 3346, 1691, 1651, 1637, 1512, 1234, 1248, 1178, 1024, 835, 752 cm⁻¹

M.P.=165.6° C.

HPLC: 99.1%

### Example 76

### Methyl {4-[6-(4-methoxy-benzylcarbamoyl)-1-methyl-2,4-dioxo-1,4-dihydro-2H-quinazolin-3-ylmethyl]-phenyl}-acetate

The compound is obtained according to the procedure of the Step 2 of the Example 34 but using as substrates the compound obtained in the Step 1 of the Example 34 and methyl 4-(bromomethyl)phenyl acetate

TLC: CH₂Cl₂/MeOH 90/10 Rf−0.80

NMR: DMSO ¹H δ(ppm) 3.55 (s,3H); 3.60 (s,3H); 3.65 (s,2H); 3.70 (s,3H); 4.40 (d,2H); 5.15 (s,2H); 6.90 (d,2H); 7.10-7.35 (m,6H); 7.55 (d,1H); 8.25 (dd,1H); 8.65 (s,1H); 9.20 (t,1H)

IR: 3370, 2951, 1707, 1655, 1639, 1616, 1509, 1328, 1251, 1157, 1036, 766 cm⁻¹

M.P.=173.2° C.

HPLC: 99.0%

### Example 77

### {4-[6-(4-Methoxy-benzylcarbamoyl)-1-methyl-2,4-dioxo-1,4-dihydro-2H-quinazolin-3-ylmethyl]phenyl}-acetic acid

The compound is obtained according to the procedure of the Step 2-4 of the Preparation B, but using as substrates the compound obtained in the Example 76.

TLC: CH₂Cl₂/MeOH 90/10 Rf=0.50

NMR: DMSO ¹H δ(ppm): 3.55 (s,2H); 3.60 (s,3H); 3.70 (s,3H); 4.40 (d,2H); 5.15 (s,2H); 6.90 (d,2H); 7.10-7.35 (m,6H); 7.55 (d,1H); 8.25 (dd,1H); 8.60 (s,1H); 9.20 (t,1H); 12.3 (bs,1H)

IR: 3378, 1706, 1653, 1640, 1616, 1508, 1330, 1249, 1149, 1032, 823, 766 cm⁻¹

M.P.=165° C.

HPLC: 96.7%

### Example 78

### 3-(4-Dimethylcarbamoylmethyl-benzyl)-1-methyl-2,4-dioxo-1,2,3,4-tetrahydro-quinazoline-6-carboxylic acid 4-methoxy-benzylamide

The compound is obtained from the compound obtained in Example 77, which is transformed in situ into the acid chloride derivate by action of oxalyle chloride and then treated with a 2M solution of dimethylamine in THF.

TLC: CH₂Cl₂/MeOH 90/10 Rf=0.50

NMR: DMSO ¹H δ(ppm): 2.80 (s,3H); 3.0 (s,3H); 3.55 (s,3H); 3.60 (s,2H); 3.75 (s,3H); 4.40 (d,2H); 5.15 (s,2H); 6.90 (d,2H); 7.15 (d,2H); 7.25 ( d,4H); 7.55 (d,1H); 8.65 (s,1H); 9.20 (t,1H).

IR: 3308, 2926, 1706, 1665, 1640, 1504, 1474, 1320, 1250, 1133, 1036, 834 cm^{31 1}

M.P.=183° C.

HPLC :93.2%

### Example 79

### 1-Methyl-2,4-dioxo-3-[(E)-3-(pyridin-3-yl)-allyl]-1,2,3,4-tetrahydro-quinazoline6-carboxylic acid 4-methoxy-benzylamide

The compound is obtained according to the procedure of the Step 2 of the Example 34 but using as substrates the compound obtained in the Step 1 of the Example 34 and 3-((E)-3-chloro-propenyl)-pyridine.

TLC: CH₂Cl₂/MeOH 90/10 Rf=0.63

NMR: DMSO 1H δ(ppm): 3.55 (s,3H); 3.75 (s,3H); 4.40 (d,2H); 4.75 (d,2H); 6.40-6.50 (m,1H); 6.50-6.60 (d,1H); 6.90 (d,2H); 7.20-7.35 (m,3H); 7.55 (d,1H); 7.85 (d,1H); 8.25 (d,1H); 8.40 (s,1H); 8.60 (d,2H); 9.20 (t,1H).

IR: 3395, 1703, 1643, 1509, 1479, 1254, 761 cm⁻¹

M.P.=200.0° C.

HPLC: 98.7%

### Example 80

### 1-Methyl-2,4-dioxo-3-[(E)-3-pyridin-4-yl)-allyl]-1,2,3,4-tetrahydro-quinazoline-6-carboxylic acid 4-methoxy-benzylamide

The compound is obtained according to the procedure of the Step 2 of the Example 34 but using as substrates the compound obtained in the Step 1 of the Example 34 and 4-((E)-3-chloro-propenyl)-pyridine.

TLC: CH₂Cl₂/MeOH 90/10 Rf=0.43

NMR: DMSO ¹H δ(ppm) 3.55 (s,3H); 3.75 (s,3H); 4.45 (d,2H); 4.80 (d,2H); 6.55 (d,1H); 6.60-6.70 (m,1H); 6.90 (d,2H); 7.25 (d,2H); 7.35 (d,2H); 7.55 (d,1H); 8.25 (dd,1H); 8.45 (d,2H); 8.65 (s,1H); 9.20 (t,1H).

IR: 3395, 1704, 1643, 1509, 1479,1332, 1254, 980, 765 cm⁻¹

M.P.=241° C.

HPLC: 98.1%

### Example 81

### 1-Methyl-2,4-dioxo-3-(4-sulfamoyl-benzyl)-1,2,3,4-tetrahydro quinazoline-6-carboxylic acid 4-methoxy-benzylamide

The compound is obtained according to the procedure of the Step 2 of the Example 34 but using as substrates the compound obtained in the Step 1 of the Example 34 and 4-bromomethyl-benzenesulfonamide.

TLC: CH₂Cl₂/MeOH 90/10 Rf=0.48

NMR: DMSO ¹H δ(ppm): 3.55 (s,3H); 3.70 (s,3H); 4.40 (d,2H); 5.20 (s,2H); 6.90 (d,2H); 7.25 (d,2H); 7.30 (s,2H); 7.50 (d,2H); 7.55 (d,1H); 7.75 (d,2H); 8.25 (d,1H); 8.60 (s,1H); 9.2 (t,1H).

IR: 3338, 1708, 1654, 1616, 1548, 1507, 1329, 1245, 1036, 825, 751 cm⁻¹

M.P.=219.0° C.

HPLC: 94.9%

### Example 82

### 3-(4-Methanesulfonyl-benzyl)-1-methyl-2,4-dioxo-1,2,3,4tetrahydro-quinazoline-6-carboxylic acid 4-methoxy-benzylamide

The compound is obtained according to the Step 1-5 to 2-5 of the preparation B using 3-(4-methanesulfonyl-benzyl)-1-methyl-2,4-dioxo-1,2,3,4-tetrahydroquinazoline-6-carboxylic acid.

NMR: DMSO ¹H δ(ppm): 3.20 (s,3H); 3.55 (s,3H); 3.70 (s,3H); 4.40 (d,2H); 5.25 (s,2H); 6.90 (d,2H); 7.15 (d,2H); 7.50-7.60 (m,3H); 7.85 (d,2H); 8.30 (dd,1H); 8.60 (s,1H); 9.20 (t,1H).

IR: 3370, 1707, 1658, 1641, 1303, 1148, 783 cm⁻¹

M.P.=210° C.

HPLC: 97.9%

### Example 83

### 3-(4-Dimethylsulfamoyl-benzyl)-1-methyl-2,4-dioxo-1,2,3,4-tetrahydro-quinazoline-6-carboxylic acid 4-methoxy-benzylamide

Step 1: Methyl 3-(4-chlorosulfonyl-benzyl)-1-methyl-2,4-dioxo-1,2,3,4-tetrahydro-quinazoline-6-carboxylate

Into a stirred round-bottomed flask protected from moisture, 3.2 ml (47.5 mmol) of chlorosulfonic acid are introduced. The mixture is cooled with an ice bath and 2.2 g (6.80 mmol) of compound obtained in the Step 1 of Preparation C are added slowly. After 3 hours stirring at room temperature, the reaction mixture is poured in an mixture of water and ice. The precipitate is filtered and dried to provide 1.8 g of the desired product.

NMR: DMSO ¹H δ(ppm): 3.55 (s, 3H); 3.90 (s,3H); 5.15 (s,2H); 7.25 (m,2H); 7.50-7.60 (m,3H); 8.25 (dd,1H); 0.60 (s, 1H).

Step 2: Methyl 3-(4-dimethylsulfamoyl-benzyl)-1-methyl-2,4-dioxo-1,2,3,4-tetrahydro-quinazoline-6-carboxylate

To a stirred solution of 0.4 g (0.94 mmol) of the compound obtained in the preceding Step 1 in 25 ml of dichloromethane are added 3.3 ml (66 mmol) of dimethylamine 2M in THF. After 1 hour, the reaction mixture is concentrated under vacuum. A chromatography on silica gel (dichloromethane/acetone: 98/2) provides 0.370 g (yield: 91%) of the desired product.

NMR: DMSO ¹H δ(ppm): 2.6 (s,6H); 3.6 (s,3H); 3.9 (s,3H); 5.25 (d,2H); 7.60 (m,3H); 7.70 (m,2H); 8.25 (dd,1H); 8.60 (s,1H).

Step 3: 3-(4-Dimethylsulfamoyl-benzyl)-1-methyl-2,4-dioxo-1,2,3,4-tetrahydro quinazoline-6-carboxylic acid

The compound is obtained according to the procedure of the Step 2-4 of Preparation B, using as substrate the compound obtained in the preceding Step 2.

NMR: DMSO ¹H δ(ppm): 2.60 (s,6H); 3.55 (s,3H); 5.25 (s,2H); 7.60 (m,3H); 7.70 (m,2H); 8.25 (dd,1H); 8.60 (s,1H); 13.20 (bs,1H).

Step 4: 3-(4-Dimethylsulfamoyl-benzyl)-1-methyl-2,4-dioxo-1,2,3,4-tetrahydro-quinazoline-6-carboxylic acid 4-methoxy-benzylamide

The compound is obtained according to the procedure of the Example 1, but using 4-methoxybenzylamine. The desired compound crystallizes in a mixture of dichloromethane/ether.

TLC: CH₂Cl₂/MeOH 90/10 Rf=0.48 NMR: DMSO ¹H δ(ppm): 2.55 (s,6H); 3.55 (s,3H); 3.70 (s,3H); 4.40 (d,2H); 5.25 (s,2H); 6.90 (d,2H); 7.25 (d,2H); 7.55-7.60 (m,3H); 7.60-7.70 (m,2H); 8.30 (d,1H); 8.65 (s,1H); 9.20 (t,1H).

IR: 1708, 1660, 1618, 1503, 1477, 1335, 1247, 1160, 952, 760, 718 cm⁻¹

M.P.=112° C.

HPLC:94.8%

### Example 84

### 3-[4-(2-Dimethylamino-ethylsulfamoyl)-benzyl]-1-methyl-2,4-dioxo-1,2,3,4-tetrahydro-quinazoline-6-carboxylic acid 4-methoxy-benzylamide

The compound is obtained according the procedure of Steps 1 to 4 of the Example 83 using N,N′-dimethylethylene diamine in the Step 2. The desired compound crystallizes in a mixture of dichloromethane/ether.

TLC: CH₂Cl₂/MeOH 90/10 Rf=0.47

NMR: DMSO ¹H δ(ppm): 2.0-2.15 (m,6H); 2.20-2.35 (m,2H); 2.75-2.85 (m,2H); 3.55 (s,3H); 3.70 (s,3H); 4.40 (d,2H); 5.20 (s,2H); 6.85 (d,2H); 7.25 (d,2H); 7.45-7.65 (m,4H); 7.65-7.80 (m,2H); 8.25 (d,1H); 8.60 (m,1H); 9.20 (m,1H).

IR: 1707, 1656, 1618, 1508, 1477, 1326, 1249, 1155 cm⁻¹

M.P.=114° C.

HPLC: 90.9%

### Example 85

### 1-Methyl-3-(4-methylsulfamoyl-benzyl)-2,4-dioxo-1,2,3,4-tetrahydro-quinazoline-6-carboxylic acid 4-methoxy-benzylamide

Step 1: Methyl 1-methyl-3-(4-methylsulfamoyl-benzyl)-2,4-dioxo-1,2,3,4-tetrahydro-quinazoline-6-carboxylate

The compound is obtained according the procedure of Steps 1 to 3 of the Example 83 using methylamine in the Step 2.

Step 2: 1-Methyl-3-(4-methylsulfamoyl-benzyl)-2,4-dioxo-1,2,3,4-tetrahydro-quinazoline-6-carboxylic acid 4-methoxy-benzylamide

0.2 g (0,5 mmol) of the compound obtained in the preceding Step 1 is dissolved in 10 ml of dichloroethane. The solution is cooled and 3.2 ml (6.4 mmol) of trimethylaluminium 2M in toluene and 0.875 g (6.4 mmol) of 4-methoxy-benzylamine are added. The solution mixture is stirred overnight at room temperature and then 24 hours at 60° C. The solution is evaporated under vacuum and a chromatography over silica gel (dichloromethane/ether) provides 0.085 g (yield 32%) of the desired product.

TLC: CH₂Cl₂/MeOH 90/10 Rf=0.60

NMR: DMSO ¹H δ(ppm): 2.40 (d,3H); 3.55 (s,3H); 3.70 (s,3H); 4.40 (d,2H); 5.20 (s,2H); 6.85 (d,2H); 7.25 (d,2H); 7.40 (q,1H); 7.50 (d,2H); 7.60 (d,1H); 7.70 (d,2H); 8.25 (d,1H); 8.65 (s,1H); 9.2 (t,1H).

IR: 3338, 1708, 1654, 1616, 1548, 1507, 1329, 1245, 1036, 825, 751 cm⁻¹

M.P.=217.0° C.

HPLC 95.0%

### Example 86

### Methyl 3-[6-(4-methoxy-benzylcarbamoyl)-1-methyl-2,4-dioxo-1,4-dihydro-2H-quinazolin-3-ylmethyl]-benzoate

The compound is obtained according to the procedure of the Step 2 of the Example 34 but using as substrates the compound obtained in the Step 1 of the Example 34 and methyl 3-(bromomethyl)benzoate.

TLC: CH₂Cl₂/MeOH 90/10 Rf=0.80

NMR: DMSO ¹H δ(ppm): 3.50 (s,3H); 3.70 (s,3H); 3.80 (s,3H); 4.40 (d,2H); 5.2 (s,2H); 6.80-6.90 (m,2H); 7.2-7.3 (m,2H); 7.4-7.5 (m,1H); 7.5-7.6 (m,1H); 7.6-7.7 (m,1H); 7.8-7.9 (m,1H); 7.95 (s,1H); 8.30 (d,1H); 8.60 (s,1H); 9.2 (t,1H).

IR: 3254, 1729, 1705, 1659, 1637, 1502, 1299, 1249, 749 cm⁻¹

M.P.=193.5° C.

HPLC: 100%

### Example 87

### 3-[6-(4-Methoxy-benzylcarbamoyl)-1-methyl-2,4-dioxo-1,4-dihydro-2H-quinazolin-3-ylmethyl]-benzoic acid

The compound is obtained according to the procedure of the Step 2-4 of the Preparation B using as substrate the compound of the Example 86.

TLC: CH₂Cl₂/MeOH 90/10 Rf=0.70

NMR: DMSO ¹H δ(ppm): 3.60 (s,3H); 3.70 (s,3H); 4.45 (d,2H); 5.20 (s,2H); 6.90 (d,2H); 7.25 (d,2H); 7.40-7.45 (m,1H); 7.5-7.65 (m,2H); 7.80 (d,1H); 7.95 (s,1H); 8.20 ( d,1H); 8.60 (s,1H); 9.2 (t,1H); 12.95 (s,1H)

IR: 3400, 3190, 1705, 1659, 1646, 1616, 1510, 1247, 1197, 750 cm⁻¹

M.P.=182° C.

HPLC: 98.8%

### Example 88

### (E) Methyl-4-[6-(4-methoxy-benzylcarbamoyl)-1-methyl-2,4-dioxo-1,4-dihydro-2H-quinazolin-3-yl]-but-2-enoate

The compound is obtained according to the procedure of the Step 2 of the Example 34 but using as substrates the compound obtained in the Step 1 of the Example 34 and methyl 4-bromocrotonate.

TLC: CH₂Cl₂/MeOH 90/10 Rf=0.75

NMR: DMSO ¹H δ(ppm): 3.55 (s,3H); 3.60 (s,3H); 3.70 (s,3H); 4.45 (d,2H); 4.75 (2H); 5.9 (d,1H); 6.80-6.90 (m,2H); 6.9-6.95 (m,1H); 7.2-7.3 (m,2H); 7.55 (d,1H); 8.25 (d,1H); 8.60 ( s,1H); 9.2 (t,1H).

IR: 3408, 1708, 1644, 1617, 1507, 1477, 1280, 1248, 1036, 765 cm⁻¹

M.P.=107.9° C.

HPLC: 96.2%

### Example 89

### 4-[6-(4-Methoxy-benzylcarbamoyl)-1-methyl-2,4-dioxo-1,4-dihydro-2H-quinazolin-3-yl]-but-2-enoic acid

The compound is obtained according to the procedure of the Step 2-4 of the Preparation B using as substrate the compound of the Example 88.

TLC: CH₂Cl₂/MeOH 90/10 Rf=0.50

NMR: DMSO ¹H δ(ppm): 3.50 (s,3H); 3.70 (s,3H); 4.30 (d,2H); 4.70 (d,2H); 5.70-5.80 (m,1H); 6.70-6.85 (m,1H); 6.90 (d,2H); 7.25 (d,2H); 7.50 (d,1H); 8.20-8.25 (m,1H); 8.60 (s,1H); 9.2 (t,1H); 12.3 (bs,1H)

IR: 3409, 1700, 1644, 1617, 1506, 1304, 1248, 767 cm⁻¹

M.P.=245.5° C.

HPLC: 91.3%

### Example 90

### Methyl 5-[6-(4-methoxy-benzylcarbamoyl)-1-methyl-2,4-dioxo-1,4-dihydro-2H-quinazolin-3-ylmethyl]-furan-2-carboxylate

The compound is obtained according to the procedure of the Step 2 of the Example 34 but using as substrates the compound obtained in the Step 1 of the Example 34 and methyl 5-(chloromethyl)-2-furoate.

TLC: CH₂Cl₂/MeOH 90/10 Rf=0.60

NMR: DMSO ¹H δ(ppm): 3.55 (s,3H); 3.70 (s,3H); 3.75 (s,3H); 4.40 (d,2H); 5.20 (s,2H); 6.55 (d,1H); 6.85 (d,2H); 7.25 (m,3H); 7.55 (d,1H); 8.25 (d,1H); 8.60 (s,1H); 9.2 (t,1H).

IR: 3249, 1711, 1664, 1636, 1503, 1446, 1299, 1250, 1148, 1023, 824, 765 cm⁻¹

M.P.=195.5° C.

HPLC: 99.2%

### Example 91

### 5-[6-(4-Methoxy-benzylcarbamoyl)-1-methyl-2,4-dioxo-1,4-dihydro-2H-quinazolin-3-ylmethyl]-furan-2-carboxylic acid

The compound is obtained by hydrolysis, in the presence of K₂CO₃ in a mixture of dioxane/water, of the compound of the Example 90.

TLC : CH₂Cl₂/MeOH 90/10 Rf=0.10

NMR: DMSO ¹H δ(ppm): 3.55 (s,3H); 3.70 (s,3H); 4.40 (s,2H); 5.20 (s,2H); 6.50 (s,1H); 6.90 (d,2H); 7.10 (s,1H); 7.25 (d,2H); 7.55 (d,1H); 8.25 (d,1H); 8.60 (s,1H); 9.2 (t,1H); 13.05 (bs,1H).

IR: 1711, 1661, 1618, 1505, 1477, 1326, 1248, 1141, 1024, 968, 824, 787 cm⁻¹

M.P.=198° C. HPLC: 100.0%

### Example 92

### Methyl 5-[6-(4-methoxy-benzylcarbamoyl)-1-methyl-2,4-dioxo-1,4-dihydro-2H-quinazolin-3-ylmethyl]-thiophene-2-carboxylate

The compound is obtained according to the procedure of the Step 2 of the Example 34 but using as substrates the compound obtained in the Step 1 of the Example 34 and methyl 5-bromomethyl-thiophene-2-carboxylate. This compound is obtained according to the procedure described in J. Med. Chem., 1998, 41 (1), 74-95.

TLC: CH₂Cl₂/MeOH 90/10 Rf=0.20

NMR: DMSO ¹H δ(ppm): 3.55 (s,3H); 3.75 (s,3H); 3.80 (s,3H); 4.40 (d,2H); 5.30 (s,2H); 6.90 (d,2H); 7.15 (d,1H); 7.25 (d,2H); 7.55 (d,1H); 7.60 (d,1H); 8.25 (d,1H); 8.60 (s,1H); 9.2 (t,1H).

IR: 3249, 1707, 1660, 1635, 1515, 1326, 1294, 1092, 1036, 625, 749 cm⁻¹

M.P.=200.5° C.

HPLC: 91.5%

### Example 93

### 5-[6-(4-Methoxy-benzylcarbamoyl)-1-methyl-2,4-dioxo-1,4-dihydro -2H-quinazolin-3-ylmethyl]-thiophene-2-carboxylic acid

The compound is obtained by hydrolysis, in the presence of K₂CO₃ in a mixture of dioxane/water, of the compound of the Example 92.

TLC: CH₂Cl₂/MeOH 90/10 Rf=0.25

NMR: DMSO ¹H δ(ppm) : 3.55 (s,3H); 3.70 ( ,3H); 4.40 (d,2H); 5.30 (s,2H); 6.90 (d,2H); 7.15 (d,1H); 7.25 (d,2H); 7.55 (m,2H); 8.25 (d,1H); 8.65 (s,1H); 9.2 (t,1H); 13.0 (m,1H).

IR: 3241, 1705, 1662, 1632, 1541, 1325, 1246, 1032, 921, 826, 783 cm⁻¹

M.P.=198.5° C.

HPLC: 92.2%

### Example 94

### 1-Methyl-3-(4-nitro-benzyl)-2,4-dioxo-1,2,3,4-tetrahydro-quinazoline -6-carboxylic acid 4-methoxy-benzylamide

The compound is obtained according to the procedure of the Step 2 of the Example 34 but using as substrates the compound obtained in the Step 1 of the Example 34 and 4-nitrobenzyl bromide.

TLC: CH₂Cl₂/MeOH 90/10 Rf=0.47

NMR: DMSO ¹H δ(ppm): 3.55 (s,3H); 3.70 (s,3H); 4.40 (d,2H); 5.25 (s,2H); 6.90 (d,2H); 7.25 (d,2H); 7.50-7.65 (m,3H); 8.15 (d,2H); 8.25 (d,1H); 8.65 (s,1H); 9.2 (t,1H).

IR: 1706, 1661, 1618, 1513, 1477, 1345, 1248, 752 cm⁻¹

M.P.=129.0° C.

HPLC: 100%

### Example 95

### 3-(4-Amino-benzyl)-1-methyl-2,4-dioxo-1,2,3,4-tetrahydro-quinazoline -6-carboxylic acid 4-methoxy-benzylamide

1 g (2.1 mmol) of the compound of Example 94 is hydrogenated with Pd/C in a mixture of dichloromethane/methanol 80/20 v/v. After 2 hours of stirring under hydrogen atmosphere, the reaction mixture is filtered. The solvent is removed under vacuum and the crude product is concretized from a mixture of dichloromethane/ether to provide 0.800 g of the desired compound (yield: 85.8%).

TLC: CH₂Cl₂/MeOH 90/10 Rf=0.19

NMR: DMSO ¹H δ(ppm): 3.55 (s,3H); 3.70 (s,3H); 4.45 (d,2H); 4.90-5.05 (m,4H); 6.45 (d,2H); 6.90 (d,2H); 7.05 (d,2H); 7.25 (d,2H); 7.50 (d,1H); 8.25 (d,1H); 8.60 (s,1H); 9.2 (t,1H).

IR: 3387, 1701, 1647, 1615, 1511, 1478, 1245, 789 cm⁻¹

M.P.=167° C.

HPLC: 99.0%

### Example 96

### 3-(4-Dimethylamino-benzyl)-1-methyl-2,4-dioxo-1,2,3,4-tetrahydro -quinazoline-6-carboxylic acid 4-methoxy-benzylamide

To a round bottom flask protected from the moisture are added successively 0.220 g (0.5 mmol) of the compound of Example 95 in 5 ml of CH₃CN, and under stirring 0.150 g (5 mmol) of powder of paraformaldehyd, 0.095 g (1.5 mmol) of NaBH₃CN and 100 μl of acetic acid. After 2 hours at room temperature and 1h30 under reflux, the reaction mixture is taken up in dichloromethane and washed with a solution of NaOH 1M. The organic phase is decanted, washed, dried and then concentrated under vacuum. The product is recrystallized from acetonitrile to provide 0.130 g (yield: 55%) of the desired compound.

TLC: CH₂Cl₂/MeOH 90/10 Rf=0.42

NMR: DMSO ¹H δ(ppm): 2.80 (s,6H); 3.50 (s,3H); 3.70 (s,3H); 4.40 (d,2H); 5.00 (s,2H); 6.60 (d,2H); 6.90 (d,2H); 7.15-7.25 (m,4H); 7.50 (d,1H); 8.20 (d,1H); 8.60 (s,1H); 9.2 (t,1H).

IR: 1699, 1654, 1640, 1616, 1508, 1324, 1324 cm⁻¹

M.P.=205.0° C.

HPLC: 98.9%

### Example97

### 3-(4-Acetylamino-benzyl)-1-methyl-2,4-dioxo-1,2,3,4-tetrahydro -quinazoline-6-carboxylic acid 4-methoxy-benzylamide

To a round bottom protected from the moisture is added 0.190 g (0.43 mmol) of the compound of Example 95 in 10 ml of dichloromethane. The solution is stirred and 36 μl (40 mg, 0.51 mmol) of acetyl chloride and 72 μl of triethylamine are added. After 1 hour at room temperature 36 μl of acetyl chloride and 72 μl of triethylamine are added. After 1 hour, the organic phase is washed with a solution of HCl 1M and dried. A chromatography over silica gel (dichloromethane/ether) provides 0.120 g (yield: 57%) of the desired product.

TLC: CH₂Cl₂/MeOH 90/10 Rf=0.17

NMR: DMSO ¹H δ(ppm) : 2.0 (s,3H); 3.55 (s,3H); 3.70 (s,3H); 4.40 (d,2H); 5.05 (s,2H); 6.90 (d,2H); 7.20-7.30 (m,4H); 7.45 (d,2H); 7.50 (d,1H); 8.25 (d,1H); 8.60 (s,1H); 9.2 (t,1H); 9.85 (s,1H).

IR: 3330, 1661, 1617, 1511, 1475, 1322, 1244, 825, 752 cm⁻¹

M.P.=251.0° C.

HPLC: 100.0%

### Example 98

3-[4-(N,N-methylsulfonylamino)-benzyl]-1-methyl-2,4-dioxo-1,2,3,4-tetrahydro-quinazoline-6-carboxylic acid 4-methoxy-benzylamide

The compound is obtained according to the procedure of the Example 97 using as substrates the compound obtained in the Example 95 and methanesulfonyl chloride.

TLC: CH₂Cl₂/MeOH 90/10 Rf=0.40

NMR: DMSO ¹H δ(ppm): 3.50 (s,6H); 3.55 (s,3H); 3.70 (s,3H); 4.40 (d,2H); 5.20 (s,2H); 6.90 (d,2H); 7.25 (d,2H); 7.40-7.50 (m,4H); 7.55 (d,1H); 8.25 (d,1H); 8.65 (s,1H); 9.2 (t,1H).

IR: 1655, 1639, 1507, 1376, 1252, 1157, 905, 761 cm⁻¹

M.P.=198° C.

HPLC: 100.0%

### Example 99

### 3-(Benzofurazan-5-ylmethyl)-1-methyl-2,4-dioxo-1,2,3,4-tetrahydro -quinazoline-6-carboxylic acid 4-methoxy-benzylamide

The compound is obtained according to the procedure of the Step 2 of Example 34 using the compound obtained in the Step 1 of the Example 34 and 5-bromomethyl benzofurazan.

TLC: CH₂Cl₂/MeOH 90/10 Rf=0.80

NMR: DMSO ¹H δ(ppm): 3.55 (s,3H); 3.70 (s,3H); 4.40 (d,2H); 5.25 (s,2H); 6.90 (d,2H); 7.25 (d,2H); 7.60 (m,2H); 7.90 (s,1H); 8.0 (d,1H); 8.25 (d,1H); 8.65 (s,1H); 9.2 (t,1H).

IR: 2370, 1701, 1653, 1617, 1499, 1477, 1326, 1243, 1181, 1028, 881, 781 cm⁻¹

M.P.=140.5° C.

HPLC: 100.0%

### Example 100

### 3-[2-(4-Fluorophenoxy)-ethyl]-1-methyl-2,4-dioxo-1,2,3,4-tetrahydro -quinazoline-6-carboxylic acid 4-methoxy-benzylamide

The compound is obtained according to the procedure of the Step 2 of Example 34 using the compound obtained in the Step 1 of the Example 34 and 4-fluorophenoxyethyl bromide.

TLC: CH₂Cl₂/MeOH 90/10 Rf=0.60

NMR: DMSO ¹H δ(ppm): 3.55 (s,3H); 3.70 (s,3H); 4.20 (d,2H); 4.3-4.4 (m,2H); 4.4-4.50 (m,2H); 6.80-7.0 (m,4H); 7.0-7.1 (m,2H); 7.2-7.30 (m,2H); 7.4-7.5 (m,1H); 8.20-8.30 (m,1H); 8.60-8.70 (m,1H); 9.2 (t,1H).

IR: 1707, 1656, 1641, 1520, 1475, 1247, 1209, 1034, 828, 752 cm⁻¹

M.P.=159.6° C.

HPLC: 99.7%

### Example 101

### 3-(2Benzenesulfonyl-ethyl)-1-methyl-2,4-dioxo-1,2,3,4-tetrahydro -quinazoline-6-carboxylic acid 4-methoxy-benzylamide

The compound is obtained according to the procedure of the Step 2 of Example 34 using the compound obtained in the Step 1 of the Example 34 and 2-chloroethyl phenyl sulphone.

TLC: CH₂Cl₂/MeOH 90/10 Rf=0.55

NMR: DMSO ¹H δ(ppm): 3.50 (s,3H); 3.6-3.70 (m,2H); 3.75 (s,3H); 4.3 (d,2H); 4.4-4.50 (m,2H); 6.90 (d,2H); 7.30 (d,2H); 7.4-7.7 (m,4H); 7.9 (d,2H); 8.20 (d,1H); 8.60 (s,1H); 9.2 (t,1H).

IR: 3274, 1708, 1663, 1638, 1514, 1499, 1249, 1147, 1034, 825, 746 cm⁻¹

M.P.=192.9° C.

HPLC: 96.0%

### Example 102

### 3-(3-fluoro-4-methoxy-benzyl)-1-methyl-2,4-dioxo-1,2,3,4-tetrahydro -quinazoline-6-carboxylic acid 4-methoxy benzylamine

The compound is obtained according to the procedure of the Step 2 of Example 34 using the compound obtained in the Step 1 of the Example 34 and 4-chloromethyl-2-fluoro-1-methoxy-benzene.

TLC: CH₂Cl₂/MeOH 90/10 Rf=0.80

NMR: DMSO ¹H δ(ppm): 3.55 (s,3H); 3.75 (s,3H); 3.80 (s,3H); 4.4 (d,2H); 5.10 (s,2H); 6.90 (d,2H); 7.20 (m,5H); 7.55 (d,1H); 8.25 (d,1H); 8.65 (s,1H); 9.2 (t,1H).

IR: 3411, 2362, 1705, 1644, 1617, 1513, 1325, 1275, 1246, 1028, 827, 786 cm⁻¹

M.P.=136° C.

HPLC: 100.0%

### Example 103

### 1-Methyl-2,4-dioxo-3-[4-(2H-tetrazol-5-yl)-benzyl]-1,2,3,4-tetrahydro -quinazoline-6-carboxylic acid 4-methoxy-benzylamide

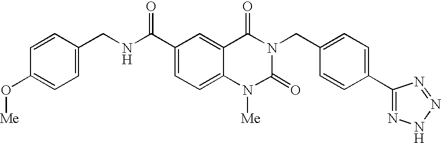

A solution of 3 g (6.6 mmol) of compound of the Example 60 in 100 ml of toluene, 1.3 g (19.8 mmol) of NaN₃ and 2.72 g (19.8 mmol) of triethylamine hydrochloride are heated at 80° C. under an inert atmosphere. After 5 hours, 10 ml of DMF are added and the reflux is maintained overnight. After cooling, the precipitate is filtered and washed successively with AcOEt, MeOH and HCl 3N. The solid obtained is treated under reflux by a mixture of AcOEt/MeOH and filtered. A chromatography over silica gel (DMF with NH₄OH 10%) provides 1.2 g of the desired compound (yield: 36%).

TLC: CH₂Cl₂/MeOH 80/20 Rf=0.30

NMR: DMSO ¹H δ(ppm): 3.50 (bs,1H);.3.55 (s,3H); 3.70 (s,3H); 4.4 (m,2H); 5.20 (s,2H); 6.90 (m,2H); 7.25 (m,2H); 7.50 (m,3H); 8.0 (m,2H); 8.3 (m,1H); 8.70 (s,1H); 9.2 (m,1H).

M.P.−286° C.

HPLC: 96.7%

### Example 104

### 1-Methyl-3-[4-(5-methyl-1,2,3,4-oxadiazol-3-yl)-benzyl]-2,4-dioxo-1,2,3,4-tetrahydro-quinazoline-6-carboxylic acid 4-methoxy-benzylamide

The compound is obtained according to the procedure of the Step 2 of Example 34 using the compound obtained in the Step 1 of the Example 34 and 3-(4-chloromethyl-phenyl)-5-methyl-[1,2,4]oxadiazole (which is obtained in 4 steps from 4-hydroxymethyl -benzonitrile).

TLC: CH₂Cl₂/MeOH 95/5 Rf=0.50

NMR: CDCl₃ ¹H δ(ppm): 2.60 (s,3H); 3.60 (s,3H); 3.80 (s,3H); 4.55 (m,2H); 5.25 (s,2H); 6.60 (s,1H); 6.85 (m,2H); 7.30 (m,3H); 7.55 (m,2H); 7.90 (m,2H); 8.3 (m,1H); 8.50 (s,1H).

M.P.=235.0° C.

HPLC: 95.1%

### Example 105

### 1-Methyl-3-[4-(3-methyl-1,2,4-oxadiazol-5-yl)-benzyl]-2,4-dioxo-1,2,3,4-tetrahydro-quinazoline-6-carboxylic acid 4-methoxy-benzylamide

To a round bottom containing 4 Å molecular sieves, 5 ml of DMF, 76 mg (1.02 mmol) of N-hydroxy-acetamidine and 25 mg (1.02 mmol) of NaH are introduced. The mixture is stirred for 15 minutes and 0.5 g (1.02 mmol) of compound of the Example 34 is added. The reaction is heated at 65° C. for 4 hours and then filtered over Celite. The filtrate is poured onto 100 ml of water. The precipitate obtained is filtered, washed successively by ethanol, water and ether, and dried to provide 0.210 g (yield: 40%) of the desired compound.

TLC: CH₂Cl₂/MeOH 95/5 Rf=0.50

NMR: DMSO ¹H δ(ppm): 3.3 (s,3H); 3.55 (s,3H); 3.70 (s,3H); 4.40 (m,2H); 5.25 (s,2H); 6.90 (m,2H); 7.25 (m,2H); 7.55 (m,3H); 8.0 (d,2H); 8.3 (m,1H); 8.60 (s,1H); 9.2 (m,1H).

M.P.=226.0° C.

HPLC: 98.6%

### Example 106

### Methyl 2-chloro-4-[6-(4-methoxy-benzylcarbamoyl)-1-methyl-2,4-dioxo -1,4-dihydro-2H-quinazolin-3-ylmethyl]-benzoate

The compound is obtained according to the procedure of the Step 2 of Example 34 using the compound obtained in the Step 1 of the Example 34 and methyl 2-chloro-4-chloromethyl-benzoate.

NMR: DMSO ¹H δ(ppm): 3.55 (s,3H); 3.70 (s,3H); 3.80 (s,3H); 4.40 (d,2H); 5.20 (s,2H); 6.90 (m,2H); 7.25 (m,3H); 7.60 (d,1H); 7.75 (d,1H); 7.95 (s,1H); 8.3 (m,1H); 8.70 (s,1H); 9.2 (m,1H).

M.P.=229.0° C.

HPLC: 98.8%

### Example 107

### 2-Chloro-4-[6-(4-methoxy-benzylcarbamoyl)-1-methyl-2,4-dioxo-1,4-dihydro-2H-quinazolin-3-ylmethyl]-benzoic acid

The compound is obtained by hydrolysis of the compound of Example 106 with a solution of aqueous methanol and K₂CO₃.

TLC: CH₂Cl₂/MeOH 90/10 Rf=0.30

NMR: DMSO ¹H δ(ppm): 3.55 (s,3H); 3.70 (s,3H); 4.40 (m,2H); 5.20 (s,2H); 6.85 (m,2H); 7.20 (m,3H); 7.60 (m,1H); 7.70 (m,1H); 7.95 (m,1H); 8.3 (m,1H); 8.60 (s,1H); 9.2 (m,1H); 13.2 (s,1H).

M.P.=216.0° C.

HPLC: 96.5%

### Example 108

### 1-Methyl-3-[4-(1-methyl-1H-tetrazol-5-yl)-benzyl]-2,4-dioxo-1,2,3,4-tetrahydro-quinazoline-6-carboxylic acid 4-methoxy-benzylamide

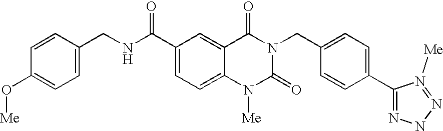

The compound is obtained according to the procedure of the Step 2 of Example 34 using the compound obtained in the Step 1 of the Example 34 and 5-(4-chloromethyl-phenyl)-1-methyl-1H-tetrazole

TLC: CH₂Cl₂/MeOH 90/10 Rf=0.40

NMR: DMSO ¹H δ(ppm): 3.55 (s,3H); 3.70 (s,3H); 4.10 (s,3H); 4.40 (m,2H); 5.20 (s,2H); 6.80 (d,2H); 7.25 (d,2H); 7.50 (m,3H); 7.80 (m,2H); 8.2 (d,1H); 8.60 (s,1H); 9.2 (s,1H).

M.P.=143.0° C.

HPLC: 100%

### Example 109

### 1-Methyl-3-[4-(2-methyl-2H-tetrazol-5-yl)-benzyl]-2,4-dioxol-1,2,3,4-tetrahydro-quinazoline-6-carboxylic acid 4-methoxy-benzylamide

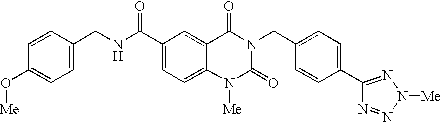

The compound is obtained according to the procedure of the Step 2 of Example 34 using the compound obtained in the Step 1 of the Example 34 and 5-(4-chloromethyl-phenyl)-2-methyl-2H-tetrazole.

TLC: CH₂Cl₂/MeOH 90/10 Rf=0.50

NMR: DMSO ¹H δ(ppm): 3.50 (s,3H); 3.70 (s,3H); 4.40 (m,5H); 5.20 (s,2H); 6.90 (m,2H); 7.25 (m,2H); 7.50 (m,3H); 8.0 (m,2H); 8.3 (d,1H); 8.60 (s,1H); 9.2 (m,1H).

M.P.=226.0° C.

HPLC: 98.2%

### Example 110

### Methyl 2-methoxy-4-[6(4-methoxy-benzylcarbamoyl)-1-methyl-2,4-dioxo-1,4-dihydro-2H-quinazolin-3-ylmethyl]benzoate

The compound is obtained according to the procedure of the Step 2 of Example 34 using the compound obtained in the Step 1 of the Example 34 and methyl 4-bromomethyl-2-methoxy-benzoate.

TLC: CH₂Cl₂/MeOH 90/10 Rf=0.60

NMR: CDCl₃ ¹H δ(ppm): 3.60 (s,3H); 3.80 (s,3H); 3.85 (s,3H); 3.90 (s,3H); 4.55 (d,2H); 5.20 (s,2H); 6.45 (m,1H); 6.80 (d,2H); 7.05 (d,1H); 7.20 (m,4H); 7.70 (d,1H); 8.3 (d,1H); 8.50 (s,1H).

M.P.=170.0° C.

HPLC: 98.6%

### Example 111

### 2-Methoxy-4-[6-(4-methoxy-benzylcarbamoyl)-1-methyl-2,4-dioxo-1,4-dihydro-2H-quinazolin-3-ylmethyl]-benzoic acid

The compound is obtained by hydrolysis of compound of the Example 110 using as reagent K₂CO₃ in a mixture of methanol and water. After acidification of the reaction mixture, the precipitate obtained is filtered off to provide the desired product.

TLC: CH₂Cl₂/MeOH 90/10 Rf=0.50

NMR: DMSO ¹H δ(ppm): 3.60 (s,3H); 3.70 (s,3H); 3.80 (s,3H); 4.40 (s,2H); 5.15 (s,2H); 6.90 (m,3H); 7.10 (s,1H); 7.30 (m,2H); 7.60 (m,2H); 8.3 (m,1H); 8.60 (s,1H); 9.2 (m,1H); 12.5 (bs,1H).

M.P.=189° C.

HPLC: 100.0%

### Example 112

### Methyl 2-hydroxy-4-[6-(4-methoxy-benzylcarbamoyl)-1-methyl-2,4-dioxo-1,4-dihydro-2H-quinazolin-3-ylmethyl]-benzoate

To a stirred solution of 1 g (1.93 mmol) of compound of the Example 111 in 15 ml of dichloromethane, maintained at 0° C., are added dropwise, under an inert atmosphere, 7.7 ml (7.7 mmol) of BCl₃ 1M/l in dichloromethane. After 15 minutes of stirring at 0° C. and 1 hour at room temperature, the reaction mixture is poured on ice and extracted by ethyl acetate. The organic phase is dried and concentrated under vacuum. The precipitate obtained is purified by chromatography over silica gel (dichloromethane/methanol: 99/1) to provide 0.460 g (yield: 47%) of the desired product.

TLC: CH₂Cl₂/MeOH 90/10 Rf=0.60

NMR: DMSO ¹H δ(ppm): 3.50 (s,3H); 3.70 (s,3H); 3.85 (s,3H); 4.40 (d,2H); 5.10 (s,2H); 6.85 (m,4H); 7.25 (d,2H); 7.55 (d,1H); 7.70 (d,1H); 8.3 (m,1H); 8.60 (s,1H); 9.2 (m,1H); 10.5 (s,1H).

M.P.=205.0° C.

HPLC: 100.0%

### Example 113

### 2-Hydroxy-4-[6-(4-methoxy-benzylcarbamoyl)-1-methyl-2,4-dioxo-1,4-dihydro-2H-quinazolin-3-ylmethyl]-benzoic acid

The compound is obtained by hydrolysis of compound of the Example 112 using as reagent K₂CO₃ in a mixture of methanol and water. After acidification of the reaction mixture, the precipitate obtained is filtered off to provide the desired product.

TLC: CH₂Cl₂/MeOH 90/10 Rf=0.60

NMR: DMSO ¹H δ(ppm): 3.50 (s,3H); 3.70 (s,3H); 4.40 (d,2H); 5.15 (s,2H); 6.80 (m,4H); 7.25 (m,2H); 7.55 (m,1H); 7.70 (d,1H); 8.3 (m,1H); 8.60 (s,1H); 9.2 (m,1H); 11.3 (bs,1H); 13.8 (s,1H).

M.P.=262.0° C.

HPLC: 98.2%

### Example 114

### Methyl 2-methyl-4-[6-(4-methoxy-benzylcarbamoyl)-1-methyl-2,4-dioxo-1,4-dihydro-2H-quinazolin-3-ylmethyl]-benzoate

The compound is obtained according to the procedure of the Step 2 of Example 34 using the compound obtained in the Step 1 of the Example 34 and methyl 4-bromomethyl-2-methyl benzoate.

TLC: CH₂Cl₂/MeOH 90/10 Rf=0.80

NMR: DMSO ¹H δ(ppm): 2.5 (s,3H); 3.50 (s,3H); 3.70 (s,3H); 3.80 (s,3H); 4.40 (s,2H); 5.10 (s,2H); 6.90 (m,2H); 7.25 (m,4H); 7.50 (d,1H); 7.70 (d,1H); 8.2 (m,1H); 8.60 (s,1H); 9.2 (s,1H).

M.P.=167.0° C.

HPLC: 100.0%

### Example 115

### 2-Methyl-4-[6-(4-methoxy-benzylcarbamoyl)-1-methyl-2-dioxo-1,4-dihydro-2H-quinazolin-3-ylmethyl]-benzoic acid

The compound is obtained by hydrolysis of compound of the Example 114 using first as reagent K₂CO₃ in a mixture of methanol and water, and secondly LiOH in reflux for 2 days. After acidification of the reaction mixture, the precipitate obtained is filtered off to provide the desired compound.

TLC: CH₂Cl₂/MeOH 90/10 Rf=0.50

NMR: DMSO ¹H δ(ppm): 2.5 (s,3H); 3.55 (s,3H); 3.80 (s,3H); 4.40 (d,2H); 5.10 (s,2H); 6.80 (d,2H); 7.25-7.1 (m,4H); 7.55 (m,1H);7.75 (m,1H); 8.2 (d,1H); 8.60 (s,1H); 9.2 (t,1H); 12.7 (s,1H)

M.P.=179.0° C.

HPLC: 95.6%

### Example 161

### 1-Methyl-2,4,-dioxo-3-(pyridin4-methyl)-1,2,3,4-tetrahydro -quinazoline-carboxylic acid (benzo[1,3]dioxol-5-ylmethyl)-amide

Step 1: Methyl 2,4-dioxo-1-methyl-3-(pyridine-4-ylmethyl)-1,2,3,4-tetrahydro -quinazoline-6-carboxylate

The compound is obtained according to the procedure of the Step 4 of Example 15 using the compound obtained in the Step 2 of the Example 20.

Step 2: 2,4-Dioxo-1-methyl-3-(pyridine-4-ylmethyl)-1,2,3,4-tetrahydro -quinazoline-6-carboxylic acid

The compound is obtained according to the procedure of the Step 2-4 of the Preparation B using the compound obtained in the preceding Step 1.

Step 3: 1-Methyl-2,4-dioxo-3-(pyridin-4-methyl)-1,2,3,4-tetrahydro -quinazoline-6-carboxylic acid (benzo[1,3]dioxol-5-ylmethyl)-amide

To a stirred solution of 0.2 g (0.65 mmol) of compound obtained in the preceding Step 2 in 7 ml of dichloromethane are added 0.113 g (0.65 mmol) of EDCI, 0.080 g (0.65 mmol) of HOBT and 0.064 g (0.060 ml, 0.65 mmol) of 3,4-methylenedioxy-benzylamine. After 20 hours of stirring at room temperature and an usual treatment, 0.140 g (yield: 48%) of the desired product are obtained.

TLC: CH₂Cl₂/MeOH 90/10 Rf=0.80

NMR: DMSO ¹H δ(ppm): 3.60 (s,3H); 4.40 (d,2H); 5.20 (s,2H); 6.0 (s,2H); 6.80-6.95 (m,3H); 7.25-7.35 (m,2H); 7.55-7.60 (m,2H); 8.25-8.35 (m,1H); 8.45-8.50 (m,2H); 8.65 (s,1H); 9.20 (t,1H).

IR: 3265, 1707, 1663, 1618, 1501, 1490, 1254, 1037, 925 cm⁻¹

M.P.=161.7° C.

HPLC: 94.6%

### Example 117

### 1-Methyl-2,4-dioxo-3-(pyridin-4-ylmethyl)-1,2,3,4-tetrahydro-quinazoline-carboxylic acid 4-methoxy-benzylamide

The compound is obtained according to the procedure of the Step 3 of Example 116 using the compound obtained in the Step 2 of the Example 116 and 4-methoxy-benzylamine. 0.280 g (yield: 25%) of the desired product is isolated after a chromatography over silica gel.

TLC: CH₂Cl₂/MeOH 90/10 Rf=0.70

NMR: DMSO ¹H δ(ppm): 3.60 (s,3H); 3.70 (s,3H); 4.40 (d,2H); 5.15 (s,2H); 6.80 (d,2H); 7.2-7.3 (m,4H); 7.55-7.60 (m,1H); 8.25-8.30 (m,1H); 8.45 ( d,2H); 8.60 (s,1H); 9.20 (m,1H).

IR: 3231, 1706, 1657, 1625, 1505, 1324, 1248, 1039, 827 cm⁻¹

M.P.=180.7° C.

HPLC: 94.3%

### Example 118

### 1-Methyl-2,4-dioxo-3-(pyridin-4-ylmethyl)-1,2,3,4-tetrahydro -quinazoline-6-carboxylic acid 4-hydroxy-benzylamide

To a stirred solution of 0.280 g (0.67 mmol) of compound of the Example 117 in 20 ml of dichloromethane, maintained at 0° C., are added, under an inert atmosphere, 1.7 g (0.63 ml, 6.7 mmol) of BBr₃ in 2 ml of dichloromethane. After 20 minutes of stirring at room temperature, the reaction mixture is poured on a saturated solution of NaHCO₃, decanted, and extracted. The organic phase is dried and concentrated under vacuum to provide 0.150 g (yield: 53.4%) of the desired product.

TLC: CH₂Cl₂/MeOH 90/10 Rf=0.60

NMR: DMSO ¹H δ(ppm): 3.60 (s,3H); 4.40 (d,2H); 5.20 (s,2H); 6.70 (d,2H); 7.15 (d,2H); 7.3 (d,2H); 7.55-7.60 (m,1H); 8.30 (d,1H); 8.50 (d,2H); 8.65 (s,1H); 9.20 (m,1H); 9.30 (s,1H)

IR: 3388, 1701, 1656, 1639, 1615, 1508, 1251, 830, 772, 751 cm⁻¹

M.P.=137.7° C.

HPLC: 91.1%

### Example 119

### Methyl 4-[6-(3-methoxy-benzylcarbamoyl)-1-methyl-2,4-dioxo-1,4-dihydro-2H-quinazolin-3-ylmethyl]-benzoate

Step 1: Benzyl 3-(4-methoxycarbonyl-benzyl)-2,4-dioxo-1,2,3,4-tetrahydro-quinazoline-6-carboxylate

The compound is obtained according to the procedure of Step 1-5 to Step 2-5 of the Preparation B using, in Step 1-5, 4-amino-isophtalic acid 1-benzylester 3-methyl ester and methyl 4-aminomethyl benzoate. The desired product is purified by reflux in methanol.

TC: CH₂Cl₂/MeOH 95/5 Rf=0.65

NMR: DMSO ¹H δ(ppm): 3.8 (s, 3H); 5.10 (s,2H); 5.35 (s,2H); 7.20-7.80 (m,8H); 7.80-7.90 (m,2H); 8.20-8.30 (m,1H); 8.50 (s,1H); 11.90 (s,1H).

HPLC: 97.0%

Step 2: Benzyl 3-(4-methoxycarbonyl-benzyl)-1-methyl-2,4-dioxo-1,2,3,4-tetrahydro-quinazoline-6-carboxylate

The compound is obtained according to the procedure of the Step 4 of the Example 15 using the compound obtained in the preceding Step 1.

TLC: CH₂Cl₂/MeOH 95/5 Rf=0.65

NMR: DMSO ¹H δ(ppm): 3.60 (s,3H); 3.80 (s,3H); 5.20 (s,2H); 5.35 (s,2H); 7.30-7.60 (m,8H); 7.80-7.90 (m,2H); 8.20-8.30 (m,1H); 8.60 (s,1H).

HPLC: 97.0%

Step 3: 3-(4-Methoxycarbonyl-benzyl)-1-methyl-2,4-dioxo-1,2,3,4-tetrahydro-quinazoline-6-carboxylic acid

To a stirred solution of 10.8 g (23.6 mmol) of the compound obtained in the preceding Step 2 in 120 ml of dichloromethane and 80 ml of methanol, are added 3.2 g of Pd/C at 10%. The reaction mixture is stirred under hydrogen atmosphere for 1 hour at room temperature, followed by filtration over Celite. The filtrate is concentrated under vacuum to give a first crystallized crop. The unsoluble part is extracted three times by a mixture of methanol/water/saturated solution of NaHCO₃. The organic phases are gathered and acidified to pH 1 by a concentrated solution of chlorhydric acid, to give to a second crop corresponding to the desired product. The two crops are put together and dried under vacuum to provide 6.9 g of the desired product (yield: 79%).

NMR: DMSO ¹H δ(ppm): 3.60 (s,3H); 3.80 (s,3H); 5.20 (s,2H); 7.40 (dd,2H); 7.60 (dd,1H); 7.90 (dd,2H); 8.30 (dd,1H); 8.60 (s,1H); 13.20 (bs,1H).

HPLC: >97.0%

Step 4: Methyl 4-[6-(3-methoxy-benzylcarbamoyl)-1-methyl-2,4-dioxo-1,4-dihydro-2H-quinazolin-3-ylmethyl]-benzoate

The compound is obtained according to the procedure of the Example 1 using the compound obtained in the preceding Step 3 and 3-methoxy-benzylamine.

TLC: CH₂Cl₂/MeOH 90/10 Rf=0.70

NMR: DMSO ¹H δ(ppm): 3.55 (s,3H); 3.70 (s,3H); 3.80 (s,3H); 4.45 (d,2H); 5.20 (s,2H); 6.80 (d,1H); 6.90 (m,2H); 7.25 (m,1H); 7.45 (d,2H); 7.55 (d,1H); 7.85 (d,2H); 8.25 (d,1H); 8.60 (s,1H); 9.25 (t,1H).

IR: 3435, 2361, 1716, 1703, 1666, 1617, 1498, 1455, 1282, 1125, 839, 749, cm⁻¹

M.P.=199.0° C.

HPLC: 98.6%

### Example 120

### 4-[6-(-Methoxy-benzylcarbamoyl)-1-methyl-2,4-dioxo-1,4-dihydro-2H-quinazolin-3-ylmethyl]-benzoic acid

The compound is obtained by hydrolysis of compound of the Example 119 using as reagent K₂CO₃ in a mixture of methanol and water under reflux for 8 hours. After acidification of the reaction mixture, the precipitate obtained is filtered off to provide the desired product.

TLC: CH₂Cl₂/MeOH 90/10 Rf=0.40

NMR: DMSO ¹H δ(ppm): 3.55 (s,3H); 3.75 (s,3H); 4.45 (d,2H); 5.20 (s,2H); 6.80 (d,1H); 6.90 (m,2H); 7.25 (t,1H); 7.45 (d,2H); 7.55 (d,1H); 7.85 (d,2H); 8.25 (d,1H); 8.65 (s,1H); 9.25 (t,1H); 12.85 (bs,1H)

IR: 3395, 2345, 1719, 1647, 1616, 1501, 1310, 1238, 1052, 839, 781, 751 cm⁻¹

M.P.=279.0° C.

HPLC: 97.4%

### Example 121

### Methyl 4-[1-methyl-6-(4-methylsulfanyl-benzylcarbamoyl)-2,4-dioxo -1,4-dihydro-2H-quinazolin-3-ylmethyl]-benzoate

The compound is obtained according to the procedure of the Example 1 using the compound obtained in the Step 3 of Example 1 19 and 4-methylthio-benzylamine.

TLC: CH₂Cl₂/MeOH 90/10 Rf=0.80

NMR: DMSO ¹H δ(ppm): 2.45 (s,3H); 3.55 (s,3H); 3.80 (s,3H); 4.45 (d,2H); 5.20 (s,2H); 7.20 (m,4H); 7.45 (d,2H); 7.55 (s,1H); 7.90 (d,2H); 8.25 (d,1H); 8.60 (s,1H); 9.20 (t,1H).

IR: 3395, 1708, 1656, 1641, 1508, 1479, 1330, 1280, 1254, 1117, 783, 749, cm⁻¹

M.P.=172° C.

HPLC: 99.2%

### Example 122

### 4-[1-Methyl-6-(4-methylsulfanyl-benzylcarbamoyl)-2,4-dioxo-1,4-dihydro-quinazolin-3-ylmethyl]-benzoic acid

The compound is obtained by hydrolysis of compound of the Example 121 using as reagent K₂CO₃ in a mixture of methanol and water under reflux for 48 hours. After acidification of the reaction mixture, the precipitate obtained is filtered off to provide the desired product.

TLC: CH₂Cl₂/MeOH 90/10 Rf=0.35

NMR: DMSO ¹H δ(ppm): 2.45 (s,3H); 3.55 (s,3H); 4.45 (d,2H); 5.20 (s,2H); 7.25 (m,4H); 7.40 (d,2H); 7.55 (d,1H); 7.85 (d,2H); 8.25 (d,1H); 8.60 (s,1H); 9.25 (t,1H); 12.85 (bs,1H);

IR: 1705, 1656, 1642, 1616, 1479, 1330, 1247, 1101, 1020, 760, 751 cm⁻¹

M.P.=171° C.

HPLC: 98.0%

### Example 123

### Methyl 4-[1-methyl-2,4-dioxo-6-(4-trifuoromethoxy-benzylcarbamoyl) -1,4-dihydro-2H-quinazolin-3-ylmethyl]-benzoate

The compound is obtained according to the procedure of the Example 1 using the compound obtained in the Step 3 of Example 119 and 4-trifluoromethoxy-benzylamine.

TLC: CH₂Cl₂/MeOH 95/5 Rf=0.35

NMR: DMSO ¹H δ(ppm): 3.55 (s,3H); 3.80 (s,3H); 4.50 (d,2H); 5.20 (s,2H); 7.30 (d,2H); 7.35-7.50 (m,4H); 7.55 (d,1H); 7.90 (d,2H); 8.25 (d,1H); 8.65 (s,1H); 9.30 (t,1H).

IR: 1712, 1656, 1639, 1506, 1274, 1156, 1104, 751 cm⁻¹

M.P.=212° C.

HPLC: 99.6%

### Example 124

### Methyl 4-[6-(4-fluoro-benzylcarbamoyl)-1-methyl-2,4-dioxo-1,4-dihydro-2H-quinazolin-3-ylmethyl]-benzoate

The compound is obtained according to the procedure of the Example 1 using the compound obtained in the preceding Step 3 and 4-fluorobenzylamine.

TLC: CH₂Cl₂/MeOH 95/5 Rf=0.45

NMR: DMSO ¹H δ(ppm): 3.55 (s,3H); 3.80 (s,3H); 4.45 (d,2H); 5.20 (s,2H); 7.10-7.20 (m,2H); 7.30-7.40 (m,2H); 7.40-7.50 (d,2H); 7.55 (d,1H); 7.85 (d,2H); 8.25 (d,1H); 8.65 (s,1H); 9.25 (t,1H).

IR: 1709, 1657, 1618, 1499, 1264, 768, 749, 716 cm⁻¹

M.P.=198° C.

HPLC: 98.2%

### Example 125

### 4-[6-(4-Fluoro-benzylcarbamoyl)-1-methyl-2,4-dioxo-1,4-dihydro-2H-quinazolin-3-ylmethyl]-benzoic acid

The compound is obtained according to the procedure of the Step 2-4 of the Preparation B using the compound obtained in the Example 124.

TLC: CH₂Cl₂/MeOH 95/5 Rf=0.25

NMR: DMSO ¹H δ(ppm): 3.55 (s,3H); 4.45 (d,2H); 5.20 (s,2H); 7.10-7.20 (m,2H); 7.30-7.40 (m,2H); 7.45 (d,2H); 7.55 (d,1H); 7.90 (d,2H); 8.25 (d,1H); 8.65 (s,1H); 9.25 (t,1H); 12.90 (bs,1H)

IR: 3661, 2765, 1710, 1649, 1617, 1505, 1224, 829, 752 cm⁻¹

M.P.=272° C.

HPLC: 98.0%

### Example 126

### Methyl 4-{6-[(benzofurazan-5-ylmethyl)-carbamoyl]-1-methyl-2,4-dioxo-1,4-dihydro-2H-quinazolin-3-ylmethyl}-benzoate

The compound is obtained according to the procedure of the Example 1 using the compound obtained in the Step 3 of Example 119 and C-benzofurazan-5-yl-methylamine, which is obtained from 5-bromomethyl-benzofurazan by reaction in a first step with sodium diformylamide in acetonitrile at 70° C. overnight, and in a second step by a treatment for 2 hours under reflux to a solution of ethanol/HCl 5%.

TLC: CH₂Cl₂/MeOH 95/5 Rf=0.70

NMR: DMSO ¹H δ(ppm): 3.55 (s,3H); 3.85 (s,3H); 4.65 (d,2H); 5.25 (s,2H); 7.45 (d,2H); 7.60 (d,2H); 7.90 (m,3H); 8.00 (d,1H); 8.30 (d,1H); 8.65 (s,1H); 9.40 (t,1H).

IR: 3257, 1731, 1702, 1659, 1619, 1506, 1419, 1281, 1109, 877, 769, 751 cm⁻¹

M.P.=234° C.

HPLC: 98.6%

### Example 127

### 4-{6-[(Benzofurazan-5-ylmethyl)-carbamoyl]-1-methyl-2,4-dioxo-1,4-dihydro-2H-quinazolin-3-ylmethyl}-benzoic acid

The compound is obtained according to the procedure of the Step 2-4 of the Preparation B using the compound obtained in the Example 126. After acidification, the precipitate is filtered off.

TLC: CH₂Cl₂ MeOH 95/5 Rf=0.35

NMR: DMSO ¹H δ(ppm): 3.55 (s,3H); 4.60 (d,2H); 5.20 (s,2H); 7.40 (d,2H); 7.60 (d,2H); 7.85 (d,3H); 8.00 (d,1H); 8.25 (d,1H); 8.65 (s,1H); 9.40 (t,1H); 12.9 (bs,1H).

IR: 3249, 1708, 1662, 1617, 1479, 1427, 1322, 1250, 1008, 879, 790, 754 cm⁻¹

M.P.=276° C.

HPLC: 97.6%

### Example 128

### Methyl 4-[6-(4-methoxy-benzylcarbamoyl)-2,4-dioxo-1,4-dihydro-2H-quinazolin-3-ylmethyl]-benzoate

Step 1: 4-Amino-isophtalic acid 3-methyl ester

The compound is obtained according to the procedure of the Step 3 of the Example 119 using as substrate 4-amino-isophtalic acid 1-benzylester 3-methyl ester.

Step 2: 6-Amino-N-(4-methoxy-benzyl)-isophtalamic acid methyl ester

The compound is obtained according to the procedure of the Example 1 using the compound obtained in the preceding Step 1 and 4-methoxy-benzylamine.

Step 3: Methyl 4-[6-(4-methoxy-benzylcarbamoyl)-2,4-dioxo-1,4-dihydro-2H-quinazolin-3-ylmethyl]-benzoate

The compound is obtained according to the procedure of the Step 1-5 to 2-5 of the Preparation B using in the Step 1-5 the compound obtained in the preceding Step 2 and methyl 4-aminomethyl benzoate.

TLC: CH₂Cl₂/MeOH 90/10 Rf=0.55

NMR: DMSO ¹H δ(ppm): 3.70 (s,3H); 3.80 (s,3H); 4.40 (d,2H); 5.15 (s,2H); 6.90 (d,2H); 7.20 (m,3H); 7.45 (d,2H); 7.90 (d,2H); 8.15 (d,1H); 8.50 (s,1H); 9.15 (t,1H); 11.8 (s,1H);

IR: 3265, 2935, 2553, 1719, 1665, 1637, 1514, 1459, 1275, 1105, 827, 751 cm⁻¹

M.P.=287.5° C.

HPLC: 98.3%

### Example 129

### Methyl 4-[1-ethyl-6-(4-methoxy-benzylcarbamoyl)-2,4-dioxo-1,4-dihydro-2H-quinazolin-3-ylmethyl]-benzoate

The compound is obtained according to the procedure of the Step 4 of the Example 15 using the compound obtained the Example 128 and iodomethane in DMF with K₂CO₃. The desired compound crystallizes in a mixture of dichloromethane/ether.

TLC: CH₂Cl₂/MeOH 90/10 Rf=0.55

NMR: DMSO ¹H δ(ppm): 1.25 (t,3H); 3.75 (s,3H); 3.85 (s,3H); 4.20 (d,2H); 4.40 (d,2H); 5.25 (s,2H); 6.90 (d,2H); 7.25 (d,2H); 7.45 (d,2H); 7.60 (d,1H); 7.90 (d,2H); 8.25 (d,1H); 8.65 (s,1H); 9.20 (t,1H).

IR: 3403, 2361, 1708, 1659, 1646, 1615, 1508, 1273, 1251, 1113, 847, 758 cm⁻¹

M.P.=190° C.

HPLC: 96.9%

### Example 130

### 4-[1-Ethyl-6-(4-methoxy-benzylcarbamoyl)-2,4-dioxo-1,4-dihydro-2H-quinazolin-3-ylmethyl]-benzoic acid

The compound is obtained by hydrolysis of compound of the Example 112 using as reagent K₂CO₃ in a mixture of methanol and water under reflux for 3 hours. After acidification of the reaction mixture, the precipitate obtained is filtered off to provide the desired product.

TLC: CH₂Cl₂/MeOH 90/10 Rf=0.45

NMR: DMSO ¹H δ(ppm): 1.25 (t,3H); 3.70 (s,3H); 4.20 (q,2H); 4.40 (d,2H); 5.20 (s,2H); 6.90 (d,2H); 7.25 (d,2H); 7.40 (d,2H); 7.60 (d,1H); 7.85 (d,2H); 8.25 (d,1H); 8.65 (s,1H); 9.20 (t,1H); 12.85 (bs,1H)

IR: 2361, 1708, 1655, 1616, 1501, 1466, 1322, 1250, 1177, 1032, 823, 754 cm⁻¹

M.P.=160° C.

HPLC: 98.2%

### Example 131

### 3-(4-Methoxy-benzyl)-1-methyl-2,4-dioxo1,2,3,4-tetrahydro-quinazoline-6-carboxylic acid (pyridin-4-ylmethyl)-amide

Step 1: Methyl 3-(4-methoxybenzyl)-1-methyl-2,4-dioxo-1,2,3,4-tetrahydro -quinazoline-6-carboxylate

The compound is obtained according to the procedure of the Step 4 of the Example 15 using the compound obtained in the Step 1 of example 16.

Step 2: 3-(4-methoxybenzyl)-1-methyl-2,4-dioxo-1,2,3,4-tetrahydroquinazoline -6-carboxylic acid

The compound is obtained according to the procedure of the Step 2-4 of the Preparation B using the compound obtained in the preceding Step 1.

Step 3: 3-(4-Methoxy-benzyl)-1-methyl-2,4-dioxo-1,2,3,4-tetrahydro-quinazoline-6-carboxylic acid (pyridin-4-ylmethyl)-amide

The compound is obtained (0.160 g, yield: 63%) according to the procedure of the Step 3 of the Example 116 using the compound obtained in the preceding Step 2 and 4-(aminomethyl)pyridine.

TLC: CH₂Cl₂ MeOH 90/10 Rf=0.70

NMR: DMSO ¹H δ(ppm): 3.55 (s,3H); 3.7 (s,3H); 4.5 (d,2H); 5.10 (s,2H); 6.80-6.90 (m,2H); 7.30-7.35 (m,4H); 7.55-7.60 (m,1H); 8.25-8.30 (m,1H); 8.38-8.42 (m,2H); 8.70 (s,1H); 9.35 (t,1H).

IR: 3269, 1705, 1659, 1644, 1615, 1510, 1245, 1180, 842, 785 cm⁻¹

M.P.=213.9° C.

HPLC: 97.8%

### Example 132

### 3-(4-Hydroxy-benzyl)-1-methyl-2,4-dioxo-1,2,3,4-tetrahydro-quinazoline-6-carboxylic acid (pyridin-4-ylmethyl)-amide

To a stirred solution of 0.630 g (1.46 mmol) of compound of the Example 131 in 50 ml of dichloromethane are added, under an inert atmosphere, 3.7 g (1.3 ml, 14.6 mmol) of BBr₃ in 5 ml of dichloromethane. After 1 hour of stirring at room temperature, the reaction mixture is cooled and poured on 100 ml of a saturated solution of NaHCO₃. The precipitate obtained is purified by chromatography over silica gel (gradient of methanol in dichloromethane) and solidified in dichloromethane to provide the desired compound.

TLC: CH₂Cl₂/MeOH 90/10 Rf=0.50

NMR: DMSO ¹H δ(ppm): 3.45 (s,3H); 4.45 (d,2H); 5.0 (s,2H); 6.60 (d,2H); 7.1 (d,2H); 7.25 (d,2H); 7.5 (d,1H); 8.20 (d,1H); 8.40 (d,2H); 8.60 (s,1H); 9.20 (s,1H); 9.20 (t,1H).

IR: 3048, 1705, 1659, 1642, 1507, 1479, 1328, 1244, 831 cm⁻¹

M.P.=262.0° C.

HPLC: 94.8%

### Example 133

### 3-(4-Cyano-benzyl)-1-methyl-2,4-dioxo-1,2,3,4-tetrahydro-quinazoline-6-carboxylic acid (pyridin-4-ylmethyl)-amide

Step 1: 1-Methyl-2,4-dioxo-1,2,3,4-tetrahydro-quinazoline-6-carboxylic acid (pyridin-4-ylmethyl)-amide

The compound is obtained according to the procedure of the Example 33 using the same substrate and 4-picolylamine in the step of amidification.

TLC: CH₂Cl₂/MeOH 90/10 Rf=0.25

NMR: DMSO ¹H δ(ppm): 3.45 (s,3H); 4.5 (d,2H); 7.3 (d,2H); 7.55 (d,1H); 8.25 (d,1H); 8.5 (d,2H); 8.6 (s,1H); 9.35 (t,1H); 11.7 (s,1H).

IR: 3185, 1686, 1618, 1479, 1417, 1326, 782 cm⁻¹

M.P.=292° C.

HPLC: 96.4%

Step 2: 3-(4-Cyano-benzyl)-1-methyl-2,4-dioxo-1,2,3,4-tetrahydro-quinazoline-6-carboxylic acid (pyridin-4-ylmethyl)-amide

The compound is obtained according to the procedure of the Step 2 of Example 34 using the compound obtained in the preceding Step 1 and α-bromo-para-toluonitrile.

TLC: AcOEt Rf=0.55

NMR: CDCl₃ ¹H δ(ppm): 3.60 (s,3H); 4.60 (d,2H); 5.30 (s,2H); 7.3 (m,3H); 7.60 (s,4H); 8.40 (m,1H); 8.45 (m,2H); 8.65 (m,1H); 8.80 (s,1H).

M.P.=258° C.

HPLC: 98.9%

### Example 134

### 1-Methyl-2,4-dioxo-3-(3-pyridin-4-yl-alyl)-1,2,3,4-tetrahydro-quinazoline-6-carboxylic acid (pyridin-4-ylmethyl)-amide

The compound is obtained according to the procedure of the Step 2 of Example 34 using the compound obtained in the Step 1 of Example 133 and 4-(3-chloro-propenyl)-pyridine hydrochloride.

TLC: CH₂Cl₂/MeOH 90/10 Rf=0.50

NMR: DMSO ¹H δ(ppm): 3.60 (s,3H); 4.50 (m,2H); 4.80 (m,2H); 6.50 (m,1H); 6.65 (m,1H); 7.3 (m,2H); 7.40 (m,2H); 7.60 (d,1H); 8.25 (d,1H); 8.50 (m,4H); 8.65 (s,1H); 9.35 (m,1H).

M.P.=117° C.

HPLC: 99.5%

### Example 135

### Methyl 4-{1-methyl-2,4-dioxo-6-[(pyridin-4-ylmethyl)-carbamoyl]-1,4-dihydro-2H-quinazolin-3-ylmethyl}-benzoate

The compound is obtained according to the procedure of the Step 2 of Example 34 using the compound obtained in the Step 1 of Example 133 and methyl-4-(bromomethyl)-benzoate.

TLC: CH₂Cl₂/MeOH 90/10 Rf=0.45

NMR: DMSO ¹H δ(ppm): 3.55 (s,3H); 3.80 (s,3H); 4.5 (d,2H); 5.20 (s,2H); 7.3 (m,2H); 7.45 (d,2H); 7.60 (d,1H); 7.90 (m,2H); 8.25 (d,1H); 8.5 (m,2H); 8.65 (s,1H); 9.35 (t,1H);

IR: 3265, 1718, 1704, 1663, 1641, 1318, 1289, 1113, 751 cm⁻¹

M.P.=236° C.

HPLC: 97.5%

### Example 136

### 4-{1-Methyl-2,4-dioxo-6-[(pyridin-4-ylmethyl)-carbamoyl]-1,4-dihydro-2H-quinazolin-3-ylmethyl}-benzoic acid

The compound is obtained according to the procedure of the Step 2-4 of the Preparation B using the compound obtained in the Example 135. The corresponding hydrochloride is obtained after dissolution of the compound in a hot solution of isopropanol/ HCl 0.1 M. The desired compound is purified by crystallization from acetonitrile.

NMR: DMSO ¹H δ(ppm): 2.4-4.40 (m,1H); 3.60 (s,3H); 4.15 (t,2H); 5.20 (s,2H); 7.40 (d,2H); 7.60 (d,1H); 7.90 (m,4H); 8.30 (d,1H); 8.70 (s,1H); 8.80 (d,1H); 9.65 (t,1H); 12.9 (bs,1H).

IR: 3265, 1718, 1704, 1663, 1641, 1318, 1289, 1113, 751 cm⁻¹

M.P.=268° C.

HPLC: 97.9%

### Example 137

### Methyl (4-{1-methyl-2,4-dioxo-6-[(pyridin-4-ylmethyl)-carbamoyl]-1,4-dihydro-2H-quinazolin-3-ylmethyl}-phenyl)-acetate

The compound is obtained according to the procedure of the Step 2 of Example 34 using the compound obtained in the Step 1 of Example 133 and methyl 4-(bromomethyl-phenyl) acetate.

TLC: CH₂Cl₂/MeOH 90/10 Rf=0.45

NMR: DMSO ¹H δ(ppm): 3.50-3.60 (s,6H); 3.65 (s,2H); 4.5 (t,2H); 5.15 (s,2H); 7.20 (m,2H); 7.20-7.35 (m,4H); 7.55 (d,1H); 8.25 (d,1H); 8.5 (d,2H); 8.65 (s,1H); 9.35 (t,1H);

IR: 3298, 1736, 1707, 1663, 1631, 1505, 1473, 1320, 1157, 751 cm⁻¹

M.P.=141° C.

HPLC: 96.4%

### Example 138

### (4-{1-Methyl-2,4-dioxo-6-[(pyridin-4-ylmethyl)-carbamoyl]-1,4-dihydro-2H-quinazolin-3-ylmethyl}-phenyl)-acetic acid

The compound is obtained according to the procedure of the Step 2-4 of Preparation B using the compound obtained in the Example 137. The corresponding hydrochloride is obtained after dissolution of the compound in a hot solution of isopropanol/ HCl 0.1 M. The desired compound is purified by crystallization from acetonitrile.

NMR: DMSO ¹H δ(ppm): 2.50-5.50 (bs,HCl+OH); 3.45-3.60 (2s,5H); 4.70 (d,2H); 5.15 (s,2H); 7.15 (d,2H); 7.25 (d,2H); 7.55 (d,1H); 7.85 (d,2H); 8.30 (d,1H); 8.65 (s,1H); 8.75 (d,2H); 9.55 (t,1H).

IR: 3298, 1736, 1707, 1663, 1631, 1505, 1473, 1320, 1157, 751 cm⁻¹

M.P.=241° C.

HPLC: 97.5%

### Example 139

### Methyl 4-{1-methyl-2,4-dioxo-6-[(1-oxy-pyridin-4ylmethyl)-carbamoyl]-1,4-dihydro-2H-quinazolin-3-ylmethyl}-benzoate

To a stirred suspension of 0.500 g (1.10 mmol) of compound of the Example 135 in 20 ml of dichloromethane, maintained at −20° C., are added 0.250 g (1.10 mmol) of meta-chloroperbenzoic acid in 5 ml of dichloromethane. After stirring overnight at room temperature, the reaction mixture is washed successively with a saturated solution of Na₂CO₃ and water. The organic phase is dried and concentrated under vacuum. A chromatography over silica gel (gradient of methanol in dichloromethane) followed by a solidification in dichloromethane/ether provides 0.300 g (yield: 57%) of the desired product.

TLC: CH₂Cl₂/MeOH 90/10 Rf=0.28

NMR: DMSO ¹H δ(ppm): 3.55 (s,3H); 3.85 (s,3H); 4.45 (d,2H); 5.25 (s,2H); 7.3 (d,2H); 7.45 (d,2H); 7.60 (d,1H); 7.90 (d,2H); 8.15 (d,2H); 8.30 (s,1H); 8.65 (s,1H); 9.35 (t,1H);

IR: 1705, 1655, 1617, 1478, 1283, 750, 711 cm⁻¹

M.P.=218° C.

HPLC: 99.1%

### Example 140

### 4-{1-Methyl-2,4-dioxo-6-[(1-oxy-pyridin-4-ylmethyl)carbamoyl]-1,4-dihydro-2H-quinazolin-3-ylmethyl}-benzoic acid

The compound is obtained according to the procedure of the Step 2-4 of Preparation B using the compound obtained in the Example 139.

NMR: DMSO ¹H δ(ppm): 3.55 (s,3H); 4.55 (d,2H); 5.20 (s,2H); 7.30-7.50 (m,4H); 7.60 (d,1H); 7.85 (d,2H); 8.25 (d,2H); 8.30 (d,1H); 8.65 (s,1H); 9.35 (t,1H); 12.9 (bs,1H).

IR: 1702, 1655, 1617, 1479, 1245, 753 cm⁻¹

M.P.=192° C.

HPLC: 98.4%

### Example 141

### Methyl{6-[(1,3-Benzodioxol-5-ylmethyl)-carbamoyl]-3-benzyl-2,4-dioxo-1,4-dihydro-2H-quinazolin-1-yl}-acetate

The compound is obtained by alkylation of the compound of Example 3 using K₂CO₃ and methylbromoacetate in DMF.

TLC: CH₂Cl₂/MeOH 95/5 Rf=0.70

NMR: DMSO ¹H δ(ppm): 3.70 (s,3H); 4.40 (d,2H); 5.05 (s,2H); 5.15 (s,2H); 6.0 (s,2H); 6.85 (m,3H); 7.30 (m,5H); 7.55 (d,1H); 8.20 (d,1H); 8.65 (s,1H); 9.20 (t,1H).

IR: 3282, 2361, 1736, 1669, 1632, 1464, 1370, 1236, 1040, 833, 776, 758 cm⁻¹

M.P.=194.0° C.

HPLC: 97.6%

### Example 142

### {6-[(1,3-Benzodioxol-5-ylmethyl)-carbamoyl]-3-benzyl-2,4-dioxo-3,4-dihydro-2H-quinazolin-1-yl}-acetic acid

The compound is obtained according to the procedure of the Step 2-4 of Preparation B using the compound obtained in the Example 141.

TLC: CH₂Cl₂/MeOH 95/5 Rf=0.70

NMR: DMSO ¹H δ(ppm): 4.35 (d,2H); 4.90 (s,2H); 5.15 (s,2H); 5.95 (s,2H); 6.80 (m,3H); 7.30 (m,5H); 7.50 (d,1H); 8.20 (d,1H); 8.60 (s,1H); 9.20 (t,1H); 13.25 (bs,1H).

IR: 3346, 2935, 1709, 1668, 1612, 1499, 1467, 1305, 1250, 1117, 1036, 873 cm⁻¹

M.P.=163.0° C.

HPLC: 99.6%

### Example 143

### Methyl 4-{6-[(1,3-benzodioxol-5-ylmethyl)-carbamoyl]-1-methyl-2,4-dioxol-1,4-dihydro-2H-quinazolin-3-ylmethyl}-benzoate

The compound is obtained according to the procedure of the Step 2 of the Example 34 using the compound obtained in the Example 37 and methyl 4-(bromomethyl)-benzoate

TLC: CH₂Cl₂/MeOH 90/10 Rf=0.80

NMR: DMSO ¹H δ(ppm): 3.60 (s,3H); 3.90 (s,3H); 4.40 (d,2H); 5.20 (s,2H); 6.0 (s,2H); 6.80-6.95 (m,3H); 7.45 (d,2H); 7.60 (d,1H); 7.85 (d,2H); 8.30 (d,1H); 8.65 (s,1H); 9.20 (t,1H).

IR: 3418, 1713, 1666, 1657, 1617, 1497, 1477, 1280, 1252, 1038, 770, 749 cm⁻¹

M.P.=233.5° C.

HPLC: 99.6%

### Example 144

### 4-{6-[(1,3-Benzodioxol-5-ylmethyl)-carbamoyl]-1-methyl-2,4-dioxo-1,4-dihydro-2H-quinazolin-3-ylmethyl}-benzoic acid

The compound is obtained according to the procedure of the Step 2-4 of Preparation B using the compound obtained in the Example 143.

TLC: CH₂Cl₂/MeOH 90/10 Rf=0.40

NMR: DMSO ¹H δ(ppm) 3.60 (s,3H); 4.40 (d,2H); 5.20 (s,2H); 5.95 (s,2H); 6.80-6.95 (m,3H); 7.40 (d,2H); 7.60 (d,1H); 7.85 (d,2H); 8.30 (d,1H); 8.60 (s,1H); 9.20 (t,1H); 12.85 (s,1H).

IR: 3377, 3233, 1717, 1698, 1665, 1649, 1502, 1481, 1236, 751 cm⁻¹

M.P.=295.7° C.

HPLC: 97.9%

### Example 145

### 3-Benzyl-1-methyl-2,4-dioxo1,2,3,4-tetrahydro-quinazoline-6-carboxylic acid 4-sulfamoyl-benzylamide

The compound is obtained according to the procedure of the Example 9 using the compound obtained in the Preparation C and 4-(aminomethyl)benzene sulfonamide hydrochlorhyde hydrate.

TLC: CH₂Cl₂/MeOH 90/10 Rf=0.37

NMR: DMSO ¹H δ(ppm): 3.60 (s,3H); 4.55 (d,2H); 5.15 (s,2H); 7.2-7.35 (m,7H); 7.50 (d,2H); 7.60 (d,1H); 7.80 (d,2H); 8.30 (d,1H); 8.65 (s,1H); 9.35 (t,1H)

IR: 3290, 1709, 1652, 1618, 1503, 1321, 1154, 702 cm⁻¹

M.P.=266° C.

HPLC: 97.5%

### Example 146

### 3-Benzyl-1-methyl-2,4-dioxo-1,2,3,4-tetrahydro-quinazoline-6-carboxylic acid [3-(pyridin-4-ylsulfanyl)-propyl]-amide

The compound is obtained according to the procedure of the Example 9 using the compound obtained in the Preparation C, 3-(pyrydin-4-ylsulfanyl)-propylamine and dichloromethane as solvent. (The reactant 3-(pyridin-4-ylsulfamyl)-propylamine is obtained according to the method described in Bioorg. Med. Chem., 1996, 4, 557-562).

TLC: CH₂Cl₂/MeOH 90/10 Rf=0.70

NMR: DMSO ¹H δ(ppm): 1.8-1.90 (m,2H); 3.1-3.20 (m,2H); 3.4-3.50 (m,2H); 3.60 (s,3H); 5.20 (s,2H); 7.2-7.40 (m,7H); 7.50-7.55 (m,1H); 8.20 (d,1H); 8.30-8.40 (m,2H) 8.60(s,1H); 8.80 (t,1H).

IR: 3308, 1705, 1662, 1636, 1578, 1509, 1447, 1321, 804, 712 cm⁻¹

M.P.=130.7° C.

HPLC: 99.2%

### Example 147

### 3-Benzyl-1-methyl-2,4-dioxo-1,2,3,4-tetrahydro-quinazoline-6-carboxylic acid (4-morpholin-4-yl-butyl)-amide

The compound is obtained according to the procedure of the Step 3 of Example 116 using the compound obtained in the Preparation C, 4-morpholin-4-yl-butylamine, and dichloromethane as solvent. (The reactant 4-morpholin-4-yl-butylamine is obtained according to the method described in J. Med. Chem., 1997, 40, 3915-3925).

TLC: CH₂Cl₂/MeOH 90/10 Rf=0.60

NMR: DMSO ¹H δ(ppm): 1.4-1.60 (m,4H); 2.2-2.35 m,6H); 3.20-3.35 (m,2H); 3.55 (s,3H); 3.5-3.60 (m,4H); 5.20 (s,2H); 7.2-7.35 (m,5H); 7.50 (d,1H); 8.20-8.25 (m,1H); 8.60 (s,1H); 8.70 (t,1H)

IR: 3402, 2942, 1707, 1645, 1476, 1327, 1118, 763 cm⁻¹

M.P.=170.6 ° C.

HPLC: 99.3%

### Example 148

### 3-Benzyl-1-methyl-2,4-dioxo-,1,2,3,4-tetrahydro-quinazoline6-carboxylic acid (1benzyl-piperidin-4-yl)-amide

The compound is obtained according to the procedure of the Example 9 using the compound obtained in the Preparation C, 4-amino-1-benzylpiperidine, and dichloromethane as solvent. The desired compound crystallizes from a mixture of dichloromethane and ether.

TLC: CH₂Cl₂/MeOH 90/10 Rf=0.50

NMR: DMSO ¹H δ(ppm): 1.60 (m,2H); 1.75 (m,2H); 2.0 (t,2H); 2.8 (d,2H); 3.45 (s,2H); 3.55 (s,3H); 3.75 (m,1H); 5.15 (s,2H); 7.30 (m,10H); 7.55 (d,1H); 8.20 (d,1H); 8.50 (d,1H); 8.60(s,1H).

IR: 3257, 2943, 2749, 1709, 1656, 1633, 1511, 1332, 1242, 1077,829, 750cm⁻¹

M.P.=219.4° C.

HPLC: 98.6%

Example 149

### 3-Benzyl-1-methyl-2,4-dioxo1,2,3,4-tetrahydro-quinazoline-6-carboxylic acid 4-hydroxy-benzylamide

To a round bottom protected from moisture and under inert atmosphere are introduced 1.9 g (4.4 mmol) of compound of Example 13 in 200 ml of dichloromethane. To the stirred solution are added dropwise 4.2 ml (11.1 g, 44 mmol) of BBr₃ in 17 ml of dichloromethane. After 30 minutes at room temperature the reaction mixture is poured to a 500 ml saturated solution of NaHCO₃, extracted with dichloromethane, dried and concentrated under vacuum. A crystallization of the crude product in methanol/ether provides 1.35 g (yield: 74%) of the desired compound.

TLC: CH₂Cl₂/MeOH 90/10 Rf=0.55

NMR: DMSO ¹H δ(ppm): 3.60 (s,3H); 4.40 (d,2H); 5.20 (s,2H); 6.7-6.75 (m,2H); 7.10-7.20 (m,2H); 7.2-7.40 (m,5H); 7.55 (d,1H); 8.25 (d,1H); 8.65 (s,1H); 9.20 (t,1H); 9.0-9.3 (bs,1H).

IR: 3314, 1698, 1635, 1622, 1500, 1480, 1453, 1255, 826, 748 cm⁻¹

M.P.=191.8 ° C.

HPLC 96.4%

### Example 150

### Ethyl (4-{[(3-benzyl-1-methyl-2,4-dioxo-1,2,3,4-tetrahydro-quinazoline-6-carbonyl)-amino]-methyl}-phenoxy)-acetate

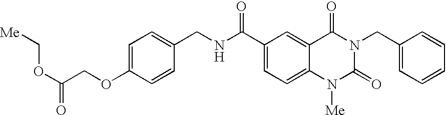

To a round bottom protected from moisture and under inert atmosphere are introduced 0.45 g (1.08 mmol) of compound of Example 149 in 13.5 ml od DMF. To the stirred solution are added 0.3 g of K₂CO₃ (2.16 mmol) and 0.24 ml (2.016 mmol) of ethyl bromoacetate. After 1 hour at 60° C. the reaction mixture is concentrated under vacuum. The crude product is taken up in dichloromethane, washed with water, dried and concentrated under vacuum to provide 0.410 g (yield: 75.8%) of the desired compound.

TLC: CH₂Cl₂/MeOH 90/10 Rf=0.70

NMR: DMSO ¹H δ(ppm): 1.2 (t,3H); 3.60 (s,3H); 4.15 (q,2H); 4.45 (d,2H); 4.80 (s,2H) 5.20 (s,2H); 6.90 (d,2H); 7.2-7.40 (m,7H); 7.5 (d,1H); 8.20 (d,1H); 8.60 (s,1H); 9.20 (t,1H)

IR: 3407, 1755, 1705, 1642, 1508, 1324, 1210, 750 cm⁻¹

M.P.=172.6° C.

HPLC: 97.8%

### Example 151

### (4-{[(3-Benzyl-1-methyl-2,4dioxo-1,2,3,4-tetrahydro-quinazoline-6-carbonyl)-amino]-methyl}-phenoxy)-acetic acid

The compound is obtained according to the procedure of the Step 2-4 of the Preparation B using the compound of the Example 150.

TLC: CH₂Cl₂/MeOH 90/10 Rf=0.70

NMR: DMSO ¹H δ(ppm): 3.60 (s,3H); 4.40 (d,2H); 4.65 (s,2H); 5.15 (s,2H); 6.85 (d,2H); 7.2-7.40 (m,7H); 7.55 (d,1H); 8.25 (d,1H); 8.65 (s,1H); 9.20 (t,1H); 12.95 (bs,1H).

IR: 3407, 1755, 1705, 1642, 1508, 1324, 1210, 750 cm⁻¹

M.P.=195.6° C.

HPLC: 98.3%

### Example 152

### 3-Benzyl-1-methyl-2,4-dioxo-1,2,3,4-tetrahydro-quinazoline-6-carboxylic acid 4-dimethylcarbamoylmethoxy-benzylamide

The compound is obtained according to the procedure of the Example 1 using the compound of Example 151 and dimethylamine 2M in solution in THF.

TLC: CH₂Cl₂/MeOH 90/10 Rf=0.70

NMR: DMSO ¹H δ(ppm): 2.80 (s,3H); 3.0 (s,3H); 3.55 (s,3H); 4.40 (d,2H); 4.80 (s,2H); 5.20 (s,2H); 6.90 (d,2H); 7.2-7.40 (m,7H); 7.50 (d,1H); 8.20 (d,1H); 8.65 (s,1H); 9.25 (t,1H).

IR: 3276, 1704, 1659, 1635, 1499, 1317, 1240, 1066, 750 cm⁻¹

M.P.=152.7° C.

HPLC: 96.5%

### Example 153

### 3-Benzyl-1-methyl-2,4-dioxo-1,2,3,4-tetrahydro-quinazoline-6-carboxylic acid (3-phenyl-allyl)-amide

The compound is obtained according to the procedure of the Example 9 using the compound of the Preparation C and 3-phenyl-allylamine hydrochloride.

TLC: CH₂Cl₂/MeOH 90/10 Rf=0.80

NMR: DMSO ¹H δ(ppm): 3.55 (s,3H); 4.10 (m,2H); 5.20 (s,2H); 6.35 (m,1H); 6.60 (m,1H); 7.20-7.35 (m,8H); 7.40 (m,2H); 7.55 (d,1H); 8.30 (d,1H); 8.70 (s,1H); 9.00 (m,1H).

M.P.=193.0° C.

HPLC: 99.7%

### Example 154

### 3-Benzyl-1-methyl-2,4-dioxo-1,2,3,4-tetrahydro-quinazoline-6-carboxylic acid 4-cyano-benzylamide

The compound is obtained according to the procedure of the Example 9 using the compound of the Preparation C and 4-amino-benzyl benzonitrile. The desired product is solidified in a mixture of dichloromethane/ether.

TLC: CH₂Cl₂/MeOH 90/10 Rf=0.46

NMR: DMSO ¹H δ(ppm): 3.55 (s,3H); 4.60 (d,2H); 5.15 (s,2H); 7.20-7.40 (m,5H); 7.45-7.60 (m,3H); 7.80 (d,2H); 8.25 (d,1H); 8.65 (s,1H); 9.40 (t,1H).

IR: 3305, 2224, 1708, 1664, 1638, 1507, 1318, 751 cm⁻¹

M.P.=245.0° C.

HPLC: 96.2%

### Example 155

### 4-{[(3-Benzyl-1-methyl-2,4-dioxo-1,2,3,4-tetrahydro-quinazoline6-carbonyl)-amino]-methyl}-benzoic acid

The compound is obtained according to the procedure of the Step 2-4 of the Preparation B using the compound of the Example 11.

TLC: CH₂Cl₂/MeOH 90/10 Rf=0.30

NMR: DMSO ¹H δ(ppm): 3.55 (s,3H); 4.55 (d,2H); 5.15 (s,2H); 7.25 (m,5H); 7.40 (d,2H); 7.55 (d,1H); 7.90(d,2H); 8.25 (d,1H); 8.65 (s,1H); 9.30 (t,1H); 12.90 (bs,1H).

IR: 3395, 1707, 1698, 1642, 1618, 1501, 1431, 1291, 1242, 938, 829, 759 cm⁻¹

M.P.=228.5° C.

HPLC: 96.9%

### Example 156

### 3-Benzyl-1-methyl-2,4-dioxo-1,2,3,4-tetrahydro-quinazoline-6-carboxylic acid 4-dimethylcarbamoyl-benzylamide

The compound is obtained according to the procedure of the Example 1 using the compound of the Example 155 and dimethylamine in solution 2M in THF.

TLC: CH₂Cl₂/MeOH 90/10 Rf=0.70

NMR: DMSO ¹H δ(ppm): 3.0 (m,6H); 3.55 (s,3H); 4.55 (d,2H); 5.15 (s,2H); 7.30 (m,9H); 7.60 (d,1H); 8.30 (d,1H); 8.65 (s,1H); 9.30 (t,1H).

IR: 3249, 2361, 1705, 1657, 1609, 1504, 1452, 1254, 1069, 1020, 839, 750 cm⁻¹

M.P.=194.7° C.

HPLC: 96.8%

### Example 157

### 3-(4-Dimethylamino-benzyl)-2,4-dioxo-1,2,3,4-tetrahydro-quinazoline-6-carboxylic acid 4-methoxy-benzylamide

The compound is obtained according to the Step 1-5 to 3-5 of the preparation B using in the Step 1-5 4-dimethylamino-benzyl isocyanate, and then according to the procedure of Example 1 using the compound obtained in the preceding step and 4-methoxy-benzylamine

NMR: DMSO ¹H δ(ppm): 2.80 (s,6H); 3.70 (s,3H); 4.40 (d,2H); 4.95 (s,2H); 6.60 (d,2H); 6.85 (d,2H); 7.15-7.25 (m,5H); 8.10 (dd,1H); 8.50 (s,1H); 9.10 (t,1H); 11.7 (s,1H).

IR: 3177, 1729, 1630, 1512, 1445, 1249, 765 cm⁻¹

M.P.=267° C.

HPLC: 98.5%

### Example 158

### 3-[4-(N-methylsulfonylamino)-benzyl]-1-methyl-2,4-dioxo-1,2,3,4-tetrahydro-quinazoline-6-carboxylic acid 4-methoxy-benzylamide

The compound is obtained according to the procedure of the Example 97 using as substrates the compound obtained in the Example 95 and 2.5 equivalents of methanesulfonyl chloride.

TLC: CH₂Cl₂/MeOH 90/10 Rf=0.22

NMR:.DMSO ¹H δ(ppm): 2.90 (s,3H); 3.55 (s,3H); 3.70 (s,3H); 4.40 (d,2H); 5.10 (s,2H); 6.90 (d,2H); 7.10 (d,2H); 7.25 (d,2H); 7.30 (d,2H); 7.55 (s,1H); 8.25 (d,1H); 8.60 (s,1H); 9.2 (t,1H); 9.70 (s,1H)

IR: 1655, 1615, 1513, 1500, 1325, 1248, 1148 cm⁻¹

M.P.−224° C.

HPLC: 98.8%

### Example 159

### tert-Butyl {5[6-(4-Methoxy-benzylcarbamoyl)-1-methyl-2,4-dioxo-1,4-dihydro-2H-quinazolin3-ylmethyl]-pyridin-2yl}-carbamate

The compound is obtained according to the procedure of the Step 2 of Example 34 using the compound obtained in the Step 1 of the Example 34 and tert-butyl (5-bromomethyl-pyridin-2-yl)-carbamate.

TLC: CH₂Cl₂/MeOH 90/10 Rf=0.80

NMR:.DMSO ¹H δ(ppm): 1.45 (s,9H); 3.55 (s,3H); 3.75 (s,3H); 4.40 (d,2H); 5.10 (s,2H); 6.90 (d,2H); 7.25 (d,2H); 7.55 (d,1H); 7.70 m,2H); 8.25-8.30 (m,2H); 8.65 (s,1H); 9.2 (t,1H); 9.70 (s,1H)

IR: 1711, 1654, 1614, 1508, 1478, 1302, 1243, 1159cm⁻¹

M.P.=204° C.

HPLC: 99.3%

### Example 160

### 3-(6-Amino-pyridin-3-ylmethyl)-1-methyl-2,4-dioxo-1,2,3,4-tetrahydro-quinazoline-6-carboxylic acid 4-methoxy-benzylamide

The compound is obtained by deprotection of compound of the Example 159 by using trifluoroacetic acid in dichloromethane.

TLC: CH₂Cl₂/MeOH 90/10 Rf=0.40

NMR:.DMSO ¹H δ(ppm): 3.55 (s,3H); 3.75 (s,3H); 4.40 (d,2H); 4.95 (s,2H); 5.80 (bs,2H); 6.35 (d,1H); 6.90 (d,2H); 7.25 (d,2H); 7.40 (dd,1H); 7.50 (d,1H); 7.95 (s,1H); 8.25 (dd,1H); 8.60 (s,1H); 9.2 (t,1H)

IR: 1704, 1648, 1615, 1509, 1477, 1245 cm⁻¹

M.P.=155° C.

HPLC: 99.5%

### Example 161

### 1,3-Dimethyl-2,4-dioxo-1,2,3,4-tetrahydro-pyrido[2,3-d]pyrimidine-6-carboxylic acid (1,3-benzodioxol-5-ylmethyl)-amide

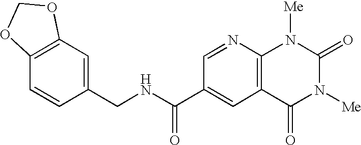

Step 1: 1,3-Dimethyl-2,4-dioxo-1,2,3,4-tetrahydro-pyrido[2,3-d]pyrimidine-6-carboxylic acid.

The compound is obtained by hydrolysis in a mixture of dioxan/water of ethyl 1,3-dimethyl-2,4-dioxo-1,2,3,4-tetrahydro-pyrido[2,3-d]pyrimidine-6-carboxylate (Heterocycles 1998, 48(12),2521-2528) in presence of LiOH.

TLC: CH₂Cl₂/MeOH 90/10 Rf=0.10

R.M.N:.DMSO ¹H δ(ppm): 3.30 (s,3H); 3.60 (s,3H); 8.70 (s,1H); 9.15 (s,1H); 13.5 (bs,1H)

Step 2: 1,3-Dimethyl-2,4-dioxo-1,2,3,4-tetrahydro-pyrido[2,3d]pyrimidine-6-carboxylic acid (1,3-benzodioxol-5-ylmethyl)-amide

The compound is obtained according to the procedure of the Example 1 using the compound obtained in the preceding Step 1 and piperonylamine.

TLC: CH₂Cl₂/MeOH 90/10 Rf=0.90

NMR:.DMSO ¹H δ(ppm): 3.35 (s,3H); 3.6 (s,3H); 4.40 (d,2H); 6.0 (s,2H); 6.75-6.85 (m,2H); 6.90 (s,1H); 8.80 (s,1H); 9.15 (s,1H); 9.30 (t,1H).

IR: 3227, 1705, 1663, 1632, 1608, 1498, 1299, 1250, 1040, 794 cm⁻¹

M.P.=218.4° C.

HPLC: 94.6%

### Example 162

### 1,3-Dimethyl-2,4-dioxo-1,2,3,4-tetrahydro-pyrido[3,4-d]pyrimidine-6-carboxylic acid (1,3-benzodioxol-5-ylmethyl)-amide

Step 1: 1,3-Dimethyl-2,4-dioxo-1,2,3,4-tetrahydro-pyrido[3,4-d]pyrimidine-6-carboxylic acid

The compound is obtained by hydrolysis in a mixture of dioxan/water of methyl 1,3-dimethyl-2,4-dioxo-1,2,3,4-tetrahydro-pyrido[3,4-d]pyrimidine-6-carboxylate (Heterocycles 1994, 37(1), 563-570) in presence of LiOH.

TLC: CH₂Cl₂/MeOH 90/10 Rf=0.01

NMR:.DMSO ¹H δ(ppm): 3.30 (s,3H); 3.60 (s,3H); 8.40 (s,1H); 9.00 (s,1H); 13.3 (bs,1H)

Step 2: 1,3-Dimethyl-2,4-dioxo-1,2,3,4-tetrahydro-pyrido[3,4-d]pyrimidine-6-carboxylic acid (1,3-benzodioxol-5-ylmethyl)-amide

The compound is obtained according to the procedure of the Example 1 using the compound obtained in the preceding Step 1 and piperonylamine.

TLC: CH₂Cl₂/MeOH 90/10 Rf=0.90

NMR:.DMSO ¹H δ(ppm): 3.35 (s,3H); 3.65 (s,3H); 4.45 (d,2H); 6.0 (s,2H); 6.80-6.90 (m,2H); 6.95 (s,1H); 8.50 (s,1H); 8.95 (s,1H); 9.25 (t,1H).

IR: 3379, 1713, 1662, 1478, 1253, 1238, 924, 750 cm⁻¹

M.P.=288.7° C.

HPLC: 96.3%

### Example 163

### 3-Benzyl-1-methyl-2,4-dioxo-1,2,3,4-tetrahydro-pyrido[2,3-d]pyrimidine-6-carboxylic acid (1,3-benzodioxol-5-ylmethyl)-amide

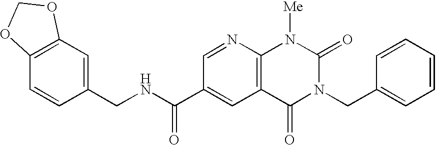

Step 1: N′-(1-Benzyl-3-methyl-2,6-dioxo-1,2,3,6-tetrahydro-pyrimidin-4-yl)-N,N-dimethyl-formamidine

0.56 g (2.5 mmol) of 6-amino-3-benzyl-1H-pyrimidine-2,4-dione (Tetrahedron Letters, 1991, 32(45), 6534-6540) in 20 ml of DMF are stirred under inert atmosphere. 1 ml (7.5 mmol) of N,N′-dimethylformamide dimethyl acetal is added to this solution and the mixture is heated to reflux for 20 minutes. After cooling and concentration under vacuum, the residue is taken up in dichloromethane, and the organic phase is washed with water, dried over Na₂SO₄, and concentrated under vacuum until a low volume. Then the crude product is precipitate by addition of ether. After filtration 0.680 g (yield 72.6%) of the desired compound is obtained.

TLC: CH₂Cl₂/MeOH 90/10 Rf0.80

NMR:.DMSO ¹H δ(ppm): 3.0 (s,3H); 3.15 (s,3H); 3.30 (s,3H); 4.90 (s,2H); 5.20 (s,1H) 7.2-7.35 (m,5H); 8.10 (s,1H)

Step 2: N′-(1-Benzyl-5-iodo-3-methyl-2,6-dioxo-1,2,3,6-tetrahydro-pyrimidin-4-yl)-N,N′-dimethyl-formamidine

To a stirred solution of 0.68 g (2.38 mmol) of the compound obtained in the preceding Step 1 in 24 ml of anhydrous dichloromethane is added 0.64 g (2.85 mmol) of N-iodosuccinimide. After 30 minutes of reflux, the reaction mixture is cooled and the organic phase is washed with water, dried over Na₂SO₄, and concentrated under vacuum. The crude product is precipitated in ether to obtain 0.680 g (yield: 69.3%) of the desired compound.

NMR:.CDCl₃ ¹H δ(ppm): 3.05 (s,3H); 3.15 (s,3H); 3.40 (s,3H); 5.20 (s,2H); 7.2-7.30 (m,3H);7.5-7.55 (m,2H); 7.7 (s,1H).

M.P.=186.3° C.

Step 3: 3-Benzyl-1-methyl-2,4-dioxo-1,2,3,4-tetrahydro-pyrido[2,3-d]pyrimidine-6-carboxylic acid ethyl ester

To a stirred solution of 0.68 g (1.65 mmol) of the compound obtained in the preceding Step 2 in 45 ml of anhydrous DMF are added successively 18 mg Pd(OAc)₂, 8 mg of Cul, 330 mg of K₂CO₃, and 0.22 ml of ethyl acrylate. After 30 minutes under reflux, the reaction mixture is concentrated under vacuum. The residue is taken up in dichloromethane. The organic phase is filtered, washed two times with water, dried over Na₂SO₄ and then concentrated under vacuum. The crude product is purified by chromatography over silica gel (dichloromethane/methanol: 97/3) and then crystallized from ether to give 0.320 g (yield: 57%) of the desired compound.

TLC: CH₂Cl₂/MeOH 97.5/2.5 Rf=0.50

NMR: CDCl₃ ¹H δ(ppm): 1.40 (t,3H); 3.70 (s,3H); 4.40 (q,2H); 5.30 (s,2H); 7.2-7.30 (m,3H); 7.5-7.55 (m,2H); 9.0 (s,1H); 9.2 (s,1H)

Step 4: 3-Benzyl-1-methyl-2,4-dioxo-1,2,3,4-tetrahydro-pyrido[2,3-d]pyrimidine-6-carboxylic acid

The compound is obtained by hydrolysis, in a mixture of dioxan/water in presence of LiOH, of the compound obtained in the preceding Step 3.

TLC: CH₂Cl₂/MeOH 90/10 Rf=0.10

NMR:.DMSO ¹H δ(ppm): 3.60 (s,3H); 5.20 (s,2H); 7.2-7.40 (m,5H); 8.75 (s,1H); 9.2 (s,1H); 13.5 (bs,1H)

HPLC=100%

Step 5: 3-Benzyl-1-methyl-2,4-dioxo-1,2,3,4-tetrahydro-pyrido[2,3-d]pyrimidine-6-carboxylic acid (1,3-benzodioxol-5-ylmethyl)-amide

The compound is obtained according to the procedure of the Example 1 using the compound obtained in the preceding Step 4 and piperonylamine.

TLC: CH₂Cl₂/MeOH 95/5 Rf=0.60

NMR:.DMSO ¹H δ(ppm): 3.60 (s,3H); 4.40 (d,2H); 5.2 (s,2H); 5.95 (s,2H); 6.75-6.95 (m,3H); 7.2-7.40 (m,5H); 8.85 (s,1H); 9.2 (s,1H); 9.25 (t,1H).

IR: 3271, 1709, 1665, 1630, 1614, 1488, 1248, 1042, 937, 795 cm⁻¹

M.P.=174.9° C.

HPLC: 97.5%

### Example 164

### 4[6-(4-Methoxy-benzylcarbomoyl)-1-methyl-2,4-dioxo-1,4-dihydro-2H-pyrido[2,3-d]pyrimidin-3-ylmethyl]-benzoic acid

Step 1: 1-Methyl-2,4-dioxo-1,2,3,4-tetrahydro-pyrido[2,3-d]pyrimidine-6-carboxylic acid

A solution of 1.3 g (4.17 mmol) of the compound obtained in the Step 4 of Example 163 and 3.1 g (23 mmol) of AlCl₃ in 44 ml of benzene is stirred 2 hours at room temperature. After addition of a mixture water/ice, the reaction mixture is extracted successively with ethyl acetate and dichloromethane. The aqueous layer is acidified at pH 1 by addition of concentrated HCl. The precipitate obtained is filtered off and washed with 10 ml of methanol and 10 ml of dichloromethane to provide the desired compound (yield: 62.9%)

NMR:.DMSO ¹H δ(ppm): 3.50 (s,3H); 8.60 (s,1H); 9.10 (s,1H); 11.9 (bs,1H); 13.5 (bs,1H)

HPLC =100%

Step 2: 1-Methyl-2,4-dioxo-1,2,3,4-tetrahydro-pyrido[2,3d]pyrimidine-6-carboxylic acid 4-methoxy-benzylamide

The compound is obtained according to the procedure of the Example 1 using the compound obtained in the preceding Step 2 and 4-methoxybenzylamine.

TLC: CH₂Cl₂/MeOH 95/5 Rf=0.45

NMR..DMSO ¹H δ(ppm): 3.50 (s,3H); 3.7 (s,3H); 4.40 (d,2H); 6.85-6.95 (m,2H); 7.30 (m,2H); 8.80 (s,1H); 9.15 (s,1H); 9.30 (t,1H); 11.85 (bs,1H)

HPLC=92%

Step 3: Methyl 4-[6-(4-Methoxy-benzylcarbamoyl)-1-methyl-2,4-dioxo-1,4-dihydro-2H-pyrido[2,3d]pyrimidin-3-ylmethyl]-benzoate

The compound is obtained according to the procedure of the Step 2 of Example 34 using the compound obtained in the preceding Step 2 and methyl-4-(bromomethyl)benzoate. After concretization in ether 0.41 g (yield: 71.1%) of the desired compound is isolated.

TLC: CH₂Cl₂/MeOH 95/5 Rf=0.80

NMR:.DMSO ¹H δ(ppm): 3.60 (s,3H); 3.80 (s,3H); 3.90 (s,3H); 4.45 (d,2H); 5.2 (s,2H); 6.90 (dd,2H); 7.30 (dd,2H); 7.50 (dd,2H); 7.90 (dd,2H); 8.90 (s,1H); 9.20 (s,1H); 9.30 (t,1H);

HPLC=96.8%

Step 4: 4-[6-(4-Methoxy-benzylcarbamoyl)-1-methyl-2,4-dioxo-1,4-dihydro-2H -pyrido[2,3-d]pyrimidin-3-ylmethyl]-benzoic acid

The compound is obtained according to the procedure of Example 35 using the compound obtained in the preceding Step 3.

NMR:.DMSO ¹H δ(ppm): 3.60 (s,3H); 3.70 (s,3H); 4.45 (d,2H); 5.20 (s,2H); 6.90 (d,2H); 7.25 (d,2H); 7.45 (d,2H); 7.90 (d,2H); 8.85 s,1H); 9.20 (s,1H); 9.30 (t,1H); 12.90 (bs,1H)

IR: 3292, 1718, 1695, 1667, 1633, 1609, 1497, 1301, 1242, 797 cm⁻¹

M.P.=229.5° C.

HPLC: 93.6%

### Example 165

### 3-(4Cyano-benzyl)-1-methyl-4dioxo-1,2,3,4-tetrahydro-pyrido [2,3-d]pyrimidine-6-carboxylic acid 4-methoxy-benzylamide

The compound is obtained (0. 11 g; yield=68.4%) according to the procedure of the Step 2 of Example 34 using the compound obtained in Step 2 of Example 164 and 4-(bromomethyl)benzonitrile.

TLC: CH₂Cl₂/MeOH 95/5 Rf=0.70

NMR:.DMSO ¹H δ(ppm): 3.60 (s,3H); 3.70 (s,3H); 4.40 (d,2H); 5.20 (s,2H); 6.90 (d,2H); 7.30 (d,2H); 7.55 (d,2H); 7.80 (d,2H); 8.85 (s,1H); 9.20 (s,1H); 9.30 (t,1H)

IR: 3230, 2230, 1710, 1673, 1635, 1609, 1494, 1303, 1252, 794 cm⁻¹

M.P.=197° C.

HPLC: 97.2%

### Example 166

### 3-(4-Fluoro-benzyl)-1-methyl-2,4-dioxo-1,2,3,4-tetrahydropyrido [2,3-d]pyrimidine-6 -carboxylic acid 4-methoxy-benzylamide

The compound is obtained according to the procedure of the Step 2 of Example 34 using the compound obtained in Step 2 of Example 164 and 4-fluorobenzyl bromide.

TLC: CH₂Cl₂/MeOH 95/5 Rf=0.70

NMR:.DMSO ¹H δ(ppm): 3.60 (s,3H); 3.70 (s,3H); 4.40 (d,2H); 5.10 (s,2H); 6.8-6.90 (m,2H); 7.1-7.2 (m,2H); 7.25-7.35 (m,2H); 7.4-7.50 (m,2H); 8.85 (s,1H); 9.15 (s,1H); 9.30 (t,1H).

IR: 3260, 1709, 1664, 1616, 1497, 1245, 1221, 1035, 796 cm⁻¹

M.P.=211.5° C.

HPLC: 98.3%

### Example 167

### 3-Benzyl-1-methyl-24-dioxo-1,2,3,4-tetrahydro-pyrido[3,4-d]pyrimidine-6-carboxylic acid (1,3-benzodioxol-5-ylmethyl)-amide

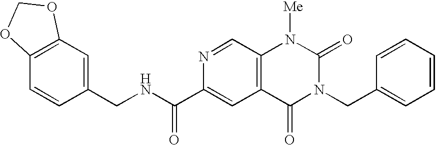

Step 1: 1-Benzyl-2,6-dioxo-1,2,3,6-tetrahydro-pyrimidine-4-carbaldehyde

A solution of 9.5 g (43.9 mmol) of 3-benzyl-6-methyl-1H-pyrimidine-2,4-dione (Synthetic Communications 1991, 2181-2188) and 129 ml of cold acetic acid are stirred 5 minutes, and 5.75 g of SeO₂ are added. The reaction mixture is heated to reflux for 2 h30, filtered and concentrated under vacuum. The residue is taken up in dichloromethane. The unsoluble part is eliminated and the filtrate is concentrated under vacuum. A chromatography over silica gel (dichloromethane/methanol: 95/5) provides 4.0 g of the desired compound (yield:39.5%).

NMR:.CDCl₃ ¹H δ(ppm): 5.20 (s,2H); 6.30 (s,1H); 7.2-7.30 (m,3H); 7.40-7.50 (m,2H); 9.0 (bs,1H); 9.60 (s,1H)

Step 2: 1-Benzyl-2,6-dioxo-1,2,3,6-tetrahydro-pyrimidine-4-carbaldehyde dimethylhydrazone

To a stirred solution of 3.6 g (15.6 mmol) of the compound obtained in the preceding Step 1 in 80 ml of anhydrous DMF are added 1.2 ml (0.94 g, 15.6 mmol) of dimethylhydrazine. After 1 hour of stirring at room temperature, the solvent is removed under vacuum and the residue is taken up in dichloromethane. The organic layer is washed, dried over Na₂SO₄ and concentrated. A chromatography over silica gel (dichloromethane/methanol: 97/3) provides 2.5 g (yield:59%) of the desired compound.

NMR:.CDCl₃ ¹H δ(ppm) 3.10 (s,6H);5.10 (s,2H); 5.55 (s,1H); 6.50 (s,1H); 7.2-7.30 (m,3H); 7.40-7.50 (m,2H); 8.50 (bs,1H)

Step 3: 1-Benzyl-2,6-dioxo-3-methyl-1,2,3,6-tetrahydro-pyrimidine-4-carbaldehyde dimethylhydrazone

To a stirred solution of 2.3 g (8.45 mmol) of the compound obtained in the preceding Step 2 in 58 ml of anhydrous DMF are added 2.3 ml (2.0 g, 1.69 mmol) of N,N′-dimethylformamide acetal. The reaction mixture is maintained at 100° C. for 10 minutes and concentrated under vacuum. The residue is taken up in dichloromethane and the product is precipitated by addition of ether to provide 1.75 g (yield:72.3%) of the desired compound.

NMR:. CDCl₃ ¹H δ(ppm) 3.20 (s,6H);3.50 (s,3H); 5.15 (s,2H); 6.10 (s,1H); 6.60 (s,1H); 7.2-7.30 (m,3H); 7.40-7.50 (m,2H)

Step 4: Methyl 1-benzyl-2,6-dioxo-3-methyl-1,2,3,6-tetrahydro-pyrimidine-4-(carbaldehyde dimethylhydrazone)-5-carboxylate

To a stirred solution of 1.7 g (5.94 mmol) of the compound obtained in the preceding Step 3 in 61 ml of anhydrous acetonitrile are added successively 1.68 g (7.1 mmol) of Pd(OAc)₂ and 0.613 g (7.1 mmol) of methyl acrylate. After 20 minutes od stirring under reflux the reaction mixture is filtered off and concentrated under vacuum. The residue is chromatographed over silica gel (dichloromethane/methanol: 97/3) to provide 1.40 g (yield:63.6%) of the desired compound.

NMR:. CDCl₃ ¹H δ(ppm): 3.20 (s,6H) ;3.55 (s,3H); 3.75 (s,3H); 5.20 (s,2H); 6.70 (s,1H); 7.1-7.70 (m,7H).

Step 5: 3-Benzyl-1-methyl-2,4-dioxo-1,2,3,4-tetrahydro-pyrido[3,4-d]pyrimidine -6-carboxylic acid methyl ester

A solution of 1.4 g (3.78 mmol) of the compound obtained in the preceding Step 4, 18 ml of chlorobenzene and 3.6 ml of acetic acid is stirred under reflux for 3 hours, and concentrated under vacuum to provide 1.4 g of a precipitate. The desired compound (0.76 g; yield:62%) is obtained by recrystallization of the crude product in 120 ml of ethyl acetate.

NMR:. CDCl₃ ¹H δ(ppm): 3.70 (s,3H);4.0 (s,3H); 5.30 (s,2H); 7.2-7.35 (m,3H); 7.45-7.55 (m,2H); 8.80 (s,1H) ; 8.85 (s,1H).

Step 6: 3-Benzyl-1-methyl-2,4-dioxo-1,2,3,4-tetrahydro-pyrido[3,4-d]pyrimidine -6-carboxylic acid

0.76 g (2.34 mmol) of the compound obtained in the preceding Step 5, 7.6 ml of methanol, 7.6 ml of water and 0.646 g (4.67 mmol) of K₂CO₃ are stirred overnight at room temperature and then heated to reflux for 5 minutes. After cooling and addition of water the acidification to pH 1 of the mixture provides a precipitate which is dissolved in a mixture of methanol/dichloromethane. The organic layer is washed with water, dried and concentrated under vacuum. The residue obtained is concretized in a mixture of dichloromethane/ether to give 0.54 g (yield: 74%) of the desired compound.

NMR:.DMSO ¹H δ(ppm) 3.60 (s,3H); 5.20 (s,2H); 7.2-7.40 (m,5H); 8.50 (s,1H); 9.0 (s,1H); 13.3 (bs,1H)

M.P.=240° C.

HPLC: 100%

Step 7: 3-Benzyl-1-methyl-2,4-dioxo-1,2,3,4-tetrahydro-pyrido[3,4-d]pyrimidine -6-carboxylic acid (1,3-benzodioxol-5-ylmethyl)-amide

The compound is obtained according to the procedure of the Example 1 using the compound obtained in the preceding Step 6 and piperonylamine.

TLC: CH₂Cl₂/MeOH 95/5 Rf=0.60

NMR:.DMSO ¹H δ(ppm): 3.65 (s,3H); 4.40 (d,2H); 5.15 (s,2H); 5.95 (s,2H); 6.75-6.85 (m,2H); 6.90 (s,1H); 7.2-7.40 (m,5H); 8.45 (s,1H); 8.90 (s,1H); 9.25 (t,1H).

IR: 3387, 1716, 1662, 14875, 1442, 1250, 1239, 1040, 789 cm⁻¹

M.P.=197.5° C.

HPLC: 100%

### Example 168

### Methyl 4-[6-(4-Methoxy-benzylcarbamoyl)-1-methyl-4-dioxo-1,4-dihydro-2H-pyrido[3,4-d]pyrimidin-3-ylmethyl]-benzoate

Step 1: 1-Methyl-2,4-dioxo-1,2,3,4-tetrahydro-pyrido[3,4-d]pyrimidine-6-carboxylic acid

3.3 g (10.6 mmol) of the compound obtained in the Step 6 of Example 167 are treated according to the procedure described in the Step 1 of Example 164 to give 2.0 g (yield: 85.3%) of the desired compound.

NMR:.DMSO ¹H δ(ppm): 3.60 (s,3H); 8.40 (s,1H); 8.95 (s,1H); 12.0 (s,1H); 12.90 (bs,1H)

HPLC: 100%

Step 2: 1-Methyl-2,4-dioxo-1,2,3,4-tetrahydro-pyrido[3,4-d]pyrimidine-6-carboxylic acid 4-methoxy-benzylamide

The compound is obtained (yield: 78%) according to the procedure of the Example 1 using the compound obtained in the preceding Step 1 and 4-methoxybenzylamine.

TLC: CH₂Cl₂/MeOH 95/5 Rf=0.50

NMR:.DMSO ¹H δ(ppm): 3.60 (s,3H); 3.75 (s,3H); 4.40 (d,2H); 6.85 (dd,2H); 7.25 (dd,2H); 8.40 (s,1H); 8.85 (s,1H); 9.20 (t,1H); 12.0 (s,1H)

HPLC:=99%

Step 3: Methyl 4-[6-(4-methoxy-benzylcarbamoyl)-1-methyl-2,4-dioxo-1,4-dihydro-2H-pyrido[3,4-d]pyrimidin-3-ylmethyl]-benzoate

The compound is obtained (0.2 g; yield:77%) according to the procedure of the Step 2 of Example 34 using the compound obtained in the preceding Step 2 and methyl-4-(bromomethyl)benzoate.

TLC: CH₂Cl₂/MeOH 95/5 Rf=0.80

NMR:.DMSO ¹H δ(ppm): 3.60 (s,3H); 3.70 (s,3H); 3.85 (s,3H); 4.50 (d,2H); 5.20 (s,2H); 6.85 (d,2H); 7.20 (d,2H); 7.50 (d,2H); 7.90 (d,2H); 8.5 (s,1H); 8.90 (s,1H); 9.20 (t,1H)

IR: 3396, 1719, 1661, 1439, 1279, 1250, 1110, 753 cm⁻¹

M.P.=211.1° C.

HPLC: 99.5%

### Example 169

### tert-Butyl 4-[6-methoxy-benzylcarbamoyl)-1-methyl-2,4-dioxo-1,4-dihydro-2H-pyrido[3,4-d]pyrimidin-3-ylmethyl]-benzoate

The compound is obtained (yield: 80.4%) according to the procedure of the Step 2 of Example 34 using the compound obtained in the Step 2 of example 168 and tert-butyl 4-bromomethyl-benzoate.

TLC: CH₂C₂/MeOH 95/5 Rf=0.80

NMR:.DMSO ¹H δ(ppm): 1.50 (s,9H); 3.65 (s,3H); 3.75 (s,3H); 4.40 (d,2H); 5.20 (s,2H); 6.85 (dd,2H); 7.25 (dd,2H); 7.45 (dd,2H); 7.85 (dd,2H); 8.50 (s,1H); 8.90 (s,1H); 9.2 (t,1H);

HPLC:=98%

### Example 170

### 4-[6-(3-Methoxy-benzylcarbamoyl)-1-methyl-2,4-dioxo-1,4-dihydro -2H-pyrido[3,4-d]pyrimidin-3-ylmethyl]-benzoic acid

Step 1: 1-Methyl-2,4-dioxo-1,2,3,4-tetrahydro-pyrido[3,4-d]pyrimidine-6-carboxylic acid 3-methoxy-benzylamide

The compound is obtained (yield: 62.4%) according to the procedure of the Example 1 using the compound obtained in the Step 1 of Example 168 and 3-methoxybenzylamine.

TLC: CH₂C₂/MeOH 95/5 Rf=0.50

NMR:.DMSO ¹H δ(ppm): 3.60 (s,3H); 3.75 (s,3H); 4.50 (d,2H); 6.75-6.95 (m,3H); 7.20-7.30 (m,1H); 8.40 (s,1H); 8.85 (s,1H); 9.25 (t,1H); 12.0 (s,1H)

HPLC:=98%

Step 2: tert-Butyl 4-[6-(3-Methoxy-benzylcarbamoyl)-1-methyl-2,4-dioxo-1,4-dihydro-2H-pyrido[3,4-d]pyrimidin-3-ylmethyl]-benzoate

The compound is obtained (yield: 80.4%) according to the procedure of the Step 2 of Example 34 using the compound obtained in the preceding Step 1 and tert-butyl 4-(bromomethyl)benzoate.

TLC: CH₂Cl₂/MeOH 95/5 Rf=0.80

NMR:.DMSO ¹H δ(ppm): 1.50 (s,9H); 3.65 (s,3H); 3.75 (s,3H); 4.50 (d,2H); 5.20 (s,2H); 6.80-6.95 (m,3H); 7.20-7.30 (m,1H); 7.5 (dd,2H); 7.85 (dd,2H); 8.50 (s,1H); 8.95 (s,1H); 9.3 (t,1H);

HPLC:=93.6%

Step 3: 4-[6-(3-Methoxy-benzylcarbamoyl)-1-methyl-2,4-dioxo-1,4-dihydro-2H-pyrido[3,4-d]pyrimidin-3-ylmethyl]-benzoic acid

The compound is obtained according to the procedure of the Step 2 of Example 169 using the compound obtained in the preceding Step 2.

TLC CH₂Cl₂/MeOH 95/5 Rf=0.60

NMR:.DMSO ¹H δ(ppm): 3.65 (s,3H); 3.75 (s,3H); 4.50 (d,2H); 5.20 (s,2H); 6.75-6.80 (s,1H); 6.90 (s,2H); 7.20-7.25 (m,1H); 7.45 (d,2H); 7.85 (d,2H); 8.5 (s,1H); 8.90 (s,1H); 9.30 (t,1H); 12.95 (bs,1H)

IR: 3378, 1712, 1660, 1600, 1439, 1266, 1056, 790 cm⁻¹

M.P.=208.1° C.

HPLC:96.6%

### Example 171

### 3-(4-Cyano-benzyl)-1-methyl-2,4-dioxo-1,2,3,4-tetrahydro-pyrido[3,4-d]pyrimidine-6-carboxylic acid 4-methoxy-benzylamide

The compound is obtained according to the procedure of the Step 2 of Example 34 using the compound obtained in the Step 2 of Example 168 and (4-bromomethyl)-benzonitrile

TLC: CH₂Cl₂/MeOH 95/5 Rf=0.80

NMR:.DMSO ¹H δ(ppm): 3.65 (s,3H); 3.75 (s,3H); 4.45 (d,2H); 5.25 (s,2H); 6.90 (d,2H); 7.25 (d,2H); 7.55 (d,2H); 7.80 (d,2H); 8.5 (s,1H); 8.95 (s,1H); 9.20 (t,1H).

IR: 3391, 2228, 1716, 1662, 1443,1331, 1251, 789 cm⁻¹

M.P.=230° C.

HPLC: 98.8%

### Example 172

### 3-Benzyl-1-methyl-6-(3-phenyl-propionyl)-1H-quinazoline-2,4-dione

The compound of the preparation C is treated by SOCl₂ in THF to give its chloride derivate which is reacted with phenetyl magnesium bromide and CuI in presence of THF. After usual treatment the desired compound is obtained.

NMR:.CDCl₃ ¹H δ(ppm): 3.0 (m,2H); 3.30 (m,2H); 3.60 (s,3H); 5.25 (s,2H); 7.10-7.35 (m,9H); 7.50 (m,2H); 8.3 (m,1H); 8.80 (s,1H)

M.P.=155° C.

HPLC: 98.0%

### Example 173

### 3-Benzyl-1-methyl-2,4-dioxo-1,2,3,4-tetrahydro-quinazoline-6-carboxylic acid (E)-3-pyridin-4-yl-allyl ester

NMR:.CDCl₃ ¹H δ(ppm) 3.60 (s,3H); 5.0 (d,2H); 5.30 (s,2H); 6.5-6.7 (m,2H); 7.15-7.35 (m,6H); 7.55 (m,2H); 8.40 (m,1H); 8.60 (m,2H); 9.0 (s,1H)

M.P.=147° C.

HPLC: 97.5%

### Example 174

### 3-Benzyl-1-methyl-2,4-dioxo-1,2,3,4-tetrahydro-quinazoline-6carboxylic acid (E)-3-pyridin-3-yl-allyl ester

NMR:.CDCl₃ ¹H δ(ppm): 3.60 (s,3H); 5.0 (d,2H); 5.30 (s,2H); 6.5 (m,1H); 6.8 (d,1H); 7.30 (m,5H); 7.60 (m,2H); 7.7 (d,1H); 8.40 (d,1H); 8.55 (m,1H); 8.70 (s,1H); 9.0 (s,1H)

M.P.=184° C.

HPLC: 99.6%

### Example 175

### 3-Benzyl-1-methyl-6-[2-(pyridin-4-ylsulfanyl)-acetyl]1H-quinazoline-2,4-dione

TLC: CH₂C₂/MeOH 98/2 Rf=0.20

NMR:.CDCl3 ¹H δ(ppm): 3.65 (s,3H); 4.45 (s,2H); 5.25 (s,2H); 7.18 (d,2H); 7.20-7.35 (m,4H); 7.50 (d,2H); 8.3 (d,1H); 8.40 (d,2H); 8.80 (s,1H).

IR :1706, 1693, 1657, 1610, 1574, 1508, 1480, 1448, 1428, 1321, 1307, 1206, 1093, 831, 810, 782, 703 cm⁻¹

M.P.=187° C.

HPLC:98.0%

### Example 176

### 3-(4-Aminomethyl-benzyl)-1-methyl-2,4-dioxo-1,2,4-tetrahydro-quinazoline-6-carboxylic acid 4-methoxy-benzylamide

The compound is obtained by catalytic hydrogenation of the compound of Example 60 using Raney Ni and NH₃ in methanol.

TLC: CH₂Cl₂/MeOH/NH₄OH 90/10/1 Rf=0.25

NMR:.CDCl₃ ¹H δ(ppm): 1.45-1.70 (m,2H); 3.6 (s,3H); 3.8 (m,5H); 4.55 (d,2H); 5.22 (s,2H); 6.74 (m,1H); 6.86 (d,2H); 7.2-7.30 (m,5H); 7.44 (d,2H); 8.28 (d,1H); 8.48 (s,1H)

IR: 3370, 1702, 1655, 1640, 1617, 1542, 1508, 1477, 1324, 1303; 1247, 1173, 1032, 829, 786, 756 cm⁻¹

M.P.=187° C.

HPLC:98.4%

### Example 177

### 3-2′-Cyano-biphenyl-4-ylmethyl)-1-methyl-2,4-dioxo-1,2,3,4 -tetrahydro-quinazoine-6-carboxylic acid 4-methoxy-benzylamide

The compound is obtained according to the procedure of the Step 2 of Example 34 using 2-(4-bromomethylphenyl)-benzonitrile.

TLC: CH₂Cl₂/MeOH 98.5/1.5 Rf=0.20

NMR:.CDCl₃ ¹H δ(ppm): 3.65 (s,3H); 3.80 (s,3H); 4.55 (d,2H); 5.30 (s,2H); 6.55-6.65 (m,1H); 6.25 (d,2H); 7.2-7.30 (m,3H); 7.35-7.50 (m,4H); 7.55-7.65 (m,3H); 7.75 (d,1H); 8.25-8.35 (m,1H); 8.45 (s,1H)

IR: 1702, 1661, 1629, 1508, 1478, 1332, 1242, 1036, 833, 766 cm⁻¹

M.P.=200° C.

HPLC: 99.8%

### Example 178

### 1-Methyl-2,4-dioxo-3-[2′-(1H-tetrazol-5-yl)-biphenyl-4-ylmethyl]-1,2,3,4-tetrahydro-quinazoline-6-carboxylic acid 4-methoxy-benzylamide

The compound is obtained according to the procedure of the Step 2 of Example 34 using 5-[(4-bromomethyl)biphenyl]-tetrazole.

TLC: CH₂Cl₂/MeOH 90/10 Rf=0.50

NMR:.DMSO ¹H δ(ppm): 3.55 (s, 3H); 3.75 (s,3H); 4.40 (d,2H); 5.15 (s,2H); 6.90 (d,2H); 7.05 (d,2H); 7.25 (d,4H); 7.45-7.70 (m,6H); 8.30 (d,1H); 8.6 (s,1H); 9.25 (m,1H)

IR: 2943, 1702, 1656, 1618, 1510, 1477, 1450, 1323, 1302, 1247, 1032, 829, 814, 782, 757 cm⁻¹

HPLC: 99.6%

### Example 179

### Methyl 4′-[6-(4methoxy-benzylcarbamoyl)-1-methyl-2,4-dioxo-1,4-dihydro-2H-quinazolin-3-ylmethyl]-biphenyl-2-carboxylate

The compound is obtained according to the procedure of the Step 2 of Example 34 using Methyl 4-(bromomethylphenyl)benzoate

TLC: CH₂Cl₂/MeOH 97/3 Rf=0.30

NMR: DMSO ¹H δ(ppm): 3.61 (s,3H); 3.62 (s,3H); 3.80 (s,3H); 4.55 (d,2H); 5.30 (s,2H); 6.65 (t,1H); 6.85(d,2H); 7.2-7.30 (m,6H); 7.35-7.40 (m,1H); 7.45-7.55 (m,3H); 7.80 (d,1H); 8.27 (d,1H); 8.47 (s,1H)

IR: 1707, 1668, 1656, 1638, 1616, 1509, 1478, 1330, 1294, 1248, 1089, 765, 754 cm⁻¹

M.P.=172° C.

HPLC: 99.7%

### Example 180

### 4′-[6-4-Methoxy-benzylcarbamoyl)-1-methyl-2,4-dioxo-1,4-dihydro-2H-quinazolin-3-ylmethyl]-biphenyl-2-carboxylic acid

The compound is obtained according to the procedure of the Step 2-4 of Preparation B using the compound of Example 179.

TLC: CH₂Cl₂/MeOH 90/10 Rf=0.40

NMR:.DMSO ¹H δ(ppm): 3.57 (s,3H); 3.72 (s,3H); 4.42 (d,2H); 5.20 (s,2H); 6.90 (d,2H); 7.25-7.45 (m,8H); 7.50-7.60 (m,2 H); 7.70 (d,1H); 8.26 (d,1H); 8.60 (s,1H); 9.17-9.27 (m,1H); 12.5-13.2 (m,1H)

IR: 1698, 1668, 1655, 1639, 1612, 1508, 1479, 1330, 1304, 1248, 765, 754 cm⁻¹

M.P.=175° C.

HPLC: 100%

### Example 181

### Ethyl 2-Fluoro,4-[6-(4-methoxy-benzylcarbamoyl)-1-methyl-2,4dioxo-1,4-dihydro-2H-quinazolin-3-ylmethyl]-benzoate

The compound is obtained according to the procedure of the Step 2 of Example 34 using Methyl 4-(bromomethyl)-2-fluoro-benzoate.

TLC: CH₂Cl₂/MeOH 90/10 Rf=0.60

NMR: CDCl₃ ¹H δ(ppm): 1.30 (t,3H); 3.60 (s,3H); 3.80 (s,3H); 4.35 (q,2H); 4.60 (m,2H); 5.30 (s,2H); 6.55 (m,1H); 6.90 (m,2H); 7.30 (m,5H); 7.90 (m,1H); 8.30 (m,1H); 8.50 (s,1H);

M.P.=156° C.

HPLC: 100%

### Example 182

### 2-Fluoro-4-[6-(4-methoxy-benzylcarbamoyl)-1-methyl-2,4-dioxo-1,4-dihydro-2H-quinazolin-3-ylmethyl]-benzoic acid

The compound is obtained according to the procedure of the Step 2-4 of Preparation B using the compound of Example 181.

TLC: CH₂Cl₂/MeOH 90/10 Rf=0.20

NMR:.DMSO ¹H δ(ppm): 3.60 (s,3H); 3.75 (s,3H); 4.40 (m,2H); 5.20 (s,2H); 6.90 (m,2H); 7.30 (m,4H); 7.60 (d,1H); 7.80 (m,1H); 8.30 (m,1H); 8.70 (s,1H); 9.2 (s,1H); 13.2 (s,1H)

M.P.=160° C.

HPLC: 100%

### Example 183

### 2-Methoxy-4-[6-(4-methoxy-benzylcarbamoyl)-1-methyl-2,4-dioxo-1,4-dihydro-2H-quinazolin-3-ylmethyl]-benzoic acid 2-dimethylamino-ethyl ester

TLC: CH₂C₂/MeOH 90/10 Rf=0.20

NMR:.CDCl₃ ¹H δ(ppm) 2.3 (s,6H); 2.60 (m,2H); 3.60 (s, 3H); 3.75 (s,3H); 3.85 (s,3H); 4.35 (m,2H); 4.55 (m,2H); 5.25 (s,2H); 6.50 (m,1H); 6.80 (m,2H); 7.10 (d,1H); 7.25 (m,4H) ; 7.70 (d,1H); 8.25 (m,1H); 8.5 (s,1H)

M.P.=130° C.

HPLC: 97.3%

### Example 184

### 4-[6-(4-Methoxy-benzylcarbamoyl)-1-methyl-2,4-dioxo-1,4-dihydro-2-quinazolin-3-ylmethyl]-2-methyl-benzoic acid 2-dimethylamino-ethyl ester

TLC: CH₂Cl₂/MeOH 90/10 Rf=0.60

NMR:.CDCl₃ ¹H δ(ppm) 2.3 (s,6H); 2.55 (s,3H); 2.70 (m,2H); 3.60 (s, 3H); 3.80 (s,3H); 4.40 (m,2H); 4.60 (m,2H); 5.20 (s,2H); 6.60 (s,1H); 6.80 (m,2H); 7.30 (m,5H); 7.80 (m,1H); 8.30 (m,1H); 8.5 (s,1H)

M.P.=146° C.

HPLC: 99%

### Example 185

### 1-Methyl-2,4-dioxo-3-[4-(5-oxo-4,5-dihydro-1,2,4-oxadiazol-3-yl)-benzyl]-1,2,3,4-tetrahydro-quinazoline-6-carboxylic acid 4-methoxy-benzylamide

TLC: CH₂Cl₂/MeOH 90/10 Rf=0.30

NMR:.DMSO ¹H δ(ppm) 3.2 (m,1H); 3.55 (s, 3H); 3.70 (s,3H); 4.40 (d,2H); 5.20 (s,2H); 6.90 (m,2H); 7.25 (m,2H); 7.40 (m,2H); 7.55 (m,1H); 7.70 (m,2H); 8.30 (m,1H); 8.60 (s,1H); 9.2 (m,1H)

M.P.=305° C.

HPLC:100%

### Example 186

### {4-[6-(4-Methoxy-benzylcarbamoyl)-1-methyl-2,4-dioxo-1,4-dihydro-2H-quinazolin-3-yl]-phenyl}-acetic acid

TLC: CH₂Cl₂/MeOH 90/10 Rf=0.35

NMR:.DMSO ¹H δ(ppm) 3.50 (m,5H); 3.70 (s,3H); 4.40 (d,2H); 6.80 (d,2H); 7.20 (m,4H); 7.40 (d,2H); 7.60 (d ,1H); 8.30 (d,1H); 8.60 (s,1H); 9.2 (t,1H)

IR=1717, 1645, 1619, 1501, 1298, 1240, 823, 750

HPLC: 100%

### Example 187

### 1-Methyl-3-(1-napthalen-1-yl-ethyl)-2,4-dioxo-1,2,3,4-tetrahydro-quinazoline-6-carboxylic acid (1,3-benzodioxol-5-ylmethyl)-amide

TLC: CH₂Cl₂/MeOH 95/5 Rf=0.58

NMR:.DMSO ¹H δ(ppm) 2.0 (d3H); 3.45 (s, 3H); 4.40 (d,2H); 6.00 (s,2H); 6.80-6.95 (m,4H); 7.4-7.50 (m,3H); 7.55 (t,1H); 7.85-8.0 (m,4H); 8.20 (d,1H); 8.6 (s,1H); 9.15 (t1H)

IR: 1656, 1618, 1503, 1440, 1254, 1040, 777, 754 cm⁻¹

M.P.=157° C.

HPLC: 96.2%

### Example 188

### 4-[6-(4-Methoxy-benzylcarbamoyl)-1-methyl-2,4-dioxo-1,4-dihydro-2H-pyrido[3,4-d]pyrimidin-3-ylmethyl]-benzoic acid

To a stirred solution of 0.5 g (0.9 mmol) of the compound obtained in the Example 169 in 50 ml of dichloromethane are added 5 ml of trifluorocetic acid. The mixture is stirred overnight at room temperature and 60 ml of ether are added. The product crystallizes and after filtration 0.44 g (yield: 100%) of the desired compound is obtained.

TLC: CH₂C₂/MeOH 95/5 Rf=0.60

NMR:.DMSO ¹H δ(ppm): 3.65 (s,3H); 3.75 (s,3H); 4.45 (d,2H); 5.25 (s,2H); 6.90 (d,2H); 7.25 (d,2H); 7.50 (d,2H); 7.90 (d,2H); 8.5 (s,1H); 8.95 (s,1H); 9.20 (t,1H); 12.85 (bs,1H)

IR : 3388, 1715, 1662, 1475, 1442, 1247, 791 cm⁻¹

M.P.=264.4° C.

HPLC: 98.9%

### Example 189

### 3-(3-Fluoro-benzyl)-1-methyl-2,4-dioxo-1,2,3,4-tetrahydro-quinazoline-6-carboxylic acid (pyridin-4-ylmethyl)-amide

To 0.5 g (1.5 mmol) of the compound of Preparation D in dimethylformamide (10 ml) are added EDAC.HCl 0.38 g (1.9 mmol), HOBT 0.27 g (1.9 mmol), followed by 4-pyridyl-benzylamine 0.21 g (1.9 mmol). The mixture is stirred 48 hours at room temperature before adding water (20 ml) and extracting with ethyl acetate (2×20 ml). The combined organic layers are washed with saturated aqueous NaCl solution (4×20 ml), and dried MgSO₄. recrystallyzed solid product in hot ethyl acetate to obtain 0.13 g (yield: 20%) of the desired compound.

MS: m/z (APCI, AP+) 419.2 [M⁻]⁺

CHN Analysis: Calcd (%) :C, 66.02; H, 4.58; N, 13.39. Found (%) :C, 65.73; H, 4.47; N, 13.36.

### Example 190

### 3-3-Fluoro-benzyl)-1-methyl-2,4-dioxo-1,2,3,4-tetrahydro-quinazoline-6-carboxylic acid (2-methoxy-pyridin-4-ylmethyl)-amide

0.10 g (yield: 17%) of the desired compound is obtained according to the procedure of Example 189, but using 2-methoxy-4-pyridyl-benzylamine.

MS: m/z (APCI, AP+) 449.2 [M⁻]⁺

CHN Analysis: C₂₄H₂₁FN₄O_{4.}0.1 H₂O

Calcd (%) :C, 64.02; H, 4.75; N, 12.44. Found (%) :C, 63.66; H, 5.07; N, 12.16.

### Example 191

### 3-(3-Fluoro-benzyl)1-methyl-2,4-dioxo-1,2,3,4-tetrahydro-quinazoline-6-carboxylic acid (pyridin-3-ylmethyl)-amide

0.11 g (yield: 26%) of the desired compound is obtained according to the procedure of Example 189, but using 3-pyridyl-benzylamine.

MS: m/z (APCI, AP+) 419.1 [M⁻]⁺

CHN Analysis: C₂₃H₁₉FN₄O₃ 1.2 H₂O Calcd (%) :C, 62.78; H, 4.90; N, 12.73. Found (%) :C, 62.75; H, 4.90; N, 12.73.

### Example 192

### 3-(3-Fluoro-benzyl)-1-methyl-2,4-dioxo-1,2,3,4-tetrahydro-quinazoline-6-carboxylic acid 4-methoxy-benzylamide

0.12 g (yield: 35%) of the desired compound is obtained according to the procedure of Example 189, but using 4-methoxy-benzylamine.

MS: m/z (APCI, AP+) 448.1 [M⁻]⁺

CHN Analysis: C₂₅H₂₂FN₃O_{4.}0.1 H₂O Calcd (%) :C, 66.84; H, 4.98; N, 9.35. Found (%) :C, 66.57; H, 4.83; N, 9.03.

### Example 193

### 3-(3-Fluoro-benzyl)-1-methyl-2,4-dioxo-1,2,3,4-tetrahydro-quinazoline-6-carboxylic acid 3-methoxy-benzylamide

0.20 g (yield: 59%) of the desired compound is obtained according to the procedure of Example 189, but using 3-methoxy-benzylamine.

MS: m/z (APCI, AP+) 448.1 [M⁻]⁺

CHN Analysis: C₂₅H₂₂FN₃O₄ Calcd (%) :C, 67.11; H, 4.96; N, 9.39. Found (%) :C, 66.82; H, 4.87; N, 9.11.

### Example 194

### 1-Ethyl-3-(3-fluoro-benzyl)-2,4-dioxo-1,2,3,4,-tetrahydro-quinazoline-6-carbolic acid (pyridina-4-ylmethyl)-amide

0.13 g (yield: 20%) of the desired compound is obtained according to the procedure of Example 189, but using the compound of the Preparation E and 4-pyridyl-benzylamine.

MS: m/z (APCI AP+) 433.2 [M⁻]⁺

CHN Analysis: Calcd (%) :C, 66.66; H, 4.89; N, 12.96. Found (%) :C, 66.26; H, 4.71; N, 12.78.

### Example 195

### 1-Ethyl-3-(3-fluoro-benzyl)-2,4-dioxo-1,2,3,4-tetrahyd-quinazoline-6-carboxylic acid (pyridin-3-ylmethyl)-amide

0.18 g (yield: 51%) of the desired compound is obtained according to the procedure of Example 189, but using the compound of Preparation E and 3-pyridyl-benzylamine.

MS: m/z (APCI, AP+) 433.1 [M⁻]⁺

CHN Analysis: Calcd (%) :C, 66.66; H, 4.89; N, 12.96. Found (%) :C, 66.43; H, 5.03; N, 12.84.

### Example 196

### 3-(4-Bromo-benzyl)-1-methyl-24-dioxo-1,2,3,4-tetrahydro-quinazoline-6-carboxylic acid 4-methoxy-benzyamide

Step 1: Methyl 3-(4-bromobenzyl)-2,4-dioxo-1,2,3,4-tetrahydro-quinazoline-6-carboxylate

4.6 g (yield: 59%) of the desired compound is obtained according to the procedure of Step 1 of Preparation D, but using 4-bromobenzyl isocyanate.

MS: m/z (APCI, AP+) 388.9 [M⁻]⁺

CHN Analysis: Calcd (%) :C, 52.46; H, 3.37; N, 7.20. Found (%) :C, 52.16; H, 3.30; N, 7.30.

Step 2: Methyl 1-methyl-3-(4-bromobenzyl)-2,4-dioxo-1,2,3,4-tetrahydro-quinazoline-6-carboxylate

1.49 g (yield: 71%) of the desired compound is obtained according to the procedure of step 2 of Preparation D, but using the compound obtained in the Preceding Step 1.

MS: m/z (APCI, AP+) 404.9 [M⁻]⁺

CHN Analysis: Calcd (%) :C, 53.62; H, 3.75; N, 6.95. Found (%) :C, 53.24; H, 3.71; N, 6.84.

Step 3: 1-Methyl-3-(4-bromobenzyl)-1,2,3,4-dioxo-1,2,3,4-tetrahydro-quinazoline-6-carboxylic acid

1.3 g (yield: 87%) of the desired compound is obtained according to the procedure of Step 2-4 of Preparation B, but using the compound obtained in the preceding Step 2.

MS: m/z (APCI, AP+) 388.9 [M⁻]⁺

CHN Analysis: Calcd (%) :C, 52.46; H, 3.37; N, 7.20. Found (%) :C, 52.12; H, 3.30; N, 7.11.

Step 4: 3-(4-Bromo-benzyl)-1-methyl-2,4-dioxo-1,2,3,4-tetrahydro-quinazoline-6-carboxylic acid 4-methoxy-benzylamide

0.24 g (yield: 76%) of the desired compound is obtained according to the procedure of Example 189, but using the compound obtained in the preceding Step 3 and 4-methoxy-benzylamine.

MS: m/z (APCI, AP+) 508 [M⁻]⁺

CHN Analysis: C₂₅H₂₂BrN₃O₄ 0.2 H₂O Calcd (%) :C, 58.65; H, 4.41; N, 8.21. Found (%) :C, 58.32; H, 4.32; N, 8.12.

### Example 197

### 3-(4-Bromo-benzyl)-1-methyl-2,4-dioxo-1,2,3,4-tetrahydro-quinazoline-6-carboxylic acid (2-methoxy-pyridin-4-ylmethyl)-amide

0.22 g (yield: 33%) of the desired compound is obtained according to the procedure of Example 189, but using the compound obtained in the preceding Step 3 and 2-methoxy-4-pyridyl-benzylamine.

NMR: DMSO ¹H δ(ppm): 3.52 (3H,s); 3.79 (3H,s); 4.43 (2H,d); 5.09 (2H,s); 6.66 (1H,s); 6.89 (1H,d); 7.26-7.56 (5H,m); 8.06 (1H,d); 8.24-8.26 (1H,m); 8.61(1H,m); 9.31 (1H,t).

MS: m/z (APCI, AP+) 509 [M⁻]⁺

### Example 198

### 3-(3,4-Difluoro-benzyl)-methyl-2,4-dioxo-1,2,3,4-tetrahydro-quinazoline-6-carboxylic acid (pyridin-3-ylmethyl)-amide

Step 1:Methyl 3-(3,4-difluoro-benzyl)-2,4-dioxo-1,2,3,4-tetrahydro-quinazoline-6-carboxylate

The compound is obtained with 51% yield according to the procedure of Step 1-5 to Step 2-5 of Preparation B using as substrates the compound of Preparation A and 3,4-difluorobenzylamine.

NMR: DMSO ¹H (ppm): 3.86 (3H,s); 5.05 (2H,s); 6.66 (1H,s); 7.18-7.43 (4H,m); 8.18 (1H,dd); 8.47 (1H,s).

MS: m/z (APCI, AP+) 347.1 [M⁻]⁺

Step 2: Methyl 1-methyl-3-(3,4-difluoro-benzyl)-2,4-dioxo-1,2,3,4-tetrahydro-quinazoline-6-carboxylate

1.5 g (yield: 72%) of the desired compound is obtained according to the procedure of Step 2 of the Preparation D, but using the compound obtained in the preceding Step 1.

MS: m/z (APCI, AP+) 361.0 [M⁻]⁺

CHN Analysis: Calcd (%) :C, 60.00; H, 3.92; N, 7.77. Found (%) :C, 60.05; H, 3.85; N, 7.72.

Step 3: 1-Methyl-3-(3,4-difluoro-benzyl)-2,4-dioxo-1,2,3,4-tetrahydroquinazoline-6-carboxylic acid

1.1 g (yield: 82%) of the desired compound is obtained according to the procedure of Step 2-4 of the Preparation B, but using the compound obtained in the preceding Step 2.

MS: m/z (APCI, AP+) 437.0 [M⁻]⁺

CHN Analysis: Calcd (%) :C, 58.96; H, 3.49; N, 8.09. Found (%) :C, 58.67; H, 3.99; N, 7.27.

Step 4: 3-(3,4-Difluoro-benzyl)-1-methyl-2,4-dioxo-1,2,3,4-tetrahydro-quinazoline-6-carboxylic acid (pyridin-3-ylmethyl)-amide

0.48 g (yield: 79%) of the desired compound is obtained according to the procedure of Example 189, but using the compound obtained in the preceding Step 3 and 3-pyridyl-benzylamine.

MS: m/z (APCI, AP+) 437.1 [M⁻]⁺

CHN Analysis: C₂₃H₈₁F₂N₄O₃ 0.2 H₂O Calcd (%) :C, 62.78; H, 4.21; N, 12.73. Found (%) :C, 62.50; H, 4.13; N, 12.82.

### Example 199

### 3-(3,4Difluoro-benzyl)-1-methyl-2,4-dioxo-1,2,3,4-tetrahydro-quinazoline-6-carboxylic acid (pyridin-4-ylmethyl)-amide

0.23 g (yield: 38%) of the desired compound is obtained according to the procedure of Example 189, but using the compound obtained in the Step 3 of the Example 198 and 4-pyridyl-benzylamine.

MS: m/z (APCI, AP+) 437.1 [M⁻]⁺

CHN Analysis: C₂₃H18F₂N₄O₃ Calcd (%) :C, 63.30; H, 4.16; N, 12.84. Found (%) :C, 63.19; H, 4.07; N, 12.81.

### Example 200

### 3-(4-Difluoro-benzy)-1-methyl-2,4-dioxo-1,2,3,4-tetrahydro-qxinazoline-6-carboxylic acid 4-methoxy-benzylamide

0.11 g (yield: 39%) of the desired compound is obtained according to the procedure of Example 189, but using the compound obtained in the Step 3 of the Example 198 and 4-methoxy-benzylamine.

MS: m/z (APCI, AP+) 466.2 [M⁻]⁺

CHN Analysis: C₂₅H₂₁F₂N₃O₄ Calcd (%) :C, 64.51; H, 4.55; N, 9.03. Found (%) :C, 64.41; H, 4.53; N, 8.87.

### Example 201

### 3(3-chloro-4-fluoro-benzyl)-1-methyl-2,4-dioxo-1,2,3,4-tetrahydro-quinazoline-6carboxylic acid (pyridin-4-ylmethy)-amide

Step 1: Methyl 3-(3-chloro-4-fluoro-benzyl)-2,4-dioxo-1,2,3,4-tetrahydro-quinazoline-6-carboxylate

The compound is obtained with 18.1% yield according to the procedure of Step 1-5 to Step 2-5 of Preparation B using as substrates the compound of Preparation A and 3-chloro-4-fluorobenzylamine.

MS: m/z (APCI, AP⁻) 361.0 [M⁻]⁺

Step 2: Methyl 1-methyl-3-(3-chloro-4-fluoro-benzyl)-2,4-dioxo-1,2,3,4-tetrahydro-quinazoline-6-carboxylate

0.5 g (yield: 72%) of the desired compound is obtained according to the procedure of Step 2 of the Preparation D, but using the compound obtained in the preceding Step 1.

MS: m/z (APCI, AP+) 377.0 [M⁻]⁺

CHN Analysis: Calcd (%) :C, 57.38; H, 3.75; N, 7.44. Found (%) :C, 57.34; H, 3.73; N, 7.27.

Step 3: 1-Methyl-3-(3-chloro-4-fluoro-benzyl)-2,4-dioxo-1,2,3,4-tetrahydro-quinazoline-6-carboxylic acid

0.45 g (yield: 92%) of the desired compound is obtained according to the procedure of Step 2-4 of the Preparation B, but using the compound obtained in the preceding Step 2.

MS: m/z (APCI, AP+) 363.0 [M⁻]⁺

CHN Analysis: Calcd (%) :C, 56.29; H, 3.33; N, 7.72. Found (%) :C, 56.24; H, 3.21; N, 7.64.

Step 4: 3-(3-chloro-4-fluoro-benzyl)-1-methyl-2,4-dioxo-1,2,3,4-tetrahydro-quinazoline-6-carboxylic acid (pyridin-4-ylmethyl)-amide

0.17 g (yield: 69%) of the desired compound is obtained according to the procedure of Example 189, but using the compound obtained in the preceding Step 3 and 4-pyridyl-benzylamine.

MS: m/z (APCI, AP+) 453.1 [M⁻]⁺

CHN Analysis: C₂₃H₁₈F₂N₄O₃ 1.1 H₂O Calcd (%) :C, 58.44; H, 4.31; N, 11.85. Found (%) :C, 58.23; H, 4.23; N, 11.75.

### Example 202

### 3-(3-Chloro-4-fluoro-benzyl)-1-methyl-2,4-dioxo-1,2,3,4-tetrahydro-quinazoline-6-carboxylic acid 4-methoxy-benzlamide

0.21 g (yield: 80%) of the desired compound is obtained according to the procedure of Example 189, but using the compound obtained in the Step 3 of the Example 201 and 4-methoxy-benzylamine.

MS: m/z (APCI, AP+) 482.1 [M⁻]⁺

CHN Analysis: C₂₅H₂₁CIFN₃O₄ Calcd (%) :C, 62.31; H, 4.39; N, 8.72. Found (%) :C, 62.12; H, 4.37; N, 8.51.

### Example 203

### 4-[6-(4Methoxy-benzylcarbamoyl)-1-methyl-2,4-dioxo-4-dihydro-2H-quinazolin-3-ylmethyl]-benzoate2-hydroxy-ethyl)-trimethyl-ammonium

A suspension of 0.5 g (1.05 mmol) of compound of the Example 34 in hot methanol is added 0.22 g (1.03 mmol) choline bicarbonate. The mixture is heated to reflux for 1 hour. Cool and concentrate. The resulting solid is recrystallized from ethanol to provide 0.41 g (yield: 68%) of the desired compound.

CHN Analysis: C₃₁H₃₆N₄O₇. 0.5 H₂O Calcd (%) :C, 63.58; H, 6.37; N, 9.57. Found (%) :C, 63.32; H, 6.58; N, 9.57.

### Example 204

### 4-[6-(4-Methoxy-benzylcarbamoyl)-1-methyl-2-dioxo-1,4-dihydro-2H-quinazolin-3-ylmethyl]-benzoic acid hemicalcium salt

A suspension of 0.5 g (1.05 mmol) of compound of the Example 34 in warm tetrahydrofuran is added 1.05 ml 1.00 N NaOH. The mixture is stirred 0.5 hour and CaCl₂ 0.058 g (0.525 mmol) is added in one portion. The mixture is stirred 2 hours and then concentrated. Add ethanol and filter. Dried at 88° C. in vacuum oven for 72 hours gives 0.49 g (yield: 94%) of the desired compound.

CHN Analysis: C₅₂H₄₄CaN₆O₁₂.1.0 H₂O Calcd (%) :C, 62.27; H, 4.62; N, 8.38. Found (%) :C, 61.95; H, 4.70; N, 8.34.

### Example 205

### 4-[6-(4-Methoxy-benzylcarbamoyl)-1-methyl-2,4-dioxo-1,4-dihydro-2H-quinazoin-3-ylmethyl]-benzoic acid hemimagnesium salt

A suspension of 0.5 g (1.05 mmol) of compound of the Example 34 in warm tetrahydrofuran is added 1.05 ml 1.00 N NaOH. The mixture is stirred 0.5 hour and MgCl₂ 0.052 g (0.525 mmol) is added in one portion. The mixture is stirred 2 hours and then concentrated. Add ethanol and filter. Dried at 88° C. in vacuum oven for 72 hours gives 049 g (yield: 96%) of the desired compound.

CHN Analysis: C₅₂H₄₄MgN₆O₁₂.1.0 H₂O Calcd (%) :C, 63.26; H, 4.70; N, 8.51. Found (%) :C, 63.07; H, 4.89; N, 8.50.

### Example 206

### 3-(4-Chloro-benzyl)-1-methyl-2,4-dioxo-1,2,3,4-tetrahydro-quinazoline-6-carboxylic acid (pyridin-4-ylmethyl)-amide

Step 1: 1-Methyl-2,4-dioxo-1,2,3,4-tetrahydro-quinazoline-6-carboxylic acid (pyridazin-4-ylmethyl)-amide

To a suspension of compound of the Step 1 of the Example 33 (1.00 g, 4.54 mmol), EDAC (1.13 g, 5.90 mmol), HOBT (0.675 g, 5.00 mmol) in 20 ml of DMF is added a solution of 4-aminomethyl-pyridine (0.507 ml, 5.00 mmol). The light orange suspension is stirred at room temperature overnight. After 24 h, the reaction mixture is concentrated affording a offwhite solid. The solids are subsequently washed with 10 ml of ethyl acetate, saturated Na₂CO₃, and 10 ml of H₂O to give 1.20 g (yield: 85.7%) of product.

MP: 141-145° C.

MS(APCI+): m/z 309.1 (MH⁻).

Step 2: 3-(4-Chloro-benzyl)-1-methyl-2,4-dioxo-1,2,3,4-tetrahydro-quinazoline-6-carboxylic acid (pyridin-4-ylmethyl)-amide

To a suspension of compound obtained in the preceding Step 1 (0.200 g, 0.645 mmol) in 6 ml of DMF is added Cs₂CO₃ (0.630 g, 1.93 mmol). After stirring at room temperature for 30 min, a solution of 4-chlorobenzyl-bromide (0.132 g, 0.645 mmol) in 2 ml of DMF is added dropwise to the reaction mixture and stirred overnight. White solids (cesium salt) are filtered and the solution was concentrated. The resulting suspension is diluted with 10 ml of ethyl acetate and filtered again. The filtrate is concentrated and tritutration with 10 ml of ethyl acetate gave 0.26 g (yield: 92.9%) of a white solid corresponding to the desired compound.

MP: 228-230° C.

CHN Analysis: C₂₃H₁₉N₄O₃Cl₁ Calcd (%): C, 63.52; H, 4.40; N, 12.88. Found (%): C, 63.40; H, 4.41; N, 12.84.

### Example 207

### 3-(4-Fluoro-benzyl)-1-methyl-2,4dioxo-1,2,3,4-tetrahydro-quinazoline-6-carboxylic acid (pyridin-4-ylmethyl)-amide

0.2 g of the desired compound (yield: 74.1%) is obtained according to the procedure of Example 206, Steps 1 to 2, but using in Step 2 4-fluorobenzyl bromide.

mp 210-212° C.;

CHN Analysis: C₂₃H₁₉N₄O₃F₁ Calcd (%): C, 66.02; H, 4.58; N, 13.39 Found(%):C, 65.74; H, 4.60; N, 13.03.

### Example 208

### 3-(4-Fluoro-benzyl)-1-methyl-2,4-dioxo-1,2,3,4-tetrahydro-quinazoline-6-carboxylic acid (pyridin-3-ylmethyl)-amide

Step 1: 1-Methyl-2,4-dioxo-1,2,3,4-tetrahydro-quinazoline-6-carboxylic acid (pyridin-3-ylmethyl)-amide

1.18 g of the desired compound (yield: 83.7%) is obtained according to the procedure of Step 1 of the Example 206, but using 3-aminomethyl pyridine.

MS(APCI+): m/z 309.1 (MH⁻);

¹H NMR (400 MHz, DMSO-d₆) δ3.43 (s, 3H, NCH₃), 4.47 (d, J=5.86 Hz, 2H, NCH₂Ar), 7.31-7.34 (m, 1H, ArH), 7.48 (d, J=8.79 Hz, 1H, ArH), 7.70 (d, J=7.82 Hz, 1H, ArH), 8.20 (dd, J=8.79, 1.95 Hz, 1H, ArH), 8.42-8.43 (m, 1H, ArH), 8.53 (d, J=2.20 Hz, 2H, ArH), 9.30 (t, J=5.62, 1H, ArH), 11.65 (s, 1H, NH);

Step 2: 3-(4-Fluoro-benzyl)-1-methyl-2,4-dioxo-1,2,3,4-tetrahydro-quinazoline -6-carboxylic acid (pyridin-3-ylmethyl)-amide

0.25 g of the desired compound (yield: 82.6%) is obtained according to the procedure of Example 206, Step 2, but using the compound obtained in the preceding Step 1 and 4-fluorobenzyl bromide.

MP: 166-168° C.

Anal. Calcd for C₂₃H₁₉N₄O₃F₁: C, 65.79; H, 4.60; N, 13.34. Found: C, 65.40; H, 4.40; N, 13.18.

### Example 209

### 3-(4-Chloro-benzyl)-1-methyl-2,4-dioxo-1,2,3,4-tetrahydroquinazoline -6-carboxylic acid (pyridin-3-ylmethyl)-amide

0.25 g of the desired compound (yield: 89.3%) is obtained according to the procedure of Example 206, Step 2, but using the compound obtained in the Step 1 of Example 208 and 4-chlorobenzyl bromide.

MP: 173-175° C.

Anal. (%) Calcd for C₂₃H₁₉N₄O₃Cl₁: C, 62.77; H, 4.48; N, 12.73. Found: C, 62.39; H, 4.46; N, 12.71.

### Example 210

### 3-(4-Fluoro-benzyl)-1-methyl-2,4-dioxo-1,2,3,4-tetrahydroquinazoline -6-carboxylic acid 3-methoxy-enzylamide

Step 1: 1-Methyl-2,4-dioxo-1,2,3,4-tetrahydro-quinazoline-6-carboxylic acid 3-methoxy-benzylamide

1.29 g of the desired compound (yield: 83.8%) is obtained according to the procedure of Example 206, Step 1, but using 3-methoxylbenzyl amine.

MP: 235-238° C.

Step 2: 3-(4-Fluoro-benzyl)-1-methyl-2,4-dioxo-1,2,3,4-tetrahydro-quinazoline -6-carboxylic acid 3-methoxy-benzylamide

0.25 g of the desired compound (yield: 95%) is obtained according to the procedure of Example 206, Steps 2, but using the compound obtained in the preceding Step 1 and 4-fluorobenzyl bromide.

MP: 176-178° C.

Anal. (%) Calcd for C₂₅H₂₂N₃O₄F₁: C, 67.11; H, 4.96; N, 9.39. Found: C, 66.99; H, 4.99; N, 9.18.

### Example 211

### 3-(4-Chloro-benzyl)-1-methyl-2,4-dioxo-1,2,3,4tetrahydroquinazoline -6carboxylic acid 3-methoxy-benzylamide

0.25 g of the desired compound (yield: 92%) is obtained according to the procedure of Example 206, Step 2, but using the compound obtained in the Step 1 of Example 210 and 4-chlorobenzyl bromide.

MP: 178-180° C.

Anal. (%) Calcd for C₂₅H₂₂N₃O₄Cl₁: C, 64.60; H, 4.79; N, 9.04. Found: C, 64.22; N, 8.84.

### Example 212

### 3-(4-Fluoro-benzyl)-1-methyl-2,4-dioxo-1,2,3,4-tetrahydroquinazoline -6-carboxylic acid (2-methoxy-pyridin-4-ylmethyl)-amide

Step 1: 1-Methyl-2,4-dioxo-1,2,3,4-tetrahydro-quinazoline-6-carboxylic acid (2-methoxy-pyridin-4-ylmethyl)-amide

1.00 g of the desired compound (yield: 76.9%) is obtained according to the procedure of Example 206, Step 1, but using (2-methoxy-pyridin-4-yl)-methylamine.

MP: 215-218° C.

MS(APCI+): m/z 339.1 (MH⁻).

Step 2: 3-(4-Fluoro-benzyl)-1-methyl-2,4-dioxo-1,2,3,4-tetrahydroquinazoline -6-carboxylic acid (2-methoxy-pyridin-4-ylmethyl)-amide

0.07 g of the desired compound (yield: 26.5%) is obtained according to the procedure of Example 206, Step 2, but using the compound obtained in the preceding Step 1 and 4-fluorobenzyl bromide.

MP: 174-175° C.

Anal. (%) Calcd for C₂₄H₂₁N₄O₄F₁: C, 64.20; H, 4.73; N, 12.48. Found: C, 63.88; H, 4.73; N, 12.08.

### Example 213

### 3-(4-Chloro-benzyl)-1-methyl-2,4-dioxol-1,2,3,4-tetrahydroquinazoline -6-carboxylic acid (2-methoxy-pyridn-4-ylmethyl)-amide

0.09 g of the desired compound (yield: 33%) is obtained according to the procedure of Example 206, Step 2, but using the compound obtained in Step 1 of Example 212 and 4-chlorobenzyl bromide.

MP: 169-170° C.

Anal. (%) Calcd for C₂₄H₂₁N₄O₄Cl₁: C, 62.02; H, 4.61; N, 11.98. Found: C, 62.01; H, 5.01; N, 11.70.

### Example 214

### tert-Butyl 1-{4-[6-(4-methoxy-benzylcarbamoyl)-1-methyl-2,4-dioxo-1,4-dihydro-2H-quinazolin-3-ylmethyl]-phenyl}-cyclopropanecarboxylate

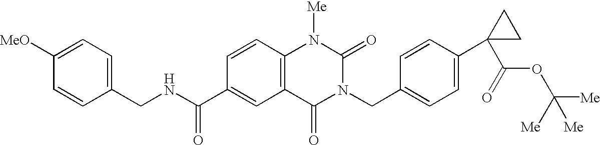

0.35 g of the desired compound (yield: 67%) is obtained according to the procedure of Example 206, Steps 1 to 2, but using in Step 1 4-methoxy-benzylamine and in Step 2 tert-butyl 1-(4-bromomethyl-phenyl)-cyclopropanecarboxylate.

MP: 148-149° C.

Anal. (%) Calcd for C₃₃H₃₅N₃O₆: C, 68.88; H, 6.24; N, 7.30. Found: C, 68.49; H, 6.29; N, 7.21.

### Example 215

### 1-{4-[6-(4-Methoxy-benzylcarbamoyl)-1-methyl-2,4-dioxo-1,4-dihydro -2H-quinazolin-3ylmethyl]-phenyl}-cyclopropanecarboxylic acid

To a solution of the compound of Example 214 (0.35 g, 0.61 mmol) in 2 ml of CH₂Cl₂ are added 2 ml of TFA. The yellow solution is stirred at room temperature for 4 hours. The reaction mixture is concentrated and trituration with diethyl ether gives 0.25 g (yield:79%) of a white solid corresponding to the desired compound.

MP: 179-181° C.

Anal. (%) Calcd for C₂₉H₂₇N₃O₆: C, 66.22; H, 5.35; N, 7.77. Found: C, 66.61; H, 5.40; N, 8.04.

### Example 216

### 3Benzyl-6-benzylsulfanyl-1-methyl-1H-quinazoline-2,4-dione

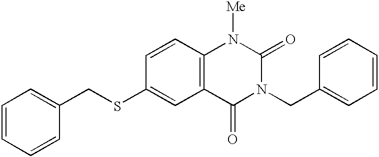

Step 1: 5-Iodo-2-methylamino-benzoic acid

To a solution of N-methylanthranilic acid (5.00 g, 3.31 mmol) in 30 ml of acetic acid are added 60 ml of H₂O and I₂ (8.39 g, 3.31 mmol) is added portionwise over a period of 5 minutes. The reaction mixture is stirred at room temperature for 2 days. After 48 hours, the product is filtered and washed with 30 ml of H₂O. The mother liquor is concentrated affording more product

Weight: 7.3 g; Yield=80%

MP: 170-172° C.

MS(APCI+): m/z 276.0 (MH⁻).

Step 2: 3-Benzyl-6-iodo-1-methyl-1H-quinazoline-2,4-dione

To a mixture of the compound obtained in the preceding Step 1 (0.50 g, 1.9 mmol), isothiocyanate (0.236 g, 1.58 mmol), and CF₃CO₂Ag (0.838 g, 3.80 mmol) is added slowly Et₃N. The reaction mixture is heated at refluxed for 1.5 hours. After cooled to room temperature, silver sulfide is filtered and the filtrate is concentrated affording a brown oil. The product is purified by chromatography on silica gel (ethyl acetate/hexane: 20/80) to give 0.300 g (48.0%) of a white solid

MP: 149-150° C.

MS(APCI+): m/z 391.0 (MH⁻).

Step 3: 3-Benzyl-6-benzylsulfanyl-1-methyl-1H-quinazoline-2,4-dione

To a mixture of KHCO₃ (0.009 g, 0.089 mmol), PPh₃ (0.007 g, 0.027 mmol), n-Bu₄NI (0.033 g, 0.089 mmol), Pd(OAc)₂ (0.002 g, 0.009 mmol), after purging with N₂ for 5 min, are added a solution of the compound of the preceding Step 2 (0.035 g, 0.089 mmol) and butyl-thiocarbamic acid S-benzyl ester (0.020 g, 0.089 mmol) in 5 ml of dioxane at room temperature. The brown solution is heated at 100° C. for overnight. After 24 hours, the reaction mixture is cooled to room temperature and diluted with 20 ml of ethyl acetate, filtered through a sheet of celite, washed with H₂O (2×5 ml), concentrated affording a yellow oil. Tritutration with diethyl gives 0.025 g (yield: 72%) of a yellow solid corresponding to the desired compound.

MP: 117-118° C.

Anal. (%) Calcd for C₂₃H₂₀N₂O₂S₁: C, 69.66; H, 5.31; N, 7.06. Found: C, 69.26; H, 5.04; N, 6.93.

### Example 217

### 3-Benzyl-1-methyl-6-phenylmethanesulfinyl-1H-quinazoline-2,4-dione

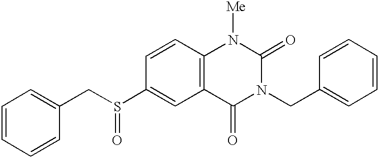

To a solution of the compound of Example 216 (0.050 g, 0.129 mmol) in 9 ml of anhydrous CH₂Cl₂ is added m-chloro-perbenzoic acid (0.029 g, 0.127 mmol) at −5° C. After stirring at −5° C. for 3 hours, the reaction mixture is quenched with 20 ml of NaHCO₃ while in the ice-bath. The organic layer is separated and the aqueous is extracted with CH₂Cl₂ (2×20 ml). The combined organic layers concentrated affording a yellow oil. The product is purified by chromatography on silica gel (ethyl acetate/hexane: 30/70) to give 0.070 g (yield: 33.7%) of a white solid corresponding to the desired compound.

MP: 182-183° C.

Anal. (%) Calcd for C₂₃H₂₀N₂O₃S₁: C, 67.84; H, 5.03; N, 6.88. Found: C, 68.13; H, 4.86; N. 6.48.

### Example 218

### 3-Benzyl-1-methyl-6-phenylmethanesulfonyl-1H-quinazoline-2,4-dione

To a solution of the compound of Example 216 (0.133 g, 0.342 mmol) in 25 ml of anhydrous CH₂Cl₂is added m-chloro-perbenzoic acid (0.153 g, 0.685 mmol) at −5° C. After stirring at −5° C. for 5 min, the ice-bath is removed and the reaction mixture is stirred at room temperature for 3 hours. The reaction is completed and quenched with 5 ml of saturated NaHCO₃. The organic layer is separated and the aqueous is extracted with CH₂Cl₂ (2×20 ml). The combined organic layers concentrated affording a yellow oil. Tritutration with ethyl acetate gives 0.80 g (yield: 56%) of a light yellow solid corresponding to the desired compound.

MP: 173-175° C.

Anal. (%) Calcd for C₂₃H₂₀N₂O₄S₁: C, 64.73; H, 4.89; N, 6.56. Found: C, 64.34; H, 4.72; N, 6.18.

### Example 219

### 4-[6-(4-methoxy-benzylcarbamoyl)-1-methyl-2,4-dioxo-1,4dihydro-2H-quinazoline-3-ylmethyl]-benzoic acid tert-butoxycarbonylmethyl ester

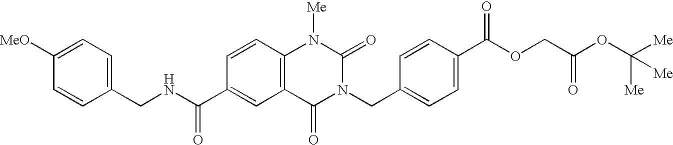

To 0.40 g (0.84 mmol) of the compound of Example 35 in dimethylformamide (10 ml) is added di-isopropylethylamine 0.13 g (10 mmol) followed by tert-butylacetyl chloride 0.18 g (1.18 mmol). The mixture is stirred overnight at room temperature before concentrating in-vacuo, then diluted with ethyl acetate (20 ml). The organic layer is washed with saturated aqueous NaCl solution (2×20 ml), dried MgSO₄; and purified by flash chromatography (EtOAC/hexane eluent) to give 0.11 g (yield: 23%) of the desired compound.

MS: m/z (APCI, AP+) 588.4 [M⁻]⁺

CHN Analysis (%): C₃₂H₃₃N₃O₈.1.8 H₂O Calcd: C, 61.97; H, 5.61; N, 6.70. Found: C, 61.58; H, 5.61; N, 6.70.

### Example 220

### 4-[6(4-methoxy-benzylcarbamoyl)-1-methyl-2,4-dioxo-1,4dihydro-2H-quinazoline-3-ylmethyl]-benzoic acid dimethylamino-dimethyl-propyl ester

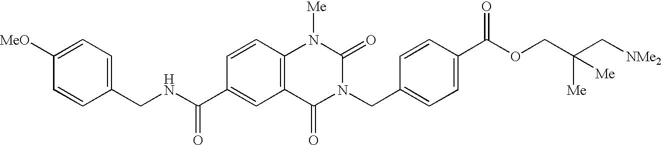

To 0.50 g (1.6 mmol) of compound of Example 35 in dimethylformamide (20 ml) is added EDAC HCl 0.39 g (2.1 mmol), HOBT 0.28 g (2.1 mmol), followed by dimethylamino-dimethyl-propan-1-ol 0.27 g (2.1 mmol). The mixture is stirred overnight at room temperature before adding water (20 ml) and extracting with ethyl acetate (2×20 ml). The combined organic layers are washed with saturated aqueous NaCl solution (4×20 ml), and dried MgSO4. The crude product is dissolved in EtOAc/MeOH and saturated ethereal HCl. is added. After concentration and solidification in EtOAc, 0.49 g (yield: 43%) of the desired compound is obtained.

MS: m/z (APCI, AP+) 587.0 [M⁻]⁺

CHN Analysis (%): C₃₃H₃₈N₄O₆ 1.0 HCl.1.2 H₂O Calcd: C, 61.40; H, 6.48; N, 8.68. Found: C, 61.01; H, 6.31; N, 8.99.

### Example 221

### 4-[6-(4-methoxy-benzylcarbamoyl)-1-methyl-2,4-dioxo-1,4-dihydro-2H-quinazoline-3-ylmethyl]-benzoic acid dimethylamino-methyl-propyl ester

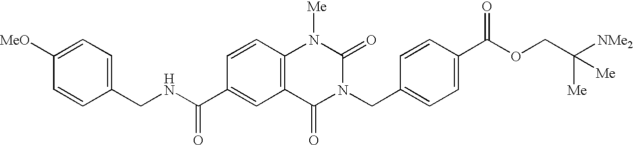

To 0.50 g (1.6 mmol) of the compound of Example 35 in dimethylformamide (20 ml) is added EDAC HCl 0.39 g (2.1 mmol), HOBT 0.28 g (2.1 mmol), followed by dimethylamino-methyl-propan-1-ol 0.24 g (2.1 mmol). The mixture is stirred overnight at room temperature before adding water (20 ml) and extracting with ethyl acetate (2×20 ml). The combined organic layers are washed with saturated aqueous NaCl solution (4×20 ml), and dried MgSO₄. The crude product is dissolved in EtOAc/MeOH and saturated ethereal HCl. is added. After concentration and solidification in EtOAc, 0.21 g (yield: 21%) of the desired compound is obtained.

MS: m/z (APCI, AP+) 573.2 [M⁻]⁺

CHN Analysis (%): C₃₂H₃₆N₄O₆ 1.0 HCl.0.48 H₂O Calcd: C, 62.22; H, 6.19; N, 9.07. Found: C, 61.82; H, 6.00; N, 9.16.

### Example 222

### 4-[6-(4-methoxy-benzylcarbamoyl)-1-2,4dioxo-1,4dihydro-2H-quinazoline-3-ylmethyl]-benzoic acid 2-dimethylamino-ethyl ester

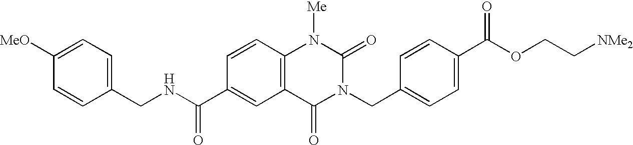

To 0.73 g (1.5 mmol) of the compound of Example 35 in dimethylformamide (10 ml) is added EDAC HCl 0.38 g (2.0 mmol), HOBT 0.27 g (2.0 mmol), followed by dimethylamino-propan-1-ol 0.18 g (2.0 mmol). The mixture is stirred overnight at room temperature before adding water (20 ml) and extracting with ethyl acetate (2×20 ml). The combined organic layers are washed with saturated aqueous NaCl solution (2×20 ml), and dried MgSO₄. the crude product is solidified in EtOAc to give 0.49 g (yield: 60%) of the desired compound.

MS: m/z (APCI, AP+) 545.3 [M⁻]⁺

CHN Analysis (%): C₃₀H₃₂N₄O₆ 0.25 H₂O Calcd: C, 65.62; H, 5.97; N, 10.20. Found: C, 65.62; H, 5.92; N, 10.23.

### Example 223

### 4-[6-(4-methoxy-benzylcarbamoyl)-1-2,4-dioxo-1,4-dihydro-2H-quinazoline-3-ylmethyl]-benzoic acid chloromethyl ester

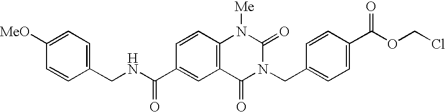

To 1.0 g (2.1 mmol) of the compound of Example 35 in dimethylformamide (15 ml) is di-isopropylethylamine 0.47 g (3.6 mmol) followed by chloro-iodomethane 1.86 g (10.5 mmol). The mixture is stirred overnight at room temperature before diluting with ethyl acetate (20 ml). The organic layer is washed with water (1×10 ml) saturated aqueous NaCl solution (2×10 ml), and dried MgSO₄. After solidification in ether 0.29 g (yield: 26%) of the desired compound is obtained.

MS: m/z (APCI, AP+) 522.2 [M⁻]⁺

CHN Analysis (%): C₂₇H₂₄ClN₃O₆ Calcd: C, 62.13; H, 4.63; N, 8.05. Found: C, 62.08; H, 4.61; N, 7.95.

### Example 224

### 4-[6-(4-methoxy-benzylcarbamoyl)-1-methyl-2,4-dioxo-1,4dihydro-2H-quinazoline-3-ylmethyl]-benzoic acid 2-tert-butoxycarbonylamino-3-methyl-1-butanoyloxymethyl ester ester

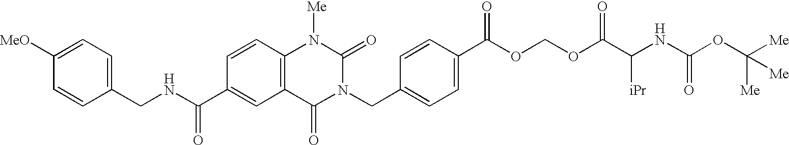

To 0.39 g (0.75 mmol) of the compound of Example 223 in dimethylformamide (10 ml) is added di-isopropylethylamine 0.12 g (0.96 mmol) followed by t-butoxycarbonyl-leucine 0.21 g (0.96 mmol). The mixture is stirred overnight at 60-70° C. for 12 hours, cooled and diluted with ethyl acetate (20 ml). The organic layer is washed with water (1×10 ml), 5% aqueous NaHCO₃ solution (1×10 ml), saturated aqueous NaCl (1×10 ml), dried MgSO₄, and purified by flash chromatography (EtOAC/hexane eluent) to give 0.14 g (yield: 25%) of the desired compound.

MS: m/z (APCI, AP+) 701.3 [M⁻Boc]⁻

CHN Analysis (%): C₃₇H₄₂N₄O₁₀ Calcd: C, 61.97; H, 5.61; N, 6.70. Found: C, 61.58; H, 5.61; N, 6.70.

Example 225

### 4-[6-(4-methoxy-benzylcarbamoyl)-1-methyl-2,4-dioxo-1,4-dihydro-2H-quinazoline-3-ylmethyl]-benzoic acid 2-amino-3-methyl-butanoyloxymethyl ester hydrochloride

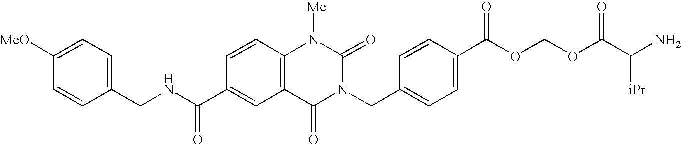

To 0.14 g (0.19 mmol) of the compound of Example 224 in dioxane (10 ml) is added 1.0 M HCl in ether (10 ml). HCl gas is bubbled through for 2 minutes then mixture is stirred 90 minutes at room temperature. After concentration and trituration in EtOAc, 0.039 g (yield: 30%) of the desired compound is obtained.

MS: m/z (APCI, AP+) 603.2 [M⁻]⁺

CHN Analysis (%): C₃₇H₄₂N₄O₁₀ Calcd: C, 61.97; H, 5.61; N, 6.70. Found: C, 61.58; 5.61; N, 6.70.

### Example 226

### 4-[6-(4-methoxy-benzylcarbamoyl)-1-methyl-2-dioxo-1,4-dihydro-2H-quinazoline-3-ylmethyl]-benzoic acid 2-(2-tert-butoxycarbonylamino-3-methyl-butanoylamino)-3-methyl-butanoyloxymethyl ester

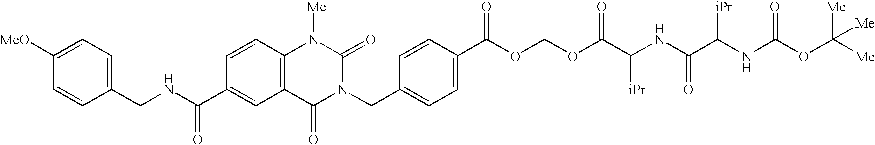

Step 1: 2-(2-tert-Butoxycarbonylamino-3-methyl-butanoylamino)-3-methyl-butyric acid methyl ester

To 1.3 g (5.9 mmol) of t-butoxycarbonyl-leucine in dimethylformamide (15 ml) is added EDAC HCl 1.4 g (7.1 mmol), HOBT 0.95 g (7.1 mmol), followed by NH₂-Leu-OMe 1.0 g (5.9 mmol). The mixture is stirred overnight at room temperature before adding water (20 ml) and extracting with ethyl acetate (2×20 ml). The combined organic layers are washed with 10% aqueous Na₂CO₃ (1×10 ml), saturated aqueous NaCl solution (2×20 ml), and dried MgSO₄. A solidification in ether gives 1.05 g (yield: 53%) of the desired compound.

MS: m/z (APCI, AP+) 331.2 [M⁻]⁺

CHN Analysis (%): C₁₆H₃₀N₂O₅ Calcd: C, 58.16; H, 9.15; N, 8.48. Found: C, 58.32; H, 9.24; N, 8.51.

Step 2: 2-(2-tert-Butoxycarbonylamino-3-methyl-butanoylamino)-3-methyl-butyric acid

To 0.4 g (1.2 mmol) of the compound obtained in the preceding step 1, in 3:1:1 methanol/water/THF (10 ml) is added LiOH H₂O, 0.06 g (1.44 mmol). The mixture is stirred overnight at room temperature. Partitioned between water (20 ml) and ethyl acetate (30 ml). The layers are separated and the aqueous layer made acidic with 2 M HCl. The product is extracted with EtOAc ( 2×20 ml) washed with saturated aqueous NaCl solution (1×20 ml), and dried MgSO₄. A solidification in ether gives 0.22 g (yield: 58%) of the desired compound.

MS: m/z (APCI, AP+) 317.2 [M⁻]⁺

CHN Analysis (%): C₁₅H₂₈N₂O₅ Calcd: C, 56.94; H, 8.92; N, 8.85. Found: C, 56.72; H, 8.89; N, 8.64

Step 3: 4-[6-(4-methoxy-benzylcarbamoyl)-1-methyl-2,4-dioxo-1,4-dihydro-2H-quinazoline-3-ylmethyl]-benzoic acid 2-(2-tert-butoxycarbonylamino-3-methyl-butanoylamino)-3-methyl-butanoyloxymethyl ester

To 0.29 g (0.56 mmol) of the compound obtained in Example 223 in dimethylformamide (10 ml) is added di-isopropylethylamine 0.092 g (0.72 mmol) followed by compound obtained in the preceding Step 2, 0.23 g (0.72 mmol) then Nal (cat.). The mixture is stirred overnight at 50° C. for 18 hours. Cool and dilute with water and extract with ethyl acetate (2×20 ml). The combined organic layer are washed with saturated aqueous NaHCO₃ solution (1×10 ml), saturated aqueous NaCl (3×10 ml) and dried MgSO₄. a solidification in a mixture of EtOAc/hexane gives 0.27 g (yield: 63%) of the desired compound.

MS: m/z (APCI, AP+) 800.4 [M⁻-Boc]⁻

CHN Analysis (%): C₃₇H₄₂N₄O₁₀ Calcd: C, 62.91; H, 6.41; N, 8.73. Found: C, 62.59; H, 6.44; N, 8.39.

### Example 227

### 4-[6-(4-methoxy-benzylcarbamoyl)-1-methyl-2,4dioxo-1,4dihydro-2H-quinazoline-3-ylmethyl]-benzoic acid 2(2-amino-3methyl-butanoylamino)-3-methyl-butanoyloxymethyl ester

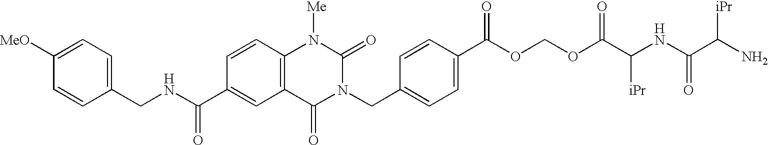

To 0.25 g (0.31 mmol) of compound of the Example 226 in dioxane (10 ml) is added 1.0 M HCl in ether (10 ml). HCl gas is bubbled through for 2 minutes then mixture is stirred 90 minutes at room temperature. After concentration and trituration in EtOAc, 0.12 g (yield: 55%) of the desired compound is obtained.

MS: m/z (APCI, AP+) 702.0 [M⁻]⁺

CHN Analysis (%): C₃₇H₄₃N₅O₉ Calcd: C, 63.33; H, 6.18; N, 9.98. Found: C, 62.99; H, 6.06; N, 9.72.

### Examples 228 to 345

These compounds were obtained according to the procedure described in the Example 168 followed by the procedure of the Example 169.

3-[2-(4-Bromo-phenoxy)-ethyl]-1-methyl-2,4-dioxo-1,2,3,4-tetrahydro-pyrido[3,4-d]pyrimidine-6-carboxylic acid (2-methoxy-pyridin-4-ylmethyl)-amide,

3-[2-(4-Fluoro-phenoxy)-ethyl]-1-methyl-2,4-dioxo-1,2,3,4-tetrahydro-pyrido[3,4-d]pyrimidine-6-carboxylic acid (2-methoxy-pyridin-4-ylmethyl)-amide,

3-[2-(4-Chloro-phenoxy)-ethyl]-1-methyl-2,4-dioxo-1,2,3,4-tetrahydro-pyrido[3,4-d]pyrimidine-6-carboxylic acid (2-methoxy-pyridin-4-ylmethyl)-amide,

3-(4-Chloro-benzyl)-1-methyl-2,4-dioxo-1,2,3 ,4tetrahydro-pyrido[3,4-d]pyrimidine-6-carboxylic acid (2-methoxy-pyridin-4-ylmethyl)-amide,

3-(4-Bromo-benzyl)-1-methyl-2,4-dioxo-1,2,3,4-tetrahydro-pyrido[3,4-d]pyrimidine-6-carboxylic acid (2-methoxy-pyridin-4-ylmethyl)-amide,

3-(4-Fluoro-benzyl)-1-methyl-2,4-dioxo-1,2,3,4-tetrahydro-pyrido[3,4-d]pyrimidine-6-carboxylic acid (2-methoxy-pyridin-4-ylmethyl)-amide,

3-(3-Chloro-benzyl)-1-methyl-2,4-dioxo-1,2,3,4-tetrahydro-pyrido[3,4-d]pyrimidine-6-carboxylic acid (2-methoxy-pyridin-4-ylmethyl)-amide,

3-(3-Bromo-benzyl)-1-methyl-2,4-dioxo-1,2,3,4-tetrahydro-pyrido[3,4-d]pyrimidine-6-carboxylic acid (2-methoxy-pyridin-4-ylmethyl)-amide,

3-(3-Fluoro-benzyl)-1-methyl-2,4-dioxo-1,2,3,4-tetrahydro-pyrido[3,4-d]pyrimidine-6-carboxylic acid (2-methoxy-pyridin-4-ylmethyl)-amide,

3-(3,4-Difluoro-benzyl)-1-methyl-2,4-dioxo-1,2,3,4-tetrahydro-pyrido[3,4-d]pyrimidine-6-carboxylic acid (2-methoxy-pyridin-4-ylmethyl)-amide,

3-(3-Chloro-4-fluoro-benzyl)-1-methyl-2,4-dioxo-1,2,3,4-tetrahydro-pyrido[3,4-d]pyrimidine-6-carboxylic acid (2-methoxy-pyridin-4-ylmethyl)-amide,

3-(3-Methoxy-benzyl)-1-methyl-2,4-dioxo-1,2,3,4-tetrahydro-pyrido[3,4-d]pyrimidine-6-carboxylic acid (2-methoxy-pyridin-4-ylmethyl)-amide,

3-(4-Methoxy-benzyl)-1-methyl-2,4-dioxo-1,2,3,4-tetrahydro-pyrido[3,4-d]pyrimidine-6-carboxylic acid (2-methoxy-pyridin-4-ylmethyl)-amide,

3-[2-(4-Bromo-phenoxy)-ethyl]-1-methyl-2,4-dioxo-1,2,3,4-tetrahydro-pyrido[3,4-d]pyrimidine-6-carboxylic acid (2-ethoxy-pyridin-4-ylmethyl)-amide,

3-[2-(4-Fluoro-phenoxy)-ethyl]-1-methyl-2,4-dioxo-1,2,3,4-tetrahydro-pyrido[3,4-d]pyrimidine-6-carboxylic acid (2-ethoxy-pyridin-4-ylmethyl)-amide,

3-[2-(4-Chloro-phenoxy)-ethyl]-1-methyl-2,4-dioxo-1,2,3,4-tetrahydro-pyrido[3,4-d]pyrimidine-6-carboxylic acid (2-ethoxy-pyridin-4-ylmethyl)-amide,

3-(4-Bromo-benzyl)-1-methyl-2,4-dioxo-1,2,3,4-tetrahydro-pyrido[3,4-d]pyrimidine-6-carboxylic acid (2-ethoxy-pyridin-4-ylmethyl)-amide,

3-(4-Fluoro-benzyl)-1-methyl-2,4-dioxo-1,2,3,4-tetrahydro-pyrido[3,4-d]pyrimidine-6-carboxylic acid (2-ethoxy-pyridin-4-ylmethyl)-amide,

3-(3-Chloro-benzyl)-1-methyl-2,4-dioxo-1,2,3,4-tetrahydro-pyrido[3,4-d]pyrimidine-6-carboxylic acid (2-ethoxy-pyridin-4-ylmethyl)-amide,

3-(3-Bromo-benzyl)-1-methyl-2,4-dioxo-1,2,3,4-tetrahydro-pyrido[3,4-d]pyrimidine-6-carboxylic acid (2-ethoxy-pyridin-4-ylmethyl)-amide,

3-(3-Fluoro-benzyl)-1-methyl-2,4-dioxo-1,2,3,4-tetrahydro-pyrido[3,4-d]pyrimidine-6-carboxylic acid (2-ethoxy-pyridin-4-ylmethyl)-amide,

3-(3,4-Difluoro-benzyl)-1-methyl-2,4-dioxo-1,2,3,4-tetrahydro-pyrido[3,4-d]pyrimidine 6 carboxylic acid (2-ethoxy-pyridin-4-ylmethyl)-amide,

3-(3-Chloro-4-fluoro-benzyl)-1-methyl-2,4-dioxo-1,2,3,4-tetrahydro-pyrido[3,4-d]pyrimidine-6-carboxylic acid (2-ethoxy-pyridin-4-ylmethyl)-amide,

3-(3-Methoxy-benzyl)-1-methyl-2,4-dioxo-1,2,3,4-tetrahydro-pyrido[3,4-d]pyrimidine-6-carboxylic acid (2-ethoxy-pyridin-4-ylmethyl)-amide,

3-(4-Methoxy-benzyl)-1-methyl-2,4-dioxo-1,2,3,4-tetrahydro-pyrido[3,4-d]pyrimidine-6-carboxylic acid (2-ethoxy-pyridin-4-ylmethyl)-amide,

3-[2-(4-Bromo-phenoxy)-ethyl]-1-methyl-2,4-dioxo-1,2,3,4-tetrahydro-pyrido[3,4-d]pyrimidine-6-carboxylic acid (pyridin-4-ylmethyl)-amide,

3-[2-(4-Fluoro-phenoxy)-ethyl]-1-methyl-2,4-dioxo-1,2,3,4-tetrahydro-pyrido[3,4-d]pyrimidine 6 carboxylic acid (pyridin-4-ylmethyl)-amide,

3-[2-(4-Chloro-phenoxy)-ethyl]-1-methyl-2,4-dioxo-1,2,3,4-tetrahydro-pyrido[3,4-d]pyrimidine-6-carboxylic acid (pyridin-4-ylmethyl)-amide,

3-(4-Chloro-benzyl)-1-methyl-2,4-dioxo-1,2,3,4-tetrahydro-pyrido[3,4-d]pyrimidine-6-carboxylic acid (pyridin-4-ylmethyl)-amide,

3-(4-Bromo-benzyl)-1-methyl-2,4-dioxo-1,2,3,4-tetrahydro-pyrido[3,4-d]pyrimidine-6-carboxylic acid (pyridin-4-ylmethyl)-amide,

3-(4-Fluoro-benzyl)-1-methyl-2,4-dioxo-1,2,3,4-tetrahydro-pyrido[3,4-d]pyrimidine-6-carboxylic acid (pyridin-4-ylmethyl)-amide,

3-(3-Chlorobenzyl) 1 methyl 2,4 dioxo 1,2,3,4 tetrahydropyrido[3,4-d]pyrimidine-6-carboxylic acid (pyridin-4-ylmethyl)-amide,

3-(3-Bromo-benzyl)-1-methyl-2,4-dioxo-1,2,3,4-tetrahydro-pyrido[3,4-d]pyrimidine-6-carboxylic acid (pyridin-4-ylmethyl)-amide,

3-(3-Fluoro-benzyl)-1-methyl-2,4-dioxo-1,2,3,4-tetrahydro-pyrido[3,4-d]pyrimidine-6-carboxylic acid (pyridin-4-ylmethyl)-amide,

3-(3,4-Difluoro-benzyl)-1-methyl-2,4-dioxo-1,2,3,4-tetrahydro-pyrido[3,4-d]pyrimidine-6-carboxylic acid (pyridin-4-ylmethyl)-amide,

3-(3-Chloro-4-fluoro-benzyl)-1-methyl-2,4-dioxo-1,2,3,4-tetrahydro-pyrido[3,4-d]pyrimidine-6-carboxylic acid (pyridin-4-ylmethyl)-amide,

3-(3-Methoxy-benzyl)-1-methyl-2,4-dioxo-1,2,3,4-tetrahydro-pyrido[3,4-d]pyrimidine-6-carboxylic acid (pyridin-4-ylmethyl)-amide,

3-(4-Methoxy-benzyl)-1-methyl-2,4-dioxo-1,2,3,4-tetrahydro-pyrido[3,4-d]pyrimidine-6-carboxylic acid (pyridin-4-ylmethyl)-amide,

3-[2-(4-Bromo-phenoxy)-ethyl]-1-methyl-2,4-dioxo-1,2,3,4-tetrahydro-pyrido[3,4-d]pyrimidine-6-carboxylic acid (2-amino-pyridin-4-ylmethyl)-amide,

3-[2-(4-Fluoro-phenoxy)-ethyl]-1-methyl-2,4-dioxo-1,2,3,4-tetrahydro-pyrido[3,4-d]pyrimidine-6-carboxylic acid (2-amino-pyridin-4-ylmethyl)-amide,

3-[2-(4-Chloro-phenoxy)-ethyl]-1-methyl-2,4-dioxo-1,2,3,4-tetrahydro-pyrido[3,4-d]pyrimidine-6-carboxylic acid (2-amino-pyridin-4-ylmethyl)-amide,

3-(4-Chloro-benzyl)-1-methyl-2,4-dioxo-1,2,3,4-tetrahydro-pyrido[3,4-d]pyrimidine-6-carboxylic acid (2-amino-pyridin-4-ylmethyl)-amide,

3-(4-Bromo-benzyl)-1-methyl-2,4-dioxo-1,2,3,4-tetrahydro-pyrido[3,4-d]pyrimidine-6-carboxylic acid (2-amino-pyridin-4-ylmethyl)-amide,

3-(4-Fluoro-benzyl)-1-methyl-2,4-dioxo-1,2,3,4-tetrahydro-pyrido[3,4-d]pyrimidine-6-carboxylic acid (2-amino-pyridin-4-ylmethyl)-amide,

3-(3-Chloro-benzyl)-1-methyl-2,4-dioxo-1,2,3,4-tetrahydro-pyrido[3,4-d]pyrimidine-6-carboxylic acid (2-amino-pyridin-4-ylmethyl)-amide,

3-(3-Bromo-benzyl)-1-methyl-2,4-dioxo-1,2,3,4-tetrahydro-pyrido[3,4-d]pyrimidine-6-carboxylic acid (2-amino-pyridin-4-ylmethyl)-amide,

3-(3-Fluoro-benzyl)-1-methyl-2,4-dioxo-1,2,3,4-tetrahydro-pyrido[3,4-d]pyrimidine-6-carboxylic acid (2-amino-pyridin-4-ylmethyl)-amide,

3-(3,4-Difluoro-benzyl)-1-methyl-2,4-dioxo-1,2,3,4-tetrahydro-pyrido[3,4-d]pyrimidine-6-carboxylic acid (2-amino-pyridin-4-ylmethyl)-amide,

3-(3-Chloro-4-fluoro-benzyl)-1-methyl-2,4-dioxo-1,2,3,4-tetrahydro-pyrido[3,4-d]pyrimidine-6-carboxylic acid (2-amino-pyridin-4-ylmethyl)-amide,

3-(3-Methoxy-benzyl)-1-methyl-2,4-dioxo-1,2,3,4-tetrahydro-pyrido[3,4-d]pyrimidine-6-carboxylic acid (2-amino-pyridin-4-ylmethyl)-amide,

3-(4-Methoxy-benzyl)-1-methyl-2,4-dioxo-1,2,3,4-tetrahydro-pyrido[3,4-d]pyrimidine-6-carboxylic acid (2-amino-pyridin-4-ylmethyl)-amide,

3-[2-(4-Bromo-phenoxy)-ethyl]-1-methyl-2,4-dioxo-1,2,3,4-tetrahydro-pyrido[3,4-d]pyrimidine-6-carboxylic acid (6-methoxy-pyridin-3-ylmethyl)-amide,

3-[2-(4-Fluoro-phenoxy)-ethyl]-1-methyl-2,4-dioxo-1,2,3,4-tetrahydro-pyrido[3,4-d]pyrimidine-6-carboxylic acid (6-methoxy-pyridin-3-ylmethyl)-amide,

3-[2-(4-Chloro-phenoxy)-ethyl]-1-methyl-2,4-dioxo 1,2,3,4 tetrahydro-pyrido[3,4-d]pyrimidine-6-carboxylic acid (6-methoxy-pyridin-3-ylmethyl)-amide,

3-(4-Chloro-benzyl)-1-methyl-2,4-dioxo-1,2,3,4-tetrahydro-pyrido[3,4-d]pyrimidine-6-carboxylic acid (6-methoxy-pyridin-3-ylmethyl)-amide,

3-(4-Bromo-benzyl)-1-methyl-2,4-dioxo-1,2,3,4-tetrahydro-pyrido[3,4-d]pyrimidine-6-carboxylic acid (6-methoxy-pyridin-3-ylmethyl)-amide,

3-(4-Fluoro-benzyl)-1-methyl-2,4-dioxo-1,2,3,4-tetrahydro-pyrido[3,4-d]pyrimidine-6-carboxylic acid (6-methoxy-pyridin-3-ylmethyl)-amide,

3-(3-Chloro-benzyl)-1-methyl-2,4-dioxo-1,2,3,4-tetrahydro-pyrido[3,4-d]pyrimidine-6-carboxylic acid (6-methoxy-pyridin-3-ylmethyl)-amide,

3-(3-Bromo-benzyl)-1-methyl-2,4-dioxo 1,2,3,4 tetrahydro-pyrido[3,4-d]pyrimidine-6-carboxylic acid (6-methoxy-pyridin-3-ylmethyl)-amide,

3-(3-Fluoro-benzyl)-1-methyl-2,4-dioxo-1,2,3,4-tetrahydro-pyrido[3,4-d]pyrimidine-6-carboxylic acid (6-methoxy-pyridin-3-ylmethyl)-amide,

3-(3,4-Difluoro-benzyl)-1-methyl-2,4-dioxo-1,2,3,4-tetrahydro-pyrido[3,4-d]pyrimidine-6-carboxylic acid (6-methoxy-pyridin-3-ylmethyl)-amide,

3-(3-Chloro-4-fluoro-benzyl)-1-methyl-2,4-dioxo-1,2,3,4-tetrahydro-pyrido[3,4-d]pyrimidine-6-carboxylic acid (6-methoxy-pyridin-3-ylmethyl)-amide,

3-(3-Methoxy-benzyl)-1-methyl-2,4-dioxo-1,2,3,4-tetrahydro-pyrido[3,4-d]pyrimidine-6-carboxylic acid (6-methoxy-pyridin-3-ylmethyl)-amide,

3-(4-Methoxy-benzyl)-1-methyl-2,4-dioxo-1,2,3,4-tetrahydro-pyrido[3,4-d]pyrimidine-6-carboxylic acid (6-methoxy-pyridin-3-ylmethyl)-amide,

3-[2-(4-Bromo-phenoxy)-ethyl]-1-methyl-2,4-dioxo-1,2,3,4-tetrahydro-pyrido[3,4-d]pyrimidine-6-carboxylic acid (6-ethoxy-pyridin-3-ylmethyl)-amide,

3-[2-(4-Fluoro-phenoxy)-ethyl]-1-methyl-2,4-dioxo-1,2,3,4-tetrahydro-pyrido[3,4-d]pyrimidine-6-carboxylic acid (6-ethoxy-pyridin-3-ylmethyl)-amide,

3-[2-(4-Chloro-phenoxy)-ethyl]-1-methyl-2,4-dioxo-1,2,3,4-tetrahydro-pyrido[3,4-d]pyrimidine-6-carboxylic acid (6-ethoxy-pyridin-3-ylmethyl)-amide,

3-(4-Chloro-benzyl)-1-methyl-2,4-dioxo-1,2,3,4-tetrahydro-pyrido[3,4-d]pyrimidine-6-carboxylic acid (6-ethoxy-pyridin-3-ylmethyl)-amide,

3-(4-Bromo-benzyl)-1-methyl-2,4-dioxo-1,2,3,4-tetrahydro-pyrido[3,4-d]pyrimidine-6-carboxylic acid (6-ethoxy-pyridin-3-ylmethyl)-amide,

3-(4-Fluoro-benzyl)-1-methyl-2,4-dioxo-1,2,3,4-tetrahydro-pyrido[3,4-d]pyrimidine-6-carboxylic acid (6-ethoxy-pyridin-3-ylmethyl)-amide,

3-(3-Chloro-benzyl)-1-methyl-2,4-dioxo-1,2,3,4-tetrahydro-pyrido[3,4-d]pyrimidine-6-carboxylic acid (6-ethoxy-pyridin-3-ylmethyl)-amide,

3-(3-Bromo-benzyl)-1-methyl-2,4-dioxo-1,2,3,4-tetrahydro-pyrido[3,4-d]pyrimidine-6-carboxylic acid (6-ethoxy-pyridin-3-ylmethyl)-amide,

3-(3-Fluoro-benzyl)-1-methyl-2,4-dioxo-1,2,3,4-tetrahydro-pyrido[3,4-d]pyrimidine-6-carboxylic acid (6-ethoxy-pyridin-3-ylmethyl)-amide,

3-(3,4-Difluoro-benzyl)-1-methyl-2,4-dioxo-1,2,3,4-tetrahydro-pyrido[3,4-d]pyrimidine-6-carboxylic acid (6-ethoxy-pyridin-3-ylmethyl)-amide,

3-(3-Chloro-4-fluoro-benzyl)-1-methyl-2,4-dioxo-1,2,3,4-tetrahydro-pyrido[3,4-d]pyrimidine-6-carboxylic acid (6-ethoxy-pyridin-3-ylmethyl)-amide,

3-(3-Methoxy-benzyl)-1-methyl-2,4-dioxo-1,2,3,4-tetrahydro-pyrido[3,4-d]pyrimidine-6-carboxylic acid (6-ethoxy-pyridin-3-ylmethyl)-amide,

3-(4-Methoxy-benzyl)-1-methyl-2,4-dioxo-1,2,3,4-tetrahydro-pyrido[3,4-d]pyrimidine-6-carboxylic acid (6-ethoxy-pyridin-3-ylmethyl)-amide,

3-[2-(4-Bromo-phenoxy)-ethyl]-1-methyl-2,4-dioxo-1,2,3,4-tetrahydro-pyrido[3,4-d]pyrimidine-6-carboxylic acid (pyridin-3-ylmethyl)-amide,

3-[2-(4-Fluoro-phenoxy)-ethyl]-1-methyl-2,4-dioxo-1,2,3,4-tetrahydro-pyrido[3,4-d]pyrimidine-6-carboxylic acid (pyridin-3-ylmethyl)-amide,

3-[2-(4-Chloro-phenoxy)-ethyl]-1-methyl-2,4-dioxo-1,2,3,4-tetrahydro-pyrido[3,4-d]pyrimidine-6-carboxylic acid (pyridin-3-ylmethyl)-amide,

3-(4-Chloro-benzyl)-1-methyl-2,4-dioxo-1,2,3,4-tetrahydro-pyrido[3,4-d]pyrimidine-6-carboxylic acid (pyridin-3-ylmethyl)-amide,

3-(4-Bromo-benzyl)-1-methyl-2,4-dioxo-1,2,3,4-tetrahydro-pyrido[3,4-d]pyrimidine-6-carboxylic acid (pyridin-3-ylmethyl)-amide,

3-(4-Fluoro-benzyl)-1-methyl-2,4-dioxo-1,2,3,4-tetrahydro-pyrido[3,4-d]pyrimidine-6-carboxylic acid (pyridin-3-ylmethyl)-amide,

3-(3-Chloro-benzyl)-1-methyl-2,4-dioxo-1,2,3,4-tetrahydro-pyrido[3,4-d]pyrimidine-6-carboxylic acid (pyridin-3-ylmethyl)-amide,

3-(3-Bromo-benzyl)-1-methyl-2,4-dioxo-1,2,3,4-tetrahydro-pyrido[3,4-d]pyrimidine-6-carboxylic acid (pyridin-3-ylmethyl)-amide,

3-(3-Fluoro-benzyl)-1-methyl-2,4-dioxo-1,2,3,4-tetrahydro-pyrido[3,4d]pyrimidine-6-carboxylic acid (pyridin-3-ylmethyl)-amide,

3-(3,4-Difluoro-benzyl)-1-methyl-2,4-dioxo-1,2,3,4-tetrahydro-pyrido[3,4-d]pyrimidine-6-carboxylic acid (pyridin-3-ylmethyl)-amide,

3-(3-Chloro-4-fluoro-benzyl)-1-methyl-2,4-dioxo-1,2,3,4-tetrahydro-pyrido[3,4-d]pyrimidine-6-carboxylic acid (pyridin-3-ylmethyl)-amide,

3-(3-Methoxy-benzyl)-1-methyl-2,4-dioxo-1,2,3,4-tetrahydro-pyrido[3,4-d]pyrimidine-6-carboxylic acid (pyridin-3-ylmethyl)-amide,

3-(4-Methoxy-benzyl)-1-methyl-2,4-dioxo-1,2,3,4-tetrahydro-pyrido[3,4-d]pyrimidine-6-carboxylic acid (pyridin-3-ylmethyl)-amide,

3-[2-(4-Bromo-phenoxy)-ethyl]-1-methyl-2,4-dioxo-1,2,3,4tetrahydro-pyrido[3,4-d]pyrimidine-6-carboxylic acid (6-amino-pyridin-3-ylmethyl)-amide,

3-[2-(4-Fluoro-phenoxy)-ethyl]-1-methyl-2,4-dioxo-1,2,3,4-tetrahydro-pyrido[3,4-d]pyrimidine-6-carboxylic acid (6-amino-pyridin-3-ylmethyl)-amide,

3-[2-(4-Chloro-phenoxy)-ethyl]-1-methyl-2,4-dioxo-1,2,3,4-tetrahydro-pyrido[3,4-d]pyrimidine-6-carboxylic acid (6-amino-pyridin-3-ylmethyl)-amide,

3-(4-Chloro-benzyl)-1-methyl-2,4-dioxo-1,2,3,4-tetrahydro-pyrido[3,4-d]pyrimidine-6-carboxylic acid (6-amino-pyridin-3-ylmethyl)-amide,

3-(4-Bromo-benzyl)-1-methyl-2,4-dioxo-1,2,3,4-tetrahydro-pyrido[3,4-d]pyrimidine-6-carboxylic acid (6-amino-pyridin-3-ylmethyl)-amide,

3-(4-Fluoro-benzyl)-1-methyl-2,4-dioxo-1,2,3,4-tetrahydro-pyrido[3,4-d]pyrimidine-6-carboxylic acid (6-amino-pyridin-3-ylmethyl)-amide,

3-(3-Chloro-benzyl)-1-methyl-2,4-dioxo-1,2,3,4-tetrahydro-pyrido[3,4-d]pyrimidine-6-carboxylic acid (6-amino-pyridin-3-ylmethyl)-amide,

3-(3-Bromo-benzyl)-1-methyl-2,4-dioxo-1,2,3,4-tetrahydro-pyrido[3,4-d]pyrimidine-6-carboxylic acid (6-amino-pyridin-3-ylmethyl)-amide,

3-(3-Fluoro-benzyl)-1-methyl-2,4-dioxo-1,2,3,4-tetrahydro-pyrido[3,4-d]pyrimidine-6-carboxylic acid (6-amino-pyridin-3-ylmethyl)-amide,

3-(3,4-Difluoro-benzyl)-1-methyl-2,4-dioxo-1,2,3,4-tetrahydro-pyrido[3,4-d]pyrimidine-6-carboxylic acid (6-amino-pyridin-3-ylmethyl)-amide,

3-(3-Chloro-4-fluoro-benzyl)-1-methyl-2,4-dioxo-1,2,3,4-tetrahydro-pyrido[3,4-d]pyrimidine-6-carboxylic acid (6-amino-pyridin-3-ylmethyl)-amide,

3-(3-Methoxy-benzyl)-1-methyl-2,4-dioxo-1,2,3,4-tetrahydro-pyrido[3,4-d]pyrimidine-6-carboxylic acid (6-amino-pyridin-3-ylmethyl)-amide,

3-(4-Methoxy-benzyl)-1-methyl-2,4-dioxo-1,2,3,4-tetrahydro-pyrido[3,4-d]pyrimidine-6-carboxylic acid (6-amino-pyridin-3-ylmethyl)-amide,

3-[2-(4-Bromo-phenoxy)-ethyl]-1-methyl-2,4-dioxo-1,2,3,4-tetrahydro-pyrido[3,4-d]pyrimidine-6-carboxylic acid (2-methylamino-pyridin-4-ylmethyl)-amide,

3-[2-(4-Bromo-phenoxy)-ethyl]-1-methyl-2,4-dioxo-1,2,3,4-tetrahydro-pyrido[3,4-d]pyrimidine-6-carboxylic acid (2-methylamino-pyridin-4-ylmethyl)-amide,

3-[2-(4-Chloro-phenoxy)-ethyl]-1-methyl-2,4-dioxo-1,2,3,4-tetrahydro-pyrido[3,4-d]pyrimidine-6-carboxylic acid (2-methylamino-pyridin-4-ylmethyl)-amide,

3-(4-Chloro-benzyl)-1-methyl-2,4-dioxo-1,2,3,4-tetrahydro-pyrido[3,4-d]pyrimidine-6-carboxylic acid (2-methylamino-pyridin-4-ylmethyl)-amide,

3-(4-Bromo-benzyl)-1-methyl-2,4-dioxo-1,2,3,4-tetrahydro-pyrido[3,4-d]pyrimidine-6-carboxylic acid (2-methylamino-pyridin-4-ylmethyl)-amide,

3-(4-Fluoro-benzyl)-1-methyl-2,4-dioxo-1,2,3,4-tetrahydro-pyrido[3,4-d]pyrimidine-6-carboxylic acid (2-methylamino-pyridin-4-ylmethyl)-amide,

3-(3-Chloro-benzyl)-1-methyl-2,4-dioxo-1,2,3,4-tetrahydro-pyrido[3,4-d]pyrimidine-6-carboxylic acid (2-methylamino-pyridin-4-ylmethyl)-amide,

3-(3-Bromo-benzyl)-1-methyl-2,4-dioxo-1,2,3,4-tetrahydro-pyrido[3,4-d]pyrimidine-6-carboxylic acid (2-methylamino-pyridin-4-ylmethyl)-amide,

3-(3-Fluoro-benzyl)-1-methyl-2,4-dioxo-1,2,3,4-tetrahydro-pyrido[3,4-d]pyrimidine 6 carboxylic acid (2-methylamino-pyridin-4-ylmethyl)-amide,

3-(3,4-Difluoro-benzyl)-1-methyl-2,4-dioxo-1,2,3,4-tetrahydro-pyrido[3,4-d]pyrimidine-6-carboxylic acid (2-methylamino-pyridin-4-ylmethyl)-amide,

3-(3-Chloro-4-fluoro-benzyl)-1-methyl-2,4-dioxo-1,2,3,4-tetrahydro-pyrido[3,4-d]pyrimidine-6-carboxylic acid (2-methylamino-pyridin-4-ylmethyl)-amide,

3-(3-Methoxy-benzyl)-1-methyl-2,4-dioxo-1,2,3,4-tetrahydro-pyrido[3,4-d]pyrimidine-6-carboxylic acid (2-methylamino-pyridin-4-ylmethyl)-amide,

3-(4-Methoxy-benzyl)-1-methyl-2,4-dioxo-1,2,3,4-tetrahydro-pyrido[3,4-d]pyrimidine-6-carboxylic acid (2-methylamino-pyridin-4-ylmethyl)-amide.

### Examples 345 to 461

These compounds were obtained according to the procedure described for Example 131:

3-(3,4-Dichloro-benzyl)-1-methyl-2,4-dioxo-1,2,3,4-tetrahydro-quinazoline-6-carboxylic acid (pyridin-4-ylmethyl)-amide,

3-(3-Chloro-benzyl)-1-methyl-2,4-dioxo-1,2,3,4-tetrahydro-quinazoline-6-carboxylic acid (pyridin-4-ylmethyl)-amide,

3-(3-Bromo-benzyl)-1-methyl-2,4-dioxo-1,2,3,4-tetrahydro-quinazoline-6-carboxylic acid (pyridin-4-ylmethyl)-amide,

3-(3-Iodo-benzyl)-1-methyl-2,4-dioxo-1,2,3,4-tetrahydro-quinazoline-6-carboxylic acid (pyridin-4-ylmethyl)-amide,

3-(3,4-Dichloro-benzyl)-1-methyl-2,4-dioxo-1,2,3,4-tetrahydro-quinazoline-6-carboxylic acid (2-methoxy-pyridin-4-ylmethyl)-amide,

3-(3,4-Difluoro-benzyl)-1-methyl-2,4-dioxo-1,2,3,4-tetrahydro-quinazoline-6-carboxylic acid (2-methoxy-pyridin-4-ylmethyl)-amide,

3-(3-Chloro-4-fluoro-benzyl)-1-methyl-2,4-dioxo-1,2,3,4-tetrahydro-quinazoline-6-carboxylic acid (2-methoxy-pyridin-4-ylmethyl)-amide,

3-(3-Chloro-4-fluoro-benzyl)-1-methyl-2,4-dioxo-1,2,3,4-tetrahydro-quinazoline-6-carboxylic acid (2-methoxy-pyridin-4-ylmethyl)-amide,

3-(3-Bromo-benzyl)-1-methyl-2,4-dioxo-1,2,3,4-tetrahydro-quinazoline-6-carboxylic acid (2-methoxy-pyridin-4-ylmethyl)-amide,

3-(3-Iodo-benzyl)-1-methyl-2,4-dioxo-1,2,3,4-tetrahydro-quinazoline-6-carboxylic acid (2-methoxy-pyridin-4-ylmethyl)-amide,

3-(4-Iodo-benzyl)-1-methyl-2,4-dioxo-1,2,3,4-tetrahydro-quinazoline-6-carboxylic acid (2-methoxy-pyridin-4-ylmethyl)-amide,

3-(3,4-Difluoro-benzyl)-1-methyl-2,4-dioxo-1,2,3,4-tetrahydro-quinazoline-6-carboxylic acid (1-hydroxy-pyridazin-4-ylmethyl)-amide,

3-(3,4-Dichloro-benzyl)-1-methyl-2,4-dioxo-1,2,3,4-tetrahydro-quinazoline-6-carboxylic acid (1-hydroxy-pyridazin-4-ylmethyl)-amide,

3-(3-Chloro4-fluoro-benzyl)-1-methyl-2,4-dioxo-1,2,3,4-tetrahydro-quinazoline-6-carboxylic acid (1-hydroxy-pyridazin-4-ylmethyl)-amide,

3-(3-Fluoro-benzyl)-1-methyl-2,4-dioxo-1,2,3,4-tetrahydro-quinazoline-6-carboxylic acid (1-hydroxy-pyridazin-4-ylmethyl)-amide,

3-(3-Chloro-benzyl)-1-methyl-2,4-dioxo-1,2,3,4-tetrahydro-quinazoline-6-carboxylic acid (1-hydroxy-pyridazin-4-ylmethyl)-amide,

3-(3-Bromo-benzyl)-1-methyl-2,4-dioxo-1,2,3,4-tetrahydro-quinazoline-6-carboxylic acid (1-hydroxy-pyridazin-4-ylmethyl)-amide,

3-(4-Fluoro-benzyl)-1-methyl-2,4-dioxo-1,2,3,4-tetrahydro-quinazoline-6-carboxylic acid (1-hydroxy-pyridazin-4-ylmethyl)-amide,

3-(4-Chloro-benzyl)-1-methyl-2,4-dioxo-1,2,3,4-tetrahydro-quinazoline-6-carboxylic acid (1-hydroxy-pyridazin-4-ylmethyl)-amide,

3-(4-Bromo-benzyl)-1-methyl-2,4-dioxo-1,2,3,4-tetrahydro-quinazoline-6-carboxylic acid (1-hydroxy-pyridazin-4-ylmethyl)-amide,

3-(3,4-Difluoro-benzyl)-1-methyl-2,4-dioxo-1,2,3,4-tetrahydro-quinazoline-6-carboxylic acid (1-methylamino-pyridazin-4-ylmethyl)-amide,

3-(3,4-Dichloro-benzyl)-1-methyl-2,4-dioxo-1,2,3,4-tetrahydro-quinazoline-6-carboxylic acid (1-methylamino-pyridazin-4-ylmethyl)-amide,

3-(3-Chloro-4-fluoro-benzyl)-1-methyl-2,4-dioxo-1,2,3,4-tetrahydro-quinazoline-6-carboxylic acid (1-methylamino-pyridazin-4-ylmethyl)-amide,

3-(3-Fluoro-benzyl)-1-methyl-2,4-dioxo-1,2,3,4-tetrahydro-quinazoline-6-carboxylic acid (1-methylamino-pyridazin-4-ylmethyl)-amide,

3-(3-Chloro-benzyl)-1-methyl-2,4-dioxo-1,2,3,4-tetrahydro-quinazoline-6-carboxylic acid (1-methylamino-pyridazin-4-ylmethyl)-amide,

3-(3-Bromo-benzyl)-1-methyl-2,4-dioxo-1,2,3,4-tetrahydro-quinazoline-6-carboxylic acid (1-methylamino-pyridazin-4-ylmethyl)-amide,

3-(4-Fluoro-benzyl)-1-methyl-2,4-dioxo-1,2,3,4-tetrahydro-quinazoline-6-carboxylic acid (1-methylamino-pyridazin-4-ylmethyl)-amide,

3-(4-Chloro-benzyl)-1-methyl-2,4-dioxo-1,2,3,4-tetrahydro-quinazoline-6-carboxylic acid (1-methylamino-pyridazin-4-ylmethyl)-amide,

3-(4-Bromo-benzyl)-1-methyl-2,4-dioxo-1,2,3,4-tetrahydro-quinazoline-6-carboxylic acid (1-methylamino-pyridazin-4-ylmethyl)-amide,

3-(3,4-Difluoro-benzyl)-1-methyl-2,4-dioxo-1,2,3,4-tetrahydro-quinazoline-6-carboxylic acid (1-methoxy-pyridazin-4-ylmethyl)-amide,

3-(3,4-Dichloro-benzyl)-1-methyl-2,4-dioxo-1,2,3,4-tetrahydro-quinazoline-6-carboxylic acid (1-methoxy-pyridazin-4-ylmethyl)-amide,

3-(3-Chloro-4-fluoro-benzyl) 1 methyl 2,4 dioxo 1,2,3,4 tetrahydroquinazoline 6 carboxylic acid (1-methoxy-pyridazin-4-ylmethyl)-amide,

3-(3-Fluoro-benzyl)-1-methyl-2,4-dioxo-1,2,3,4-tetrahydro-quinazoline-6-carboxylic acid (1-methoxy-pyridazin-4-ylmethyl)-amide,

3-(3-Chloro-benzyl)-1-methyl-2,4-dioxo-1,2,3,4-tetrahydro-quinazoline-6-carboxylic acid (1-methoxy-pyridazin-4-ylmethyl)-amide,

3-(3-Bromo-benzyl)-1-methyl-2,4-dioxo-1,2,3,4-tetrahydro-quinazoline-6-carboxylic acid (1-methoxy-pyridazin-4-ylmethyl)-amide,

3-(3-Iodo-benzyl)-1-methyl-2,4-dioxo-1,2,3,4-tetrahydro-quinazoline-6-carboxylic acid (1-methoxy-pyridazin-4-ylmethyl)-amide,

3-(4-Fluoro-benzyl)-1-methyl-2,4-dioxo-1,2,3,4-tetrahydro-quinazoline-6-carboxylic acid (1-methoxy-pyridazin-4-ylmethyl)-amide,

3-(4-Chloro-benzyl)-1-methyl-2,4-dioxo-1,2,3,4-tetrahydro-quinazoline-6-carboxylic acid (1-methoxy-pyridazin-4-ylmethyl)-amide,

3-(4-Bromo-benzyl)-1-methyl-2,4-dioxo-1,2,3,4-tetrahydro-quinazoline-6-carboxylic acid (1-methoxy-pyridazin-4-ylmethyl)-amide,

3-(4lodo-benzyl)-1-methyl-2,4-dioxo-1,2,3,4-tetrahydro-quinazoline-6-carboxylic acid (1-methoxy-pyridazin-4-ylmethyl)-amide,

3-(3,4-Difluoro-benzyl)-1-methyl-2,4-dioxo-1,2,3,4-tetrahydro-quinazoline-6-carboxylic acid (2-hydroxy-pyridazin-4-ylmethyl)-amide,

3-(3,4-Dichloro-benzyl)-1-methyl-2,4-dioxo-1,2,3,4-tetrahydro-quinazoline-6-carboxylic acid (2-hydroxy-pyridazin-4-ylmethyl)-amide,

3-(3-Chloro-4-fluoro-benzyl)-1-methyl-2,4-dioxo-1,2,3,4-tetrahydro-quinazoline-6-carboxylic acid (2-hydroxy-pyridazin-4-ylmethyl)-amide,

3-(3-Fluoro-benzyl)-1-methyl-2,4-dioxo-1,2,3,4-tetrahydro-quinazoline-6-carboxylic acid (2-hydroxy-pyridazin-4-ylmethyl)-amide,

3-(3-Chloro-benzyl)-1-methyl-2,4-dioxo-1,2,3,4-tetrahydro-quinazoline-6-carboxylic acid (2-hydroxy-pyridazin-4-ylmethyl)-amide,

3-(3-Bromo-benzyl)-1-methyl-2,4-dioxo-1,2,3,4-tetrahydro-quinazoline-6-carboxylic acid (2-hydroxy-pyridazin-4-ylmethyl)-amide,

3-(4-Fluoro-benzyl)-1-methyl-2,4-dioxo-1,2,3,4-tetrahydro-quinazoline-6-carboxylic acid (2-hydroxy-pyridazin-4-ylmethyl)-amide,

3-(4-Chloro-benzyl)-1-methyl-2,4-dioxo-1,2,3,4-tetrahydro-quinazoline-6-carboxylic acid (2-hydroxy-pyridazin-4-ylmethyl)-amide,

3-(4-Bromo-benzyl)-1-methyl-2,4-dioxo-1,2,3,4-tetrahydro-quinazoline-6-carboxylic acid (2-hydroxy-pyridazin-4-ylmethyl)-amide,

3-(3,4-Difluoro-benzyl)-1-methyl-2,4-dioxo-1,2,3,4-tetrahydro-quinazoline-6-carboxylic acid (1-amino-pyridazin-4-ylmethyl)-amide,

3-(3,4-Dichloro-benzyl)-1-methyl-2,4-dioxo-1,2,3,4tetrahydro-quinazoline-6-carboxylic acid (1-amino-pyridazin-4-ylmethyl)-amide,

3-(3-Chloro-4-fluoro-benzyl)-1-methyl-2,4-dioxo-1,2,3,4-tetrahydro-quinazoline-6-carboxylic acid (1-amino-pyridazin-4-ylmethyl)-amide,

3-(3-Fluoro-benzyl)-1-methyl-2,4-dioxo-1,2,3,4-tetrahydro-quinazoline-6-carboxylic acid (1-amino-pyridazin-4-ylmethyl)-amide,

3-(3-Chloro-benzyl)-1-methyl-2,4-dioxo-1,2,3,4-tetrahydro-quinazoline-6-carboxylic acid (1-amino-pyridazin-4-ylmethyl)-amide,

3-(3-Bromo-benzyl)-1-methyl-2,4-dioxo-1,2,3,4-tetrahydro-quinazoline-6-carboxylic acid (1amino-pyridazin-4-ylmethyl)-amide,

3-(4-Fluoro-benzyl)-1-methyl-2,4-dioxo-1,2,3,4-tetrahydro-quinazoline-6-carboxylic acid (1-amino-pyridazin-4-ylmethyl)-amide,

3-(4-Chloro-benzyl)-1-methyl-2,4-dioxo-1,2,3,4-tetrahydro-quinazoline-6-carboxylic acid (1-amino-pyridazin-4-ylmethyl)-amide,

3-(4-Bromo-benzyl)-1-methyl-2,4-dioxo-1,2,3,4-tetrahydro-quinazoline-6-carboxylic acid (1-amino-pyridazin-4-ylmethyl)-amide,

3-(3,4-Difluoro-benzyl)-1-methyl-2,4-dioxo-1,2,3,4-tetrahydro-quinazoline-6-carboxylic acid (1-ethoxy-pyridazin-4-ylmethyl)-amide,

3-(3,4-Dichloro-benzyl)-1-methyl-2,4-dioxo-1,2,3,4-tetrahydro-quinazoline-6-carboxylic acid (1-ethoxy-pyridazin-4-ylmethyl)-amide,

3-(3-Chloro-4-fluoro-benzyl)-1-methyl-2,4-dioxo-1,2,3,4-tetrahydro-quinazoline-6-carboxylic acid (1-ethoxy-pyridazin-4-ylmethyl)-amide,

3-(3-Fluoro-benzyl)-1-methyl-2,4-dioxo-1,2,3,4-tetrahydro-quinazoline-6-carboxylic acid (1-ethoxy-pyridazin-4-ylmethyl)-amide,

3-(3-Chloro-benzyl)-1-methyl-2,4-dioxo-1,2,3,4-tetrahydro-quinazoline-6-carboxylic acid (1-ethoxy-pyridazin-4-ylmethyl)-amide,

3-(3-Bromo-benzyl)-1-methyl-2,4-dioxo-1,2,3,4-tetrahydro-quinazoline-6-carboxylic acid (1-ethoxy-pyridazin-4-ylmethyl)-amide,

3-(3-Iodo-benzyl)-1-methyl-2,4-dioxo-1,2,3,4-tetrahydro-quinazoline-6-carboxylic acid (1-ethoxy-pyridazin-4-ylmethyl)-amide,

3-(4-Fluoro-benzyl)-1-methyl-2,4-dioxo-1,2,3,4-tetrahydro-quinazoline-6-carboxylic acid (1-ethoxy-pyridazin-4-ylmethyl)-amide,

3-(4-Chloro-benzyl)-1-methyl-2,4-dioxo-1,2,3,4-tetrahydro-quinazoline-6-carboxylic acid (1-ethoxy-pyridazin-4-ylmethyl)-amide,

3-(4-Bromo-benzyl)-1-methyl-2,4-dioxo-1,2,3,4-tetrahydro-quinazoline-6-carboxylic acid (1-ethoxy-pyridazin-4-ylmethyl)-amide,

3-(4-Iodo-benzyl)-1-methyl-2,4-dioxo-1,2,3,4-tetrahydro-quinazoline-6-carboxylic acid (1-ethoxy-pyridazin-4-ylmethyl)-amide,

3-(3,4-Difluoro-benzyl)-1-methyl-2,4-dioxo-1,2,3,4-tetrahydro-quinazoline-6-carboxylic acid (2-methylamino-pyridazin-4-ylmethyl)-amide,

3-(3,4-Dichloro-benzyl)-1-methyl-2,4-dioxo-1,2,3,4-tetrahydro-quinazoline-6-carboxylic acid (2-methylamino-pyridazin-4-ylmethyl)-amide,

3-(3-Fluoro-benzyl)-1-methyl-2,4-dioxo-1,2,3,4-tetrahydro-quinazoline-6-carboxylic acid (2-methylamino-pyridazin-4-ylmethyl)-amide,

3-(3-Chloro-benzyl)-1-methyl-2,4-dioxo-1,2,3,4-tetrahydro-quinazoline-6-carboxylic acid (2-methylamino-pyridazin-4-ylmethyl)-amide,

3-(3-Bromo-benzyl)-1-methyl-2,4-dioxo-1,2,3,4-tetrahydro-quinazoline-6-carboxylic acid (2-methylamino-pyridazin-4-ylmethyl)-amide,

3-(4-Fluoro-benzyl)-1-methyl-2,4-dioxo-1,2,3,4-tetrahydro-quinazoline-6-carboxylic acid (2-methylamino-pyridazin-4-ylmethyl)-amide,

3-(4-Chloro-benzyl)-1-methyl-2,4-dioxo-1,2,3,4-tetrahydro-quinazoline-6-carboxylic acid (2-methylamino-pyridazin-4-ylmethyl)-amide,

3-(4-Bromo-benzyl)-1-methyl-2,4-dioxo-1,2,3,4-tetrahydro-quinazoline-6-carboxylic acid (2-methylamino-pyridazin-4-ylmethyl)-amide,

3-(3,4-Difluoro-benzyl)-1-methyl-2,4-dioxo-1,2,3,4-tetrahydro-quinazoline-6-carboxylic acid (1-methyl-pyridazin-4-ylmethyl)-amide,

3-(3,4-Dichloro-benzyl)-1-methyl-2,4-dioxo-1,2,3,4-tetrahydro-quinazoline-6-carboxylic acid (1-amino-pyridazin-4-ylmethyl)-amide,

3-(3-Chloro-4-fluoro-benzyl)-1-methyl-2,4-dioxo-1,2,3,4-tetrahydro-quinazoline-6-carboxylic acid (1-methyl-pyridazin-4-ylmethyl)-amide,

3-(3-Fluoro-benzyl)-1-methyl-2,4-dioxo-1,2,3,4-tetrahydro-quinazoline-6-carboxylic acid (1-methyl-pyridazin-4-ylmethyl)-amide,

3-(3-Chloro-benzyl)-1-methyl-2,4-dioxo-1,2,3,4-tetrahydro-quinazoline-6-carboxylic acid (1-methyl-pyridazin-4-ylmethyl)-amide,

3-(3-Bromo-benzyl)-1-methyl-2,4-dioxo-1,2,3,4-tetrahydro-quinazoline-6-carboxylic acid (1-methyl-pyridazin-4-ylmethyl)-amide,

3-(4-Fluoro-benzyl)-1-methyl-2,4-dioxo-1,2,3,4-tetrahydro-quinazoline-6-carboxylic acid (1-methyl-pyridazin-4-ylmethyl)-amide,

3-(4-Chloro-benzyl)-1-methyl-2,4-dioxo-1,2,3,4-tetrahydro-quinazoline-6-carboxylic acid (1-methyl-pyridazin-4-ylmethyl)-amide,

3-(4-Bromo-benzyl)-1-methyl-2,4-dioxo-1,2,3,4-tetrahydro-quinazoline-6-carboxylic acid (1-methyl-pyridazin-4-ylmethyl)-amide,

3-(3,4-Difluoro-benzyl)-1-methyl-2,4-dioxo-1,2,3,4-tetrahydro-quinazoline-6-carboxylic acid (2-ethoxy-pyridazin-4-ylmethyl)-amide,

3-(3-Chloro-4-fluoro-benzyl)-1-methyl-2,4-dioxo-1,2,3,4-tetrahydro-quinazoline-6-carboxylic acid (2-ethoxy-pyridazin-4-ylmethyl)-amide,

3-(3-Fluoro-benzyl)-1-methyl-2,4-dioxo-1,2,3,4-tetrahydro-quinazoline-6-carboxylic acid (2-ethoxy-pyridazin-4-ylmethyl)-amide,

3-(3-Chloro-benzyl)-1-methyl-2,4-dioxo-1,2,3,4-tetrahydro-quinazoline-6-carboxylic acid (2-ethoxy-pyridazin-4-ylmethyl)-amide,

3-(3-Bromo-benzyl)-1-methyl-2,4-dioxo-1,2,3,4-tetrahydro-quinazoline-6-carboxylic acid (2-ethoxy-pyridazin-4-ylmethyl)-amide,

3-(3-Iodo-benzyl)-1-methyl-2,4-dioxo-1,2,3,4-tetrahydro-quinazoline-6-carboxylic acid (2-ethoxy-pyridazin-4-ylmethyl)-amide,

3-(4-Fluoro-benzyl)-1-methyl-2,4-dioxo-1,2,3,4-tetrahydro-quinazoline-6-carboxylic acid (2-ethoxy-pyridazin-4-ylmethyl)-amide,

3-(4-Chloro-benzyl)-1-methyl-2,4-dioxo-1,2,3,4-tetrahydro-quinazoline-6-carboxylic acid (2-ethoxy-pyridazin-4-ylmethyl)-amide,

3-(4-Bromo-benzyl)-1-methyl-2,4-dioxo-1,2,3,4-tetrahydro-quinazoline-6-carboxylic acid (2-ethoxy-pyridazin-4-ylmethyl)-amide,

3-(4-Iodo-benzyl)-1-methyl-2,4-dioxo-1,2,3,4-tetrahydro-quinazoline-6-carboxylic acid (2-ethoxy-pyridazin-4-ylmethyl)-amide,

3-(3,4-Difluoro-benzyl)-1-methyl-2,4-dioxo-1,2,3,4-tetrahydro-quinazoline-6-carboxylic acid (2-amino-pyridazin-4-ylmethyl)-amide,

3-(3,4-Dichloro-benzyl)-1-methyl-2,4-dioxo-1,2,3,4-tetrahydroquinazoline-6-carboxylic acid (2-amino-pyridazin-4-ylmethyl)-amide,

3-(3-Chloro-4-fluoro-benzyl)-1-methyl-2,4-dioxo-1,2,3,4-tetrahydro-quinazoline-6-carboxylic acid (2-amino-pyridazin-4-ylmethyl)-amide,

3-(3-Fluoro-benzyl)-1-methyl-2,4-dioxo-1,2,3,4-tetrahydro-quinazoline-6-carboxylic acid (2-amino-pyridazin-4-ylmethyl)-amide,

3-(3-Chloro-benzyl)-1-methyl-2,4-dioxo-1,2,3,4-tetrahydro-quinazoline-6-carboxylic acid (2-amino-pyridazin-4-ylmethyl)-amide,

3-(3-Bromo-benzyl)-1-methyl-2,4-dioxo-1,2,3,4-tetrahydro-quinazoline-6-carboxylic acid (2-amino-pyridazin-4-ylmethyl)-amide,

3-(4-Fluoro-benzyl)-1-methyl-2,4-dioxo-1,2,3,4-tetrahydro-quinazoline-6-carboxylic acid (2-amino-pyridazin-4-ylmethyl)-amide,

3-(4-Chloro-benzyl)-1-methyl-2,4-dioxo-1,2,3,4-tetrahydro-quinazoline-6-carboxylic acid (2-amino-pyridazin-4-ylmethyl)-amide,

3-(4-Bromo-benzyl)-1-methyl-2,4-dioxo-1,2,3,4-tetrahydro-quinazoline-6-carboxylic acid (2-amino-pyridazin-4-ylmethyl)-amide,

3-(3,4-Difluoro-benzyl)-1-methyl-2,4-dioxo-1,2,3,4-tetrahydro-quinazoline-6-carboxylic acid (2-methyl-pyridazin-4-ylmethyl)-amide,

3-(3,4-Dichloro-benzyl)-1-methyl-2,4-dioxo-1,2,3,4-tetrahydro-quinazoline-6-carboxylic acid (2-methyl-pyridazin-4-ylmethyl)-amide,

3-(3-Chloro-4-fluoro-benzyl)-1-methyl-2,4-dioxo-1,2,3,4-tetrahydro-quinazoline-6-carboxylic acid (2-methyl-pyridazin-4-ylmethyl)-amide,

3-(3-Fluoro-benzyl)-1-methyl-2,4-dioxo-1,2,3,4-tetrahydro-quinazoline-6-carboxylic acid (2-methyl-pyridazin-4-ylmethyl)-amide,

3-(3-Chloro-benzyl)-1-methyl-2,4-dioxo-1,2,3,4-tetrahydro-quinazoline-6-carboxylic acid (2-methyl-pyridazin-4-ylmethyl)-amide,

3-(3-Bromo-benzyl)-1-methyl-2,4-dioxo-1,2,3 ,4-tetrahydro-quinazoline-6-carboxylic acid (2-methyl-pyridazin-4-ylmethyl)-amide,

3-(4-Fluoro-benzyl)-1-methyl-2,4-dioxo-1,2,3,4-tetrahydro-quinazoline-6-carboxylic acid (2-methyl-pyridazin-4-ylmethyl)-amide,

3-(4-Chloro-benzyl)-1-methyl-2,4-dioxo-1,2,3,4-tetrahydro-quinazoline-6-carboxylic acid (2-methyl-pyridazin-4-ylmethyl)-amide,

and 3-(4-Bromo-benzyl)-1-methyl-2,4-dioxo-1,2,3,4-tetrahydro-quinazoline-6-carboxylic acid (2-methyl-pyridazin-4-ylmethyl)-amide.

### Example 462

Evaluation of the in vitro Activity of the Compounds of Formula (I) According to the Invention

The ability of the compounds of formula (I) of the invention to inhibit matrix metalloprotease 13 was evaluated by measuring their IC₅₀ value (concentration required to inhibit 50% of the enzymatic activity) according to the protocol described below. MMP13CD Thiopeptolide Assay: Proteolysis of the thiopeptolide substrate Ac-Pro-Leu-Gly-thioester-Leu-Leu-Gly-OEt is used as the primary screen to determine IC₅₀ values for MMP13 inhibitors. A 100 μl reaction contains 50 mM HEPES, 10 mM CaCl₂, pH 7.0 (RT), 1 mM 5,5′-dithiobis(2-nitrobenzoic acid) (DTNB), 100 μM substrate, inhibitor in 2.0% DMSO and 2.5 nM human collagenase-3 catalytic domain enzyme. Inhibitors are screened from 100 μM to 0.5 nM. The change in absorbance at 405 nm is monitored on a microplate reader at room temperature continuously for 10-15 minutes. Percentage of control velocity in inhibited treatments is plotted against inhibitor concentration to calculate IC₅₀ values.

TABLE 1

| Example | IC₅₀ (μM) |
|---|---|
| 5 | 0.193 |
| 6 | 0.183 |
| 7 | 0.021 |
| 8 | 1.87 |
| 9 | 0.366 |
| 10 | 0.049 |
| 11 | 0.167 |
| 12 | 1.32 |
| 13 | 0.005 |
| 14 | 0.057 |
| 15 | 2.25 |
| 16 | 0.042 |
| 17 | 0.012 |
| 18 | 0.051 |
| 19d | 0.7 |
| 20 | 0.015 |
| 21 | 0.009 |
| 22b | 0.01 |
| 24 | 0.051 |
| 25 | 0.3 |
| 26 | 0.096 |
| 27 | 0.029 |
| 28 | 0.009 |
| 29 | 0.028 |
| 30 | 0.009 |
| 31 | 1.7 |
| 32 | 0.017 |
| 33 | 0.003 |
| 34 | 0.026 |
| 35 | 0.157 |
| 36 | 0.6 |
| 37 | 0.75 |
| 38b | 0.004 |
| 39 | 0.001 |
| 40 | 0.028 |
| 41 | 0.029 |
| 42 | 0.031 |
| 43 | 0.011 |
| 44 | 0.004 |
| 45 | 0.007 |
| 46 | 0.0025 |
| 47 | 1.21 |
| 48 | 0.016 |
| 49 | 0.007 |
| 50 | 0.096 |
| 51 | 0.062 |
| 52 | 0.014 |

Examination of the results of Table 1 shows that the products of the invention tested in the assay effectively inhibit matrix metalloprotease 13.

The protocol described above was also used to measure the activity of the compounds of the invention against MMP1, MMP2, MMP3, MMP7, MMP9, MMP12 and MMP14. The IC₅₀ values obtained on these MMPs were often greater than 100 μM. These results the compounds of the invention are selective MMP13 inhibitors.

### BIBLIOGRAPHIC REFERENCES

MONTANA J. and BAXTER A., Current opinion in drug discovery and development, 2000, 3 (4), 353-361.

CLARK I M et al., Current opinion in anti-inflammatory and immunomodulatory investigational drugs, 2000, 2 (1), 16-25.

1- A compound selected from those of formula (I): 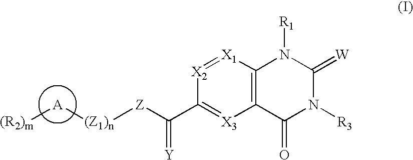
in which:
R₁ represents a group selected from:
hydrogen, amino,
(C₁-C₆)alkyl, (C₃-C₆)alkenyl, (C₃-C₆)alkynyl, mono(C₁-C₆)alkylamino(C₁-C₆)alkyl, di(C₁-C₆)alkylamino(C₁-C₆)alkyl, aryl, aryl(C₁-C₆)alkyl, heterocycle, and 3- to 6-membered cycloalkyl(C₁-C₆)alkyl, these groups being unsubstituted or substituted with one or more groups, which may be identical or different, selected from amino, (C₁-C₆)alkyl, cyano, halo(C₁-C₆)alkyl, C(═O)OR₄, OR₄ and SR₄, in which R₄ represents hydrogen or (C₁-C₆)alkyl,
W represents an oxygen atom, a sulphur atom, or a group ═N—R′, in which R′ represents (C₁-C₆)alkyl, hydroxyl, or cyano,
X₁, X₂ and X₃ represent, independently of each other, a nitrogen atom or a group —C—R₆ in which R₆ represents a group selected from hydrogen, (C₁-C₆)alkyl, amino, mono(C₁-C₆)alkylamino, di(C₁-C₆)alkylamino, hydroxyl, (C₁-C₆)alkoxy, and halogen,
with the proviso that not more than two of the groups X₁, X₂ and X₃ simultaneously represent a nitrogen atom,
Y represents a group selected from oxygen atom, sulphur atom, —NH, and —N(C₁-C₆)alkyl,
Z represents:
an oxygen atom, a sulphur atom,
or a group —NR₇ in which R₇ represents a group selected from hydrogen, (C₁-C₆)alkyl, aryl(C₁-C₆)alkyl, cycloalkyl, aryl, and heteroaryl, and
when Y is an oxygen atom, a sulphur atom, or a group —N(C₁-C₆)alkyl, Z optionally represents a carbon atom which is unsubstituted or substituted with a (C₁-C₆)alkyl, an aryl, an aryl(C₁-C₆)alkyl, an aromatic or non-aromatic heterocycle or a cycloalkyl,
n is an integer from 1 to 8 inclusive,
Z₁ represents —CR₈R₉ wherein R₈ and R₉, independently of each other, represent a group selected from hydrogen, (C₁-C₆)alkyl, halo(C₁-C₆)alkyl, halogen, amino, OR₄, SR₄ or C(═O)OR₄ in which R₄ represents a hydrogen or (C₁-C₆)alkyl, and
when n is greater than or equal to 2, the hydrocarbon chain Z₁ optionally contains one or more multiple bonds,
and/or one of the carbon atoms in the hydrocarbon chain Z₁ may be replaced with an oxygen atom, a sulphur atom which is unsubstituted or substituted with one or two oxygen atoms, or a nitrogen atom which is unsubstituted or substituted with a (C₁-C₆)alkyl,
and when one of the carbon atoms in the hydrocarbon chain Z₁ is replaced with a sulphur atom which is unsubstituted or substituted with one or two oxygen atoms, then the group —C(═Y)—Z— optionally may be absent in the general formula (I),
A represents a group selected from:
aromatic or non-aromatic, 5- or 6-membered monocycle comprising from 0 to 4 heteroatoms selected from nitrogen, oxygen and sulphur, and
bicycle, composed of two aromatic or non-aromatic, 5- or 6-membered rings, which may be identical or different, comprising from 0 to 4 heteroatoms selected from nitrogen, oxygen and sulphur,
m is an integer from 0 to 7 inclusive,
the group(s) R₂, which may be identical or different, is (are) selected from (C₁-C₆)alkyl, halogen, —CN, NO₂, SCF₃, —CF₃, —OCF₃, —NR₁₀R₁₁, —OR₁₀, —SR₁₀, SOR₁₀, —SO₂R₁₀, —(CH₂)ₖSO₂NR₁₀R₁₁, —X₅(CH₂)ₖC(═O)OR₁₀, —(CH₂)ₖC(═O)OR₁₀, —X₅(CH₂)ₖC(═O)NR₁₀R₁₁, —(CH₂)ₖC(═O)NR₁₀R₁₁, and —X₄—R₁₂ in which:
X₅ represents a group selected from oxygen, sulphur optionally substituted by one or two oxygen atoms, and nitrogen substituted by hydrogen or (C₁-C₆)alkyl,
k is an integer from 0 to 3 inclusive,
R₁₀, and R₁₁, which may be identical or different, are selected from hydrogen and (C₁-C₆)alkyl,
X₄ represents a group selected from single bond, —CH₂—, oxygen atom, sulphur atom optionally substituted by one or two oxygen atoms, and nitrogen atom substituted by hydrogen atom or (C₁-C₆)alkyl group,
R₁₂ represents an aromatic or non-aromatic, heterocyclic or non-heterocyclic, 5- or 6-membered ring which is unsubstituted or substituted with one or more groups, which may be identical or different, selected from (C₁-C₆)alkyl, halogen, hydroxyl and amino, and when the ring is heterocyclic, it comprises from 1 to 4 heteroatoms selected from nitrogen, oxygen and sulphur;
R₃ represents a group selected from:
hydrogen,
(C₁-C₆)alkyl, (C₃-C₆)alkenyl, (C₃-C₆)alkynyl, these groups being unsubstituted or substituted with one or more groups, which may be identical or different, selected from amino, cyano, halo(C₁-C₆)alkyl, cycloalkyl, —C(═O)NR₁₀R₁₁, —C(═O)OR₁₀, OR₁₀, and SR₁₀, in which R₁₀ and R₁₁, which may be identical or different, represent hydrogen or (C₁-C₆)alkyl,
and the group of formula: 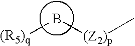
in which p is an integer from 0 to 8 inclusive,
Z₂ represents —CR₁₃R₁₄ wherein R₁₃ and R₁₄, independently of each other, represent a group selected from hydrogen, (C₁-C₆)alkyl, phenyl, halo(C₁-C₆)alkyl, halogen, amino, OR₄, SR₄ and —C(═O)OR₄ in which R₄ represents hydrogen or (C₁-C₆)alkyl, and
when p is greater than or equal to 2, the hydrocarbon chain Z₂ optionally contains one or more multiple bonds,
and/or one of the carbon atoms in the hydrocarbon chain Z₂ may be replaced with an oxygen atom, a sulphur atom which is unsubstituted or substituted with one or two oxygen atoms, a nitrogen atom which is unsubstituted or substituted with a (C₁-C₆)alkyl, or a carbonyl group,
B represents a group selected from:
an aromatic or non-aromatic 5- or 6-membered monocycle comprising from 0 to 4 heteroatoms selected from nitrogen, oxygen and sulphur, and
a bicycle, composed of two aromatic or non-aromatic, 5- or 6-membered rings, which may be identical or different, comprising from 0 to 4 heteroatoms selected from nitrogen, oxygen and sulphur,
q is an integer from 0 to 7 inclusive,
the group(s) R₅, which may be identical or different, is (are) selected from (C₁-C₆)alkyl, halogen, CN, NO₂, CF₃, OCF₃, —(CH₂)ₖNR₁₅R₁₆, —N(R₁₅)C(═O)R₁₆, —N(R₁₅)C(═O)OR₁₆, —N(R₁₅)SO₂R₁₆, —N(SO₂R₁₅)₂, —OR₁₅, —S(O)ₖ₁R₁₅, —SO₂—N(R₁₅)—(CH₂)ₖ₂—NR₁₆R₁₇, —(CH₂)ₖSO₂NR₁₅R₁₆, —X₇(CH₂)ₖC(═O)OR₁₅, —(CH₂)ₖC(═O)OR₁₅, —C(═O)O—(CH₂)ₖ₂—NR₁₅R₁₆, —C(═O)O—(CH₂)ₖ₂—C(═O)OR₁₈, —X₇(CH₂)ₖC(═O)NR₁₅R₁₆, —(CH₂)ₖC(═O)NR₁₅R₁₆, —R₁₉—C(═O)OR₁₅, —X₆—R₂₀, and —C(═O)—R₂₁—NR₁₅R₁₆ in which:
X₇ represents a group selected from oxygen atom, sulphur atom optionally substituted by one or two oxygen atoms, and nitrogen atom substituted by a hydrogen atom or a (C₁-C₆)alkyl group,
k is an integer from 0 to 3 inclusive,
k1 is an integer from 0 to 2 inclusive,
k2 is an integer from 1 to 4 inclusive,
R₁₅, R₁₆ and R₁₇, which may be identical or different, are selected from hydrogen and (C₁-C₆)alkyl,
R₁₈ represents a group selected from (C₁-C₆)alkyl, —R₂₁—NR₁₅R₁₆, —R₂₁—NR₁₅—C(═O)—R₂₁—NR₁₆R₁₇, and —C(═O)O—R₂₁—NR₁₅R₁₆ in which R₂₁ represents a linear or branched (C₁-C₆)alkylene group, and R₁₅, R₁₆ and R₁₇ are as defined hereinbefore,
R₁₉ represents a (C₃-C₆)cycloalkyl group,
X₆ represents a group selected from single bond, —CH₂—, oxygen atom, sulphur atom optionally substituted by one or two oxygen atoms, and nitrogen atom substituted by hydrogen atom or (C₁-C₆)alkyl group,
R₂₀ represents an aromatic or non-aromatic, heterocyclic or non-heterocyclic, 5- or 6-membered ring, which is unsubstituted or substituted with one or more groups, which may be identical or different, selected from (C₁-C₆)alkyl, halogen, hydroxyl, oxo, cyano, tetrazole, amino, and —C(═O)OR₄ wherein R₄ represents hydrogen or (C₁-C₆)alkyl, and, when the ring is heterocyclic, it comprises from 1 to 4 heteroatoms selected from nitrogen, oxygen and sulphur,
with the proviso that when X₁ represents a nitrogen atom, X₂ cannot represent a carbon atom substituted with a methyl group or with NH—CH₃,
optionally, the racemic forms thereof, isomers thereof, N-oxydes thereof, and the pharmaceutically acceptable salts thereof.

2- A compound of formula (I) according to claim 1 characterized in that:
R₁ represents hydrogen, (C₁-C₆)alkyl, aryl(C₁-C₆)alkyl or 3- to 6-membered cycloalkyl(C₁-C₆)alkyl,
W represents an oxygen atom or a sulphur atom,
X₁ represents a nitrogen atom or —C—R₆ in which R₆ represents a hydrogen atom,
X₂ and X₃ represent each —C—R₆ in which R₆ represents a hydrogen atom,
Y represents an oxygen atom,
Z represents an oxygen atom or —NR₇ in which R₇ represents a hydrogen atom,
optionally, the racemic forms thereof, isomers thereof, N-oxydes thereof, and the pharmaceutically acceptable salts thereof.

3- A compound of formula (I) according to claim 1 characterized in that:
n is an integer from 1 to 6 inclusive,
Z₁ represents —CR₈R₉ wherein R₈ represents a hydrogen atom and R₉ represents a hydrogen atom or a methyl group, and
when n is greater than or equal to 2, the hydrocarbon chain Z₁ optionally contains a double bond,
or, one of the carbon atoms in the hydrocarbon chain Z₁ may be replaced with an oxygen atom, or a sulphur atom which is unsubstituted or substituted with one or two oxygens,
A represents a group selected from phenyl, pyridyl, thienyl, imidazolyl, furyl, piperidyl, 1,3-benzodioxolyl, benzodioxinyl, benzothienyl, benzofuryl, benzofurazanyl, 2,1,3-benzothiadiazolyl, and indolyl,
m is an integer from 0 to 7 inclusive,
the group(s) R₂, which may be identical or different, is (are) selected from (C₁-C₆)alkyl, halogen, —CN, —CF₃, —OCF₃, —NR₁₀R₁₁, —OR₁₀, —SR₁₀, —SO₂R₁₀, —(CH₂)ₖSO₂NR₁₀R₁₁, —X₅(CH₂)ₖC(═O)OR₁₀, —(CH₂)ₖC(═O)OR₁₀, —X₅(CH₂)ₖC(═O)NR₁₀R₁₁, —(CH₂)ₖC(═O)NR₁₀R₁₁, and —X₄—R₁₂ in which:
X₅ represents O, S or NH,
k is an integer from 0 to 3 inclusive,
R₁₀ and R₁₁, identical or different, are selected from hydrogen and (C₁-C₆)alkyl,
X₄ represents —CH₂—, or an oxygen atom,
R₁₂ represents a phenyl group which is unsubstituted or substituted with one or more groups, which may be identical or different, selected from (C₁-C₆)alkyl, halogen, hydroxyl and amino,
optionally, the racemic forms thereof, isomers thereof, N-oxydes thereof, and the pharmaceutically acceptable salts thereof.

4- A compound of formula (I) according to claim 1 characterized in that:
R₃ represents hydrogen, (C₁-C₆)alkyl or the group of formula: 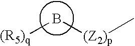
in which p is an integer from 0 to 3 inclusive,
Z₂ represents —CR₁₃R₁₄ wherein R₁₃ and R₁₄, independently of each other, represent a group selected from hydrogen, methyl, or phenyl, and
when p is greater than or equal to 2, the hydrocarbon chain Z₂ optionally contains one double bond,
or one of the carbon atoms in the hydrocarbon chain Z₂ may be replaced with an oxygen atom, a sulphur atom which is unsubstituted or substituted with one or two oxygen atoms, a nitrogen atom which is unsubstituted or substituted with a (C₁-C₆)alkyl, or a carbonyl group,
B represents a group selected from phenyl, pyridyl, thienyl, imidazolyl, furyl, 1,3-benzodioxolyl, benzodioxinyl, benzothienyl, benzofuryl, 2,1,3-benzothiadiazolyl, benzofurazanyl, naphthyl, and indolyl,
q is an integer from 0 to 3 inclusive,
the group(s) R₅, which may be identical or different, is (are) selected from (C₁-C₆)alkyl, halogen, CN, NO₂, CF₃, OCF₃, —(CH₂)ₖNR₁₅R₁₆, —N(R₁₅)C(═O)R₁₆, —N(R₁₅)C(═O)OR₁₆, —N(R₁₅)SO₂R₁₆, —N(SO₂R₁₅)₂, —OR₁₅, —S(O)ₖ₁R₁₅, —SO₂—N(R₁₅)—(CH₂)ₖ₂—NR₁₆R₁₇, —(CH₂)ₖSO₂NR₁₅R₁₆, —X₇(CH₂)ₖC(═O)OR₁₅, —(CH₂)ₖC(═O)OR₁₅, —C(═O)O—(CH₂)ₖ₂—NR₁₅R₁₆, —X₇(CH₂)ₖC(═O)NR₁₅R₁₆, and —(CH₂)ₖC(═O)NR₁₅R₁₆ in which:
X₇ is S, O or NH,
k is an integer from 0 to 3 inclusive,
k1 is an integer from 0 to 2 inclusive,
k2 is an integer from 1 to 4 inclusive,
R₁₅, R₁₆ and R₁₇, which may be identical or different, are selected from hydrogen and (C₁-C₆)alkyl,
optionally, the racemic forms thereof, isomers thereof, N-oxydes thereof, and the pharmaceutically acceptable salts thereof.

5- A compound of formula (I) according to claim 1 characterized in that:
R₁ represents a group selected from:
hydrogen, amino,
(C₁-C₆)alkyl, (C₃-C₆)alkenyl, (C₃-C₆)alkynyl, mono(C₁-C₆)alkylamino(C₁-C₆)alkyl, di(C₁-C₆)alkylamino(C₁-C₆)alkyl, aryl, aryl(C₁-C₆)alkyl, heterocycle, and 3- to 6-membered cycloalkyl(C₁-C₆)alkyl, these groups being unsubstituted or substituted with one or more groups, which may be identical or different, selected from amino, (C₁-C₆)alkyl, cyano, halo(C₁-C₆)alkyl, C(═O)OR₄, OR₄ and SR₄, in which R₄ represents hydrogen or (C₁-C₆)alkyl,
W represents an oxygen atom, a sulphur atom, or a group ═N—R′, in which R′ represents (C₁-C₆)alkyl, hydroxyl, or cyano,
X₁ represents a nitrogen atom or a group —C—R₆ in which R₆ represents a hydrogen atom,
X₂ and X₃ represent, independently of each other, a group —C—R₆ in which R₆ represents a group selected from hydrogen, (C₁-C₆)alkyl, amino, hydroxyl and halogen,
Y represents an oxygen atom,
Z represents an oxygen atom, or a group —NR₇ in which R₇ represents a group selected from hydrogen, and (C₁-C₆)alkyl,
n is an integer from 1 to 6 inclusive,
Z₁ represents —CR₈R₉ wherein R₈ and R₉, independently of each other, represent a group selected from hydrogen, (C₁-C₆)alkyl and hydroxyl, and
when n is greater than or equal to 2, the hydrocarbon chain Z₁ optionally contains one or more multiple bonds,
or one of the carbon atoms in the hydrocarbon chain Z₁ may be replaced with an oxygen atom, a sulphur atom which is unsubstituted or substituted with one or two oxygen atoms, or a nitrogen atom which is unsubstituted or substituted with a (C₁-C₆)alkyl,
A represents a group selected from phenyl, pyridyl, thienyl, imidazolyl, furyl, 1,3-benzodioxolyl, benzodioxinyl, benzothienyl, benzofuryl, benzofurazanyl, 2,1 ,3-benzothiadiazolyl, and indolyl;
m is an integer from 0 to 3 inclusive,
the group(s) R₂, which may be identical or different, is (are) selected from (C₁-C₆)alkyl, halogen, —CN, —CF₃, —OCF₃, —NR₁₀R₁₁, —OR₁₀, —SR₁₀, —SO₂R₁₀, —(CH₂)ₖSO₂NR₁₀R₁₁, —X₅(CH₂)ₖC(═O)OR₁₀, —(CH₂)ₖC(═O)OR₁₀, —X₅(CH₂)ₖC(═O)NR₁₀R₁₁, —(CH₂)ₖC(═O)NR₁₀R₁₁, and —X₄—R₁₂ in which:
X₅ represents O, S or NH,
k is an integer from 0 to 3 inclusive,
R₁₀ and R₁₁, which may be identical or different, are selected from hydrogen and (C₁-C₆)alkyl,
X₄ represents —CH₂—, or an oxygen atom,
R₁₂ represents phenyl which is unsubstituted or substituted with one or more groups, which may be identical or different, selected from (C₁-C₆)alkyl, halogen, and hydroxyl,
R₃ represents a group selected from hydrogen, (C₁-C₆)alkyl, and the group of formula: 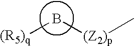
in which p is an integer from 0 to 6 inclusive,
Z₂ represents —CR₁₃R₁₄ wherein R₁₃ and R₁₄, independently of each other, represent a group selected from hydrogen, (C₁-C₆)alkyl, and hydroxy, and
when p is greater than or equal to 2, the hydrocarbon chain Z₂ optionally contains one or more multiple bonds,
or one of the carbon atoms in the hydrocarbon chain Z₂ may be replaced with an oxygen atom, a sulphur atom which is unsubstituted or substituted with one or two oxygen atoms, a nitrogen atom which is unsubstituted or substituted with a (C₁-C₆)alkyl,
B represents a group selected from phenyl, pyridyl, thienyl, imidazolyl, furyl, 1,3-benzodioxolyl, benzodioxinyl, benzothienyl, benzofuryl, 2,1,3-benzothiadiazolyl, benzofurazanyl, naphthyl, and indolyl,
q is an integer from 0 to 3 inclusive,
the group(s) R₅, which may be identical or different, is (are) selected from (C₁-C₆)alkyl, halogen, CN, NO₂, CF₃, OCF₃, —(CH₂)ₖNR₁₅R₁₆, —N(R₁₅)C(═O)R₁₆, —N(R₁₅)C(═O)OR₁₆, —N(R₁₅)SO₂R₁₆, —N(SO₂R₁₅)₂, —OR₁₅, —S(O)ₖ₁R₁₅, —SO₂—N(R₁₅)—(CH₂)ₖ₂—NR₁₆R₁₇, —(CH₂)ₖSO₂NR₁₅R₁₆, —X₇(CH₂)ₖC(═O)OR₁₅, —(CH₂)ₖC(═O)OR₁₅, —C(═O)O—(CH₂)ₖ₂—NR₁₅R₁₆, —X₇(CH₂)ₖC(═O)NR₁₅R₁₆, —(CH₂)ₖC(═O)NR₁₅R₁₆, and —X₆—R₂₀ in which:
X₇ is S, O or NH,
k is an integer from 0 to 3 inclusive,
k1 is an integer from 0 to 2 inclusive,
k2 is an integer from 1 to 4 inclusive,
R₁₅, R₁₆ and R₁₇, which may be identical or different, are selected from hydrogen and (C₁-C₆)alkyl,
X₆ represents a single bond, —CH₂—, an oxygen atom or a sulphur atom which is unsubstituted or substituted with one or two oxygen atom,
R₂₀ represents an aromatic or non-aromatic, heterocyclic or non-heterocyclic, 5- or 6-membered ring, which is unsubstituted or substituted with one or more groups, which may be identical or different, selected from (C₁-C₆)alkyl, halogen, hydroxyl, and amino, and, when the ring is heterocyclic, it comprises from 1 to 4 heteroatoms selected from nitrogen, oxygen and sulphur,
optionally, the racemic forms thereof, isomers thereof, N-oxydes thereof, and the pharmaceutically acceptable salts thereof.

6- A compound of formula (I) according to claim 1 characterized in that:
R₁ represents a group selected from hydrogen, mono(C₁-C₆)alkylamino(C₁-C₆)alkyl, di(C₁-C₆)alkylamino(C₁-C₆)alkyl, (C₁-C₆)alkyl, (C₃-C₆)alkenyl, (C₃-C₆)alkynyl, aryl, aryl(C₁-C₆)alkyl, and 3- to 6-membered cycloalkyl(C₁-C₆)alkyl,
W represents an oxygen atom, or a sulphur atom,
X₁ represents a nitrogen atom or a —CH group,
X₂ and X₃ represent a —CH group,
Y represents a group selected from oxygen atom, sulphur atom, —NH, and —N(C₁-C₆)alkyl,
Z represents an oxygen atom or a —NH group,
n is an integer from 1 to 3 inclusive,
Z₁ represents —CR₈R₉ wherein R₈ and R₉, independently of each other, represent a group selected from hydrogen, (C₁-C₆)alkyl and hydroxy, and
when n is greater than or equal to 2, the hydrocarbon chain Z₁ optionally contains one double bond,
or one of the carbon atoms in the hydrocarbon chain Z₁ may be replaced with an oxygen atom, a sulphur atom which is unsubstituted or substituted with one or two oxygen atoms, or a —NH group,
A represents a group selected from phenyl, pyridyl, thienyl, imidazolyl, furyl, 1,3-benzodioxolyl, benzodioxinyl, benzothienyl, benzofuryl, 2,1,3-benzothiadiazolyl, benzofurazanyl, naphthyl and indolyl,
m is an integer from 0 to 3 inclusive,
the group(s) R₂, which may be identical or different, is (are) selected from (C₁-C₆)alkyl, halogen, —CN, —CF₃, —OCF₃, —NR₁₀R₁₁, —OR₁₀, —SR₁₀, —SO₂R₁₀, —(CH₂)ₖSO₂NR₁₀R₁₁, —X₅(CH₂)ₖC(═O)OR₁₀, —(CH₂)ₖC(═O)OR₁₀, —X₅(CH₂)ₖC(═O)NR₁₀R₁₁, —(CH₂)ₖC(═O)NR₁₀R₁₁, and —X₄—R₁₂ in which:
X₅ represents O, S or NH,
k is an integer from 0 to 3 inclusive,
R₁₀ and R₁₁, which may be identical or different, are selected from hydrogen and (C₁-C₆)alkyl,
X₄ represents —CH₂—, or an oxygen atom,
R₁₂ represents phenyl which is unsubstituted or substituted with one or more groups, which may be identical or different, selected from (C₁-C₆)alkyl, halogen, and hydroxyl,
R₃ represents a group selected from methyl and the group of formula: 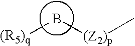
in which p is an integer from 0 to 3 inclusive,
Z₂ represents —CR₁₃R₁₄ wherein R₁₃ and R₁₄, independently of each other, represent a group selected from hydrogen, (C₁-C₆)alkyl, and hydroxy, and
when p is greater than or equal to 2, the hydrocarbon chain Z₂ optionally contains one double bond,
or one of the carbon atoms in the hydrocarbon chain Z₂ may be replaced with an oxygen atom, a sulphur atom which is unsubstituted or substituted with one or two oxygen atoms, a nitrogen atom which is unsubstituted or substituted with a (C₁-C₆)alkyl,
B represents a group selected from phenyl, pyridyl, thienyl, imidazolyl, furyl, 1,3-benzodioxolyl, benzodioxinyl, benzothienyl, benzofuryl, 2,1,3-benzothiadiazolyl, benzofurazanyl, naphthyl and indolyl,
q is an integer from 0 to 3 inclusive,
the group(s) R₅, which may be identical or different, is (are) selected from (C₁-C₆)alkyl, halogen, CN, NO₂, CF₃, OCF₃, —(CH₂)ₖNR₁₅R₁₆, —N(R₁₅)C(═O)R₁₆, —N(R₁₅)C(═O)OR₁₆, —N(R₁₅)SO₂R₁₆, —N(SO₂R₁₅)₂, —OR₁₅, —S(O)ₖ₁R₁₅, —SO₂—N(R₁₅)—(CH₂)ₖ₂—NR₁₆R₁₇, —(CH₂)ₖSO₂NR₁₅R₁₆, —X₇(CH₂)ₖC(═O)OR₁₅, —(CH₂)ₖC(═O)OR₁₅, —C(═O)O—(CH₂)ₖ₂—NR₁₅R₁₆, —X₇(CH₂)ₖC(═O)NR₁₅R₁₆, —(CH₂)ₖC(═O)NR₁₅R₁₆, and —X₆—R₂₀ in which:
X₇ is S, O or NH,
k is an integer from 0 to 3 inclusive,
k1 is an integer from 0 to 2 inclusive,
k2 is an integer from 1 to 4 inclusive,
R₁₅, R₁₆ and R₁₇, which may be identical or different, are selected from hydrogen and (C₁-C₆)alkyl,
X₆ represents a single bond, CH₂, an oxygen atom or a sulphur atom which is unsubstituted or substituted with one or two oxygen atom,
R₂₀ represents an aromatic or non-aromatic, heterocyclic or non-heterocyclic, 5- or 6-membered ring, which is unsubstituted or substituted with one or more groups, which may be identical or different, selected from (C₁-C₆)alkyl, halogen, hydroxyl, and amino, and, when the ring is heterocyclic, it comprises from 1 to 4 heteroatoms selected from nitrogen, oxygen and sulphur,
optionally, the racemic forms thereof, isomers thereof, N-oxydes thereof, and the pharmaceutically acceptable salts thereof.

7- A compound of formula (I) according to claim 1 characterized in that:
R₁ represents hydrogen, (C₁-C₆)alkyl, (C₃-C₆)alkenyl, aryl(C₁-C₆)alkyl, 3- to 6-membered cycloalkyl(C₁-C₆)alkyl,
W represents an oxygen atom,
X₁ represents —CH group or nitrogen atom , and X₂ and X₃ represent each —CH group;
Y represents an oxygen atom,
Z represents an oxygen atom or a —NH group,
n is an integer from 1 to 3 inclusive,
Z₁ represents —CR₈R₉ wherein R₈ and R₉, independently of each other, represent a group selected from hydrogen and methyl, and
when n is greater than or equal to 2, the hydrocarbon chain Z₁ optionally contains one double bond,
or one of the carbon atoms in the hydrocarbon chain Z₁ may be replaced with an oxygen atom, a sulphur atom which is unsubstituted or substituted with one or two oxygen atoms, or a —NH group,
A represents a group selected from phenyl, pyridyl, thienyl, imidazolyl, furyl, and 1,3-benzodioxolyl,
m is an integer from 0 to 3 inclusive,
the group(s) R₂, which may be identical or different, is (are) selected from (C₁-C₆)alkyl, halogen, —CN, —CF₃, —OCF₃, —NR₁₀R₁₁, —OR₁₀, —SR₁₀, —SO₂R₁₀, —(CH₂)ₖSO₂NR₁₀R₁₁, —X₅(CH₂)ₖC(═O)OR₁₀, —(CH₂)ₖC(═O)OR₁₀, —X₅(CH₂)ₖC(═O)NR₁₀R₁₁, and —(CH₂)ₖC(═O)NR₁₀OR₁₁, in which:
X₅ represents O, S or NH,
k is an integer from 0 to 3 inclusive,
R₁₀ and R₁₁, which may be identical or different, are selected from hydrogen and (C₁-C₆)alkyl,
R₃ represents the group of formula: 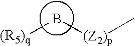
in which p is an integer from 0 to 3 inclusive,
Z₂ represents —CR₁₃R₁₄ wherein R₁₃ and R₁₄, independently of each other, represent a group selected from hydrogen, and methyl, and
when p is greater than or equal to 2, the hydrocarbon chain Z₂ optionally contains one double bond,
or one of the carbon atoms in the hydrocarbon chain Z₂ may be replaced with an oxygen atom, a sulphur atom which is unsubstituted or substituted with one or two oxygen atoms, a nitrogen atom which is unsubstituted or substituted with a (C₁-C₆)alkyl,
B represents a group selected from phenyl, pyridyl, thienyl, imidazolyl, furyl, and 1,3-benzodioxolyl,
q is an integer from 0 to 3 inclusive,
the group(s) R₅, which may be identical or different, is (are) selected from (C₁-C₆)alkyl, halogen, CN, NO₂, CF₃, OCF₃, —(CH₂)ₖNR₁₅R₁₆, —N(R₁₅)C(═O)R₁₆, —N(R₁₅)C(═O)OR₁₆, —N(R₁₅)SO₂R₁₆, —N(SO₂R₁₅)₂, —OR₁₅, —S(O)ₖ₁R₁₅, —SO₂—N(R₁₅)—(CH₂)ₖ₂—NR₁₆R₁₇, —(CH₂)ₖSO₂NR₁₅R₁₆, —X₇(CH₂)ₖC(═O)OR₁₅, —(CH₂)ₖC(═O)OR₁₅, —C(═O)O—(CH₂)ₖ₂—NR₁₅R₁₆, —X₇(CH₂)ₖC(═O)NR₁₅R₁₆, and —(CH₂)ₖC(═O)NR₁₅R₁₆, in which:
X₇ is S, O or NH,
k is an integer from 0 to 3 inclusive,
k1 is an integer from 0 to 2 inclusive,
k2 is an integer from 1 to 4 inclusive,
R₁₅, R₁₆ and R₁₇, which may be identical or different, are selected from hydrogen and (C₁-C₆)alkyl,
optionally, the racemic forms thereof, isomers thereof, N-oxydes thereof, and the pharmaceutically acceptable salts thereof.

8- A compound of formula (I) according to claim 1 characterized in that R₁ represents a hydrogen atom or a (C₁-C₆)alkyl group, optionally, the racemic forms thereof, isomers thereof, N-oxydes thereof, and the pharmaceutically acceptable salts thereof.

9- A compound of formula (I) according to claim 1 characterized in that:
W represents an oxygen atom,
Y represents an oxygen atom,
Z represents a NH group,
Z₁ represents a methylene group,
and n is equal to one,
optionally, the racemic forms thereof, isomers thereof, N-oxydes thereof, and the pharmaceutically acceptable salts thereof.

10- A compound of formula (I) according to claim 1 characterized in that:
X₁ represents a —CH group or a nitrogen atom,
and X₂ and X₃ represent each a —CH group,
optionally, the racemic forms thereof, isomers thereof, N-oxydes thereof, and the pharmaceutically acceptable salts thereof.

11- A compound of formula (I) according to claim 1 characterized in that:
X₁ and X₃ represent each a —CH group,
and X₂ represents a —CH group or a nitrogen atom,
optionally, the racemic forms thereof, isomers thereof, N-oxydes thereof, and the pharmaceutically acceptable salts thereof.

12- A compound of formula (I) according to claim 1 characterized in that:
X₁ and X₃ represent each a —CH group,
and X₂ represents a nitrogen atom,
optionally, the racemic forms thereof, isomers thereof, N-oxydes thereof, and the pharmaceutically acceptable salts thereof.

13- A compound of formula (I) according to claim 1 characterized in that:
A represents a group selected from phenyl, pyridyl, 1,3-benzodioxolyl, and benzofurazanyl,
m is equal to 0 or 1,
and R₂ represents a group selected from (C₁-C₆)alkoxy, hydroxy, halogen, and (C₁-C₆)thioalkoxy,
optionally, the racemic forms thereof, isomers thereof, N-oxydes thereof, and the pharmaceutically acceptable salts thereof.

14- A compound of formula (I) according to claim 1 characterized in that R₃ represents a group of formula: 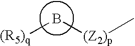
in which:
p is equal to 1,
Z₂ represents a methylen group,
B represents a group selected from phenyl, pyridyl, 1,3-benzodioxolyl, and benzofurazanyl,
q is an integer from 0 to 2 inclusive,
and R₅ represent(s) a group selected from halogen, CN, —(CH₂)ₖNR₁₅R₁₆, —S(O)ₖ₁R₁₅, —(CH₂)ₖSO₂NR₁₅R₁₆, —(CH₂)ₖC(═O)OR₁₅, —(CH₂)ₖC(═O)NR₁₅R₁₆, and —X₆—R₂₀, in which:
k is an integer from 0 to 1 inclusive,
k1 is an integer from 0 to 2 inclusive, p2 R₁₅ and R₁₆, which may be identical or different, are selected from hydrogen and (C₁-C₆)alkyl,
X₆ represents a bond,
—R₂₀ represents a 5-membered heterocyclic ring comprising from 3 to 4 heteroatoms selected from oxygen and nitrogen and optionally substituted with a methyl group or an oxo group,
optionally, the racemic forms thereof, isomers thereof, N-oxydes thereof, and the pharmaceutically acceptable salts thereof.

15- A compound of formula (I) according to claim 1, which is:
3-Benzyl-2,4-dioxo-1,2,3,4-tetrahydroquinazoline-6-carboxylic acid benzylamide,
3-Benzyl-2,4-dioxo-1,2,3,4-tetrahydroquinazoline-6-carboxylic acid (4-pyridylmethyl)amide,
3-Benzyl-2,4-dioxo-1,2,3,4-tetrahydroquinazoline-6-carboxylic acid (benzo[1,3]dioxol-5-ylmethyl)amide,
3-Benzyl-2,4-dioxo-1,2,3,4-tetrahydroquinazoline-6-carboxylic acid (2-thienylmethyl)amide,
3-Benzyl-2,4-dioxo-1,2,3,4-tetrahydroquinazoline-6-carboxylic acid (3-pyridylmethyl)amide,
3-Benzyl-2,4-dioxo-1,2,3,4-tetrahydroquinazoline-6-carboxylic acid 4-methoxybenzyl amide,
3-Benzyl-2,4-dioxo-1,2,3,4-tetrahydroquinazoline-6-carboxylic acid 4-chlorobenzyl amide,
3-Benzyl-2,4-dioxo-1,2,3,4-tetrahydroquinazoline-6-carboxylic acid 4-methylbenzyl amide,
3-Benzyl-1-methyl-2,4-dioxo-1,2,3,4-tetrahydroquinazoline-6-carboxylic acid (benzo[1,3]dioxol-5-ylmethyl)amide,
3-Benzyl-1-methyl-2,4-dioxo-1,2,3,4-tetrahydroquinazoline-6-carboxylic acid benzylamide,
Methyl 4-({[1-(3-benzyl-1-methyl-2,4-dioxo-1,2,3,4-tetrahydroquinazolin-6-yl)methanoyl]amino}methyl)benzoate,
3-Benzyl-1-methyl-2,4-dioxo-1,2,3,4-tetrahydroquinazoline-6-carboxylic acid 4-hydroxy-3-methoxybenzylamide,
3-Benzyl-1-methyl-2,4-dioxo-1,2,3,4-tetrahydroquinazoline-6-carboxylic acid 4-methoxy benzylamide,
3-Benzyl-1-methyl-2,4-dioxo-1,2,3,4-tetrahydroquinazoline-6-carboxylic acid (4-pyridylmethyl)amide,
1-Methyl-2,4-dioxo-3-phenethyl-1,2,3,4-tetrahydroquinazoline-6-carboxylic acid (benzo[1,3]dioxol-5-ylmethyl)amide,
3-(4-Methoxybenzyl)-2,4-dioxo-1,2,3 ,4-tetrahydroquinazoline-6-carboxylic acid (benzo[1,3]dioxol-5-ylmethyl)amide,
3-(4-Methoxybenzyl)-1-methyl-2,4-dioxo-1,2,3,4-tetrahydroquinazoline-6-carboxylic acid (benzo[1,3]dioxol-5-ylmethyl)amide,
3-(4-Methoxybenzyl)-1-methyl-2,4-dioxo-1,2,3,4-tetrahydroquinazoline-6-carboxylic acid 4-methoxybenzylamide,
3-(1-Naphth-1-ylethyl)-2,4-dioxo-1,2,3,4-tetrahydroquinazoline-6-carboxylic acid (benzo[1,3]dioxol-5-ylmethyl)amide,
2,4-Dioxo-3-(pyrid-4-ylmethyl)-1,2,3,4-tetrahydroquinazoline-6-carboxylic acid (benzo[1,3]dioxol-5-ylmethyl)amide,
2,4-Dioxo-3-(thien-2-ylmethyl)-1,2,3,4-tetrahydroquinazoline-6-carboxylic acid benzylamide,
1-Methyl-2,4-dioxo-3-(thien-2-ylmethyl)-1,2,3,4-tetrahydroquinazoline-6-carboxylic acid benzylamide,
2,4-Dioxo-3-(thien-2-ylmethyl)-1,2,3,4-tetrahydroquinazoline-6-carboxylic acid (benzo[1,3]dioxol-5-ylmethyl)amide,
1-Methyl-2,4-dioxo-3-(thien-2-ylmethyl)-1,2,3,4-tetrahydroquinazoline-6-carboxylic acid (benzo[1,3]dioxol-5-ylmethyl)amide,
3-(4-Chlorobenzyl)-2,4-dioxo-1,2,3,4-tetrahydroquinazoline-6-carboxylic acid (benzo[1,3]dioxol-5-ylmethyl)amide,
3-(4-Chlorobenzyl)-1-methyl-2,4-dioxo-1,2,3,4-tetrahydroquinazoline-6-carboxylic acid (benzo[1,3]dioxol-5-ylmethyl)amide,
1,3-Dimethyl-2,4-dioxo-1,2,3,4-tetrahydroquinazoline-6-carboxylic acid benzo[1,3]dioxol-5-ylmethyl)amide,
3-(Benzo[1,3]dioxol-5-ylmethyl)-2,4-dioxo-1,2,3,4-tetrahydroquinazoline -6-carboxylic acid (benzo[1,3]dioxol-5-ylmethyl)amide,
3-(Benzo[1,3]dioxol-5-ylmethyl)-1-methyl-2,4-dioxo-1,2,3,4-tetrahydroquinazoline-6-carboxylic acid (benzo[ 1,3]dioxol-5-ylmethyl)amide,
3-Benzyl-1-ethyl-2,4-dioxo-1,2,3,4-tetrahydroquinazoline-6-carboxylic acid (benzo[1,3]dioxol-5-ylmethyl)amide,
3-Benzyl-1-cyclopropylmethyl-2,4-dioxo-1,2,3,4-tetrahydroquinazoline-6-carboxylic acid (benzo[1,3]dioxol-5-ylmethyl)amide,
3-Benzyl-1-isobutyl-2,4-dioxo-1,2,3,4-tetrahydroquinazoline-6-carboxylic acid (benzo[1,3]dioxol-5-ylmethyl)amide,
1-Methyl-2,4-dioxo-1,2,3,4-tetrahydroquinazoline-6-carboxylic acid (benzo[1,3]dioxol-5-ylmethyl)amide,
Methyl 4-[6-(4-methoxy-benzylcarbamoyl)-1-methyl-2,4-dioxo-1,4-dihydro-2H-quinazolin-3-ylmethyl]-benzoate,
4-[6-(4-Methoxy-benzylcarbamoyl)-1-methyl-2,4-dioxo-1,4-dihydro-2H]-quinazolin-3-ylmethyl]-benzoic acid,
1-Methyl-2,4-dioxo-3-((E)-3-phenylallyl)-1,2,3,4-tetrahydroquinazoline-6-carboxylic acid (benzo[1,3]dioxol-5-ylmethyl)amide,
Benzyl 3-benzyl-2,4-dioxo-1,2,3,4-tetrahydroquinazoline-6-carboxylate,
Benzyl 3-benzyl-1-methyl-2,4-dioxo-1,2,3,4-tetrahydroquinazoline-6-carboxylate,
4-Pyridylmethyl 3-benzyl-2,4-dioxo-1,2,3,4-tetrahydroquinazoline-6-carboxylate,
4-Pyridylmethyl 3-benzyl-1-methyl-2,4-dioxo-1,2,3,4-tetrahydroquinazoline -6-carboxylate,
Benzo[1,3]dioxol-5-ylmethyl 3-benzyl-2,4-dioxo-1,2,3,4-tetrahydroquinazoline-6-carboxylate,
Benzo[1,3]dioxol-5-ylmethyl 3-benzyl-1-methyl-2,4-dioxo-1,2,3,4-tetrahydro quinazoline -6-carboxylate,
Benzyl 1-benzyl-2,4-dioxo-3-pyrid-4-ylmethyl-1,2,3,4-tetrahydroquinazoline-6-carboxylate,
4-Pyridylmethyl 2,4-dioxo-3-(thien-2-ylmethyl)-1,2,3,4-tetrahydroquinazoline-6-carboxylate,
4-Pyridylmethyl 3-(benzo[1,3]dioxol-5-ylmethyl)-2,4-dioxo-1,2,3,4-tetrahydro quinazoline-6-carboxylate,
Benzyl 3-benzyl-2,4-dioxo-1,2,3,4-tetrahydropyrido[2,3-d]pyrimidine-6-carboxylate
4-Pyridylmethyl 3-benzyl-2,4-dioxo-1,2,3,4-tetrahydropyrido[2,3-d]pyrimidine-6-carboxylate,
3-Benzyl-4-oxo-2-thioxo-1,2,3,4-tetrahydroquinazoline-6-carboxylic acid (benzo[1,3]dioxol-5-ylmethyl)amide,
4-[6-(4-Hydroxy-benzylcarbamoyl)-1-methyl-2,4-dioxo-1,4-dihydro-2H-quinazolin-3-ylmethyl]-benzoic acid,
3-(4-Dimethylcarbamoyl-benzyl)-1-methyl-2,4-dioxo-1,2,3,4-tetrahydroquinazoline-6-carboxylic acid 4-methoxy-benzylamide,
1-Methyl-3-(4-methylcarbamoyl-benzyl)-2,4-dioxo-1,2,3,4-tetrahydro-quinazoline-6-carboxylic acid 4-methoxy-benzylamide,
3-Allyl-1-methyl-2,4-dioxo-1,2,3,4-tetrahydro-quinazoline-6-carboxylic acid 4-methoxy-benzylamide,
1-Methyl-2,4-dioxo-3-(2-pyrrol-1-yl-ethyl)-1,2,3,4-tetrahydro-quinazoline-6-carboxylic acid 4-methoxy-benzylamide,
1-Methyl-2,4-dioxo-3-prop-2-ynyl-1,2,3,4-tetrahydro-quinazoline-6-carboxylic acid 4-methoxy-benzylamide,
1-Methyl-3-(3-methyl-but-2-enyl)-2,4-dioxo-1,2,3,4-tetrahydro-quinazoline-6-carboxylic acid 4-methoxy-benzylamide,
1-Methyl-2,4-dioxo-3-pyridin-2-ylmethyl-1,2,3,4-tetrahydro-quinazoline-6-carboxylic acid 4-methoxy-benzylamide,
3-Carbamoylmethyl-1-methyl-2,4-dioxo-1,2,3,4-tetrahydro-quinazoline-6-carboxylic acid 4-methoxy-benzylamide,
1-Methyl-2,4-dioxo-3-pyridin-3-ylmethyl-1,2,3,4-tetrahydro-quinazoline-6-carboxylic acid 4-methoxy-benzylamide,
1-Methyl-3-(1-methyl-piperidin-3-ylmethyl)-2,4-dioxo-1,2,3,4-tetrahydroquinazoline-6-carboxylic acid 4-methoxy-benzylamide,
3-(4-Cyano-benzyl)-1-methyl-2,4-dioxo-1,2,3,4-tetrahydro-quinazoline-6-carboxylic acid 4-methoxy-benzylamide,
3-(3-Cyano-benzyl)-1-methyl-2,4-dioxo-1,2,3,4-tetrahydro-quinazoline-6-carboxylic acid 4-methoxy-benzylamide,
3-(2-Methoxy-ethyl)-1-methyl-2,4-dioxo-1,2,3,4-tetrahydro-quinazoline-6-carboxylic acid 4-methoxy-benzylamide,
3-(3-Methoxy-benzyl)-1-methyl-2,4-dioxo-1,2,3,4-tetrahydro-quinazoline-6-carboxylic acid 4-methoxy-benzylamide,
3-Cyclopropylmethyl-1-methyl-2,4-dioxo-1,2,3,4-tetrahydro-quinazoline-6-carboxylic acid 4-methoxy-benzylamide,
1-Methyl-3-(2-morpholin-4-yl-ethyl)-2,4-dioxo-1,2,3,4-tetrahydro-quinazoline-6-carboxylic acid 4-methoxy-benzylamide,
3-Cyclohexylmethyl-1-methyl-2,4-dioxo-1,2,3,4-tetrahydro-quinazoline-6-carboxylic acid 4-methoxy-benzylamide,
1-Methyl-2,4-dioxo-3-(3-phenyl-propyl)-1,2,3,4-tetrahydro-quinazoline-6-carboxylic acid 4-methoxy-benzylamide,
3-(4-Fluoro-benzyl)-1-methyl-2,4-dioxo-1,2,3,4-tetrahydro-quinazoline-6-carboxylic acid 4-methoxy-benzylamide,
3-[2-(4-Diethylamino-phenyl)-2-oxo-ethyl]-1-methyl-2,4-dioxo-1,2,3,4-tetrahydroquinazoline-6-carboxylic acid 4-methoxy-benzylamide,
Ethyl [6-(4-methoxy-benzylcarbamoyl)-1-methyl-2,4-dioxo-1,4-dihydro-2H-quinazolin-3-yl]-acetate,
3-(2-Hydroxy-ethyl)-1-methyl-2,4-dioxo-1,2,3,4-tetrahydroquinazoline-6-carboxylic acid 4-methoxy-benzylamide,
Methyl 3-[6-(4-methoxy-benzylcarbamoyl)-1-methyl-2,4-dioxo-1,4-dihydro-2H-quinazolin-3-yl]-propionate,
3-[6-(4-Methoxy-benzylcarbamoyl)-1-methyl-2,4-dioxo-1,4-dihydro-2H-quinazolin-3-yl]-propionic acid,
Ethyl 4-[6-(4-methoxy-benzylcarbamoyl)-1-methyl-2,4-dioxo-1,4-dihydro-2H-quinazolin-3-yl]-butyrate,
4-[6-(4-Methoxy-benzylcarbamoyl)-1-methyl-2,4-dioxo-1,4-dihydro-2H-quinazolin-3-yl]-butyric acid,
Methyl {4-[6-(4-methoxy-benzylcarbamoyl)-1-methyl-2,4-dioxo-1,4-dihydro-2H-quinazolin-3-ylmethyl]-phenyl}-acetate,
{4-[6-(4-Methoxy-benzylcarbamoyl)-1-methyl-2,4-dioxo-1,4-dihydro-2H-quinazolin-3-ylmethyl]-phenyl}-acetic acid,
3-(4-Dimethylcarbamoylmethyl-benzyl)-1-methyl-2,4-dioxo-1,2,3,4-tetrahydroquinazoline-6-carboxylic acid 4-methoxy-benzylamide,
1-Methyl-2,4-dioxo-3-[(E)-3-(pyridin-3-yl)-allyl]-1,2,3,4-tetrahydro-quinazoline-6-carboxylic acid 4-methoxy-benzylamide,
1-Methyl-2,4-dioxo-3-[(E)-3-(pyridin-4-yl)-allyl]-1,2,3,4-tetrahydro-quinazoline-6-carboxylic acid 4-methoxy-benzylamide,
1-Methyl-2,4-dioxo-3-(4-sulfamoyl-benzyl)-1,2,3,4-tetrahydroquinazoline-6-carboxylic acid 4-methoxy-benzylamide,
3-(4-Methanesulfonyl-benzyl)-1-methyl-2,4-dioxo-1,2,3,4-tetrahydro-quinazoline-6-carboxylic acid 4-methoxy-benzylamide,
3-(4-Dimethylsulfamoyl-benzyl)-1-methyl-2,4-dioxo-1,2,3,4-tetrahydro-quinazoline-6-carboxylic acid 4-methoxy-benzylamide,
3-[4-(2-Dimethylamino-ethylsulfamoyl)-benzyl]-1-methyl-2,4-dioxo-1,2,3,4-tetrahydroquinazoline-6-carboxylic acid 4-methoxy-benzylamide,
1-Methyl-3-(4-methylsulfamoyl-benzyl)-2,4-dioxo-1,2,3,4-tetrahydro-quinazoline-6-carboxylic acid 4-methoxy-benzylamide,
Methyl 3-[6-(4-Methoxy-benzylcarbamoyl)-1-methyl-2,4-dioxo-1,4-dihydro-2H-quinazolin-3-ylmethyl]-benzoate,
3-[6-(4-Methoxy-benzylcarbamoyl)-1-methyl-2,4-dioxo-1,4-dihydro-2H-quinazolin-3-ylmethyl]-benzoic acid,
(E) Methyl-4-[6-(4-methoxy-benzylcarbamoyl)-1-methyl-2,4-dioxo-1,4-dihydro-2H-quinazolin-3-yl]-but-2-enoate,
4-[6-(4-Methoxy-benzylcarbamoyl)-1-methyl-2,4-dioxo-1,4-dihydro-2H-quinazolin-3-yl]-but-2-enoic acid,
Methyl 5-[6-(4-methoxy-benzylcarbamoyl)-1-methyl-2,4-dioxo-1,4-dihydro-2H-quinazolin-3-ylmethyl]-furan-2-carboxylate,
5-[6-(4-Methoxy-benzylcarbamoyl)-1-methyl-2,4-dioxo-1,4-dihydro-2H-quinazolin-3-ylmethyl]-furan-2-carboxylic acid,
Methyl 5-[6-(4-methoxy-benzylcarbamoyl)-1-methyl-2,4-dioxo-1,4-dihydro-2H-quinazolin-3-ylmethyl]-thiophene-2-carboxylate,
5-[6-(4-Methoxy-benzylcarbamoyl)-1-methyl-2,4-dioxo-1,4-dihydro-2H-quinazolin-3-ylmethyl]-thiophene-2-carboxylic acid,
1-Methyl-3-(4-nitro-benzyl)-2,4-dioxo-1,2,3,4-tetrahydro-quinazoline-6-carboxylic acid 4-methoxy-benzylamide,
3-(4-Amino-benzyl)-1-methyl-2,4-dioxo-1,2,3,4-tetrahydro-quinazoline-6-carboxylic acid 4-methoxy-benzylamide,
3-(4-Dimethylamino-benzyl)-1-methyl-2,4-dioxo-1,2,3,4-tetrahydro-quinazoline-6-carboxylic acid 4-methoxy-benzylamide,
3-(4-Acetylamino-benzyl)-1-methyl-2,4-dioxo-1,2,3,4-tetrahydro-quinazoline-6-carboxylic acid 4-methoxy-benzylamide,
3-[4-(N,N-methylsulfonylamino)-benzyl]-1-methyl-2,4-dioxo-1,2,3,4-tetrahydroquinazoline-6-carboxylic acid 4-methoxy-benzylamide,
3-Benzofurazan-5-ylmethyl-1-methyl-2,4-dioxo-1,2,3,4-tetrahydro-quinazoline-6-carboxylic acid 4-methoxy-benzylamide,
3-[2-(4-Fluorophenoxy)-ethyl]-1-methyl-2,4-dioxo-1,2,3,4-tetrahydro-quinazoline-6-carboxylic acid 4-methoxy-benzylamide,
3-(2-Benzenesulfonyl-ethyl)-1-methyl-2,4-dioxo-1,2,3,4-tetrahydro-quinazoline-6-carboxylic acid 4-methoxy-benzylamide,
3-(3-fluoro-4-methoxy-benzyl)-1-methyl-2,4-dioxo-1,2,3,4-tetrahydro-quinazoline-6-carboxylic acid 4-methoxy benzylamine,
1-Methyl-2,4-dioxo-3-[4-(2H-tetrazol-5-yl)-benzyl]-1,2,3,4-tetrahydro-quinazoline-6-carboxylic acid 4-methoxy-benzylamide,
1-Methyl-3-[4-(5-methyl-1,2,4-oxadiazol-3-yl)-benzyl]-2,4-dioxo-1,2,3,4-tetrahydroquinazoline-6-carboxylic acid 4-methoxy-benzylamide,
1-Methyl-3-[4-(3-methyl-1,2,4-oxadiazol-5-yl)-benzyl]-2,4-dioxo-1,2,3,4-tetrahydroquinazoline-6-carboxylic acid 4-methoxy-benzylamide,
Methyl 2-chloro-4-[6-(4-methoxy-benzylcarbamoyl)-1-methyl-2,4-dioxo-1,4-dihydro-2H-quinazolin-3-ylmethyl]-benzoate,
2-Chloro-4-[6-(4-methoxy-benzylcarbamoyl)-1-methyl-2,4-dioxo-1,4-dihydro-2H-quinazolin-3-ylmethyl]-benzoic acid,
1-Methyl-3-[4-(1-methyl-1H-tetrazol-5-yl)-benzyl]-2,4-dioxo-1,2,3,4-tetrahydroquinazoline-6-carboxylic acid 4-methoxy-benzylamide,
1-Methyl-3-[4-(2-methyl-2H-tetrazol-5-yl)-benzyl]-2,4-dioxo-1,2,3,4-tetrahydroquinazoline-6-carboxylic acid 4-methoxy-benzylamide,
Methyl 2-methoxy-4-[6-(4-methoxy-benzylcarbamoyl)-1-methyl-2,4-dioxo-1,4-dihydro-2H-quinazolin-3-ylmethyl]-benzoate,
2-Methoxy-4-[6-(4-methoxy-benzylcarbamoyl)-1-methyl-2,4-dioxo-1,4-dihydro-2H-quinazolin-3-ylmethyl]-benzoic acid,
Methyl 2-hydroxy-4-[6-(4-methoxy-benzylcarbamoyl)-1-methyl-2,4-dioxo-1,4-dihydro-2H-quinazolin-3-ylmethyl]-benzoate,
2-Hydroxy-4-[6-(4-methoxy-benzylcarbamoyl)-1-methyl-2,4-dioxo-1,4-dihydro-2H-quinazolin-3-ylmethyl]-benzoic acid,
Methyl 2-methyl-4-[6-(4-methoxy-benzylcarbamoyl)-1-methyl-2,4-dioxo-1,4-dihydro-2H-quinazolin-3-ylmethyl]-benzoate,
2-Methyl-4-[6-(4-methoxy-benzylcarbamoyl)-1-methyl-2,4-dioxo-1,4-dihydro-2H-quinazolin-3-ylmethyl]-benzoic acid,
1-Methyl-2,4-dioxo-3-(pyridin-4-methyl)-1,2,3,4-tetrahydro-quinazoline-carboxylic acid (benzo[1,3]dioxol-5-ylmethyl)-amide,
1-Methyl-2,4-dioxo-3-(pyridin-4-ylmethyl)-1,2,3,4-tetrahydro-quinazoline-carboxylic acid 4-methoxy-benzylamide,
1-Methyl-2,4-dioxo-3-(pyridin-4-ylmethyl)-1,2,3,4-tetrahydro-quinazoline-6-carboxylic acid 4-hydroxy-benzylamide,
Methyl 4-[6-(3-methoxy-benzylcarbamoyl)-1-methyl-2,4-dioxo-1,4-dihydro-2H-quinazolin-3-ylmethyl]-benzoate,
4-[6-(3-Methoxy-benzylcarbamoyl)-1-methyl-2,4-dioxo-1,4-dihydro-2H-quinazolin-3-ylmethyl]-benzoic acid,
Methyl 4-[1-methyl-6-(4-methylsulfanyl-benzylcarbamoyl)-2,4-dioxo-1,4-dihydro-2H-quinazolin-3-ylmethyl]-benzoate,
4-[1-Methyl-6-(4-methylsulfanyl-benzylcarbamoyl)-2,4-dioxo-1,4-dihydro-2H-quinazolin-3-ylmethyl]-benzoic acid,
Methyl 4-[1-ethyl-2,4-dioxo-6-(4-trifluoromethoxy-benzylcarbamoyl)-1,4-dihydro-2H-quinazolin-3-ylmethyl]-benzoate,
Methyl 4-[6-(4-fluoro-benzylcarbamoyl)-1-methyl-2,4-dioxo-1,4-dihydro-2H-quinazolin-3-ylmethyl]-benzoate,
4-[6-(4-Fluoro-benzylcarbamoyl)-1-methyl-2,4-dioxo-1,4-dihydro-2H-quinazolin-3-ylmethyl]-benzoic acid,
Methyl 4-{6-[(benzofurazan-5-ylmethyl)-carbamoyl]-1-methyl-2,4-dioxo-1,4-dihydro-2H-quinazolin-3-ylmethyl}-benzoate,
4-{6-[(Benzofurazan-5-ylmethyl)-carbamoyl]-1-methyl-2,4-dioxo-1,4-dihydro-2H-quinazolin-3-ylmethyl}-benzoic acid,
Methyl 4-[6-(4-methoxy-benzylcarbamoyl)-2,4-dioxo-1,4-dihydro-2H-quinazolin-3-ylmethyl]-benzoate,
Methyl 4-[1-ethyl-6-(4-methoxy-benzylcarbamoyl)-2,4-dioxo-1,4-dihydro-2H-quinazolin-3-ylmethyl]-benzoate,
4-[1-Ethyl-6-(4-methoxy-benzylcarbamoyl)-2,4-dioxo-1,4-dihydro-2H-quinazolin-3-ylmethyl]-benzoic acid,
3-(4-Methoxy-benzyl)-1-methyl-2,4-dioxo-1,2,3,4-tetrahydroquinazoline-6-carboxylic acid (pyridin-4-ylmethyl)-amide,
3-(4-Hydroxy-benzyl)-1-methyl-2,4-dioxo-1,2,3,4-tetrahydro-quinazoline-6-carboxylic acid (pyridin-4-ylmethyl)-amide,
3-(4-Cyano-benzyl)-1-methyl-2,4-dioxo-1,2,3,4-tetrahydro-quinazoline-6-carboxylic acid (pyridin-4-ylmethyl)-amide,
1-Methyl-2,4-dioxo-3-(3-pyridin-4-yl-allyl)-1,2,3,4-tetrahydro-quinazoline-6-carboxylic acid (pyridin-4-ylmethyl)-amide,
Methyl-4-{1-methyl-2,4-dioxo-6-[(pyridin-4-ylmethyl)-carbamoyl]-1,4-dihydro-2H-quinazolin-3-ylmethyl}-benzoate,
4-{1-Methyl-2,4-dioxo-6-[(pyridin-4-ylmethyl)-carbamoyl]-1,4-dihydro-2H-quinazolin-3-ylmethyl}-benzoic acid,
Methyl (4-{1-methyl-2,4-dioxo-6-[(pyridin-4-ylmethyl)-carbamoyl]-1,4-dihydro-2H-quinazolin-3-ylmethyl}-phenyl)-acetate,
(4-{1-Methyl-2,4-dioxo-6-[(pyridin-4-ylmethyl)-carbamoyl]-1,4-dihydro-2H-quinazolin-3-ylmethyl}-phenyl)-acetic acid,
Methyl 4-{1-methyl-2,4-dioxo-6-[(1-oxy-pyridin-4-ylmethyl)carbamoyl]-1,4-dihydro-2H-quinazolin-3-ylmethyl}-benzoate,
4-{1-Methyl-2,4-dioxo-6-[(1-oxy-pyridin-4-ylmethyl)-carbamoyl]-1,4-dihydro-2H-quinazolin-3-ylmethyl}-benzoic acid,
Methyl {6-[(1,3-Benzodioxol-5-ylmethyl)-carbamoyl]-3-benzyl-2,4-dioxo-1,4-dihydro-2H-quinazolin-1-yl}-acetate,
{6-[(1,3-Benzodioxol-5-ylmethyl)-carbamoyl]-3-benzyl-2,4-dioxo-3,4-dihydro-2H-quinazolin-1-yl}-acetic acid,
Methyl 4-{6-[(1,3-benzodioxol-5-ylmethyl)-carbamoyl]-1-methyl-2,4-dioxo-1,4-dihydro -2H-quinazolin-3-ylmethyl}-benzoate,
4-{6-[(1,3-Benzodioxol-5-ylmethyl)-carbamoyl]-1-methyl-2,4-dioxo-1,4-dihydro-2H-quinazolin-3-ylmethyl}-benzoic acid,
3-Benzyl-1-methyl-2,4-dioxo-1,2,3,4-tetrahydro-quinazoline-6-carboxylic acid 4-sulfamoyl-benzylamide,
3-Benzyl-1-methyl-2,4-dioxo-1,2,3,4-tetrahydro-quinazoline-6-carboxylic acid[3-(pyridin-4-ylsulfanyl)-propyl]-amide,
3-Benzyl-1-methyl-2,4-dioxo-1,2,3,4-tetrahydro-quinazoline-6-carboxylic acid (4-morpholin-4-yl-butyl)-amide,
3-Benzyl-1-methyl-2,4-dioxo-1,2,3,4-tetrahydro-quinazoline-6-carboxylic acid (1-benzyl-piperidin-4-yl)-amide,
3-Benzyl-1-methyl-2,4-dioxo-1,2,3,4-tetrahydro-quinazoline-6-carboxylic acid 4-hydroxy-benzylamine,
Ethyl (4-{[(3-benzyl-1-methyl-2,4-dioxo-1,2,3,4-tetrahydro-quinazoline-6-carbonyl)-amino]-methyl}-phenoxy)-acetate,
(4-{[(3-Benzyl-1-methyl-2,4-dioxo-1,2,3,4-tetrahydro-quinazoline-6-carbonyl)amino]-methyl}-phenoxy)-acetic acid,
3-Benzyl-1-methyl-2,4-dioxo-1,2,3,4-tetrahydro-quinazoline-6-carboxylic acid 4-dimethylcarbamoylmethoxy-benzylamide,
3-Benzyl-1-methyl-2,4-dioxo-1,2,3,4-tetrahydro-quinazoline-6-carboxylic acid (3-phenyl-allyl)-amide,
3-Benzyl-1-methyl-2,4-dioxo-1,2,3,4-tetrahydro-quinazoline-6-carboxylic acid 4-cyano-benzylamide,
4-{[(3-Benzyl-1-methyl-2,4-dioxo-1,2,3,4-tetrahydro-quinazoline-6-carbonyl)-amino]-methyl}-benzoic acid,
3-Benzyl-1-methyl-2,4-dioxo-1,2,3,4-tetrahydro-quinazoline-6-carboxylic acid 4-dimethylcarbamoyl-benzylamide,
3-(4-Dimethylamino-benzyl)-2,4-dioxo-1,2,3,4-tetrahydro-quinazoline-6-carboxylic acid 4-methoxy-benzylamide,
3-[4-(N-methylsulfonylamino)-benzyl]-1-methyl-2,4-dioxo-1,2,3,4-tetrahydroquinazoline-6-carboxylic acid 4-methoxy-benzylamide,
tert-Butyl{5-[6-(4-Methoxy-benzylcarbamoyl)-1-methyl-2,4-dioxo-1,4-dihydro-2H-quinazolin-3-ylmethyl]-pyridin-2-yl}-carbamate,
3-(6-Amino-pyridin-3-ylmethyl)-1-methyl-2,4-dioxo-1,2,3,4-tetrahydro-quinazoline-6-carboxylic acid 4-methoxy-benzylamide,
1,3-Dimethyl-2,4-dioxo-1,2,3,4-tetrahydro-pyrido[2,3-d]pyrimidine-6-carboxylic acid (1,3-benzodioxol-5-ylmethyl)-amide,
1,3-Dimethyl-2,4-dioxo-1,2,3,4-tetrahydro-pyrido[3,4-d]pyrimidine-6-carboxylic acid (1,3-benzodioxol-5-ylmethyl)-amide,
3-Benzyl-1-methyl-2,4-dioxo-1,2,3,4-tetrahydro-pyrido[2,3-d]pyrimidine-6-carboxylic acid (1,3-benzodioxol-5-ylmethyl)-amide,
4-[6-(4-Methoxy-benzylcarbamoyl)-1-methyl-2,4-dioxo-1,4-dihydro-2H-pyrido[2,3-d]pyrimidin-3-ylmethyl]-benzoic acid,
3-(4-Cyano-benzyl)-1-methyl-2,4-dioxo-1,2,3,4-tetrahydro-pyrido[2,3-d]pyrimidine-6-carboxylic acid 4-methoxy-benzylamide,
3-(4-Fluoro-benzyl)-1-methyl-2,4-dioxo-1,2,3,4-tetrahydro-pyrido[2,3-d]pyrimidine-6-carboxylic acid 4-methoxy-benzylamide,
3-Benzyl-1-methyl-2,4-dioxo-1,2,3,4-tetrahydro-pyrido[3,4-d]pyrimidine-6-carboxylic acid (1,3-benzodioxol-5-ylmethyl)-amide,
Methyl 4-[6-(4-Methoxy-benzylcarbamoyl)-1-methyl-2,4-dioxo-1,4-dihydro-2H-pyrido[3,4-d]pyrimidin-3-ylmethyl]-benzoate,
4-[6-(4-Methoxy-benzylcarbamoyl)-1-methyl-2,4-dioxo-1,4-dihydro-2H-pyrido[3,4-d]pyrimidin-3-ylmethyl]-benzoic acid,
4-[6-(3-Methoxy-benzylcarbamoyl)-1-methyl-2,4-dioxo-1,4-dihydro-2H-pyrido[3,4-d]pyrimidin-3-ylmethyl]-benzoic acid,
3-(4-Cyano-benzyl)-1-methyl-2,4-dioxo-1,2,3,4-tetrahydro-pyrido[3,4-d]pyrimidine-6-carboxylic acid 4-methoxy-benzylamide,
3-Benzyl-1-methyl-6-(3-phenyl-propionyl)-1H-quinazoline-2,4-dione,
3-Benzyl-1-methyl-2,4-dioxo-1,2,3,4-tetrahydro-quinazoline-6-carboxylic acid (E)-3-pyridin-4-yl-allyl ester,
3-Benzyl-1-methyl-2,4-dioxo-1,2,3,4-tetrahydro-quinazoline-6-carboxylic acid (E)-3-pyridin-3-yl-allyl ester,
3-Benzyl-1-methyl-6-[2-(pyridin-4-ylsulfanyl)-acetyl]-1H-quinazoline-2,4-dione,
3-(4-Aminomethyl-benzyl)-1-methyl-2,4-dioxo-1,2,3,4-tetrahydro-quinazoline-6-carboxylic acid 4-methoxy-benzylamide,
3-(2′-Cyano-biphenyl-4-ylmethyl)-1-methyl-2,4-dioxo-1,2,3,4-tetrahydroquinazoline-6-carboxylic acid 4-methoxy-benzylamide,
1-(Methyl-2,4-dioxo-3-[2′-(1H-tetrazol-5-yl)-biphenyl-4-ylmethyl]-1,2,3,4-tetrahydroquinazoline-6-carboxylic acid 4-methoxy-benzylamide,
Methyl 4′-[6-(4-methoxy-benzylcarbamoyl)-1-methyl-2,4-dioxo-1,4-dihydro-2H-quinazolin-3-ylmethyl]-biphenyl-2-carboxylate,
4′-[6-(4-Methoxy-benzylcarbamoyl)-1-methyl-2,4-dioxo-1,4-dihydro-2H-quinazolin-3-ylmethyl]-biphenyl-2-carboxylic acid,
Ethyl 2-Fluoro-4-[6-(4-methoxy-benzylcarbamoyl)-1-methyl-2,4-dioxo-1,4-dihydro-2H-quinazolin-3-ylmethyl]-benzoate,
2-Fluoro-4-[6-(4-methoxy-benzylcarbamoyl)-1-methyl-2,4-dioxo-1,4-dihydro-2H-quinazolin-3-ylmethyl]-benzoic acid,
2-Methoxy-4-[6-(4-methoxy-benzylcarbamoyl)-1-methyl-2,4-dioxo-1,4-dihydro-2H-quinazolin-3-ylmethyl]-benzoic acid 2-dimethylamino-ethyl ester,
4-[6-(4-Methoxy-benzylcarbamoyl)-1-methyl-2,4-dioxo-1,4-dihydro-2H-quinazolin-3-ylmethyl]-2-methyl-benzoic acid 2-dimethylamino-ethyl ester,
1-Methyl-2,4-dioxo-3-[4-(5-oxo-4,5-dihydro-1,2,4-oxadiazol-3-yl)-benzyl]-1,2,3,4-tetrahydro-quinazoline-6-carboxylic acid 4-methoxy-benzylamide,
{4-[6-(4-Methoxy-benzylcarbamoyl)-1-methyl-2,4-dioxo-1,4-dihydro-2H-quinazolin-3-yl]-phenyl}-acetic acid,
1-Methyl-3-(1-naphthalen-1-yl-ethyl)-2,4-dioxo-1,2,3,4-tetrahydro-quinazoline-6-carboxylic acid (1,3-benzodioxol-5-ylmethyl)-amide,
3-(3-Fluoro-benzyl)-1-methyl-2,4-dioxo-1,2,3,4-tetrahydro-quinazoline-6-carboxylic acid (pyridin-4-ylmethyl)-amide,
3-(3-Fluoro-benzyl)-1-methyl-2,4-dioxo-1,2,3,4-tetrahydro-quinazoline-6-carboxylic acid (2-methoxy-pyridin-4-ylmethyl)-amide,
3-(3-Fluoro-benzyl)-1-methyl-2,4-dioxo-1,2,3,4-tetrahydro-quinazoline-6-carboxylic acid (pyridin-3-ylmethyl)-amide,
3-(3-Fluoro-benzyl)-1-methyl-2,4-dioxo-1,2,3,4-tetrahydro-quinazoline-6-carboxylic acid 4-methoxy-benzylamide,
3-(3-Fluoro-benzyl)-1-methyl-2,4-dioxo-1,2,3,4-tetrahydro-quinazoline-6-carboxylic acid 3-methoxy-benzylamide,
1-Ethyl-3-(3-fluoro-benzyl)-2,4-dioxo-1,2,3,4-tetrahydro-quinazoline-6-carboxylic acid (pyridin-4-ylmethyl)-amide,
1-Ethyl-3-(3-fluoro-benzyl)-2,4-dioxo-1,2,3,4-tetrahydro-quinazoline-6-carboxylic acid (pyridin-3-ylmethyl)-amide,
3-(4-Bromo-benzyl)-1-methyl-2,4-dioxo-1,2,3,4-tetrahydro-quinazoline-6-carboxylic acid 4-methoxy-benzylamide,
3-(4-Bromo-benzyl)-1-methyl-2,4-dioxo-1,2,3,4-tetrahydro-quinazoline-6-carboxylic acid (2-methoxy-pyridin-4-ylmethyl)-amide,
3-(3,4-Difluoro-benzyl)-1-methyl-2,4-dioxo-1,2,3,4-tetrahydro-quinazoline-6-carboxylic acid (pyridin-3-ylmethyl)-amide,
3-(3,4-Difluoro-benzyl)-1-methyl-2,4-dioxo-1,2,3,4-tetrahydro-quinazoline-6-carboxylic acid (pyridin-4-ylmethyl)-amide,
3-(3,4-Difluoro-benzyl)-1-methyl-2,4-dioxo-1,2,3,4-tetrahydro-quinazoline-6-carboxylic acid 4-methoxy-benzylamide,
3-(3-chloro-4-fluoro-benzyl)-1-methyl-2,4-dioxo-1,2,3,4-tetrahydro-quinazoline-6-carboxylic acid (pyridin-4-ylmethyl)-amide,
3-(3-Chloro-4-fluoro-benzyl)-1-methyl-2,4-dioxo-1,2,3,4-tetrahydro-quinazoline-6-carboxylic acid 4-methoxy-benzylamide,
4-[6-(4-Methoxy-benzylcarbamoyl)-1-methyl-2,4-dioxo-1,4-dihydro-2H-quinazolin-3-ylmethyl]-benzoate(2-hydroxy-ethyl)-trimethyl-ammonium,
4-[6-(4-Methoxy-benzylcarbamoyl)-1-methyl-2,4-dioxo-1,4-dihydro-2H-quinazolin-3-ylmethyl]-benzoic acid hemicalcium,
4-[6-(4-Methoxy-benzylcarbamoyl)-1-methyl-2,4-dioxo-1,4-dihydro-2H-quinazolin-3-ylmethyl]-benzoic acid hemimagnesium,
3-(4-Chloro-benzyl)-1-methyl-2,4-dioxo-1,2,3,4-tetrahydro-quinazoline-6-carboxylic acid (pyridin-4-ylmethyl)-amide,
3-(4-Fluoro-benzyl)-1-methyl-2,4-dioxo-1,2,3,4-tetrahydro-quinazoline-6-carboxylic acid (pyridin-4-ylmethyl)-amide,
3-(4-Fluoro-benzyl)-1-methyl-2,4-dioxo-1,2,3,4-tetrahydro-quinazoline-6-carboxylic acid (pyridin-3-ylmethyl)-amide,
3-(4-Chloro-benzyl)-1-methyl-2,4-dioxo-1,2,3,4-tetrahydroquinazoline-6-carboxylic acid (pyridin-3-ylmethyl)-amide,
3-(4-Fluoro-benzyl)-1-methyl-2,4-dioxo-1,2,3,4-tetrahydroquinazoline-6-carboxylic acid 3-methoxy-benzylamide,
3-(4-Chloro-benzyl)-1-methyl-2,4-dioxo-1,2,3,4-tetrahydroquinazoline-6-carboxylic acid 3-methoxy-benzylamide,
3-(4-Fluoro-benzyl)-1-methyl-2,4-dioxo-1,2,3,4-tetrahydroquinazoline-6-carboxylic acid (2-methoxy-pyridin-4-ylmethyl)-amide,
3-(4-Chloro-benzyl)-1-methyl-2,4-dioxo-1,2,3,4-tetrahydroquinazoline-6-carboxylic acid (2-methoxy-pyridin-4-ylmethyl)-amide,
tert-Butyl 1-{4-[6-(4-methoxy-benzylcarbamoyl)-1-methyl-2,4-dioxo-1,4-dihydro-2H-quinazolin-3-ylmethyl]-phenyl}-cyclopropanecarboxylate,
1-{4-[6-(4-Methoxy-benzylcarbamoyl)-1-methyl-2,4-dioxo-1,4-dihydro-2H-quinazolin-3-ylmethyl]-phenyl}-cyclopropanecarboxylic acid,
3-Benzyl-6-benzylsulfanyl-1-methyl-1H-quinazoline-2,4-dione,
3-Benzyl-1-methyl-6-phenylmethanesulfinyl-H-quinazoline-2,4-dione,
3-Benzyl-1-methyl-6-phenylmethanesulfonyl-1H-quinazoline-2,4-dione,
4-[6-(4-methoxy-benzylcarbamoyl)-1-methyl-2,4-dioxo-1,4-dihydro-2H-quinazoline-3-ylmethyl]-benzoic acid tert-butoxycarbonylmethyl ester,
4-[6-(4-methoxy-benzylcarbamoyl)-1-methyl-2,4-dioxo-1,4-dihydro-2H-quinazoline-3-ylmethyl]-benzoic acid dimethylamino-dimethyl-propyl ester,
4-[6-(4-methoxy-benzylcarbamoyl)-1-methyl-2,4-dioxo-1,4-dihydro-2H-quinazoline-3-ylmethyl]-benzoic acid dimethylamino-methyl-propyl ester,
4-[6-(4-methoxy-benzylcarbamoyl)-1-methyl-2,4-dioxo-1,4-dihydro-2H-quinazoline-3-ylmethyl]-benzoic acid 2-dimethylamino-ethyl ester,
4-[6-(4-methoxy-benzylcarbamoyl)-1-methyl-2,4-dioxo-1,4-dihydro-2H-quinazoline-3-ylmethyl]-benzoic acid chloromethyl ester,
4-[6-(4-methoxy-benzylcarbamoyl)-1-methyl-2,4-dioxo-1,4-dihydro-2H-quinazoline-3-ylmethyl]-benzoic acid 2-tert-butoxycarbonylamino-3-methyl-1-butanoyloxymethyl ester,
4-[6-(4-methoxy-benzylcarbamoyl)-1-methyl-2,4-dioxo-1,4-dihydro-2H-quinazoline-3-ylmethyl]-benzoic acid 2-amino-3-methyl-butanoyloxymethyl ester hydrochloride,
4-[6-(4-methoxy-benzylcarbamoyl)-1-methyl-2,4-dioxo-1,4-dihydro-2H-quinazoline-3-ylmethyl]-benzoic acid 2-(2-tert-butoxycarbonylamino-3-methyl-butanoylamino)-3-methyl-butanoyloxymethyl ester,
and 4-[6-(4-methoxy-benzylcarbamoyl)-1-methyl-2,4-dioxo-1,4-dihydro-2H-quinazoline-3-ylmethyl]-benzoic acid 2-(2-amino-3-methyl-butanoylamino)-3-methyl-butanoyloxymethyl ester.

16- A compound of formula (I) according to claim 1 which is:
4-[6-(4-Methoxy-benzylcarbamoyl)-1-methyl-2,4-dioxo-1,4-dihydro-2H-pyrido[3,4-d]pyrimidin-3-ylmethyl]-benzoic acid,
3-Benzyl-1-methyl-2,4-dioxo-1,2,3,4-tetrahydro-pyrido[3,4-d]pyrimidine-6-carboxylic acid (1,3-benzodioxol-5-ylmethyl)-amide,
4-[6-(4-Fluoro-benzylcarbamoyl)-1-methyl-2,4-dioxo-1,4-dihydro-2H-quinazolin-3-ylmethyl]-benzoic acid,
1-Methyl-2,4-dioxo-3-[4-(5-oxo-4,5-dihydro-1,2,4-oxadiazol-3-yl)-benzyl]-1,2,3,4-tetrahydro-quinazoline-6-carboxylic acid 4-methoxy-benzylamide,
4-[6-(4-Methoxy-benzylcarbamoyl)-1-methyl-2,4-dioxo-1,4-dihydro-2H-quinazolin-3-ylmethyl]-benzoic acid hemicalcium salt,
Methyl 4-[6-(4-Methoxy-benzylcarbamoyl)-1-methyl-2,4-dioxo-1,4-dihydro-2H-pyrido[3,4-d]pyrimidin-3-ylmethyl]-benzoate,
4-[6-(3-Methoxy-benzylcarbamoyl)-1-methyl-2,4-dioxo-1,4-dihydro-2H quinazolin-3-ylmethyl]-benzoic acid,
1-Methyl-2,4-dioxo-3-[4-(2H-tetrazol-5-yl)-benzyl]-1,2,3,4-tetrahydro-quinazoline-6-carboxylic acid 4-methoxy-benzylamide,
Methyl 2-hydroxy-4-[6-(4-methoxy-benzylcarbamoyl)-1-methyl-2,4-dioxo-1,4-dihydro-2H-quinazolin-3-ylmethyl]-benzoate,
3-(4-Chloro-benzyl)-1-methyl-2,4-dioxo-1,2,3,4tetrahydroquinazoline-6-carboxylic acid 3-methoxy-benzylamide,
4-{6-[(1,3-Benzodioxol-5-ylmethyl)-carbamoyl]-1-methyl-2,4-dioxo-1,4-dihydro-2H-quinazolin-3-ylmethyl}-benzoic acid,
2-Hydroxy-4-[6-(4-methoxy-benzylcarbamoyl)-1-methyl-2,4-dioxo-1,4-dihydro-2H-quinazolin-3-ylmethyl]-benzoic acid,
Methyl 4-[6-(3-methoxy-benzylcarbamoyl)-1-methyl-2,4-dioxo-1,4-dihydro-2H-quinazolin-3-ylmethyl]-benzoate,
3-(3-Fluoro-benzyl)-1-methyl-2,4-dioxo-1,2,3,4-tetrahydro-quinazoline-6-carboxylic acid 3-methoxy-benzylamide,
4-Pyridylmethyl 3-benzyl-2,4-dioxo-1,2,3,4-tetrahydroquinazoline-6-carboxylate,
Methyl 4-{6-[(1,3-benzodioxol-5-ylmethyl)-carbamoyl]-1-methyl-2,4-dioxo-1,4-dihydro-2H-quinazolin-3-ylmethyl}-benzoate,
1-Methyl-3-[4-(5-methyl-1,2,4-oxadiazol-3-yl)-benzyl]-2,4-dioxo-1,2,3,4-tetrahydro-quinazoline-6-carboxylic acid 4-methoxy-benzylamide,
1-Methyl-3-[4-(3-methyl-1,2,4-oxadiazol-5-yl)-benzyl]-2,4-dioxo-1,2,3,4-tetrahydro-quinazoline-6-carboxylic acid 4-methoxy-benzylamide,
3-(3-Fluoro-benzyl)-1-methyl-2,4-dioxo-1,2,3,4-tetrahydro-quinazoline-6-carboxylic acid (2-methoxy-pyridin-4-ylmethyl)-amide,
4-[6-(4-Methoxy-benzylcarbamoyl)-1-methyl-2,4-dioxo-1,4-dihydro-2H]-quinazolin-3-ylmethyl]-benzoic acid,
1-{4-[6-(4-Methoxy-benzylcarbamoyl)-1-methyl-2,4-dioxo-1,4-dihydro-2H-quinazolin-3-ylmethyl]-phenyl}-cyclopropanecarboxylic acid,
4-Pyridylmethyl 3-benzyl-1-methyl-2,4-dioxo-1,2,3,4-tetrahydroquinazoline-6-carboxylate,
3-(4-Fluoro-benzyl)-1-methyl-2,4-dioxo-1,2,3,4-tetrahydroquinazoline-6-carboxylic acid 3-methoxy-benzylamide,
3-(3,4-Difluoro-benzyl)-1-methyl-2,4-dioxo-1,2,3,4-tetrahydro-quinazoline-6-carboxylic acid 4-methoxy-benzylamide,
3-(4-Dimethylcarbamoyl-benzyl)-1-methyl-2,4-dioxo-1,2,3,4-tetrahydroquinazoline-6-carboxylic acid 4-methoxy-benzylamide,
1-Methyl-3-[4-(2-methyl-2H-tetrazol-5-yl)-benzyl]-2,4-dioxo-1,2,3,4-tetrahydro-quinazoline-6-carboxylic acid 4-methoxy-benzylamide,
3-(4-Bromo-benzyl)-1-methyl-2,4-dioxo-1,2,3,4-tetrahydro-quinazoline-6-carboxylic acid (2-methoxy-pyridin-4-ylmethyl)-amide,
3-(3,4-Difluoro-benzyl)-1-methyl-2,4-dioxo-1,2,3,4-tetrahydro-quinazoline-6-carboxylic acid (pyridin-3-ylmethyl)-amide,
Benzo[1,3]dioxol-5-ylmethyl-3-benzyl-1-methyl-2,4-dioxo-1,2,3,4-tetrahydroquinazoline-6-carboxylate,
3-Benzyl-1-methyl-2,4-dioxo-1,2,3,4-tetrahydroquinazoline-6-carboxylic acid (benzo[1,3]dioxol-5-ylmethyl)amide,
1-Methyl-3-(4-methylcarbamoyl-benzyl)-2,4-dioxo-1,2,3,4-tetrahydroquinazoline-6-carboxylic acid 4-methoxy-benzylamide,
3-(3-Fluoro-benzyl)-1-methyl-2,4-dioxo-1,2,3,4-tetrahydro-quinazoline-6-carboxylic acid 4-methoxy-benzylamide,
4-[6-(4-Hydroxy-benzylcarbamoyl)-1-methyl-2,4-dioxo-1,4-dihydro-2H-quinazolin-3-ylmethyl]-benzoic acid,
Methyl 4-[6-(4-fluoro-benzylcarbamoyl)-1-methyl-2,4-dioxo-1,4-dihydro-2H-quinazolin-3-ylmethyl]-benzoate,
3-(4-Chlorobenzyl)-2,4-dioxo-1,2,3,4-tetrahydroquinazoline-6-carboxylic acid (benzo[1,3]dioxol-5-ylmethyl)amide,
1-Methyl-3-[4-(1-methyl-1H-tetrazol-5-yl)-benzyl]-2,4-dioxo-1,2,3,4-tetrahydro-quinazoline-6-carboxylic acid 4-methoxy-benzylamide,
3-(4-Methoxybenzyl)-1-methyl-2,4-dioxo-1,2,3,4-tetrahydroquinazoline-6-carboxylic acid 4-methoxybenzylamide,
4-Pyridylmethyl 3-(benzo[1,3]dioxol-5-ylmethyl)-2,4-dioxo-1,2,3,4-tetrahydroquinazoline-6-carboxylate,
Methyl 4-[6-(4-methoxy-benzylcarbamoyl)-1-methyl-2,4-dioxo-1,4-dihydro-2H-quinazolin-3-ylmethyl]-benzoate,
1-Methyl-2,4-dioxo-3-pyridin-4-ylmethyl-1,2,3,4-tetrahydro-quinazoline-carboxylic acid 4-methoxy-benzylamide,
3-(4-Amino-benzyl)-1-methyl-2,4-dioxo-1,2,3,4-tetrahydro-quinazoline-6-carboxylic acid 4-methoxy-benzylamide,
1-Methyl-3-(4-nitro-benzyl)-2,4-dioxo-1,2,3,4-tetrahydro-quinazoline-6-carboxylic acid 4-methoxy-benzylamide,
2-Methoxy-4-[6-(4-methoxy-benzylcarbamoyl)-1-methyl-2,4-dioxo-1,4-dihydro-2H-quinazolin-3-ylmethyl]-benzoic acid,
1-Methyl-3-(4-methylsulfamoyl-benzyl)-2,4-dioxo-1,2,3,4-tetrahydroquinazoline-6-carboxylic acid 4-methoxy-benzylamide,
1-Methyl-2,4-dioxo-3-(4-sulfamoyl-benzyl)-1,2,3,4-tetrahydro-quinazoline-6-carboxylic acid 4-methoxy-benzylamide,
3-(4-Fluoro-benzyl)-1-methyl-2,4-dioxo-1,2,3,4-tetrahydro-quinazoline-6-carboxylic acid 4-methoxy-benzylamide,
3-(4-Fluoro-benzyl)-1-methyl-2,4-dioxo-1,2,3,4-tetrahydro-quinazoline-6-carboxylic acid (pyridin-4-ylmethyl)-amide,
3-(4-Methoxy-benzyl)-1-methyl-2,4-dioxo-1,2,3,4-tetrahydro-quinazoline-6-carboxylic acid (pyridin-4-ylmethyl)-amide,
2-Methyl-4-[6-(4-methoxy-benzylcarbamoyl)-1-methyl-2,4-dioxo-1,4-dihydro-2H-quinazolin-3-ylmethyl]-benzoic acid,
3-(4-Cyano-benzyl)-1-methyl-2,4-dioxo-1,2,3,4-tetrahydro-quinazoline-6-carboxylic acid 4-methoxy-benzylamide,
4-{1-Methyl-2,4-dioxo-6-[(pyridin-4-ylmethyl)-carbamoyl]-1,4-dihydro-2H-quinazolin-3-ylmethyl}-benzoic acid,
3-(3-fluoro-4-methoxy-benzyl)-1-methyl-2,4-dioxo-1,2,3,4-tetrahydroquinazoline-6-carboxylic acid 4-methoxy benzylamine,
4-[1-Ethyl-6-(4-methoxy-benzylcarbamoyl)-2,4-dioxo-1,4-dihydro-2H-quinazolin-3-ylmethyl]-benzoic acid,
3-(Benzo[1,3]dioxol-5-ylmethyl)-2,4-dioxo-1,2,3,4-tetrahydroquinazoline-6-carboxylic acid (benzo[1,3]dioxol-5-ylmethyl)amide,
3-(2′-Cyano-biphenyl-4-ylmethyl)-1-methyl-2,4-dioxo-1,2,3,4-tetrahydroquinazoline-6-carboxylic acid 4-methoxy-benzylamide,
4-[1-Methyl-6-(4-methylsulfanyl-benzylcarbamoyl)-2,4-dioxo-1,4-dihydro-2H-quinazolin-3-ylmethyl]-benzoic acid,
4-{6-[(Benzofurazan-5-ylmethyl)-carbamoyl]-1-methyl-2,4-dioxo-1,4-dihydro-2H-quinazolin-3-ylmethyl}-benzoic acid,
Methyl 2-methyl-4-[6-(4-methoxy-benzylcarbamoyl)-1-methyl-2,4-dioxo-1,4-dihydro-2H-quinazolin-3-ylmethyl]-benzoate,
3-(4-Acetylamino-benzyl)-1-methyl-2,4-dioxo-1,2,3,4-tetrahydro-quinazoline-6-carboxylic acid 4-methoxy-benzylamide,
3-(Benzo[1,3]dioxol-5-ylmethyl)-1-methyl-2,4-dioxo-1,2,3,4-tetrahydroquinazoline-6-carboxylic acid (benzo[1,3]dioxol-5-ylmethyl)amide,
3-(4-Dimethylcarbamoylmethyl-benzyl)-1-methyl-2,4-dioxo-1,2,3,4-tetrahydroquinazoline-6-carboxylic acid 4-methoxy-benzylamide,
Benzo[1,3]dioxol-5-ylmethyl 3-benzyl-2,4-dioxo-1,2,3,4-tetrahydroquinazoline-6-carboxylate,
{4-[6-(4-Methoxy-benzylcarbamoyl)-1-methyl-2,4-dioxo-1,4-dihydro-2H-quinazolin-3-ylmethyl]-phenyl}-acetic acid,
(4-{1-Methyl-2,4-dioxo-6-[(pyridin-4-ylmethyl)-carbamoyl]-1,4-dihydro-2H-quinazolin-3-ylmethyl}-phenyl)-acetic acid,
3-Benzyl-2,4-dioxo-1,2,3,4-tetrahydroquinazoline-6-carboxylic acid 4-methoxybenzylamide,
Methyl {4-[6-(4-methoxy-benzylcarbamoyl)-1-methyl-2,4-dioxo-1,4-dihydro-2H-quinazolin-3-ylmethyl]-phenyl}-acetate,
3-(3-Fluoro-benzyl)-1-methyl-2,4-dioxo-1,2,3,4-tetrahydro-quinazoline-6-carboxylic acid (pyridin-4-ylmethyl)-amide,
2,4-Dioxo-3-(thien-2-ylmethyl)-1,2,3,4-tetrahydroquinazoline-6-carboxylic acid (benzo[1,3]dioxol-5-ylmethyl)amide,
1-Methyl-3-(4-methylsulfamoyl-benzyl)-2,4-dioxo-1,2,3,4-tetrahydroquinazoline-6-carboxylic acid 4-methoxy-benzylamide,
Methyl 4-{1-methyl-2,4-dioxo-6-[(pyridin-4-ylmethyl)-carbamoyl]-1,4-dihydro-2H-quinazolin-3-ylmethyl}-benzoate,
2-Fluoro-4-[6-(4-methoxy-benzylcarbamoyl)-1-methyl-2,4-dioxo-1,4-dihydro-2H-quinazolin-3-ylmethyl]-benzoic acid,
3-(4-Cyano-benzyl)-1-methyl-2,4-dioxo-1,2,3,4-tetrahydro-pyrido[3,4-d]pyrimidine-6-carboxylic acid 4-methoxy-benzylamide,
4-[6-(3-Methoxy-benzylcarbamoyl)-1-methyl-2,4-dioxo-1,4-dihydro-2H-pyrido[3,4-d]pyrimidin-3-ylmethyl]-benzoic acid,
4-[6-(4-Methoxy-benzylcarbamoyl)-1-methyl-2,4-dioxo-1,4-dihydro-2H-quinazolin-3-ylmethyl]-benzoic acid hemimagnesium salt,
4-[6-(4-Methoxy-benzylcarbamoyl)-1-methyl-2,4-dioxo-1,4-dihydro-2H-pyrido[2,3-d]pyrimidin-3-ylmethyl]-benzoic acid,
3-[4-(N-methylsulfonylamino)-benzyl]-1-methyl-2,4-dioxo-1,2,3,4-tetrahydroquinazoline-6-carboxylic acid 4-methoxy-benzylamide,
Ethyl 2-Fluoro-4-[6-(4-methoxy-benzylcarbamoyl)-1-methyl-2,4-dioxo-1,4-dihydro-2H-quinazolin-3-ylmethyl]-benzoate,
3-(4-Dimethylsulfamoyl-benzyl)-1-methyl-2,4-dioxo-1,2,3,4-tetrahydroquinazoline-6-carboxylic acid 4-methoxy-benzylamide,
and 3-(4-Methoxybenzyl)-1-methyl-2,4-dioxo-1,2,3,4-tetrahydroquinazoline-6-carboxylic acid (benzo[1,3]dioxol-5-ylmethyl)amide.

17- Intermediate compound of formula (III): 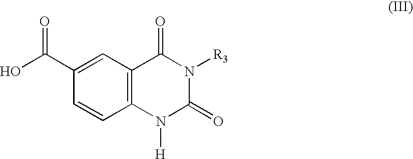
in which R₃ is as defined in the compound of formula (I).

18- Intermediate compound of formula (IV): 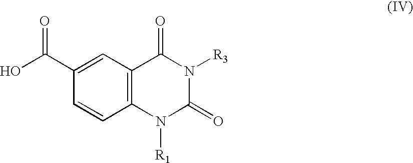
in which R₁ et R₃ are as defined in the compound of formula (I).

19- Process for manufacturing a compound of general formula (I): 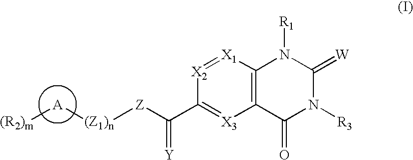
in which R₂, R₃, Z₁, A, n and m are as defined in claim 1, R₁ is H, X₁, X₂ and X₃ are CH, Y is O, Z is N—R₇ and W is O,
the said process being characterized in that it comprises the reaction of a compound of formula (II): 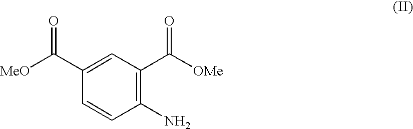
with pyridine and the compound of general formula (V):
O═C═N—R₃ (V)
in which R₃ is as defined in claim 1,
to give the compound of general formula (VI): 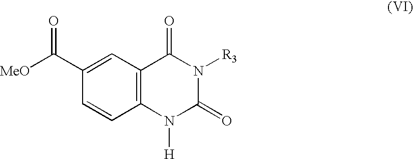
in which R₃ is as defined hereinbefore,
followed by reacting the compound of general formula (VI) in the presence of LiOH to give the compound of general formula (III) in which R₃ is as defined hereinbefore: 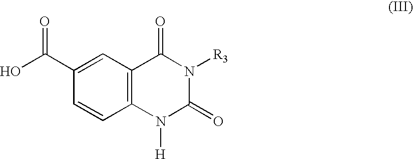
the said compound of general formula (III) is reacted, in the presence of an acid activator such as TOTU, with the compound of general formula (VII): 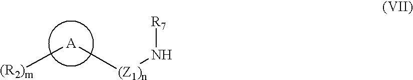
in which R₇ is selected from hydrogen, (C₁-C₆)alkyl, aryl(C₁-C₆)alkyl, cycloalkyl, aryl and heteroaryl, and A, R₂, Z₁, m and n are as defined in claim 1,
to give the compound of general formula (I) in which R₁ represents hydrogen, X₁, X₂ and X₃ are CH, Y is O, Z is N—R₇, W is O, and A, R₂, R₃, Z₁, m and n are as defined hereinbefore.

20- Process for manufacturing a compound of general formula (I): 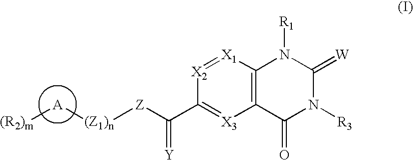
in which R₁, R₂, R₃, A, Z₁, m and n are as defined in claim 1, X₁, X₂ and X₃ are CH, W is O, Y is O and Z is N—R₇,
the said process being characterized in that a compound of general formula (VI): 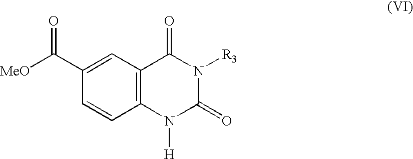
in which R₃ is as defined in claim 1,
is reacted, in the presence of a base, with compound (VIII) of general formula X—R₁, in which R₁ is as defined in claim 1 and X is a leaving group such as halogen, to give the compound of general formula (IX): 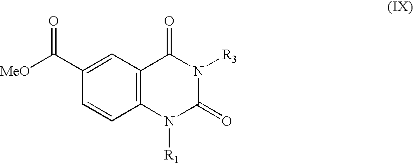
in which R₁ and R₃ are as defined hereinbefore,
said compound of general formula (IX) is reacted in the presence of LiOH to give the compound of general formula (IV): 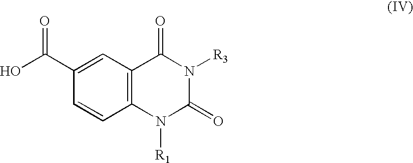
in which R₁ and R₃ are as defined hereinbefore,
said compound of general formula (IV) is reacted, in the presence of an acid activator such as TOTU, with the compound of general formula (VII): 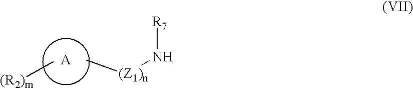
in which R₇ is selected from hydrogen, (C₁-C₆)alkyl, aryl(C₁-C₆)alkyl, cycloalkyl, aryl and heteroaryl, and A, R₂, Z₁, m and n are as defined in the summary of the invention,
to give the compound of general formula (I): 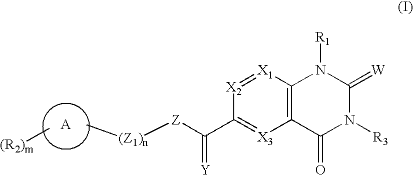
in which R₁, R₂, R₃, A, Z₁, m and n are as defined in the claim 1, X₁, X₂ and X₃ are CH, W is O, Y is O and Z is N—R₇.

21- Process for manufacturing the compound of general formula (I) in which R₁, R₂, R₃, W, X₁, X₂, X₃, A, Z₁, m and n are as defined in claim 1, Y is 0 and Z is N—R₇, characterized in that a compound of general formula (I): 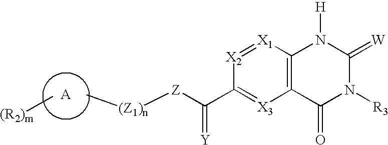
in which R₁ is H, and R₂, R₃, W, Y, Z, X₁, X₂, X₃, A, Z₁, m and n are as defined hereinbefore,
is reacted, in the presence of a base, with a compound (VIII) of general formula X—R₁, in which R₁ is as defined in claim 1 and X is a leaving group such as halogen, to give the compound of general formula (I) in which R₁ is as defined in claim 1.

22- Process for manufacturing a compound of general formula (I) in which X₁, X₂ and X₃ are CH, W is O, Y is O, Z is N—R₇, R₃ is H, and R₁, R₂, A, Z₁, m and n are as defined in claim 1 characterized in that a compound of general formula (XI): 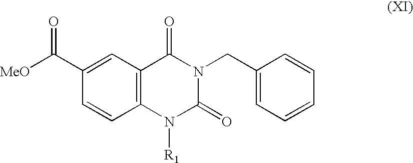
in which R₁ is as defined hereinbefore,
is reacted with AlCl₃ in a solvent such as benzene, to give the compound of general formula (XII): 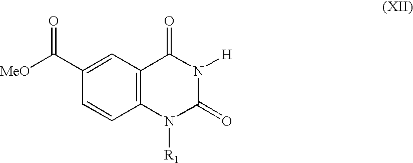
in which R₁ is as defined hereinbefore,
said compound of general formula (XII) is reacted in the presence of LiOH and a mixture of dioxane/H₂O to give the compound of general formula (XIII): 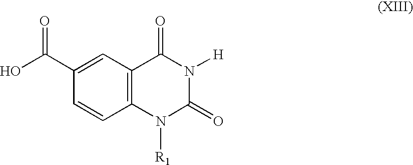
in which R₁ is as defined hereinbefore,
said compound of general formula (XIII) is reacted, in the presence of an acid activator such as TOTU with the compound of general formula (VII): 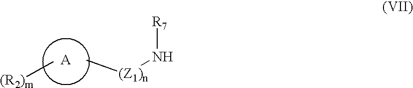
in which R₇ is selected from hydrogen, (C₁-C₆)alkyl, aryl(C₁-C₆)alkyl, cycloalkyl, aryl and heteroaryl, and A, R₂, Z₁, m and n are as defined in claim 1, to give the compound of general formula (XIV): 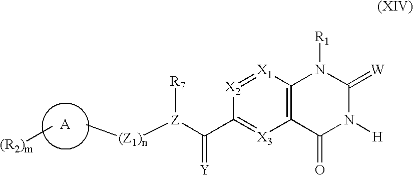
in which X₁, X₂ and X₃ are CH, W is O, Y is O, and R₇, A, R₂, R₁, Z₁, m and n are as defined hereinbefore.

23- The process for manufacturing a compound of general formula (I) characterized in that it comprises a step in which the compound of general formula (XIV): 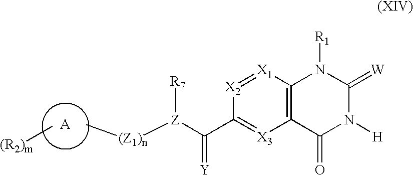
in which X₁, X₂ and X₃ are CH, W is O, Y is O, and R₇, A, R₂, R₁, Z₁, m and n are as defined in claim 1,
is reacted with compound (XV) of general formula X—R₃, in which R₃ is as defined in claim 1 and X is a leaving group such as halogen,
to give the compound of general formula (I): 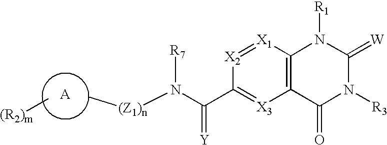
in which X₁, X₂ and X₃ are CH, W is O, Y is O, and R₇, A, R₂, R₃, Rₗ, Z₁, m and n are as defined in claim 1,

24- Process for manufacturing a compound of general formula (I) in which X₁, X₂ and X₃ are CH, W is O, Y is O and Z is O, characterized in that a compound of general formula (III): 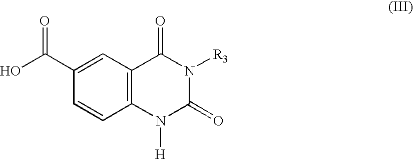
in which R₃ is as defined in claim 1,
is reacted with a compound of general formula (XVI): 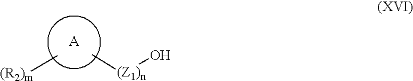
in which A, R₂, Z₁, m and n are as defined in claim 1,
to give a compound of general formula (XVII): 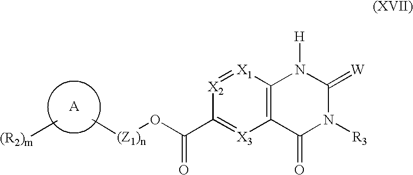
in which A, R₂, R₃, Z₁, m and n are as defined hereinbefore, X₁, X₂ and X₃ are CH, and W is O.

25- Process for manufacturing a compound of general formula (I), the said process is characterized in that the compound of formula (XVII): 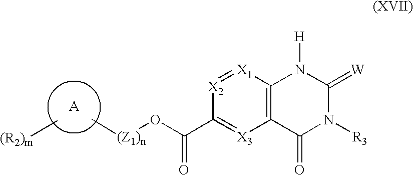
in which A, R₂, R₃, Z₁, m and n are as defined in claim 1, X₁, X₂ and X₃ are CH, and W is O,
is reacted, in the presence of a base, with compound (VIII) of general formula X—R₁, in which R₁ is as defined in claim 1 and X is a leaving group such as halogen, to give the compound of general formula (I): 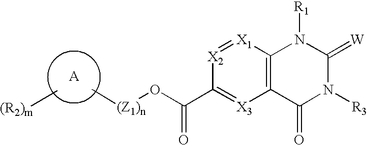
in which A, R₁, R₂, R₃, Z₁, m and n are as defined in hereinbefore, X₁, X₂ and X₃ are CH, and W is O.

26- Process for manufacturing a compound of general formula (I) in which X₂ and X₃ are CH, X₁ is N, Z is O, Y is O, R₁ is H, W is O, and A, R₂, R₃, Zₗ, m and n are as defined in claim 1,
characterized in that the said process comprises a step in which a compound of general formula (XIX): 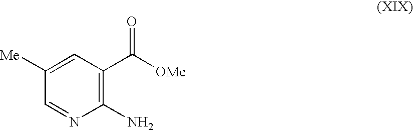
is reacted with pyridine and a compound (V) of general formula O═C═N—R₃ in which R₃ is as defined in claim 1,
to give a compound of general formula (XX): 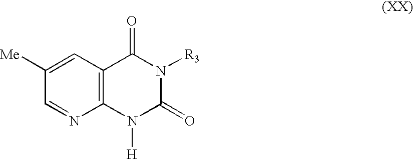
in which R₃ is as defined hereinbefore,
said compound of general formula (XX) is reacted in the presence of KMnO₄ to give the compound of general formula (XXI): 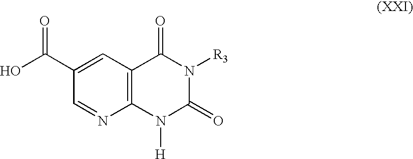
in which R₃ is as defined hereinbefore,
said compound of general formula (XXI) is reacted in the presence of SOCl₂ and optionally of a solvant to give the compound of general formula (XXII): 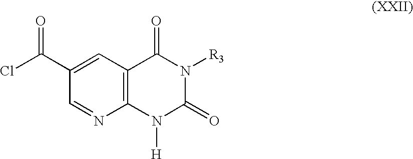
in which R₃ is as defined hereinbefore,
said compound of formula (XXII) is reacted with the compound of general formula (XVI): 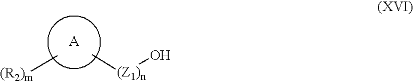
in which A, R₂, Z₁, n and m are as defined in claim 1,
to give the compound of general formula (XXIV): 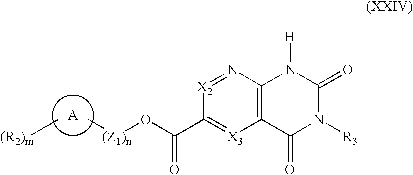
in which X₂ and X₃ are CH and A, n, m, Z₁, R₂ and R₃ are as defined hereinbefore.

27- A process for manufacturing a compound of general formula (I) in which X₂ and X₃ are CH, X₁ is N, Z is —NR₇ in which R₇ is as defined in the compound of formula (I), W is O, and Y is O, characterized in that the said process comprises a step in which a compound of general (XXV): 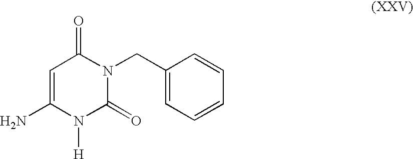
is reacted in a first step with N,N′-dimethylformamide dimethyl acetal under reflux of DMF, and in a second step with N-iodosuccinimide, to give a compound of formula (XXVI): 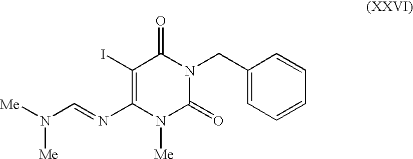
followed by reacting th compound of formula (XXVI) whith ethyl acrylate in the presence of palladium diacetate, CuI and a base, to give the compound of general formula (XXVII): 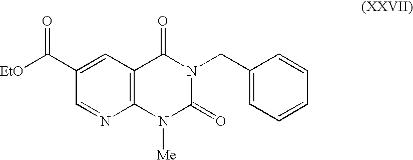
followed by reacting the compound of formula (XXVII) in the presence of LiOH to give the compound of general formula (XXVIII): 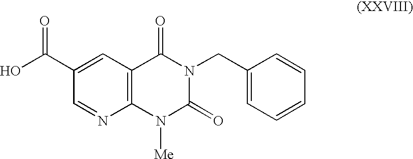
the said compound of formula (XXVIII):
either is reacted, in the presence of an acid activator such as TOTU, with the compound of formula (VII): 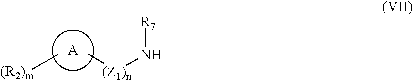
in which R₇ is selected from hydrogen, (C₁-C₆)alkyl, aryl(C₁-C₆)alkyl, cycloalkyl, aryl and heteroaryl, and A, R₂, Z₁, m and n are as defined in the summary of the invention,
to give the compound of general formula (XXIX): 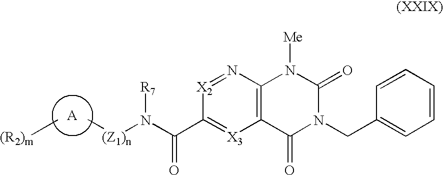
in which A, R₂, R₇, Z₁, m and n are as defined hereinbefore, and X₂ and X₃ represents each —CH group,
or is reacted in a first step with AlCl₃ in the presence of benzene, and in a second step in the presence of an acid activator such as TOTU, with the compound of formula (VII): 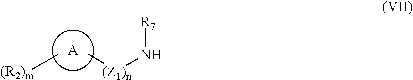
in which R₇ is selected from hydrogen, (C₁-C₆)alkyl, aryl(C₁-C₆)alkyl, cycloalkyl, aryl and heteroaryl, and A, R₂, Z₁, m and n are as defined in the summary of the invention, to give the compound of general formula (XXX): 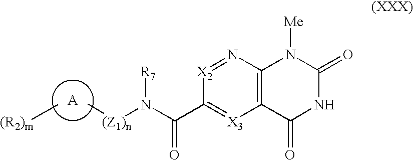
in which A, R₂, R₇, Z₁, m and n are as defined hereinbefore, and X₂ and X₃ represents each —CH group,
followed by reacting the compound of formula (XXX) with a compound of formula R₃—X in which R₃ is as defined in the compound of general formula (I), in the presence of a base,
to give the compound of formula (XXXI):

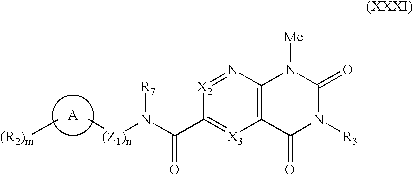

28- A process for manufacturing a compound of general formula (I) in which X₁ and X₃ are CH, X₂ is N, Z is —NR₇ in which R₇ is as defined in the compound of formula (I), W is O, and Y is O, characterized in that the said process comprises a step in which a compound of general (XXXII): 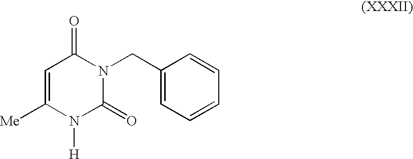
is reacted in a first step with selenium dioxide in the presence of acetic acid, in a second step with dimethylhydrazine, and in a third step with N,N′-dimethylformamide dimethylacetal under reflux of DMF, to give a compound of formula (XXXIII): 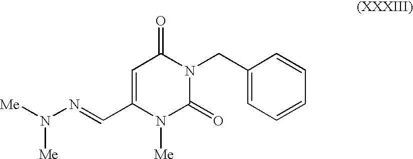
followed by reacting th compound of formula (XXXIII) whith methyl acrylate in the presence of palladium diacetate, to give the compound of general formula (XXXIV): 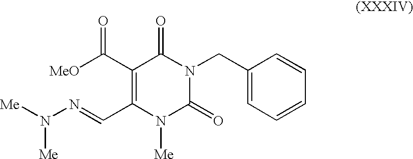
followed by reacting the compound of formula (XXXIV) whith chlorobenzene and acetic acid to give the compound of formula (XXXV): 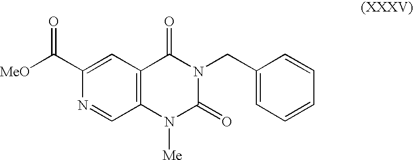
followed by reacting the compound of formula (XXXV) in the presence of a base to give the compound of general formula (XXXVI): 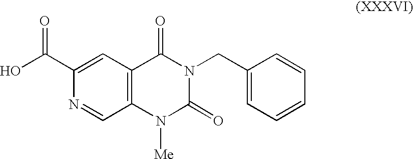
the said compound of formula (XXXVI):
either is reacted, in the presence of an acid activator such as TOTU, with the compound of formula (VII): 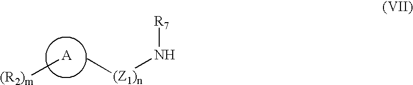
in which R₇ is selected from hydrogen, (C₁-C₆)alkyl, aryl(C₁-C₆)alkyl, cycloalkyl, aryl and heteroaryl, and A, R₂, Z₁, m and n are as defined in the summary of the invention, to give the compound of general formula (XXXVII): 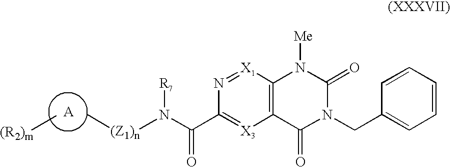
in which A, R₂, R₇, Z₁, m and n are as defined hereinbefore, and X₁ and X₃ represents each —CH group,
or is reacted in a first step with AlCl₃ in the presence of benzene, and in a second step in the presence of an acid activator such as TOTU, with the compound of formula (VII): 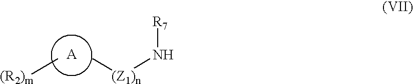
in which R₇ is selected from hydrogen, (C₁-C₆)alkyl, aryl(C₁-C₆)alkyl, cycloalkyl, aryl and heteroaryl, and A, R₂, Z₁, m and n are as defined in the summary of the invention,
to give the compound of general formula (XXXVIII): 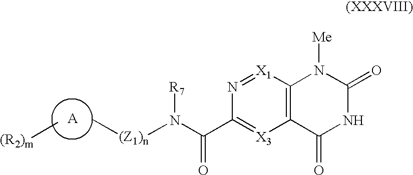
in which A, R₂, R₇, Z₁, m and n are as defined hereinbefore, and X₁ and X₃ represents each —CH group,
followed by reacting the compound of formula (XXXVIII) with a compound of formula R₃—X in which R₃ is as defined in the compound of general formula (I), in the presence of a base, to give the compound of formula (XXXIX):

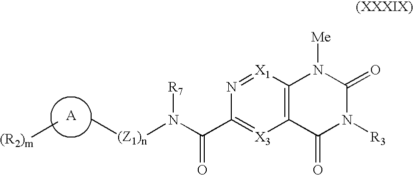

29- Pharmaceutical composition comprising a compound according to any one of claims 1 to 15 and a pharmaceutically acceptable excipient.

30- Use of a compound according to any one of claims 1 to 16, for the preparation of a medicinal product intended for treating a disease or complaint involving therapy by inhibition of type-13 matrix metalloprotease.

31- Use according to claim 30, characterized in that the disease is arthritis, rheumatoid arthritis, osteoarthritis, osteoporosis, periodontal diseases, inflammatory bowel disease, psoriasis, multiple sclerosis, cardiac insufficiency, atherosclerosis, asthma, chronic obstructive pulmonary disease (COPD), age-related macular degeneration (ARMD) and cancers.

32- Use according to claim 31, characterized in that the disease is arthritis.

33- Use according to claim 31, characterized in that the disease is osteoarthritis.

34- Use according to claim 31, characterized in that the disease is rheumatoid arthritis.

35- A method for treating a disease or complaint involving a therapy by inhibition of MMP-13, the said method comprising the administration of an effective amount of a compound according to any one of claims 1 to 16 to a patient.

36- A method for treating according to claim 35 charactherized in that the disease or the complaint are selected from arthritis, rheumatoid arthritis, osteoarthritis, osteoporosis, periodontal diseases, inflammatory bowel disease, psoriasis, multiple sclerosis, cardiac insufficiency, atherosclerosis, asthma, chronic obstructive pulmonary disease (COPD), age-related macular degeneration (ARMD) and cancers.

37- A method for treating according to claim 35 charactherized in that the disease is arthritis.

38- A method for treating according to claim 35 charactherized in that the disease is osteoarthritis.

39- A method for treating according to claim 40 charactherized in that the disease is rheumatoid arthritis.
